(19) United States
(12) Patent Application Publication
(10) Pub. No.: US 2023/0414764 A1
(43) Pub. Date: Dec. 28, 2023

(54) Template Assembly by Proximity-Enhanced Reactivity via Metabolic Labeling

(71) Applicant: The General Hospital Corporation, Boston, MA (US)

(72) Inventors: James T. Kurnick, Quincy, MA (US); Ian Seymour Dunn, Beverly, MA (US); Matthew M. Lawler, Madison, WI (US)

(21) Appl. No.: 18/035,912

(22) PCT Filed: Nov. 5, 2021
(86) PCT No.: PCT/US2021/058314

§ 371 Date: May 8, 2023

Related U.S. Application Data
(60) Provisional application No. 63/111,401, filed on Nov. 9, 2020.

(51) Int. Cl. A61K 47/54

(52) U.S. Cl. CPC A61K 47/549

(57) ABSTRACT

The present disclosure provides methods of metabolically labeling cells to provide a substrate for assembly of desired molecules, and to bifunctional compounds used in such methods.

### CROSS-REFERENCE TO RELATED APPLICATIONS

This application claims priority to U.S. Provisional Patent Application No. 63/111,401, filed Nov. 9, 2020. The contents of this application are incorporated herein by reference in its entirety.

### FIELD OF THE INVENTION

The present disclosure is directed, in part, to methods of metabolically labeling cells to provide a substrate for assembly of desired molecules, and to compounds used in such methods.

### BACKGROUND

A goal of drug development is delivering bio-therapeutic interventions to pathogenic cells, including virus infected cells, neoplastic cells, cells producing an autoimmune response, and other dysregulated or dysfunctional cells. Examples of bio-therapeutic interventions capable of combating pathogenic cells include toxins, pro-apoptotic agents, and immunotherapy approaches that re-direct immune cells to eliminate pathogenic cells. Unfortunately, developing these agents is extremely difficult because of the high risk of toxicity to adjacent normal cells or the overall health of the patient.

Methods that have emerged to allow delivery of interventions to pathogenic cells while mitigating toxicity to normal cells include targeting of therapeutics by directing them against molecular markers specific for pathogenic cells. Targeted therapeutics have shown extraordinary clinical results in restricted cases, but are currently limited in their applicability due to a lack of accessible markers for targeted therapy. It is extremely difficult, and often impossible, to discover protein markers for many pathogenic cell types.

More recently, therapies targeted to nucleic acid molecules specific to pathogenic cells have been developed. Existing nucleic acid-targeted therapies, such as siRNA, are able to down-modulate expression of potentially dangerous genes, but do not deliver potent cytotoxic or cytostatic interventions and, thus, are not particularly efficient at eliminating the dangerous cells themselves. Hence, there exists a need to combat the poor efficacy and/or severe side effects of existing bio-therapeutic interventions.

### SUMMARY

The present disclosure provides methods of labeling a cell with a substrate for a templated assembly reaction, the method comprising the steps: a) contacting the cell with an azide-modified sugar; and b) contacting the cell with the substrate for the templated assembly reaction, wherein the substrate comprises: i) a nucleic acid template; and ii) an azide reactive molecule linked to the nucleic acid template at the 5′- or 3′-end of the nucleic acid template, wherein the azide reactive molecule is chemically reactable with the azide of the azide-modified sugar.

The present disclosure also provides methods of labeling a cell with a substrate for a templated assembly reaction, the method comprising the steps: a) contacting the cell with an azide-modified sugar; and b) contacting the cell with a nucleic acid template, wherein the nucleic acid template comprises: i) a first hybridization region and a second hybridization region separated by a loop region, wherein the first hybridization region is complementary to the second hybridization region; and ii) a azide reactive molecule at the 3′-end of the nucleic acid template and at the 5′-end of the nucleic acid template, wherein both azide reactive molecules are chemically reactable with an azide group.

The present disclosure also provides methods of metabolically labeling the surface of a specific target cell, the method comprising the steps: a) contacting the cell with a nucleic acid molecule, wherein the nucleic acid molecule comprises: i) a short terminal RNA segment comprising a terminal azide-modified sugar; and ii) a longer modified RNA segment linked to the short terminal RNA segment, wherein the terminal end of the longer modified RNA segment is complementary to the short terminal RNA segment; wherein the longer modified RNA segment is complementary to a specific transcript target within the specific targeted cell; and wherein the longer modified RNA segment is modified to be nuclease resistant.

The present disclosure also provides methods of metabolically labeling the surface of a cell, the method comprising the steps: a) contacting the cell with a nucleic acid molecule, wherein the nucleic acid molecule comprises: i) a short terminal RNA segment comprising a terminal azide-modified sugar; ii) a longer modified RNA segment, wherein a first terminal end of the longer modified RNA segment is complementary to the short terminal RNA segment; and iii) a short linker nucleic acid having a first terminal end linked to the terminal end of the short terminal RNA segment that does not comprise the azide-modified sugar wherein the short linker nucleic acid is complementary to a second terminal end of the longer modified RNA segment that is not complementary to the short terminal RNA segment; wherein the longer modified RNA segment is complementary to a specific transcript target; and wherein the longer modified RNA segment is modified to be nuclease resistant.

In some embodiments, these methods further comprise performing a Template Assembly by Proximity-Enhanced Reactivity (TAPER) reaction using the nucleic acid template as a target nucleic acid sequence for the TAPER reaction. In some embodiments, the TAPER reaction comprises contacting the cell with a first haplomer and a second haplomer, wherein: a) the first haplomer comprises: a first polynucleotide that is complementary to a first region of the nucleic acid template; a first effector partial moiety, wherein the first effector partial moiety is linked to the first polynucleotide; and a first selectively-reactive moiety, wherein the first selectively-reactive moiety is linked to the first effector partial moiety; b) the second haplomer comprises: a second polynucleotide that is complementary to a second region of the nucleic acid template; a second effector partial moiety, wherein the second effector partial moiety is linked to the second polynucleotide; and a second selectively-reactive moiety, wherein the second selectively-reactive moiety is linked to the second effector partial moiety; wherein: the first selectively-reactive moiety and the second selectively-reactive moiety chemically react with each other when in sufficient proximity; the first region of the nucleic acid template is in sufficient proximity to the second region of the nucleic acid template to allow the first selectively-reactive moiety and the second selectively-reactive moiety to chemically react with each other; and the first effector partial moiety and the second effector partial moiety form an active effector agent when in sufficient proximity.

In some embodiments, these methods further comprise performing a Ligand Directed TAPER (LD-TAPER) reaction using the nucleic acid template as a target nucleic acid sequence for the LD-TAPER reaction. In some embodiments, the LD-TAPER reaction comprises: contacting the nucleic acid template with a first haplomer-ligand complex, wherein the first haplomer-ligand complex comprises a first haplomer, wherein the first haplomer comprises a first polynucleotide, and a first small molecule ligand linked to the 5′ or 3′ terminus of the first haplomer, wherein the first small molecule ligand comprises a first small molecule ligand partner binding site; contacting the nucleic acid template with a second haplomer-ligand complex, wherein the second haplomer-ligand complex comprises a second haplomer, wherein the second haplomer comprises a second polynucleotide, and a second small molecule ligand linked to the 5′ or 3′ terminus of the second haplomer, wherein the second small molecule ligand comprises a second small molecule ligand partner binding site; contacting the first haplomer-ligand complex with a first fusion protein, wherein the first fusion protein comprises a first fragment of a protein of interest fused to a first ligand binding domain for a small molecule ligand; contacting the second haplomer-ligand complex with a second fusion protein, wherein the second fusion protein comprises a second fragment of the protein of interest fused to a second ligand binding domain for a small molecule ligand; wherein the first ligand of the first haplomer-ligand complex is linked to the 5′ terminus of the first polynucleotide of the first haplomer-ligand complex; wherein the second ligand of the second haplomer-ligand complex is linked to the 3′ terminus of the second polynucleotide of the second haplomer-ligand complex; wherein the first polynucleotide of the first haplomer-ligand complex is substantially complementary to the nucleic acid template; wherein the second polynucleotide of the second haplomer-ligand complex is substantially complementary to the nucleic acid template at a site in spatial proximity to the first polynucleotide of the first haplomer-ligand complex; wherein the first small molecule ligand of the first haplomer-ligand complex and the first ligand binding domain of the first fusion protein can interact; and wherein the second small molecule ligand of the second haplomer-ligand complex and the second ligand binding domain of the second fusion protein can interact; thereby resulting in the folding or dimerization of the first fragment of the protein of interest of the first fusion protein with the second fragment of the protein of interest of the second fusion protein.

In some embodiments, these methods further comprise performing a Split Protein TAPER (SP-TAPER) reaction using the nucleic acid template as a target nucleic acid sequence for the TAPER reaction. In some embodiments, the SP-TAPER reaction comprises: contacting the cell with a first haplomer comprising a first polynucleotide linked to the C-terminus of an N-terminal protein fragment; and contacting the cell with a second haplomer comprising a second polynucleotide linked to the N-terminus of a C-terminal protein fragment; wherein: the polynucleotide of one of the first or second haplomers is linked at its 5′ terminus to the protein fragment, and the other of the first and second haplomers is linked at its 3′ terminus to the protein fragment; the N-terminal protein fragment and the C-terminal protein fragment are derived from a single active effector agent; and wherein: the first polyriuclemide of the lira haplomer is substantially complementary to the nucleic acid template, and the second polynucleotide of the second haplomer is substantially complementary to the nucleic acid template at a site in spatial proximity to the first polynucleotide of the first haplomer; or the first polynucleotide of the first haplomer is substantially complementary to a portion of the nucleic acid template 5′ adjacent to a stem-loop structure, and the second polynucleotide of the second haplomer is substantially complementary to a portion of the nucleic acid template 3′ adjacent to the stem-loop structure; or the first polynucleotide of the first haplomer is substantially complementary to a 5′ portion of a loop of a stem-loop structure of the nucleic acid template, and the second polynucleotide of the second haplomer is substantially complementary to a 3′ portion of the loop of the stem-loop structure of the nucleic acid template; thereby resulting in the assembly of the protein from the N-terminal protein fragment and the C-terminal protein fragment.

In some embodiments, these methods further comprise performing a Locked TAPER reaction using the nucleic acid template as a target nucleic acid sequence for the TAPER reaction. In some embodiments, the Locked TAPER reaction comprises: contacting the nucleic acid template with a first haplomer, wherein the first haplomer comprises: a) a first polynucleotide comprising: i) a first stem portion comprising from about 10 to about 20 nucleotide bases; ii) an anti-target loop portion comprising from about 16 to about 40 nucleotide bases and having a first end to which the first stem portion is linked, wherein the anti-target loop portion is substantially complementary to the nucleic acid template; and iii) a second stem portion comprising from about 10 to about 20 nucleotide bases linked to a second end of the anti-target loop portion, wherein the first stem portion is substantially complementary to the second stem portion; and b) a first effector partial moiety linked to either the first stem portion or the second stem portion; wherein the Tₘ of the anti-target loop portion:nucleic acid template is greater than the Tₘ of the first stem portion:second stem portion; and contacting the first haplomer with a second haplomer, wherein the second haplomer comprises: a) a second polynucleotide comprising a nucleotide portion that is substantially complementary to the stem portion of the first polynucleotide that is linked to the first effector partial moiety; and b) a second effector partial moiety linked to the second polynucleotide, wherein the second effector partial moiety can chemically interact with the first effector partial moiety of the first haplomer; wherein the Tₘ of the second polynucleotide:first or second stem portion linked to the first effector partial moiety is less than or equal to the Tₘ of the first stem portion:second stem portion; wherein the first effector partial moiety and the second effector partial moiety form an active effector agent when in sufficient proximity.

The present disclosure also provides methods of labeling a cell surface with a quantifiable reverse template, the method comprising the steps: a) contacting the cell with an azide-modified sugar; b) contacting the cell with two ligation-template oligonucleotides, wherein: i) the first ligation-template oligonucleotide (LT1) comprises a 5′-azide reactive molecule that is chemically reactable with an azide group; and ii) the second ligation-template oligonucleotide (LT2) comprises a 5′-phosphate, and a 3′-azide reactive molecule that is chemically reactable with an azide group; c) contacting the cell with a nuclease resistant oligonucleotide, wherein the nuclease resistant oligonucleotide is non-overlap complementary to both LT1 and LT2; wherein upon close proximity, the 3′-OH of LT1 and the 5′-phosphate of LT2 are ligatable; and d) contacting the cell with a ligase, thereby generating a reverse template formed from LT1 and LT2 that can be amplified and quantified.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a representative metabolic labeling process of cell surface glycoproteins with Azido-N-acetylmannosamine (AzNAM).

FIG. 2 shows placement of surface template by DBCO-modification of cells displaying azide-modified surface glycans.

FIG. 3 shows 5 a representative reverse-template assay for cell surface glycan density and reaction of metabolically-labeled cells with surface azide with DBCO-modified ligation-template (LT) phosphorothioate oligos.

FIG. 4 shows a representative reverse-template assay for cell surface glycan density and annealing/bridging of surface LT oligos with 2′-O-methyl template, sealing of gap with Splint ligase, and quantitative amplification.

FIG. 5 shows representative surface placement of a bifunctional compound on a cell having an azide-modified sugar on its surface.

FIG. 6 shows representative surface placement of an FKBP-binding compound-polynucleotide complex on a cell having a bifunctional compound bound thereto.

FIG. 7 shows a template system that becomes accessible to haplomer hybridization only after DBCO-mediated reactions on a cell surface with high-density glycans, modified with surface azide.

FIG. 8 shows a representative AzNAM-6-phosphate structure.

FIG. 9 shows depiction of process for coupling of metabolic labeling with specific transcriptomic targets and liberation of AzNAM by cellular RNases.

FIG. 10 shows a representative process for coupling of metabolic labeling with specific transcriptomic targets and liberation of AzNAM by means of an introduced phosphorothioate complementary oligonucleotide and cellular RNase H.

FIG. 11 shows a representative alternative modular configuration for AzNAM-labeled RNA-modified nucleic acid hybrid, with partial complementarity to the cellular transcript-targeting oligonucleotide, such that a loop structure is formed upon hybridization.

FIG. 12 shows numerous representative AzNAM-6,3′-diribocytosinylate structures.

FIG. 13 shows a representative AzNAM-6,5′-diribocytosinylate structure.

FIG. 14 shows azide metabolic labeling of HeLa cells, demonstrated by subsequent reaction with DBCO-FAM.

FIG. 15 shows flow detection of HeLa cells metabolically labeled with AzNAM and then reacted with DBCO-FAM, in comparison to fluorometric measurement (fluorescent plate reader) for the same cells.

FIG. 16 shows principles of joining of ligation-template oligonucleotides for qPCR analysis of surface 2′-O-Me-template.

FIG. 17 shows surface assembly of ligation-template oligonucleotides on cells bearing surface 2′-O-methyl templates placed via reaction between template-conjugated DBCO groups with metabolically-generated cell surface azide groups.

FIG. 18 shows use of bilabeled FAM Probe to Detect Surface Template Placed Via AzNAM Treatment of Cells.

FIG. 19 shows example of a qPCR standard curve with oligonucleotide target corresponding to LT1/LT2 templated ligation products (code #593) as used in FIG. 17 (Example 2); the Y-axis indicates the CT value (the threshold cycle number for detection); the X-axis indicates log 10 (number of known molecules in triplicate wells in a dilution series of the oligonucleotide standard); the equation for this standard curve is y=−1.472 ln(x)+41.917; the R2 (correlation coefficient)=0.99797.

FIG. 20A shows flow cytometric analysis of human Jurkat cells after treatment with 100 ng/ml human IFN-beta for 3 days and supplemented with AzNAM before harvest.

FIG. 20B shows flow cytometric analysis of human Jurkat cells after treatment with 100 ng/ml human IFN-beta for 3 days and supplemented with control solvent only before harvest.

FIG. 20C shows flow cytometric analysis of human Jurkat cells after no-treatment control for 3 days and supplemented with AzNAM before harvest.

FIG. 20D shows flow cytometric analysis of human Jurkat cells after no-treatment control for 3 days and supplemented with control solvent only before harvest.

FIG. 21A shows flow cytometric analysis of human Jurkat cells after treatment with 75 ng/ml human IFN-beta for 3 days and supplemented with AzNAM before harvest.

FIG. 21B shows flow cytometric analysis of human Jurkat cells after treatment with 75 ng/ml human IFN-beta for 3 days and supplemented with control solvent only before harvest.

FIG. 21C shows flow cytometric analysis of human Jurkat cells after treatment with no-treatment control for 3 days and supplemented with AzNAM before harvest.

FIG. 21D shows flow cytometric analysis of human Jurkat cells after treatment with no-treatment control for 3 days and supplemented with control solvent only before harvest.

FIG. 22A shows flow cytometric analysis of murine ID8 cells after treatment with 100 ng/ml murine IFN-beta for 3 days and supplemented with AzNAM before harvest.

FIG. 22B shows flow cytometric analysis of murine ID8 cells after treatment with 100 ng/ml murine IFN-beta for 3 days and supplemented with control solvent only before harvest.

FIG. 22C shows flow cytometric analysis of murine ID8 cells after treatment with no-treatment control for 3 days and supplemented with AzNAM before harvest.

FIG. 22D shows flow cytometric analysis of murine ID8 cells after treatment with no-treatment control for 3 days and supplemented with control solvent only before harvest.

FIG. 23A shows flow cytometric analysis of human Jurkat cells after treatment with 50 μM STING agonist 1 (STAG1) for 3 days and supplemented with AzNAM before harvest.

FIG. 23B shows flow cytometric analysis of human Jurkat cells after treatment with 50 μM STING agonist 1 (STAG1) for 3 days and supplemented with control solvent only before harvest.

FIG. 23C shows flow cytometric analysis of human Jurkat cells after treatment with no-treatment control for 3 days and supplemented with AzNAM before harvest.

FIG. 23D shows flow cytometric analysis of human Jurkat cells after treatment with no-treatment control for 3 days and supplemented with control solvent only before harvest.

FIG. 24A shows flow cytometric analysis of murine ID8 cells after treatment with 20 μM murine STING agonist DMXAA for 3 days and supplemented with AzNAM before harvest.

FIG. 24B shows flow cytometric analysis of murine ID8 cells after treatment with 20 μM murine STING agonist DMXAA for 3 days and supplemented with control solvent only before harvest.

FIG. 24C shows flow cytometric analysis of murine ID8 cells after treatment with no-treatment control for 3 days and supplemented with AzNAM before harvest.

FIG. 24D shows flow cytometric analysis of murine ID8 cells after treatment with no-treatment control for 3 days and supplemented with control solvent only before harvest.

### DETAILED DESCRIPTION

Numerous techniques exist for assembly of a molecule on the surface of a cell or within a cell. For example, several processes for the templated assembly of molecules by proximity-enhanced reactivity have been described (see, PCT Publications WO 14/197547, WO 17/205277, WO 18/94070, WO 18/94195, WO 18/93978, and, WO 19/032942).

For example novel structures can be assembled on cellular nucleic acid templates which define pathogenic or otherwise undesirable cell classes. Such templated assembly processes can be used to target the cell types of interest for destruction. Pairs of modified oligonucleotides carrying specially tailored and mutually reactive groups can assemble molecules with predetermined functions following co-annealing in spatial proximity on a target cellular template (i.e., Template Assembly by Proximity-Enhanced Reactivity (TAPER). Proteins can be assembled via folding or dimerization using nucleic acid molecule templates which can be used to combat pathogenic or otherwise undesirable cells or cell products. Such templated assembly processes can be used to target the cell types of interest for destruction. Pairs of modified oligonucleotides carrying specially tailored and mutually reactive ligands can assemble proteins with predetermined functions following templated assembly. Unlike other forms of protein complementation (such as the alpha-complementation of beta-galactosidase) where pre-folded subunits interact, some of the methods described herein involve split-protein approaches characterized by the facilitation of mature folding pathways through enforced spatial proximity. Consequently, split-protein fragments in isolation cannot recapitulate the functional profile of their corresponding parental protein, and fragment background functional levels are accordingly extremely low.

In many of these processes, a template polynucleotide is delivered to the cell, such as to the surface of the cell, whereby the template polynucleotide can serve as a substrate by which to target the cell for the assembly of a molecule. The assembled molecule can then serve, for example, as a means to destroy the cell (such as by being a toxin) or by acting as a target for a therapeutic compound (such as by being an antigen for a therapeutic antibody). Numerous methods for the delivery of the template polynucleotide are set forth in the foregoing processes. Additional methods for the delivery of the template polynucleotide are set forth herein.

The terminology used herein is for the purpose of describing particular embodiments only and is not intended to be limiting. The terms used in this disclosure adhere to standard definitions generally accepted by those having ordinary skill in the art. In case any further explanation might be needed, some terms have been further elucidated below.

As used herein, the singular forms “a,” “an,” and “the” include plural references unless the content clearly dictates otherwise.

As used herein, the phrases “active effector agent’” and “effector agent” are used interchangeably to refer to an active portion of a templated assembly product produced upon combination of reactive effector moieties that triggers a desired effect.

As used herein, the term “alkyl” means a saturated hydrocarbon group which is straight-chained or branched. In some embodiments, the alkyl group has from 1 to 20 carbon atoms, from 2 to 20 carbon atoms, from 2 to 16 carbon atoms, from 4 to 12 carbon atoms, from 4 to 16 carbon atoms, from 4 to 10 carbon atoms, from 1 to 10 carbon atoms, from 2 to 10 carbon atoms, from 1 to 8 carbon atoms, from 2 to 8 carbon atoms, from 1 to 6 carbon atoms, from 2 to 6 carbon atoms, from 1 to 4 carbon atoms, from 2 to 4 carbon atoms, from 1 to 3 carbon atoms, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, octyl, nonyl, 4,4 dimethylpentyl, decyl, undecyl, dodecyl, 2,2,4-trimethylpentyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the phrase “anti-target loop portion” refers to a portion of a haplomer or bottle haplomer that facilitates sequence-specific binding to a nucleic acid template.

As used herein, the term “base” refers to a molecule containing a purine or pyrimidine group, or an artificial analogue, that forms a binding pair with another corresponding base via Watson-Crick or Hoogsteen bonding interactions. Bases further contain groups that facilitate covalently joining multiple bases together in a polymer, such as an oligomer. Non-limiting examples include nucleotides, nucleosides, peptide nucleic acid residues, or morpholino residues.

As used herein, the terms “bind,” “binds,” “binding,” and “bound” refer to a stable interaction between two molecules that are close to one another. The terms include physical interactions, such as chemical bonds (either directly linked or through intermediate structures), as well as non-physical interactions and attractive forces, such as electrostatic attraction, hydrogen bonding, and van der Waals/dispersion forces.

As used herein, the phrase “bioconjugation chemistry” refers to the chemical synthesis strategies and reagents that ligate common functional groups together under mild conditions, facilitating the modular construction of multi-moiety compounds.

As used herein, the phrase “chemical linker” refers to a molecule that binds one haplomer to another haplomer or one moiety to another moiety on different compounds. A linker may be comprised of branched or unbranched covalently bonded molecular chains.

As used herein, the terms “comprising” (and any form of comprising, such as “comprise”, “comprises”, and “comprised”) and “having” (and any form of having, such as “have” and “has”) are inclusive and open-ended and include the options following the terms, and do not exclude additional, unrecited elements, or method steps.

As used herein, the term “contacting” means bringing together a compound and a cell, or a compound with another compound in an in vitro system or an in vivo system.

As used herein, the phrase “dosage unit form” refers to physically discrete units suited as unitary dosages for the subjects to be treated.

As used herein, the phrase “effector partial moiety,” means a portion of a haplomer that contributes to the chemical structure of the effector agent in a product formed by nucleic acid templated assembly. An effector partial moiety may be a distinct portion of the reactant, or may include or be comprised of part or all of the nucleic acid recognition moiety and/or the selectively-reactive moiety.

As used herein, the phrase “ethylene glycol unit” means a polymer of —(O—CH₂—CH₂)ₙ—O—, wherein n is from 1 to about 20. A polyethylene glycol (PEG) having 4 ethylene glycol units (i.e., —(O—CH₂—CH₂)₄—O—) is referred to herein as PEG4.

As used herein, the term “haplomer” refers to nucleic acid molecules, such as nucleic acid recognition moieties, linked to a fragment of a protein or ligand that binds to a nucleic acid template in a sequence-specific manner and participates in protein or product formation during nucleic acid templated assembly. Also included herein are “derivatives” or “analogs” such as salts, hydrates, solvates thereof, or other molecules that have been subjected to chemical modification and maintain the same biological activity or lack of biological activity, and/or ability to act as a haplomer, or function in a manner consistent with a haplomer.

As used herein, the phrase “nucleic acid recognition moiety” means a compound that facilitates sequence-specific binding to a nucleic acid template.

As used herein, the phrase “nucleic acid templated assembly” means the synthesis of an active effector agent or agents on a nucleic acid template, such that the effector agent formation can be facilitated by haplomers being assembled in proximity when bound to the nucleic acid template. In some embodiments, the active effector agent formation can be dimerization or folding of a protein on a nucleic acid template or production of a protein on a nucleic acid template.

As used herein, the terms “oligomer” and “oligo” refer to a molecule comprised of multiple units where some or all of the units are bases capable of forming Watson-Crick or Hoogsteen base-pairing interactions, allowing sequence-specific binding to nucleic acid molecules in a duplex or multiplex structure. Non-limiting examples include, but are not limited to, oligonucleotides, peptide nucleic acid oligomers, and morpholino oligomers.

As used herein, the phrase “pathogenic cell” can refer to a cell that is capable of causing or promoting a diseased or an abnormal condition, such as a cell infected with a virus, a tumor cell, and a cell infected with a microbe, or a cell that produces a molecule that induces or mediates diseases that include, but are not limited to allergy, anaphylaxis, inflammation and autoimmunity.

As used herein, the phrase “pharmaceutically acceptable” refers to a material that is not biologically or otherwise unacceptable, that can be incorporated into a composition and administered to a patient without causing unacceptable biological effects or interacting in an unacceptable manner with other components of the composition. Such pharmaceutically acceptable materials typically have met the required standards of toxicological and manufacturing testing, and include those materials identified as suitable inactive ingredients by the U.S. Food and Drug Administration.

As used herein, the phrase “pharmaceutically acceptable salt” means a salt prepared from a base or an acid which is acceptable for administration to a patient, such as a mammal (e.g., salts having acceptable mammalian safety for a given dosage regime). However, it is understood that the salts covered herein are not required to be pharmaceutically acceptable salts, such as salts of the haplomers that are not intended for administration to a patient. Pharmaceutically acceptable salts can be derived from pharmaceutically acceptable inorganic or organic bases and from pharmaceutically acceptable inorganic or organic acids. In addition, when a haplomer contains both a basic moiety, such as an amine, and an acidic moiety such as a carboxylic acid, zwitterions may be formed and are included within the term “salt” as used herein. Salts derived from pharmaceutically acceptable inorganic bases can include ammonium, calcium, copper, ferric, ferrous, lithium, magnesium, manganic, manganous, potassium, sodium, and zinc salts, and the like. Salts derived from pharmaceutically acceptable organic bases can include salts of primary, secondary and tertiary amines, including substituted amines, cyclic amines, naturally-occurring amines and the like, such as arginine, betaine, caffeine, choline, N,N-dibenzylethylenediamine, diethylamine, 2-diethylaminoethanol, ethanolamine, 2-dimethylaminoethanol, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids can include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids can include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucoronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like.

As used herein, the phrase “reactive effector moiety” refers to a portion of a haplomer that enables formation of an active effector agent, such as through a chemical reaction with a corresponding haplomer on an adjacent templated assembly. For example, a reactive effector moiety can react readily with a corresponding reactive effector moiety, but does not readily react with natural biomolecules.

As used herein, the term “sample” refers to any system that haplomers can be administered into, where nucleic acid templated assembly may occur. Examples of samples include, but are not limited to, fixed or preserved cells, whole organisms, tissues, tumors, lysates, or in vitro assay systems.

As used herein, the phrase “selectively-reactive moiety” refers to the portion of a haplomer that enables formation of product, such as through a chemical reaction with a corresponding haplomer, on an adjacent templated assembly. For example, a selectively-reactive moiety can react readily with a corresponding selectively-reactive moiety, but does not readily react with natural biomolecules.

As used herein, the phrases “set of corresponding reactants” or “corresponding haplomers” refer to haplomers that come together on a single nucleic acid template to take part in a templated assembly reaction.

As used herein, the phrase “target compartment” refers to a cell, virus, tissue, tumor, lysate, other biological structure, spatial region, or sample that contains nucleic acid template(s), or a different amount of nucleic acid templates than a non-target compartment.

As used herein, the phrase “templated assembly product,” refers to the active effector agent or agents formed by interaction, binding, reaction, binding, or dimerization of one or more nucleic acid haplomers.

At various places herein, substituents of compounds may be disclosed in groups or in ranges. Designation of a range of values includes all integers within or defining the range (including the two endpoint values), and all subranges defined by integers within the range. It is specifically intended that the disclosure include each and every individual subcombination of the members of such groups and ranges. For example, the term “C₁₋₆alkyl” is specifically intended to individually disclose methyl, ethyl, propyl, C₄alkyl, C₅alkyl, and C₆alkyl.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

The structures depicted herein may omit necessary hydrogen atoms to complete the appropriate valency. Thus, in some instances a carbon atom or nitrogen atom may appear to have an open valency (i.e., a carbon atom with only two bonds showing would implicitly also be bonded to two hydrogen atoms; in addition, a nitrogen atom with a single bond depicted would implicitly also be bonded to two hydrogen atoms). For example, “—N” would be considered by one skilled in the art to be “—NH₂.” Thus, in any structure depicted herein wherein a valency is open, one or more hydrogen atoms, as appropriate, is implicit, and is only omitted for brevity.

The compounds described herein can also include all isotopes of atoms occurring in the intermediates or final compounds. Isotopes include those atoms having the same atomic number but different mass numbers. For example, isotopes of hydrogen include tritium and deuterium. Carbon (¹²C) can be replaced at any position with ¹³C or ¹⁴C. Nitrogen (¹⁴N) can be replaced with ¹⁵N. Oxygen (¹⁶O) can be replaced at any position with ¹⁷O or ¹⁸O. Sulfur (³²S) can be replaced with ³³S, ³⁴S or ³⁶S. Chlorine (³⁵Cl) can be replaced with ³⁷Cl. Bromine (⁷⁹Br) can be replaced with ⁸¹Br.

In some embodiments, the compounds, or salts thereof, are substantially isolated. Partial separation can include, for example, a composition enriched in any one or more of the compounds described herein. Substantial separation can include compositions containing at least about 50%, at least about 60%, at least about 70%, at least about 80%, at least about 90%, at least about 95%, at least about 97%, or at least about 99% by weight of any one or more of the compounds described herein, or salt thereof. Methods for isolating compounds and their salts are routine in the art.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Since the sialic acid and other sugar-based metabolic pathways are ubiquitous in mammalian cellular and molecular physiologies, placement of azides or other unnatural compounds on cell surfaces by such means alone is not restricted to pathological circumstances. Nevertheless, where the density of surface glycans (which display modified azide-bearing sugars) is abnormally high, a differential between normal and aberrant conditions is thereby provided. High surface glycan density in tumor cells in turn results in corresponding increased density of metabolically-placed surface azides or other artificial moieties of interest. The latter unnatural surface groups act as molecular determinants for phenomena that inherently rely on molecular proximity, such as template-directed assembly of haplomers (see, PCT Publication WO 14/197547). This assertion follows from the observation that average molecular proximity (P) of a surface marker is directly correlated with its average surface density (D), (such that P a D). To exploit this principle, either templates or haplomers themselves can be equipped with chemical groups that show exclusive bio-orthogonal reactivity with the metabolic surface-displayed moieties. On cells where surface azides are displayed, templates or haplomers may be immobilized on cell surfaces if they are modified with moieties that are bio-orthogonally reactive with azides, such as DBCO.

It would be of particular practical importance to further restrict metabolic labeling itself to pathological cells of interest. In such cases, surface targeting of templates or haplomers could likewise be focused on the desired cellular targets, by specific chemical reactivities between modified cell surfaces and co-modified template/haplomer systems. Such an exquisite targeting process would occur even in the absence of a significant density differential between normal and aberrant cells.

The advent of next-generation sequencing and related technologies has revealed that tumor cells typically show a wealth of transcriptionally aberrant expressed RNA sequences, a great many of which could act as potential discriminatory targets between the pathological and corresponding normal cellular states. Accordingly, a powerful approach towards realizing the restriction of metabolic labeling to specific aberrant target cells would be to devise a means for coupling of specific transcriptionally expressed products with the ability of cells to incorporate labeled artificial sugar precursors into desired metabolic pathways, that ultimately lead to surface positioning of the chemical moiety of interest.

The present disclosure describes applications of cellular metabolic labeling with sugars modified by selectively-reactive moieties for displaying bio-orthogonally reactive groups (including, but not limited to azides) on cell surfaces to enable novel applications of templated assembly (TAPER technology). The present disclosure also provides methods whereby metabolic labeling and novel assembly processes can form the basis of multiple assays for assessing the relative surface densities of cellular glycoproteins. Increased surface density of such structures can be a feature in distinguishing cancer cells from their normal counterparts, as well as for implementing TAPER-based specific assembly of desired products for ultimately killing the pathological cells.

Sugar modifications with selectively-reactive moieties for cell-surface metabolic labeling include, but are not limited to, azides, alkynes, halogens, short-chain alkyl groups, halogenated alkyl groups, sulfhydryls, thiomethyl groups, cyclopropenes, and cyclopentenes. In some embodiments, the selectively-reactive moiety of the sugar modification is an azide. In some embodiments, the selectively-reactive moiety of the sugar modification is an alkyne. In some embodiments, the selectively-reactive moiety of the sugar modification is a halogen. In some embodiments, the selectively-reactive moiety of the sugar modification is a short-chain alkyl group. In some embodiments, the selectively-reactive moiety of the sugar modification is a halogenated alkyl group. In some embodiments, the selectively-reactive moiety of the sugar modification is a sulfhydryl. In some embodiments, the selectively-reactive moiety of the sugar modification is a thiomethyl group. In some embodiments, the selectively-reactive moiety of the sugar modification is a cyclopropene. In some embodiments, the selectively-reactive moiety of the sugar modification is a cyclopentene.

The present disclosure provides methods of labeling a cell with a substrate for a templated assembly reaction, the method comprising the steps: a) contacting the cell with an azide-modified sugar; and b) contacting the cell with the substrate for the templated assembly reaction, wherein the substrate comprises: i) a nucleic acid template; and ii) an azide reactive molecule linked to the nucleic acid template at the 5′- or 3′-end of the nucleic acid template, wherein the azide reactive molecule is chemically reactable with the azide of the azide-modified sugar.

In any of the embodiments described herein, the azide-modified sugar can be replaced with an alkyne-modified sugar, a halogen-modified sugar, a short-chain alkyl group-modified sugar, a halogenated alkyl group-modified sugar, a sulfhydryl-modified sugar, a thiomethyl group-modified sugar, a cyclopropene-modified sugar, or a cyclopentene-modified sugar. In such embodiments, an appropriate reactive molecule linked to the nucleic acid template at the 5′- or 3′-end of the nucleic acid template is used (i.e., a molecule reactive with a cyclopentene group, etc.).

In some embodiments, the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA). In some embodiments, the azide-modified sugar is AzNAM. In some embodiments, the azide-modified sugar is AzGlcNAc. In some embodiments, the azide-modified sugar AGalNAc. In some embodiments, the azide-modified sugar is AzNANA. In some embodiments, the azide-modified sugar is acetylated at 1, 2, 3, or 4 positions. In some embodiments, the azide-modified sugar is acetylated at 1 position. In some embodiments, the azide-modified sugar is acetylated at 2 positions. In some embodiments, the azide-modified sugar is acetylated at 3 positions. In some embodiments, the azide-modified sugar is acetylated at 4 positions.

In some embodiments, the azide reactive molecule is dibenzocyclooctyne (DBCO), bicyclo[6.1.0]nonyne (BCN), methyltetrazine, or trans-cyclooctene (TCO). In some embodiments, the azide reactive molecule is DBCO. In some embodiments, the azide reactive molecule is BCN. In some embodiments, the azide reactive molecule is methyltetrazine. In some embodiments, the azide reactive molecule is TCO.

In some embodiments, the nucleic acid template is chosen from a cancer-specific polynucleotide, a viral polynucleotide, a microbial-specific polynucleotide, a differentially expressed gene, and a disease-specific polynucleotide. In some embodiments, the nucleic acid template is a cancer-specific polynucleotide. In some embodiments, the nucleic acid template is a viral polynucleotide. In some embodiments, the nucleic acid template is a microbial-specific polynucleotide. In some embodiments, the nucleic acid template is a differentially expressed gene. In some embodiments, the nucleic acid template is a disease-specific polynucleotide.

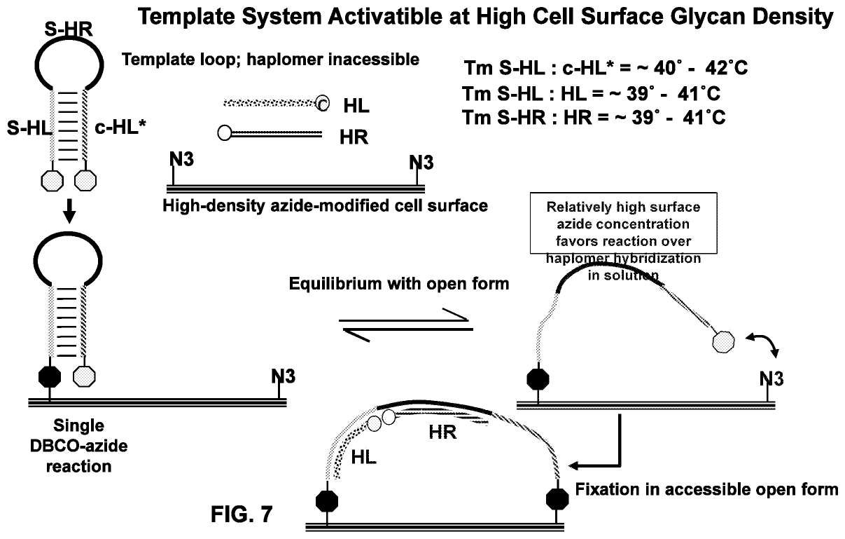

In some embodiments, it may be desired to design templates that are only available for haplomer interaction on high glycan density tumor cell surfaces. For instance, success of therapeutic treatment can be enhanced by preventing haplomer assembly on circulating in vivo templates before the templates home in on target cells. This can be approached by designing a template bearing the usual contiguous haplomer hybridization sites, and where another segment of the template is complementary to one of the haplomer sites (left haplomer or HL, as depicted in FIG. 7). By appropriate sequence design, the template forms a tight loop, where the remaining “right” haplomer (HR) is precluded from stable hybridization within the loop and opening of the stem through its relatively lower Tm (see, FIG. 7). Another feature of the surface-activatable template is conjugation of both 5′ and 3′ ends with DBCO or other selectively-reactive moieties, each thus positioned at defined stem boundaries (see, FIG. 7).

When this form of template encounters an azide-labeled surface, one of the DBCO groups will react to form the usual triazole click product (see, FIG. 7, depicted as dark-shaded octagons). With appropriate design of thermal stability of the stem-region of the template, at 37° C. the stem/loop structure will be in equilibrium between the annealed (loop-forming) and open configurations, with a preponderance towards the loop form. In solution, this disfavors haplomer reactivity, since only the open form is compatible with haplomer hybridization. On a cell surface with high-density azide modifications, however, there is an enhanced probability of the open form becoming fixed through reaction of the second DBCO group, which spatially disfavors reformation of the original stem-loop configuration (see, FIG. 7). When this has taken place, the bi-attached surface template is accessible for subsequent haplomer hybridization and reactivity (see, FIG. 7).

The present disclosure also provides methods of labeling a cell with a substrate for a templated assembly reaction, the method comprising the steps: a) contacting the cell with an azide-modified sugar; and b) contacting the cell with a nucleic acid template, wherein the nucleic acid template comprises: i) a first hybridization region and a second hybridization region separated by a loop region, wherein the first hybridization region is complementary to the second hybridization region; and ii) an azide reactive molecule at the 3′-end of the nucleic acid template and at the 5′-end of the nucleic acid template, wherein both azide reactive molecules are chemically reactable with an azide group. The azide-modified sugars, azide reactive molecules, and nucleic acid templates can be any of those described herein.

In some embodiments, it may be desirable to couple cellular metabolic labeling with specific transcriptomes. This coupling process ensures that cell surface bio-orthogonal modifications are manifested solely on cells expressing a target RNA molecule of interest. Consequently, this provides a means for linking aberrant transcripts unique to cancer cells with an artificially induced surface state, where such surface-modified cells can be targeted for destruction.

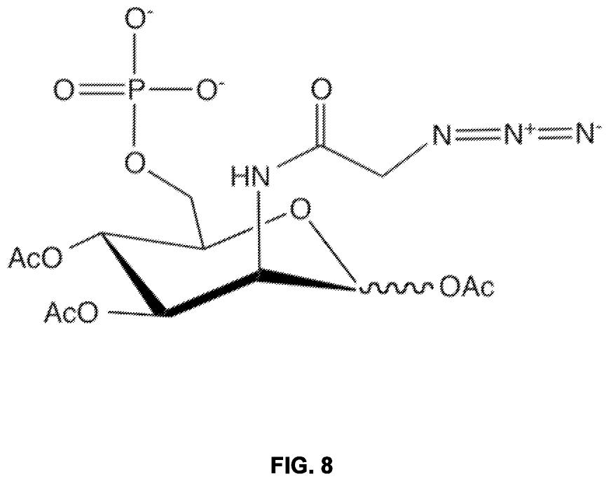

In some embodiments, methods are developed whereby metabolic labeling is induced only in cells of interest, taking advantage of the unique expression of specific RNA transcripts in certain pathological cells, including tumors. The general principle involves conjugation of AzNAM (or other click-labeled and metabolically compatible sugars) with a nucleic acid whose structure is differentially and specifically modified in the presence of a target transcript. This target-induced structural modification is then designed to enable the release of the conjugated AzNAM moiety from its carrier nucleic acid molecule, after which the free AzNAM becomes accessible to metabolic processing and ultimate surface display on modified glycans. Conjugation of AzNAM to nucleic acid molecules can be affected through the 6-position of the sugar molecule, through a 6-phosphate group (shown for AzNAM-6-phosphate; see, FIG. 8) linking to a specific nucleic acid sequence of desired length.

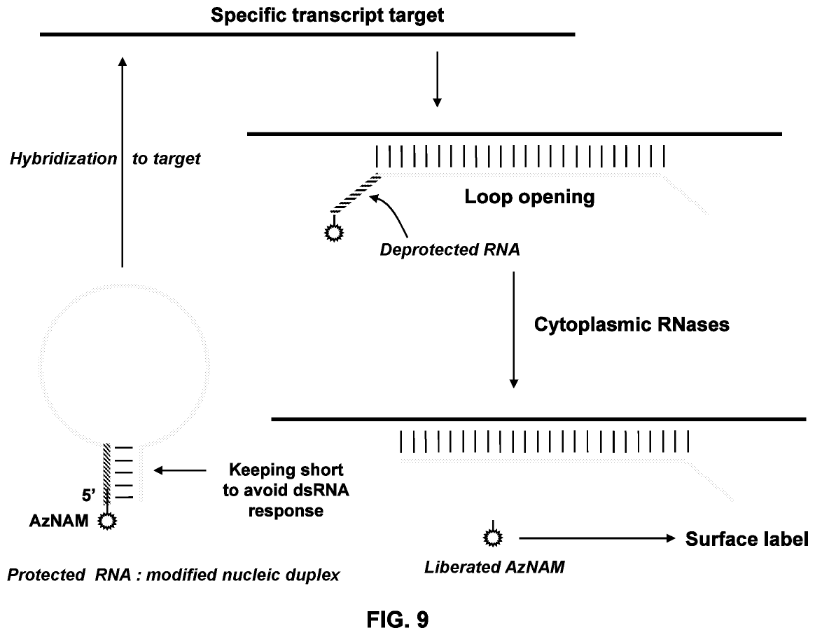

To elicit the structural change in the nucleic acid molecule bearing the AzNAM moiety, in some embodiments the nucleic acid sequence is a hybrid between a short RNA segment and a longer modified nucleic acid segment, where a region of the latter is complementary to the RNA segment, which itself is conjugated with AzNAM (see, FIG. 9). The RNA:modified nucleic acid complementarity permits the formation of a duplex region, which in turn defines a loop comprised of modified nucleic acid residues designed to be complementary to the target transcript of interest. Another feature of the RNA:modified nucleic acid duplex is its conferring retardation of attack on the RNA strand by many cellular RNases. The modified nucleic acid region of the AzNAM-conjugated nucleic acid can comprise modified nucleotides such as, for example, 2′-O-methyl RNA or locked nucleic acids (LNAs).

Following entry of a loop-structured hybrid oligonucleotide with terminally-conjugated AzNAM into a specific cell of interest, a target complementary transcript is capable of invading the loop and (by virtue of its greater thermal stability) forcing the foreign oligonucleotide into its open configuration (see, FIG. 9), in a manner analogous to the Locked-TAPER technology (see, PCT Publication WO 18/94070). Once this occurs, the RNA segment bearing the conjugated AzNAM moiety becomes single-stranded and vulnerable to attack by multiple cellular RNases. Released AzNAM is then able to be metabolically shunted into the sialic acid pathway, leading to its final display on surface glycans.

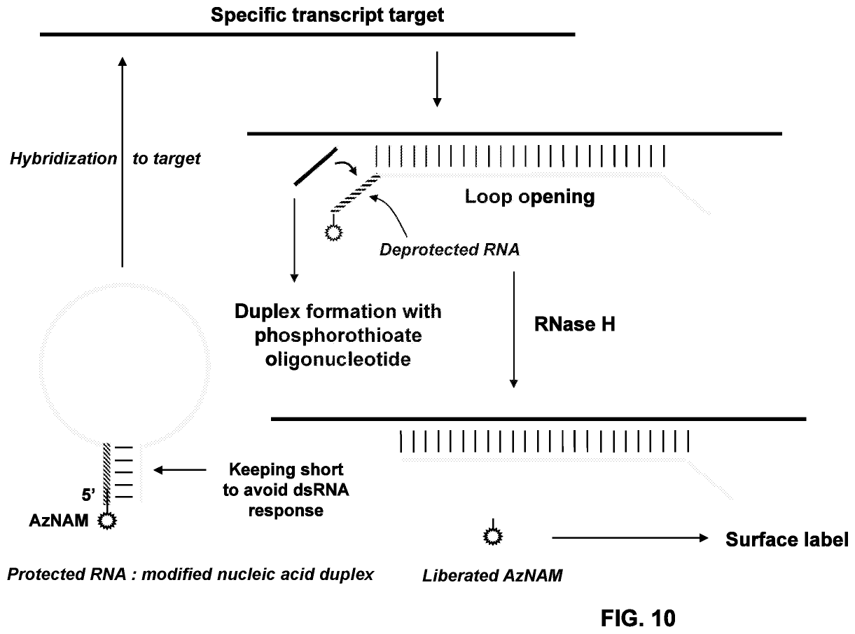

In some embodiments, the process of RNA segment hydrolysis is assisted by co-provision of a short complementary nucleic acid strand with a phosphorothioate backbone (see, FIG. 10). This short modified strand is thereby nuclease resistant itself while still promoting efficient attack on a complementary RNA strand by RNase H, when two strands of this nature form a duplex (see, Iwamoto et al., Nat. Biotechnol., 2017, 35, 845-851). AzNAM is then released as a consequence of ubiquitous RNase H activity (see, FIG. 10).

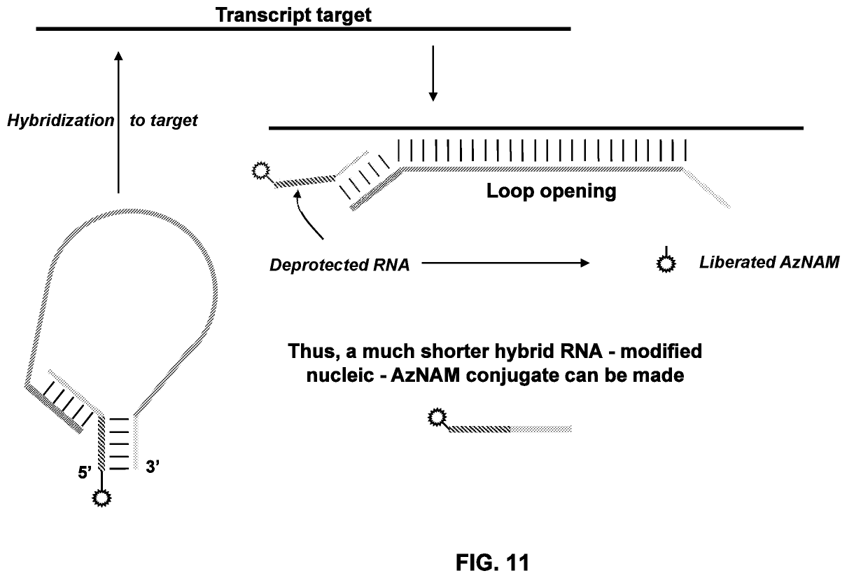

To improve the modularity of this system, in some embodiments the AzNAM-tagged segment is a discrete smaller nucleic acid composed of an RNA tract (to which the AzNAM is directly conjugated) and a contiguous tract of nuclease-resistant modified nucleic acid. The latter tract is complementary to one end of a larger modified oligonucleotide that serves as the recognition element for a cellular transcriptomic target (see, FIG. 11). The AzNAM-coupled RNA segment itself is complementary to the opposite end of the transcript recognition oligonucleotide, thus allowing the formation of a loop structure (see, FIG. 11). The behavior of this structure in the presence of a specific target transcript is then comparable to the original simpler design as described above (see, FIG. 10 and FIG. 11). But the shorter hybrid RNA-AzNAM molecule allows modular switching between different transcript recognition oligonucleotides, provided the latter are equipped with common 5′ and 3′ segments for loop formation when complexed with the smaller hybrid RNA-AzNAM species.

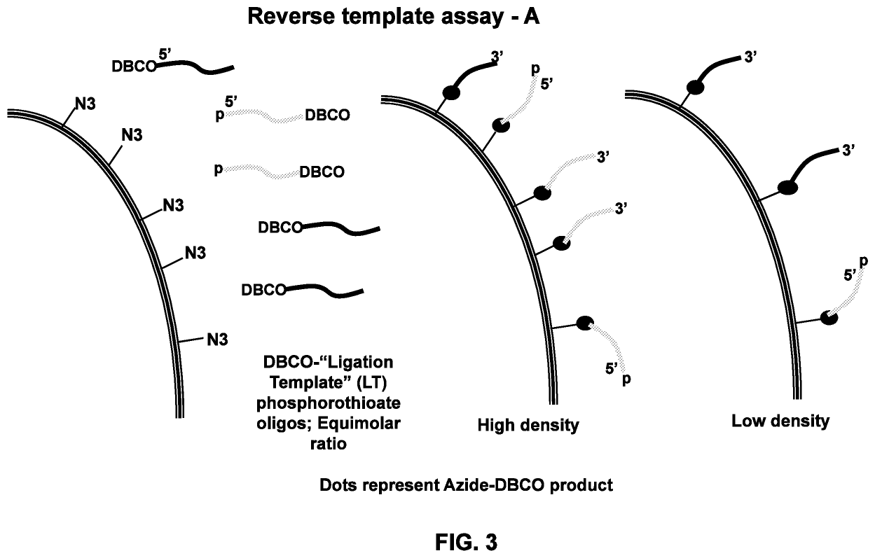

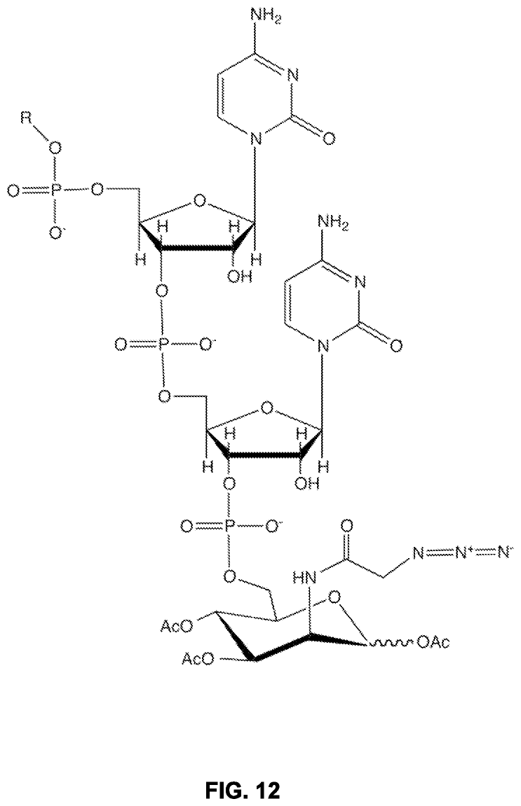

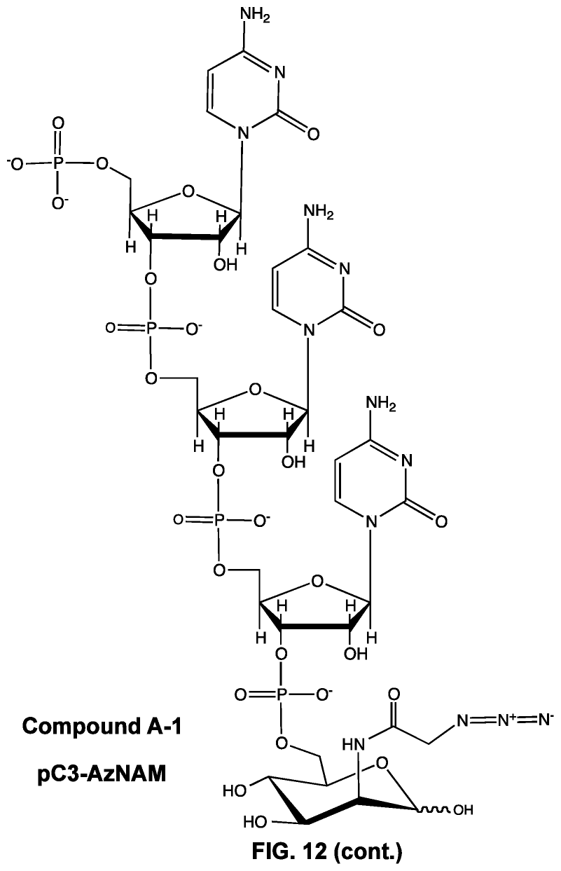

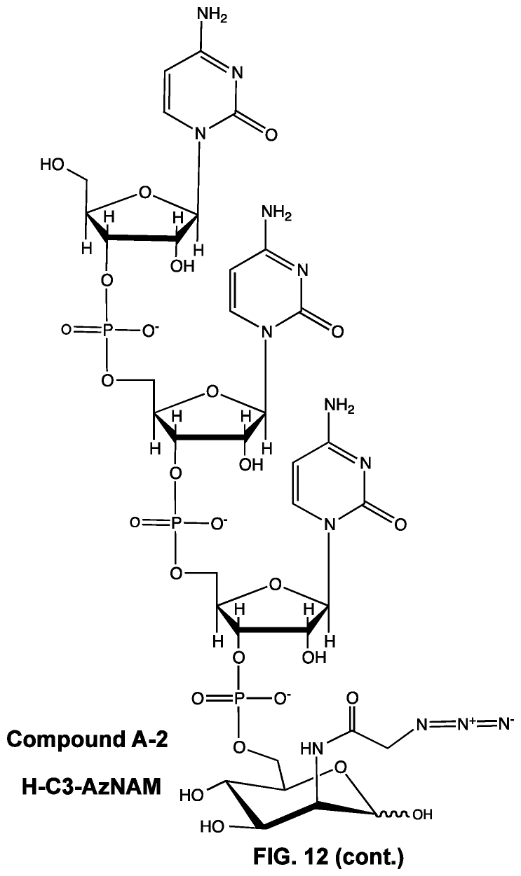

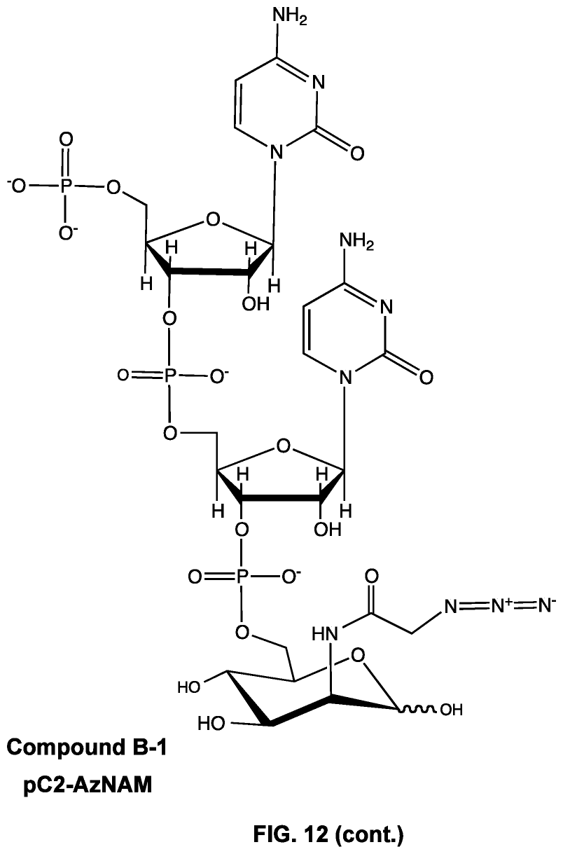

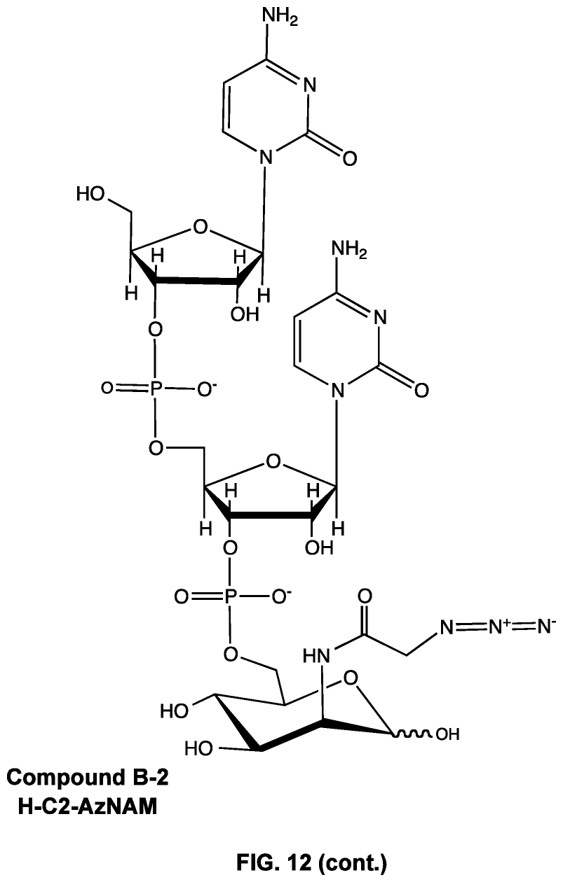

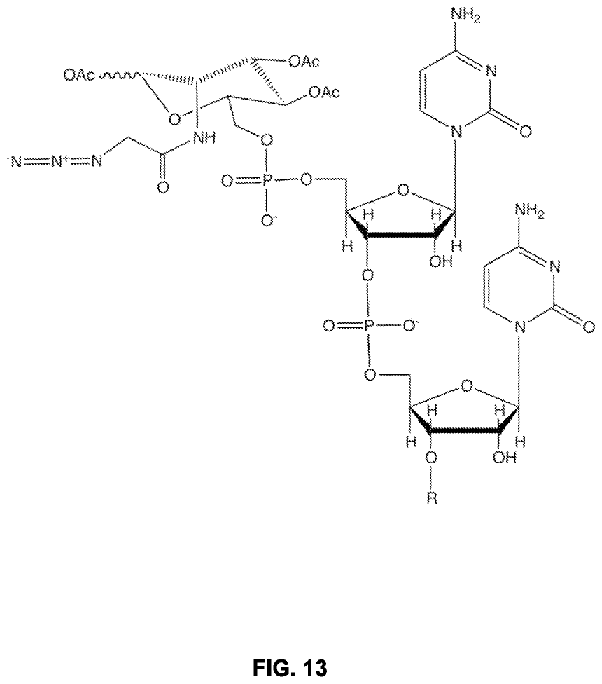

In some embodiments, the AzNAM moiety is synthetically joined to a short ribonucleotide moiety, shown as a non-limiting example for a diribocytosinyl conjugate with AzNAM at either the 3′-end (see, FIG. 12) or 5′-end (see, FIG. 13). Such a product is not directly functional in the transcript-metabolic labeling coupling process (see, FIG. 9 and FIG. 10), but is advantageous for its ready and modular adaptation into the necessary longer oligonucleotide derivatives, via enzymatic linkage through the action of RNA ligases. Thus, the 3′-hydroxyl of an RNA oligonucleotide or RNA-2′-O-Methyl hybrid molecule can be ligated to AzNAM-6,3′-diribocytosinylate with a 5′-phosphate (see, FIG. 12), or the 5′-phosphate of an RNA oligonucleotide or RNA-2′-O-Methyl hybrid molecule can be ligated to the 3′-hydroxyl of AzNAM-6,5′-diribocytosinylate, in both cases by RNA ligase I. The sites of such ligated nucleic acids are indicated by R groups for the 5′-phosphate of FIGS. 12 and 3′-phosphate of FIG. 13.

The present disclosure also provides methods of metabolically labeling the surface of a specific target cell, the method comprising the steps: a) contacting the cell with a nucleic acid molecule, wherein the nucleic acid molecule comprises: i) a short terminal RNA segment comprising a terminal azide-modified sugar; and ii) a longer modified RNA segment linked to the short terminal RNA segment, wherein the terminal end of the longer modified RNA segment is complementary to the short terminal RNA segment; wherein the longer modified RNA segment is complementary to a specific transcript target within the specific targeted cell; and wherein the longer modified RNA segment is modified to be nuclease resistant. The azide-modified sugars can be any of those described herein.

In some embodiments, the longer modified RNA segment comprises a phosphorothioate backbone, a phosphoramidate backbone, a morpholino backbone, a bridged nucleic acid backbone, a locked nucleic acid (LNA) backbone, or a 2′ modification. In some embodiments, the longer modified RNA segment comprises a phosphorothioate backbone. In some embodiments, the longer modified RNA segment comprises a phosphoramidate backbone. In some embodiments, the longer modified RNA segment comprises a morpholino backbone. In some embodiments, the longer modified RNA segment comprises a bridged nucleic acid backbone. In some embodiments, the longer modified RNA segment comprises an LNA backbone.

In some embodiments, the longer modified RNA segment comprises a 2′ modification. In some embodiments, the 2′ modification is chosen from —O((CH₂)ₙO)ₘCH₃, —O(CH₂)ₙOCH₃, —O(CH₂)ₙNH₂, —O(CH₂)ₙCH₃, —O(CH₂)ₙ—ONH₂, and —O(CH₂)ₙON((CH₂)ₙCH₃))₂, where n and m are, independently, from 0 to about 10. In some embodiments, the 2′ modification is —O((CH₂)ₙO)ₙ,CH₃, where n and m are, independently, from 0 to about 10. In some embodiments, the 2′ modification is —O(CH₂)ₙOCH₃, where n is from 0 to about 10. In some embodiments, the 2′ modification is —O(CH₂)ₙNH₂, where n is from 0 to about 10. In some embodiments, the 2′ modification is —O(CH₂)ₙCH₃, where n is from 0 to about 10. In some embodiments, the 2′ modification is —O(CH₂)ₙ—ONH₂, where n is from 0 to about 10. In some embodiments, the 2′ modification is —O(CH₂)ₙON((CH₂)ₙCH₃))₂, where each n is, independently, from 0 to about 10. In some embodiments, the 2′ modification is a 2′-O-methyl group.

In some embodiments, the method further comprises contacting the cell with an oligonucleotide, wherein the oligonucleotide is complementary to the short terminal RNA segment, and wherein the oligonucleotide is modified to be nuclease resistant. In some embodiments, the oligonucleotide comprises a phosphorothioate backbone, a phosphoramidate backbone, a morpholino backbone, a bridged nucleic acid backbone, a locked nucleic acid (LNA) backbone, or a 2′ modification. In some embodiments, the oligonucleotide comprises a phosphorothioate backbone. In some embodiments, the oligonucleotide comprises a phosphoramidate backbone. In some embodiments, the oligonucleotide comprises a morpholino backbone. In some embodiments, the oligonucleotide comprises a bridged nucleic acid backbone. In some embodiments, the oligonucleotide comprises an LNA backbone.

In some embodiments, the oligonucleotide comprises a 2′ modification. In some embodiments, the 2′ modification is chosen from —O((CH₂)ₙO)ₘCH₃, —O(CH₂)ₙOCH₃, —O(CH₂)ₙNH₂, —O(CH₂)ₙCH₃, —O(CH₂)ₙ—ONH₂, and —O(CH₂)ₙON((CH₂)ₙCH₃))₂, where n and m are, independently, from 0 to about 10. In some embodiments, the 2′ modification is —O((CH₂)ₙO)ₘCH₃, where n and m are, independently, from 0 to about 10. In some embodiments, the 2′ modification is —O(CH₂)ₙOCH₃, where n is from 0 to about 10. In some embodiments, the 2′ modification is —O(CH₂)ₙNH₂, where n is from 0 to about 10. In some embodiments, the 2′ modification is —O(CH₂)ₙCH₃, where n is from 0 to about 10. In some embodiments, the 2′ modification is —O(CH₂)ₙ—ONH₂, where n is from 0 to about 10. In some embodiments, the 2′ modification is —O(CH₂)ₙON((CH₂)ₙCH₃))₂, where each n is, independently, from 0 to about 10. In some embodiments, the 2′ modification is a 2′-O-methyl group.

The present disclosure also provides methods of metabolically labeling the surface of a cell, the method comprising the steps: a) contacting the cell with a nucleic acid molecule, wherein the nucleic acid molecule comprises: i) a short terminal RNA segment comprising a terminal azide-modified sugar; ii) a longer modified RNA segment, wherein a first terminal end of the longer modified RNA segment is complementary to the short terminal RNA segment; and iii) a short linker nucleic acid having a first terminal end linked to the terminal end of the short terminal RNA segment that does not comprise the azide-modified sugar wherein the short linker nucleic acid is complementary to a second terminal end of the longer modified RNA segment that is not complementary to the short terminal RNA segment; wherein the longer modified RNA segment is complementary to a specific transcript target; and wherein the longer modified RNA segment is modified to be nuclease resistant. The azide-modified sugars can be any of those described herein.

In some embodiments, the longer modified RNA segment comprises a phosphorothioate backbone, a phosphoramidate backbone, a morpholino backbone, a bridged nucleic acid backbone, a locked nucleic acid (LNA) backbone, or a 2′ modification. In some embodiments, the longer modified RNA segment comprises a phosphorothioate backbone. In some embodiments, the longer modified RNA segment comprises a phosphoramidate backbone. In some embodiments, the longer modified RNA segment comprises a morpholino backbone. In some embodiments, the longer modified RNA segment comprises a bridged nucleic acid backbone. In some embodiments, the longer modified RNA segment comprises an LNA backbone.

In some embodiments, the longer modified RNA segment comprises a 2′ modification. In some embodiments, the 2′ modification is chosen from —O((CH₂)ₙO)ₘCH₃, —O(CH₂)ₙOCH₃, —O(CH₂)ₙNH₂, —O(CH₂)ₙCH₃, —O(CH₂)ₙ—ONH₂, and —O(CH₂)ₙON((CH₂)ₙCH₃))₂, where n and m are, independently, from 0 to about 10. In some embodiments, the 2′ modification is —O((CH₂)ₙO)ₙ,CH₃, where n and m are, independently, from 0 to about 10. In some embodiments, the 2′ modification is —O(CH₂)ₙOCH₃, where n is from 0 to about 10. In some embodiments, the 2′ modification is —O(CH₂)ₙNH₂, where n is from 0 to about 10. In some embodiments, the 2′ modification is —O(CH₂)ₙCH₃, where n is from 0 to about 10. In some embodiments, the 2′ modification is —O(CH₂)ₙ—ONH₂, where n is from 0 to about 10. In some embodiments, the 2′ modification is —O(CH₂)ₙON((CH₂)ₙCH₃))₂, where each n is, independently, from 0 to about 10. In some embodiments, the 2′ modification is a 2′-O-methyl group.

The present disclosure also provides reverse templating assays. For instance, the usual initial provision of template is reversed in favor of a primary treatment of AzNAM-treated cells with DBCO-labeled ligation-template (LT) oligonucleotides (see, FIG. 3). These are synthesized with, for example, phosphorothioate backbones in order to confer nuclease resistance. To AzNAM-labeled cells, an equimolar mixture of two LT oligonucleotides is applied, where one has a 5′-DBCO modification, and the other is modified with both a 5′-phosphate and a 3′-DBCO moiety. Following incubations with the azide-labeled cells, the DBCO groups on the LT oligonucleotides react with azides to effect positioning of the oligonucleotides on the cell surface (see, FIG. 3).

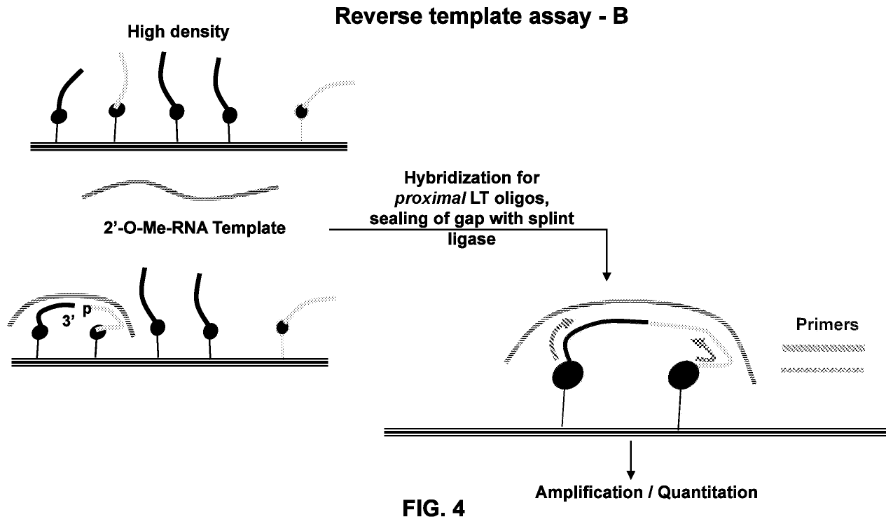

These LT oligonucleotides are both complementary to a longer 2′-O-methyl template, which can hybridize to both oligonucleotides. However, only in conditions of high-density surface distribution of LT oligonucleotides are both accessible for assembly on the same template molecule (see, FIG. 4), such that the 5′-phosphate of one can be ligated with the 3′-hydroxyl of the other. Such ligations upon 2′-O-methyl templates can be effected with Splint® ligase (unpublished observations). When such ligation is accomplished, the LT oligonucleotides form a contiguous sequence which is amenable to PCR amplification, since phosphorothioate templates can support primer extension with normal deoxynucleotide triphosphates (Jung et al., Anal. Bioanal. Chem. 2016, 408, 8583-8591). Amplification of ligated LT oligonucleotides is fully compatible with quantitation by means of qPCR (unpublished data).

The present disclosure also provides methods of labeling a cell surface with a quantifiable reverse template, the method comprising the steps: a) contacting the cell with an azide-modified sugar; b) contacting the cell with two ligation-template oligonucleotides, wherein: i) the first ligation-template oligonucleotide (LT1) comprises a 5′-azide reactive molecule that is chemically reactable with an azide group; and ii) the second ligation-template oligonucleotide (LT2) comprises a 5′-phosphate, and a 3′-azide reactive molecule that is chemically reactable with an azide group; c) contacting the cell with a nuclease resistant oligonucleotide, wherein the nuclease resistant oligonucleotide is non-overlap complementary to both LT1 and LT2; wherein upon close proximity, the 3′—OH of LT1 and the 5′-phosphate of LT2 are ligatable; and d) contacting the cell with a ligase, thereby generating a reverse template formed from LT1 and LT2 that can be amplified and quantified. The azide-modified sugars and the azide reactive molecules can be any of those described herein.

In some embodiments, one or both of the LT1 and/or LT2 comprise a phosphorothioate backbone, a phosphoramidate backbone, a morpholino backbone, a bridged nucleic acid backbone, a locked nucleic acid (LNA) backbone, or a 2′ modification. In some embodiments, one or both of the LT1 and/or LT2 comprise a phosphorothioate backbone. In some embodiments, one or both of the LT1 and/or LT2 comprise a phosphoramidate backbone. In some embodiments, one or both of the LT1 and/or LT2 comprise a morpholino backbone. In some embodiments, one or both of the LT1 and/or LT2 comprise a bridged nucleic acid backbone. In some embodiments, one or both of the LT1 and/or LT2 comprise an LNA backbone. In some embodiments, the cell is contacted with an equimolar mixture of the two ligation-template oligonucleotides.

In some embodiments, the nuclease resistant oligonucleotide comprises a plurality of 2′ modifications. In some embodiments, the plurality of 2′ modifications are chosen from —O((CH₂)ₙO)ₘCH₃, —O(CH₂)ₙOCH₃, —O(CH₂)ₙNH₂, —O(CH₂)ₙCH₃, —O(CH₂)ₙ—ONH₂, and —O(CH₂)ₙON((CH₂)ₙCH₃))₂, or any combination thereof, where n and m are, independently, from 0 to about 10. In some embodiments, the plurality of 2′ modifications are —O((CH₂)ₙO)ₘCH₃, where n and m are, independently, from 0 to about 10. In some embodiments, the plurality of 2′ modifications are —O(CH₂)ₙOCH₃, where n is from 0 to about 10. In some embodiments, the plurality of 2′ modifications are is —O(CH₂)ₙNH₂, where n is from 0 to about 10. In some embodiments, the plurality of 2′ modifications are —O(CH₂)ₙCH₃, where n is from 0 to about 10. In some embodiments, the plurality of 2′ modifications are —O(CH₂)ₙ—ONH₂, where n is from 0 to about 10. In some embodiments, the plurality of 2′ modifications are —O(CH₂)ₙON((CH₂)ₙCH₃))₂, where each n is, independently, from 0 to about 10. In some embodiments, the plurality of 2′ modifications are 2′-O-methyl groups.

In some embodiments, the ligase is Splint® ligase.

In some embodiments, the methods further comprise amplifying the reverse template. In some embodiments, the methods further comprise quantifying the reverse template. In some embodiments, the methods further comprise amplifying and quantifying the reverse template.

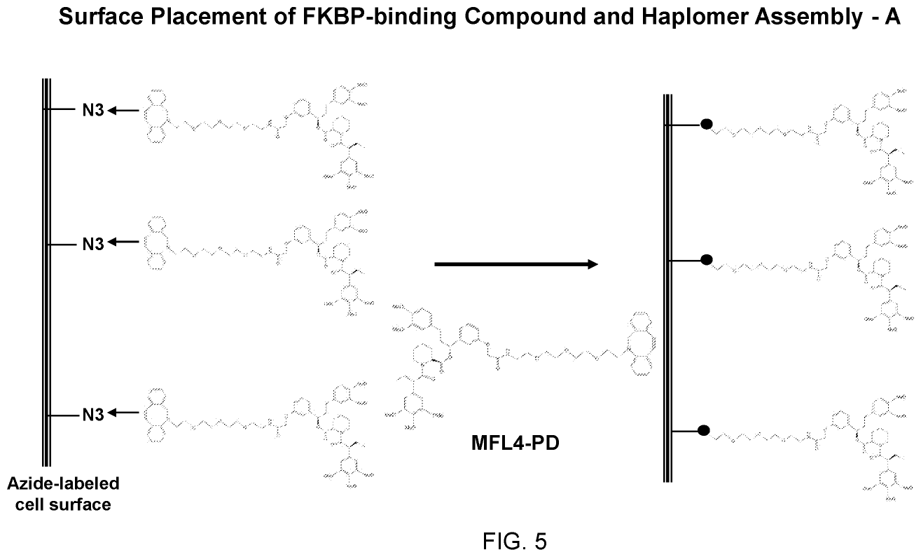

The present disclosure also provides methods of surface placement of FKBP-binding compounds and haplomer assembly. In some embodiments, cell surfaces bearing metabolically-placed azide groups are treated with a bifunctional compound. Such compounds can be used to react with surface azides and thereby position the FKBP-binding moiety on cell surfaces (see, FIG. 5, shown with the representative MFL4-PD bearing a DBCO group).

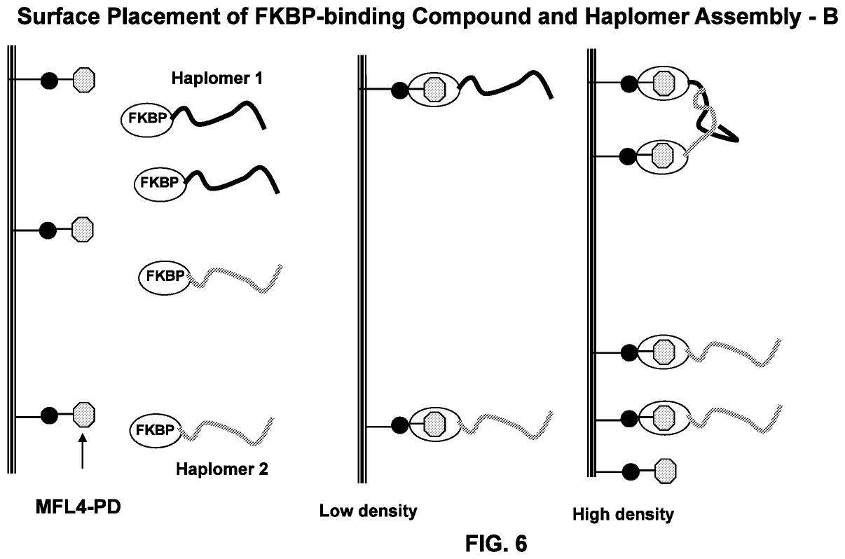

Subsequent to the labeling of a cell surface with the FKBP-binding moiety, cells can be treated with, for example, two separate split-protein haplomers where each is fused with the FKBP domain, including, but not limited to, variants of FKBP where the cysteine at position 22 is replaced by a valine residue (see, PCT Publication WO 18/94195). At conditions of low glycan density, surface haplomers are infrequently in close proximity, whereas the frequency of haplomer association rises in proportion to density (see, FIG. 6; as associated with surface azide labeling). Close associations between only two N-terminal or two C-terminal haplomers are non-productive, but the higher the surface labeling, the higher the probability of productive associations occurring (see, FIG. 6), where these are measurable through a reporter assay, such as Gaussia luminescence assay.

In some embodiments, the bifunctional compounds comprise the formula:

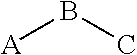

wherein: A is a small molecule ligand that Binds to an FKBP binding site; B is a chemical linker chosen from an alkyl, an alkenyl, an amide, an ester, a thioester, a ketone, an ether, a thioether, a disulfide, an ethylene glycol unit: a cycloalkyl, a benzyl, a heterocyclic, a maleimidyl, a hydrazone, a urethane, an azole, an imine, a haloalkyl, or a carbamate, or any combination thereof; and C is an azide reactive molecule chosen from a cyclooctyne, a norbornene, an oxanorbornadiene, a phosphine, a dialkyl phosphine, a trialkyl phosphine, a phosphinothiol, a phosphinophenol, a cyclooctene, a tetrazine, a tetrazole, or a quadricyclane.

In some embodiments, the cell is contacted with the azide-modified sugar prior to contacting the cell with the bifunctional compound. In some embodiments, the azide-modified sugar is AzNAM, AzGlcNAc, AGalNAc, or AzNANA. In some embodiments, the azide-modified sugar is AzNAM. In some embodiments, the azide-modified sugar is AzGlcNAc. In some embodiments, the azide-modified sugar is AGalNAc. In some embodiments, the azide-modified sugar is AzNANA. In some embodiments, the azide-modified sugar is acetylated at 1, 2, 3, or 4 positions. In some embodiments, the azide-modified sugar is acetylated at 1 position. In some embodiments, the azide-modified sugar is acetylated at 2 positions. In some embodiments, the azide-modified sugar is acetylated at 3 positions. In some embodiments, the azide-modified sugar is acetylated at 4 positions.

In some embodiments, the methods comprise contacting the cell having an azide-modified sugar on its surface with a complex. In some embodiments, the complex comprises an FKBP binding site linked to a polynucleotide, peptide, or small molecule. In some embodiments, the complex comprises an FKBP binding site linked to a polynucleotide. In some embodiments, the complex comprises an FKBP binding site linked to a peptide. In some embodiments, the complex comprises an FKBP binding site linked to a small molecule. In some embodiments, the FKBP binding site is the FK506-FKBP binding site or the mutant (F36V) FKBP binding site. In some embodiments, the FKBP binding site is the FK506-FKBP binding site. In some embodiments, the FKBP binding site is the mutant (F36V) FKBP binding site. In some embodiments, the complex comprises the FK506-FKBP binding site linked to a polynucleotide. In some embodiments, the complex comprises the mutant (F36V) FKBP binding site linked to a polynucleotide. The polynucleotide portion of the complex can serve as, for example, a template polynucleotide for a template assembly by proximity-enhanced reactivity process to occur.

In any of the methods described herein, the FKBP binding site to which the small molecule ligand A binds is the FK506-FKBP binding site or the mutant (F36V) FKBP binding site. In some embodiments, the FKBP binding site is the FK506-FKBP binding site. In some embodiments, the FKBP binding site is the mutant (F36V) FKBP binding site. In some embodiments, the small molecule ligand is

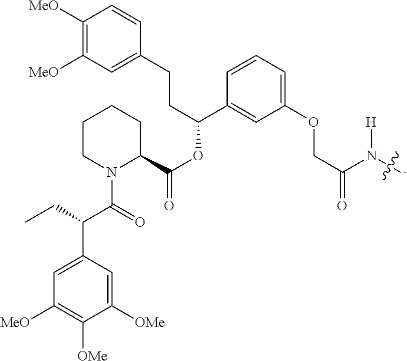

In any of the methods described herein, the chemical linker B is chosen from an alkyl, an alkenyl, an amide, an ester, a thioester, a ketone, an ether, a thioether, a disulfide, an ethylene glycol unit, a cycloalkyl, a benzyl, a heterocyclic, a maleimidyl, a hydrazone, a urethane, an azole, an imine, a haloalkyl, or a carbamate, or any combination thereof. In some embodiments, the chemical linker is chosen from an alkyl, an alkenyl, an amide, an ester, a thioester, a ketone, an ether, a thioether, a disulfide, an ethylene glycol unit, a haloalkyl, or a carbamate. In some embodiments, the chemical linker is chosen from an alkyl, an alkenyl, an amide, an ester; a thioester, a disulfide, an ethylene glycol unit, or a haloalkyl. In some embodiments, the chemical linker is chosen from an alkyl, an alkenyl, an amide, an ethylene glycol unit, or a haloalkyl. In some embodiments, the chemical linker is an alkyl or an ethylene glycol unit. In some embodiments, the chemical linker is an alkyl. In some embodiments, the alkyl is a C₂-C₁₆alkyl. In some embodiments, the alkyl is a C₄-C₁₂alkyl or a C₄-C₁₆alkyl. In some embodiments, the alkyl is a C₄-C₁₀alkyl. In some embodiments, the alkyl is C₄alkyl or C₁₀alkyl. In some embodiments, the chemical linker is an ethylene glycol unit. In some embodiments, the ethylene glycol unit is a polyethylene glycol (PEG). In some embodiments, the ethylene glycol unit is PEG2 to PEG16. In some embodiments, the ethylene glycol unit is PEG2, PEG3, or PEG4.

In any of the methods described herein, the azide reactive molecule C is chosen from a cyclooctyne, a norbomene, an oxanorhornadiene, a phosphine, a dialkyl phosphine, a trialkyl phosphine, a phosphinothiol, a phosphinophenol, a cyclooctene, a tetrazine, a tetrazole, or a quadricyclane. In some embodiments, the azide reactive molecule is chosen from a cyclooctyne, a norbomene, a phosphine, a dialkyl phosphine, a trialkyl phosphine, a cyclooctene, a tetrazine, a tetrazole, or a quadricyclane. In some embodiments, the azide reactive molecule is chosen from a cyclooctyne, a norbornene, a phosphine, a cyclooctene, a tetrazine, or a tetrazole. In some embodiments, the azide reactive molecule is chosen from a cyclooctyne, a cyclooctene, and a tetrazine. In some embodiments, the cyclooctyne is DBCO, BCN, monofluorinated cyclooctyne, difluorocyclooctyne, dimethoxyazacyclooctyne, dibenzoazacyclooctyne, biarylazacyclooctynone, 2,3,6,7-tetramethoxy-dibenzocyclooctyne, sulfonylated dibenzocyclooctyne, carboxymethylmonobenzocyclooctyne, or pyrrolocyclooctyne. In some embodiments, the cyclooctyne is DBCO or BCN. In some embodiments, the cyclooctyne is DBCO. In some embodiments, the cyclooctyne is BCN. In some embodiments, the cyclooctene is TCO. In some embodiments, the tetrazine is methyltetrazine, diphenyltetrazine, 3,6-di-(2-pyridyl)-s-tetrazine, 3,6-diphenyl-s-tetrazine, 3(5-aminopyridin-2-yl)-6-(pyridin-2-yl)-s-tetrazine, or N-benzoyl-3-(5-aminopyridin-2-yl-6-(pyridin-2-yl)-s-tetrazine. In some embodiments, the tetrazine is methyltetrazine or diphenyltetrazine. In some embodiments, the tetrazine is methyltetrazine.

In any of the methods described herein, for the bifunctional compound, the FKBP binding site is the FK506-FKBP binding site or the mutant (F36V) FKBP binding site; the chemical linker is an alkyl or an ethylene glycol unit; and the azide reactive molecule is chosen from a cyclooctyne, a cyclooctene, and a tetrazine.

In any of the methods described herein, for the bifunctional compound, the FKBP binding site is the FK506-FKBP binding site or the mutant (F36V) FKBP binding site; the chemical linker is a C₂-C₁₆alkyl or a polyethylene glycol which is PEG2 to PEG16; and the azide reactive molecule is DBCO, BCN. TCO, or methyltetrazine.

In any of the methods described herein, for the bifunctional compound, the FKBP binding site is the mutant (F36V) FKBP binding site; the chemical linker is a C₄-C₁₀alkyl or a polyethylene glycol which is PEG2, PEG3, or PEG4; and the azide reactive molecule is DBCO, BCN, TCO, or methyltetrazine.

In any of the methods described herein, for the bifunctional compound, the small molecule ligand is

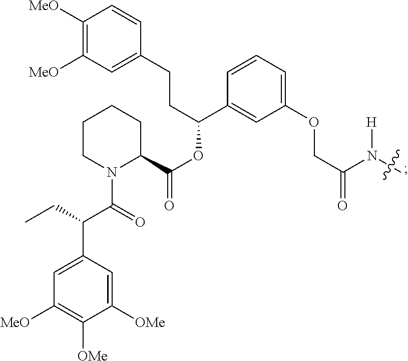

the chemical linker is C₄alkyl, C₁₀alkyl, or PEG3; and the azide reactive molecule is DBCO or BCN.

In any of the methods described herein, the bifunctional compound comprises the formula:

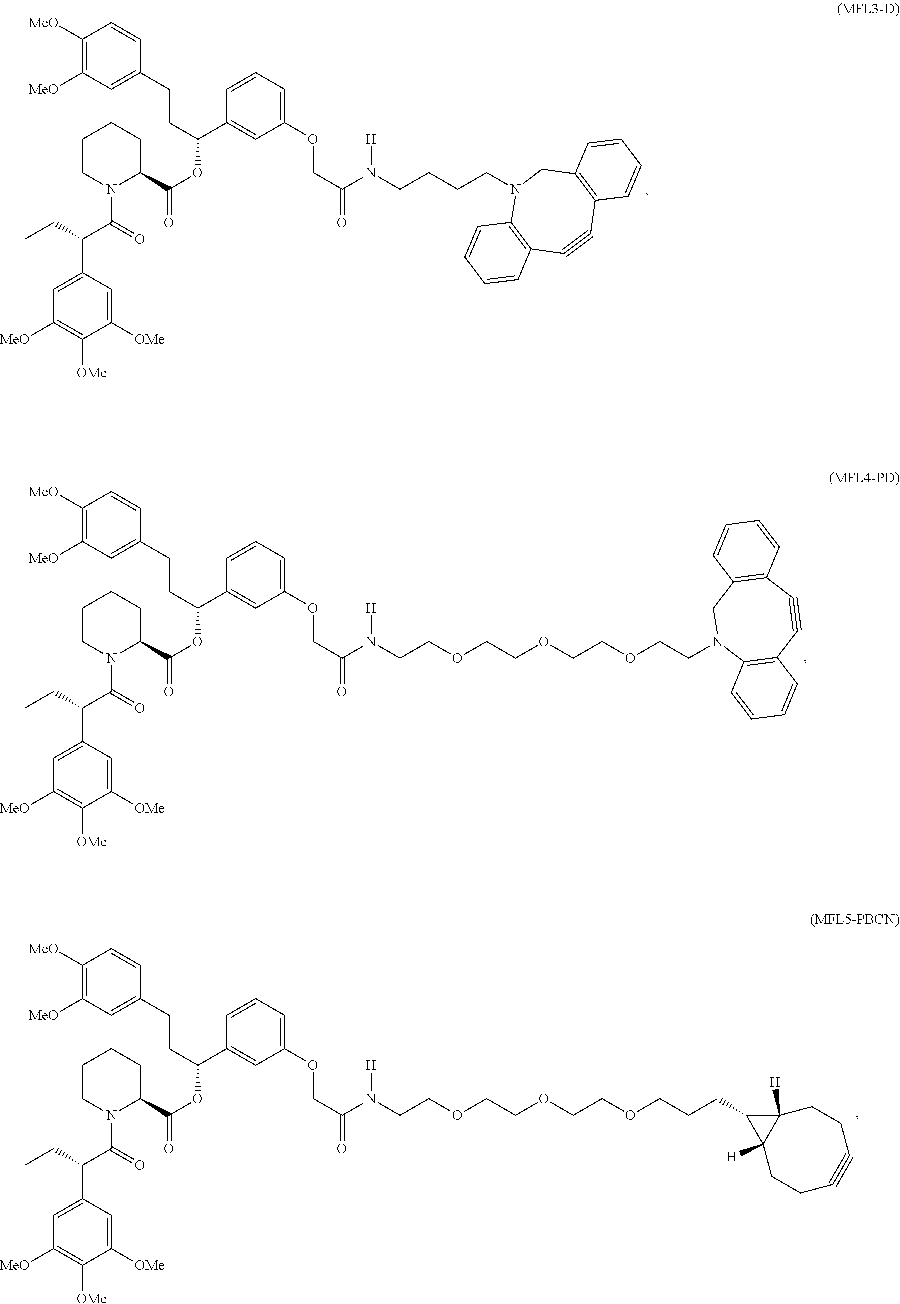 [SMILES: CC[C@H](C(=O)N1CCCC[C@H]1C(=O)O[C@H](CCc1ccc(OC)c(OC)c1)c1cccc(OCC(=O)NCCCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCC[C@@H]2[C@@H]3CCC#CCC[C@@H]32)c1)c1cc(OC)c(OC)c(OC)c1]

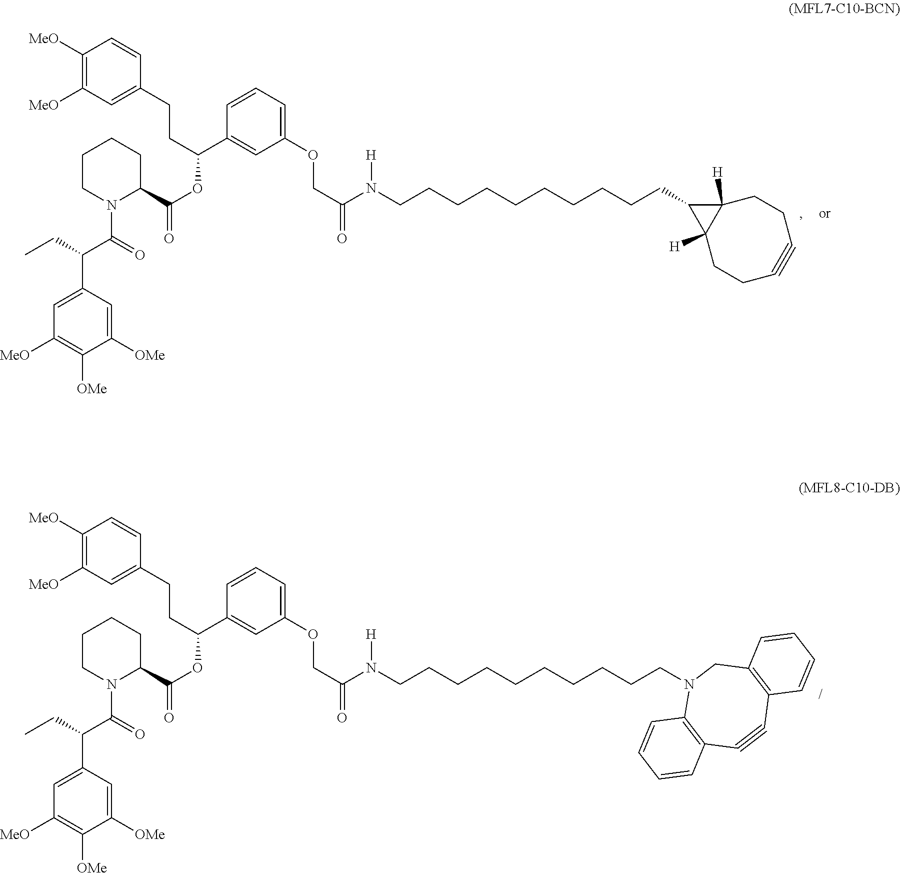 [SMILES: CC[C@H](C(=O)N1CCCC[C@H]1C(=O)O[C@H](CCc1ccc(OC)c(OC)c1)c1cccc(OCC(=O)NCCCCCCCCCC[C@@H]2[C@@H]3CCC#CCC[C@@H]32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCCCCCCCCCN2Cc3ccccc3C#Cc3ccccc32)c1)c1cc(OC)c(OC)c(OC)c1]

In some embodiments, the complex comprising the FKBP binding site linked to a polynucleotide, peptide, or small molecule can be pre-incubated with a bifunctional compound (such as an excess amount of bifunctional compound) prior to exposure to the target cells displaying surface azide. The resulting complex-bifunctional compound can then used to treat cells having surface azide.

In any of the methods described herein, the cell can be any desired target cell. In some embodiments, the cell is a virus infected cell, a tumor cell, a cell infected with a microbe, or a cell that produces a molecule that leads to a disease, such as a cell that produces an antibody that induces allergy, anaphylaxis, or autoimmune disease, or a cytokine that mediates a disease. The cells described herein can be contacted with any of the azide-modified sugars described herein either in vitro or in vivo. The cells described herein can also be contacted with any of the bifunctional compounds described herein either in vitro or in vivo. The cells described herein can also be contacted with any of the complexes described herein either in vitro or in vivo.

In some embodiments, the methods described herein further comprise contacting the cell with a substrate for a templated assembly reaction, wherein the substrate comprises: i) a nucleic acid template; and ii) an azide reactive molecule linked to the nucleic acid template at the 5′- or 3′-end of the nucleic acid template, wherein the azide reactive molecule is chemically reactable with the azide of the azide-modified sugar on the surface of the cell.

In some embodiments, the nucleic acid template is chosen from a cancer-specific polynucleotide, a viral polynucleotide, a microbial-specific polynucleotide, a differentially expressed gene, and a disease-specific polynucleotide. In some embodiments, the nucleic acid template is a cancer-specific polynucleotide. In some embodiments, the nucleic acid template is a viral polynucleotide. In some embodiments, the nucleic acid template is a microbial-specific polynucleotide. In some embodiments, the nucleic acid template is a differentially expressed gene. In some embodiments, the nucleic acid template is a disease-specific polynucleotide.

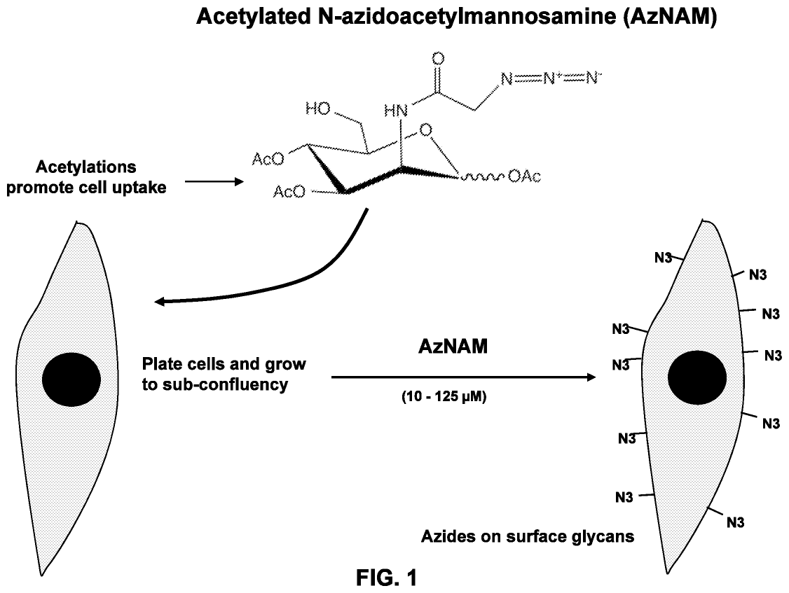

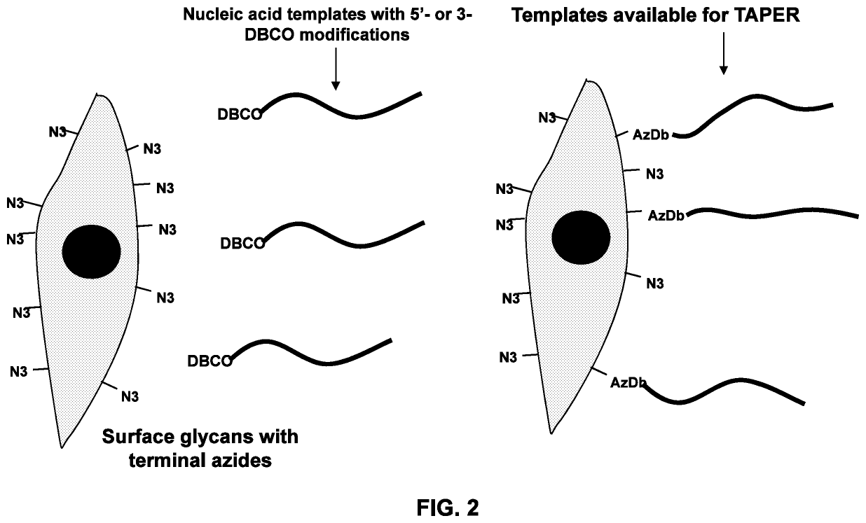

The present disclosure provides methods of displaying molecular entities on cells that are thereby targeted for destruction/removal. In some embodiments, target cells are treated with acetylated azido-N-acetylmannosamine (AzNAM; see, FIG. 1), and then with nucleic acid templates labeled with a selectively-reactive moiety to allow a bio-orthogonal reaction between the metabolically derived surface azide and the selectively-reactive moiety. Such selectively-reactive moieties can include, but are not limited to, DBCO, BCN, methyltetrazine, and TCO. Treatment of cells with surface azide following growth in the presence of AzNAM is depicted in FIG. 2, with DBCO as a representative selectively-reactive moiety. Surface placement of a template in this manner allows TAPER reactions to occur (see, PCT Publication WO 14/197547), including split-protein (see, PCT Publication WO 16/89958) and ligand-dependent forms of TAPER (PCT Publication WO 18/94195). Any nucleic acid molecule can be a possible nucleic acid template for nucleic acid templated assembly provided that at least some sequence information is available and is sufficient to bind a nucleic acid recognition moiety of the haplomer either directly or indirectly. Examples of nucleic acid recognition moieties include polynucleotide or oligonucleotides, peptide nucleic acid oligomers, and morpholino oligomers. Examples of nucleic acid templates include, but are not limited to, mRNA, genomic or organellar DNA, episomal or plasmid DNA, viral DNA or RNA, miRNA, rRNA, snRNA, tRNA, or any other biological or artificial nucleic acid sequence.

In some embodiments, the nucleic acid template can be present in a target compartment but absent in a non-target compartment. An example of this embodiment includes nucleic acid molecules present in a pathogenic or diseased cell, but absent in a healthy cell.

Any cell, virus, tissue, spatial region, lysate, or other subcomponent of a sample that contains a nucleic acid template can be targeted. Target compartments that contain the nucleic acid template include, but are not limited to, pathogenic cells, cancer cells, viruses, host cells infected by a virus or other pathogen, or cells of the immune system that are contributing to autoimmunity such as cells of the adaptive or innate immune systems, transplant rejection, or an allergic response. In some embodiments, a nucleic acid template can be present in a virus or cell infected by a virus, but absent in healthy cells. Examples of viruses include, but are not limited to, DNA viruses, RNA viruses, or reverse transcribing viruses. In some embodiments, a nucleic acid template can be present in a tumor or cancerous cell, but absent in healthy cells. Examples of cancers can include, but are not limited to, those caused by oncoviruses, such as the human papilloma viruses, Epstein-Barr virus, hepatitis B virus, hepatitis C virus, human T-lymphotropic viruses, Merkel cell polyoma virus, and Kaposi's sarcoma-associated herpesvirus. In some embodiments, a nucleic acid template can be present in an infectious agent or microbe, or a cell infected by an infectious agent or microbe but is absent in healthy cells. Examples of infectious agents or microbes include, but are not limited to, viruses, bacteria, fungi, protists, prions, and eukaryotic parasites.

Nucleic acid templates can also be a fragment, portion or part of a gene, such as an oncogene, a mutant gene, an oncoviral gene, a viral nucleic acid sequence, a microbial nucleic acid sequence, a differentially expressed gene, and a nucleic acid gene product thereof.

Examples of virus-specific nucleic acid templates include, but are not limited to, sequences present in DNA viruses, RNA viruses, and reverse transcribing viruses. Examples of cancer-specific nucleic acid templates include, but are not limited to, sequences derived from oncoviruses, including, but not limited to, human papilloma virus, Epstein-Barr virus, hepatitis B virus, hepatitis C virus, human T-lymphotropic virus, Merkel cell polyoma virus, and Kaposi's sarcoma-associated herpesvirus. Examples of cancer-specific nucleic acid templates include mutant oncogenes, such as, but not limited to, mutated ras, HRAS, KRAS, NRAS, BRAF, EGFR, FLT1, FLT4, KDR, PDGFRA, PDGFRB, ABL1, PDGFB, MYC, CCND1, CDK2, CDK4, or SRC genes; mutant tumor suppressor genes, such as TP53, TP63, TP73, MDM1, MDM2, ATM, RBI, RBL1, RBL2, PTEN, APC, DCC, WT1, IRF1, CDK2AP1, CDKN1A, CDKN1B, CDKN2A, TRIM3, BRCA1, or BRCA2 genes; and genes expressed in cancer cells, where the gene may not be mutated or genetically altered, but is not expressed in healthy cells of a sample at the time of administration, such as carcinoembryonic antigen.

In some embodiments, the nucleic acid template can be present in a differential amounts or concentrations in the target compartments as compared to the non-target compartments. Examples of nucleic acid templates include, but are not limited to, genes expressed at a different level in cancer cells than in healthy cells, such as myc, telomerase, HER2, or cyclin-dependent kinases. In some embodiments, the nucleic acid template is a gene that is at least 1.5× fold differentially expressed in the target versus the non-target compartments. Examples of these include, but are not limited to, genes related to mediating Type I allergic responses, for which target RNA molecules contain immunoglobulin epsilon heavy chain sequences; genes expressed in T cell subsets, such as specific T cell receptors (TCRs) which recognize self-antigens in the context of particular major histocompatibility (MHC) proteins like proinsulin-derived peptide and clonally-specific mRNAs containing a or β variable-region sequences, derived from diabetogenic CD8⁺ T cells; and cytokines whose production may have adverse outcomes through exacerbation of inflammatory responses, including but not limited to TNF-alpha, TNF-beta, IL-1, IL-2, IL-4, IL-6, IL-8, IL-10, IL-12, IL-15, IL-17, IL-18, IL-21, IL-22, IL-27, IL-31, IFN-gamma, OSM, and LIF.

In some embodiments, the nucleic acid template is present in target compartments and an acceptable subgroup of non-target compartments, but not in a different or distinct subgroup of non-target compartments. Examples include, but are not limited to, genes expressed in cancer cells and limited to classes of healthy cells, such as cancer-testis antigens, survivin, prostate-specific antigen, carcinoembryonic antigen (CEA), alpha-fetoprotein and other onco-fetal proteins. Also, many tissues and organs are not essential to otherwise healthy life in the face of serious disease. For example, melanocyte antigens, such as Melan-A/MART-1 and gp100 are expressed on many malignant melanomas as well as normal melanocytes, and therapies that target these antigens can destroy both tumors and normal melanocytes, resulting in vitiligo, but major tumor reduction. Likewise, the reproductive organs may be surgically removed, such as testis, ovary and uterus, as well as associated organs such as breast and prostate may be targeted when tumors of these tissues arise, and destruction of normal tissues within these organs may be a tolerable consequence of therapy. Furthermore, some cells that produce hormones, such as thyroxine and insulin can be replaced with the relevant protein, allowing potential targeting of normal cells that may exist in the presence of tumors of these origins.

Nucleic acid templates also include novel sequences, not previously identified. In some embodiments, a sample or samples can be evaluated by sequence analysis, such as next-generation sequencing, whole-transcriptome (RNA-seq) or whole-genome sequencing, microarray profiling, serial analysis of gene expression (SAGE), to determine the genetic makeup of the sample. Nucleic acid templates can be identified as those present in target compartments, but not present in non-target compartments, or present in differential amounts or concentrations in target compartments as compared to non-target compartments. Nucleic acid templates identified by this method can then serve as nucleic acid templates for TAPER reactions.

The active effector agent is the trigger that drives a desired action in the sample. Examples of active effector agents can include, but are not limited to, those that induce an immune response, programmed cell death, apoptosis, non-specific or programmed necrosis, lysis, growth inhibition, inhibition of viral infection, inhibition of viral replication, inhibition of oncogene expression, modification of gene expression, inhibition of microbial infection, and inhibition of microbe replication, as well as combinations of these biological activities. In some embodiments, the active effector agent can serve as a ligand for an antibody to induce an immune response at the site of the pathogenic cells, or to localize antibody-directed therapies, such as an antibody bearing a therapeutic payload, to the site of the pathogenic cells. In some embodiments, the active effector agent can modulate expression of a target gene. In some embodiments, the active effector agent can regulate enzyme activity, gene/protein expression, molecular signaling, and molecular interactions.

An active effector agent is a product of a combination of haplomers, or a combination of portions of haplomers, that produces a desired activity in a sample. The active effector agent can possess a targeted activity or an elevated level of activity as compared to either or both of the effector partial moieties individually. In some embodiments, the active effector agent can possess a new or substantially different activity than the individual effector partial moieties individually.

A diverse array of active effector agents can be produced by nucleic acid templated assembly. Any active product can serve as an active effector agent as long as such an agent can be produced by the templated assembly of relatively inactive effector partial moieties that can be combined by reaction of corresponding selectively-reactive moieties. Thus, any compound that can be reconstituted from separate portions (i.e., effector partial moieties) by formation of an amide bond, triazole linkage, phosphine oxide linkage, or other bio-orthogonal ligation product as described herein can serve as an active effector agent. Furthermore, such agents can be assembled on virtually any accessible nucleic acid template, thus allowing assembly in a very diverse set of samples.

Active effector agents also include proteins, peptides containing standard or non-standard amino acids, peptidomimetic structures, and drugs or other bioactive compounds that permit or require the interaction or incorporation of the both effector partial moieties.

In some embodiments, active effector agents can be liberated from the other moieties in the templated assembly product by cleavage of the bonds connecting the effector partial moiety to the remainder of the product. Cleavage can be achieved by hydrolysis of the connecting bonds, or by enzymatic cleavage by proteins or other compounds endogenous to the sample. Examples of these cleavable bonds include, but are not limited to, esters, thioesters, imines, hydrazones, cleavage motifs of cellular proteases, or substrates of cellular enzymes. Cleavable groups can be introduced by their incorporation into a haplomer, linker, or accessory group during synthesis, or can be generated during the ligation reaction. In some embodiments, post-ligation cleavage or other in situ chemical modification of the active effector agent may be required for the active effector agent to trigger a desired activity.

An active effector agent can also trigger activity by acting within a target compartment (for example, within a cell), at the surface of a target compartment (for example, at the cell surface), in the vicinity of the target compartment (for example, when the active effector agent is actively exported from the cell, leaks from the cell, or released upon cell death), or diffuse or be carried to a distant region of the sample to trigger a response. In some embodiments, active effector agents can be targeted to their active sites by incorporation of targeting groups in the haplomer. Examples of targeting groups can include endoplasmic reticulum transport signals, golgi apparatus transport signals, nuclear transport signals, mitochondrial transport signals, ubiquitination motifs, other proteosome targeting motifs, or glycosylphosphatidylinositol anchor motifs. Targeting groups can be introduced by their incorporation into a haplomer, chemical linker, or accessory group during synthesis, or can be generated during the ligation reaction.

In some embodiments, the active effector agent can be presented on the surface of a target compartment. In some embodiments, the active effector agent can be presented on the surface of a cell as a ligand bound to a major histocompatibility complex (MHC) molecule.

In some embodiments, the active effector agents can be endogenous peptides, or their analogs, or completely synthetic structures which are targets for other agents, such as antibodies. Availability of nucleic acid templates can limit production of active effector agents, therefore it may be desirable to have active effector agents that exert activity when present at low levels.

Active effector agents can also be produced by templating on accessible nucleic acid templates in a highly diverse set of samples, and combinations of active effector agents can be produced on the same nucleic acid template, or on different nucleic acid templates that are simultaneously present within a sample, such as a cell. Thus, a single active effector agent can be assembled on different nucleic acid templates within the same sample, or several active effector agents can be assembled on the same nucleic acid template, or several nucleic acid templates within the same sample, producing more copies of a particular active effector agent, as well as a more diverse array of active effector agents on available nucleic acid templates within a sample.

Specific cellular populations can be modulated through the generation of active effector agents, which ultimately result in the destruction or alteration of designated cellular target compartments. Active effector agent-generated activity can be designed to delete undesired cellular target compartments. For example, FIG. 8 of PCT Publication WO 14/197547 illustrates exemplary active effector agents employing different mechanisms to induce apoptosis, such as cytotoxic T-lymphocytes, therapeutic antibodies, intracellular receptors, and direct cellular interaction.

In some embodiments, killing or growth inhibition of target cells can be induced by direct interaction with cytotoxic, microbicidal, or virucidal active effector agents. Numerous toxic molecules known in the art can be produced. In some embodiments, traceless bio-orthogonal reactive chemistry can produce toxic peptides such as, for example, bee melittin, conotoxins, cathelicidins, defensins, protegrins, and NK-lysin.

In some embodiments, killing or growth inhibition of target cells can be induced by pro-apoptotic active effector agents. For example, peptides produced using traceless bio-orthogonal chemistry include pro-apoptotic peptides such as, for example, prion protein fragment 106-126 (PrP 106-126), Bax-derived minimum poropeptides associated with the caspase cascade including Bax 106-134, and pro-apoptotic peptide (KLAKLAKKLAKLAK; SEQ ID NO:1).

In some embodiments, the active effector agent produced can be thrombogenic, in that it induces activation of various components of the clotting cascade of proteins, or activation of proteins, or activation and/or aggregation of platelets, or endothelial damage that can lead to a biologically active process in which a region containing pathogenic cells can be selectively thrombosed to limit the blood supply to a tumor or other pathogenic cell. These types of active effector agents can also induce clotting, or prevent clotting, or prevent platelet activation and aggregation in and around targeted pathogenic cells.

In some embodiments, active effector agents mediate killing or growth inhibition of target cells or viruses by activating molecules, pathways, or cells associated with the immune system. Active effector agents can engage the innate immune system, the adaptive immune system, and/or both.

In some embodiments, active effector agents can mediate killing or growth inhibition of cells or viruses by stimulation of the innate immune system. In some embodiments, active effector agents include pathogen-associated molecular patterns (PAMPs), damage-associated molecular patterns (DAMPs), and synthetic analogs thereof. In some embodiments, the innate immune system can be engaged by active effector agents that activate the complement system. An example of a complement activating active effector agents is the C3a fragment of complement protein C3.

In some embodiments, active effector agents are agonists of formylated peptide receptors. In some embodiments, the formylated tripeptide formyl-Met-Leu-Phe can be produced using traceless bio-orthogonal chemistry. In some embodiments, small peptide agonists of the formylated peptide receptor such as the peptide Trp-Lys-Tyr-Met-Val-(D-Met) can be produced.

In some embodiments, active effector agents with natural or synthetic ligands of Toll-Like Receptors (TLR) can be produced. In some embodiments, an active effector agent can include peptide fragments of heat shock proteins (hsp) known to be TLR agonists.

In some embodiments, traceless bio-orthogonal chemistry can be used to produce the muramyl dipeptide agonist of the NOD2 receptor to activate an inflammatory response.

In some embodiments, active effector agents can mediate killing or growth inhibition of cells or viruses by activating molecules or cells of the adaptive immune system. Unique to the adaptive immune system, molecules or cells can be engineered to recognize an extraordinary variety of structures, thus removing the constraint that the active effector agents must be intrinsically active or bind to an endogenous protein.

Because of the modularity of the system, a single engineered molecule or cell of the adaptive immune system can be utilized for therapy of any target compartments or nucleic acid templates, since the same active effector agents can be produced in the presence of any nucleic acid template. This is an advantage over the current state of the art, where new molecules or cells must be engineered to treat any new target, involving significant time, difficulty, and cost.

In some embodiments, active effector agents can be a ligand for an antibody or antibody fragment (including but not limited to Fab, Fv, and scFv). Traceless bio-orthogonal approaches can be used to produce a peptide or other epitope that is bound by an existing antibody, or an antibody can be developed to recognize active effector agents created by any selectively reactive or bio-orthogonal approach.

In some embodiments, an antibody may be used for detection of active effector agents in vivo, thus localizing a target compartment within a subject.

In some embodiments, active effector agents can activate T-cells. Activation of T-cells can be achieved by active effector agents binding to a T-cell receptor (TCR). In some embodiments, active effector agents can be presented on the surface of a target cell bound to an MHC molecule, facilitating binding of a T-cell receptor. Active effector agents can be bound by MHC class I or MHC class II molecules. In some embodiments, active effector agents are bound by MHC class I molecules. The structure that binds to the TCR can be a conventional peptide antigen, or a “superantigen” that binds to a broad subset of T cells that express a particular variable (V) region. As opposed to a TCR that is selected to interact with specific antigen, a superantigen can activate a large number of T cell populations that have receptors capable of binding to different antigen-MHC complexes, and induce a strong inflammatory response to set off a cascade of inflammatory mediators. Thus, a superantigen or superantigen mimetic can be produced as active effector agents that can recruit large numbers of T cells to a pathogenic cell, and lead to destruction or limitation in the growth of such cells.

Natural ligands bound to MHC class I molecules are typically peptides of 8 to 10 amino acids in length, though other lengths are permissible. Natural ligands bound to MHC class II molecules are typically peptides of 15 to 24 amino acids in length, though other lengths are permissible. Active effector agents can be produced using traceless bio-orthogonal chemistry. A peptide that is a known MHC ligand can be utilized as an active effector agent, or a novel peptide can be produced. Assays for evaluating binding of peptides to MHC molecules are known in the art, and can be used to evaluate candidate active effector agents for MHC binding Peptidomimetic active effector agents can be designed based on a natural ligand known to bind MHC and activate a T-cell receptor (as in the examples above). Alternately, peptidomimetic active effector agents can be an entirely new structure, and a new T-cell clone or antibody-TCR chimera (T-body) can be developed for use as active effector agents. This approach offers the benefit of using highly non-self, non-cross-reactive active effector agents which can increase activity while reducing undesired side-effects during therapy.

In some embodiments, natural peptide or peptidomimetic MHC-binding active effector agents can be utilized in conjunction with adoptive T-cell therapy. An adoptive T cell therapy provides a patient with exogeneous T cells which can accomplish a therapeutically desirable immunoreaction.

Once active effector agents have been selected, appropriate selectively-reactive moieties and effector partial moieties can be designed for incorporation into the haplomers. These moieties are designed such that they can reconstitute the active effector agent when a templated assembly reaction occurs.

A haplomer includes at least one nucleic acid recognition moiety. The nucleic acid recognition moiety is the targeting component of the haplomer that recognizes a nucleic acid template that serves as a target sequence and interacts in a sequence-specific manner with the nucleic acid template via Watson-Crick or Hoogsteen base-pairing interactions. The nucleic acid recognition moiety can bind to the nucleic acid template or facilitate binding to the nucleic acid template. In some embodiments, the nucleic acid recognition moiety binds directly to the nucleic acid template.

In some embodiments, the nucleic acid recognition moiety is a polynucleotide that binds to a nucleic acid template. The binding can be through direct hybridization of the nucleic acid recognition moiety with the nucleic acid template or indirectly through an intermediate, such as a linker, that binds both the nucleic acid recognition moiety and the nucleic acid template.

The polynucleotide sequence can be DNA nucleotides, RNA nucleotides, phosphorothioate-modified nucleotides, phosphoramidate-modified nucleotides, 2-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, bridged nucleic acid backbone, other nucleic acid analogues capable of base-pair formation, or combinations thereof. In some embodiment, the nucleic acid recognition moiety includes nucleic acids and hybridizes to mRNA targets.

The polynucleotides can also incorporate, interact with or be bound to specialized units. For example, when using the nucleic acid recognition moieties in the presence of nucleases that degrade standard DNA or RNA, such as in live cells or lysates, it may be desirable to incorporate nuclease-resistant bases into the polynucleotide. Examples include, but are not limited to, phosphorothioate bases, 2-O-alkylated or 2-halogenated RNA bases, locked nucleic acids, peptide nucleic acids, morpholinos or a chimera including at least one of these. Unlike antisense probes that depend on RNase H activity, internal bases of the oligomer need not induce RNase H hydrolysis of a target RNA transcript. Thus, there is no requirement for RNase H-inducing bases at any position in the nucleic acid recognition moiety.

The sequence of bases in a nucleic acid recognition moiety can be complementary to a hybridization site on a nucleic acid template, allowing sequence-specific binding of the nucleic acid recognition moiety to the nucleic acid template. In some embodiments, the hybridization site is selected such that its sequence is not similar to sequences known to be present in non-target nucleic acid templates. In some embodiments, the hybridization site includes one or more mutations found within the nucleic acid template, allowing specific binding of nucleic acid recognition moiety to the nucleic acid template but not to non-target nucleic acids that do not contain the mutation. In some embodiments, the nucleic acid recognition moiety can be designed as a stem-loop structure, with possible improvement in the desired binding interaction with nucleic acid templates.

The binding site on the nucleic acid template can be anywhere from about 5 to about 100 bases in length. In some embodiments, the binding site on the nucleic acid template can be from about 5 to about 50 bases in length. In some embodiments, the binding site on the nucleic acid template can be from about 5 to about 40 bases in length. In some embodiments, the binding site on the nucleic acid template can be from about 10 to about 30 bases in length.

Likewise, the nucleic acid recognition moiety can be a polynucleotide that can bind to the nucleic acid template. The polynucleotide can be from about 5 to about 100 bases in length. In some embodiments, the polynucleotide can be from about 5 to about 50 bases in length. In some embodiments, the polynucleotide can be from about 5 to about 40 bases in length. In some embodiments, the polynucleotide can be from about 10 to about 30 bases in length.

The nucleic acid recognition moiety can also be optimized to provide a desired interaction with the nucleic acid template. The length of the nucleic acid template that the nucleic acid recognition moiety binds can be selected based on chemical properties of the complementary sequence of the nucleic acid template. Such properties include the melting and annealing temperatures of the complementary sequence. The melting temperature, Tₘ, is defined as the temperature in degrees Celsius, at which 50% of all molecules of a given nucleic acid sequence are hybridized into a double strand, and 50% are present as single strands. The annealing temperature is generally 5° C. lower than the melting temperature.

The Tₘ of the complementary sequence of the nucleic acid template can be from about 10° C. below to about 40° C. above the temperature of the conditions in which the haplomer will be used. For example, if haplomers are to be used at 37° C., the nucleic acid recognition moiety can be designed with an expected Tₘ between 27° C. to 77° C. In some embodiments, the haplomers can be used at approximately 37° C., and the Tₘ of the complementary sequence used in the nucleic acid recognition moiety can be designed to be from about 37° C. to about 52° C.

In some embodiments, the nucleic acid recognition moiety can be designed such that the Tₘ to bind the nucleic acid template is substantially different from the Tₘ to bind a similar non-target nucleic acid. For example, the nucleic acid recognition moiety can be designed such that the hybridization site it binds to on a nucleic acid template includes the site of a mutation. In some embodiments, the Tₘ of the nucleic acid recognition moiety binding to the nucleic acid template is at or above the temperature at which the haplomer will be used, while the Tₘ of the nucleic acid recognition moiety binding to the non-target nucleic acid is below the temperature at which the haplomer will be used. The nucleic acid recognition moiety will then bind to the mutant nucleic acid template, but not to the non-target, non-mutant sequence.

Binding or hybridization sites of the nucleic acid recognition moieties of members of a set of corresponding haplomers can be on the same nucleic acid template. In some embodiments, the binding or hybridization sites can be found on the same nucleic acid template but separated by about 0 to about 100 bases on the nucleic acid template. In some embodiments, the binding or hybridization sites can be separated by about 0 to about 30 bases on the nucleic acid template. In some embodiments, the binding or hybridization sites can be separated by distances greater than 30 bases on the same nucleic acid template, but be brought into closer proximity through secondary or tertiary structure formation of the nucleic acid template. In some embodiments, the binding or hybridization sites can be separated by a distance greater than 100 bases and brought into closer proximity through secondary or tertiary structure formation of the nucleic acid template.

Commercially available derivatized bases can be incorporated into the polynucleotides to introduce functional groups including, but not limited to, amines, hydrazides, thiols, carboxylic acids, isocyanates, aldehydes which can then be conjugated with active functional groups on other moieties using standard techniques of bioconjugation chemistry to facilitate synthesis of the complete haplomer.

A haplomer also includes at least one effector partial moiety. The effector partial moiety is a portion of an active effector agent, such that when a set of corresponding haplomers take part in a templated reaction, their effector partial moieties combine to produce the desired active effector agent in the templated assembly ligation product. Thus, the effector partial moiety contributes to the chemical structure of the active effector agent. The effector partial moiety can be a distinct portion of the haplomer, or can include part or all of the nucleic acid recognition moiety and/or part or all of the selectively-reactive moiety.

The effector partial moiety does not possess the targeted activity or the same level of activity associated with the active effector agent. In some instances, the effector partial moiety is substantially inactive compared to the active effector agent. In some embodiments, the individual effector partial moieties can possess separate activity, but binding the effector partials moieties together creates an activity not possessed by them individually. For example, a bivalent active effector agent that binds two different antibodies (each binds to an effector partial moiety), making the active effector agent suitable for detection in a sandwich ELISA regarding the nucleic acid templated assembly diagnostic evaluation assay.

In some embodiments, a single effector partial moiety can be present as part of the haplomer. However, a single effector partial moiety alone does not produce an active effector agent. An effector partial moiety can be positioned between the nucleic acid recognition moiety and the selectively-reactive moiety, or attached to the selectively-reactive moiety so that the selectively-reactive moiety is between the effector partial moiety and the nucleic acid recognition moiety, or both.

In some embodiments, more than one effector partial moiety can be present as part of a single haplomer. Assembly of the haplomer allows one effector partial moiety to bind to a separate effector partial moiety, that results in the production of the active effector agent. More than one effector partial moiety can be attached to the selectively-reactive moiety so that the selectively-reactive moiety is between the effector partial moieties and the nucleic acid recognition moiety. In some embodiments, the effector partial moiety includes a chemical linker capable of binding the selectively-reactive moiety.

In some embodiments, multiple haplomers can be present to produce the active effector agent. More than one haplomer can be assembled and positioned within close proximity of one another (see, FIG. 9 and FIG. 1B in PCT Publication WO 14/197547). The selectively-reactive moieties on the adjacent haplomer bind, through a chemical reaction such as a bio-orthogonal reaction, and the effector partial moieties are positioned to allow the production of the active effector agent.

Both efficiency of nucleic acid templated assembly reactions and efficiency of delivery of reactants to target compartments in a sample generally decrease with increasing size of the reactants. In some embodiments, one or more effector partial moieties are selected such that they are minimal in size while still producing an active effector agent. In some embodiments, the molecular size of an effector partial moiety is less than about 20 kDa. In some embodiments, the molecular size of an effector partial moiety is less than about 10 kDa.

The effector partial moiety can also be conjugated to other moieties on a haplomer such that the active effector agent produced can be cleaved from the templated assembly ligation product after the reaction has occurred. Cleavage can occur via hydrolysis of a bond, or be catalyzed by enzymes or other molecules within a cell. Examples of cleavage linkages include, but are not limited to, esters, thioesters, imines, hydrazones, cleavage motifs of cellular proteases, and substrates of cellular enzymes.

In embodiments in which a traceless bio-orthogonal reactive group forms a native amide bond in the active effector agent, the effector partial moiety can include a non-active portion of an active peptide, or a non-active portion of a non-peptide drug or endogenous bioactive compound that can be reconstituted via an amide bond to a corresponding portion. In embodiments in which a non-traceless bio-orthogonal reactive group incorporates a phosphine oxide, triazole, or other bio-orthogonal ligation residue, effector partial moieties can include a non-active portion of a peptidomimetic structure or non-active portion of a drug or other bioactive compound. In these embodiments, the ligated residue from the bio-orthogonal reaction can be integrated into the active effector agent.

Due to the diverse nature of effector partial moieties, various methods may be necessary for synthesis. In some embodiments, peptides are used, and effector partial moieties can be synthesized using standard Merrifield solid-phase synthesis. Synthesis approaches for other effector partial moieties are dictated by the specific chemical structure of the particular moiety.

Chemical linkers can also be incorporated into the haplomers. The chemical linkers can be included between any of the moieties. Chemical linkers can optionally connect two or more of the moieties to introduce additional functionality or facilitate synthesis. The chemical linker can be a bond between any of the moieties. In some embodiments, the chemical linker is between the nucleic acid recognition moiety and the selectively-reactive moiety, or between the selectively-reactive moiety and the effector partial moiety. In some embodiments, the effector partial moiety includes a chemical linker capable of interacting with the selectively-reactive moiety to produce the active effector agent. The bond can include a physical interaction, such as chemical bonds (either directly linked or through intermediate structures), or a non-physical interaction or attractive force, such as electrostatic attraction, hydrogen bonding, and van der Waals/dispersion forces.

The chemical linkers can aid in facilitating spatial separation of the moieties, increasing flexibility of the moieties relative to each other, introducing a cleavage site or modification site to the haplomer, facilitating synthesis of the haplomer, improving physical or functional characteristics (such as solubility, hydrophobicity, charge, cell-permeability, toxicity, biodistribution, or stability) of the haplomer, or any combination of the above. In some embodiments, the chemical linker is derived from a cross-linker that facilitates connecting the haplomer components via bioconjugation chemistry. Due to mild reaction conditions, bioconjugate chemistry approaches can be suitable for ligating biomolecules, such as nucleic acids, peptides, or polysaccharides. Examples include, but are not limited to, chains of one or more of the following: alkyl groups, alkenyl groups, amides, esters, thioesters, ketones, ethers, thioethers, disulfides, ethylene glycol, cycloalkyl groups, benzyl groups, heterocyclic groups, maleimidyl groups, hydrazones, urethanes, azoles, imines, haloalkyl groups, and carbamates, or any combination thereof.

In addition to chemical linkers between moieties, additional functionality can optionally be introduced to haplomers by the addition of accessory groups to the moieties. Examples of accessory groups include, but are not limited to, appending a chemical tag or fluorophore to track the location of a haplomer or ligation product, or appending an agent that improves delivery of a haplomer to target compartments, such as cell-penetrating peptides, or stabilizing polyethylene glycol groups. Examples of attachment points of accessory groups on suitable moieties are described herein. In some embodiments, any one or more of the nucleic acid recognition moiety, the selectively-reactive moiety, and the effector partial moiety can be functionalized with a chemical linker.

The nucleic acid recognition moiety of a haplomer can be attached to a chemical linker, effector partial moiety, or selective-reactive moiety at either end of the nucleic acid recognition moiety, or an internal portion of the nucleic acid recognition moiety. In some embodiments, the attachment point can be at one end of a nucleic acid recognition moiety, attached to a terminal unit of the nucleic acid recognition moiety directly or via a chemical linker, to prevent steric blockage of hybridization. In some embodiments, the attachment point can be at an internal point of the nucleic acid recognition moiety that does not interfere with hybridization, such as the polynucleotide backbone, or a part of a base. For example, the N-7 position of a guanine base can serve as the attachment point since it does not participate in base-pairing. The above attachment points can also be suitable positions for attachment of accessory groups to add functionality to the haplomer.

The method of synthesizing a haplomer includes generating at least one nucleic acid recognition moiety that is capable of binding a nucleic acid template, generating at least one selectively-reactive moiety that is capable of binding the nucleic acid recognition moiety, and generating an effector partial moiety. These moieties are bound together using methods known in the art, such as bioconjugate chemistry, to produce a complete haplomer. Moieties in different haplomers can be bound or attached together in different configurations, provided that the haplomers maintain proper activity. In some embodiments, attachment points of other moieties to the nucleic acid recognition moiety in corresponding haplomers can be designed so that the selectively-reactive moieties are brought into close spatial proximity upon hybridization of the corresponding haplomers to the nucleic acid template. For example, when two corresponding nucleic acid recognition moieties hybridize to a nucleic acid template, a terminal unit of one nucleic acid recognition moiety will be in close proximity to a terminal unit of the other nucleic acid recognition moiety. These terminal units serve as the point of attachment for additional moieties (see FIG. 9 in PCT Publication WO 14/197547).

To synthesize haplomers, three general approaches can be employed to bind the moieties. First, a functional moiety can be bound to another by direct incorporation of one moiety into the other during synthesis. For example, alkyne functionalized nucleotides can be incorporated into a nucleic acid recognition moiety during solid phase phosphoramidite oligonucleotide synthesis. Azide and alkyne functionalized amino acids are also commercially available, which can be incorporated into effector partial moieties during solid phase Merrifield peptide synthesis, or incorporated into peptide nucleic acids in a nucleic acid recognition moiety utilizing the same chemistry. Second, a functional group contained in one pre-synthesized moiety can be chemically converted to create an additional moiety in situ. For example, a primary amine contained in a nucleic acid recognition moiety or effector partial moiety can be converted to an azide by diazotransfer. Third, separate pre-synthesized moieties can be joined using bioconjugate chemistry techniques to covalently link suitable functional groups on the moieties. These functional groups can be present naturally on a moiety, or can be introduced by incorporation of a derivatized group during synthesis of a moiety.

The present disclosure provides methods according to any of the metabolic labeling methods described herein, further comprising performing a Template Assembly by Proximity-Enhanced Reactivity (TAPER) reaction using the nucleic acid template as a target nucleic acid sequence for the TAPER reaction. In some embodiments, the TAPER reaction comprises contacting the cell with a first haplomer and a second haplomer, wherein: a) the first haplomer comprises: a first polynucleotide that is complementary to a first region of the nucleic acid template; a first effector partial moiety, wherein the first effector partial moiety is linked to the first polynucleotide; and a first selectively-reactive moiety, wherein the first selectively-reactive moiety is linked to the first effector partial moiety; and b) the second haplomer comprises: a second polynucleotide that is complementary to a second region of the nucleic acid template; a second effector partial moiety, wherein the second effector partial moiety is linked to the second polynucleotide; and a second selectively-reactive moiety, wherein the second selectively-reactive moiety is linked to the second effector partial moiety; wherein: the first selectively-reactive moiety and the second selectively-reactive moiety chemically react with each other when in sufficient proximity; the first region of the nucleic acid template is in sufficient proximity to the second region of the nucleic acid template to allow the first selectively-reactive moiety and the second selectively-reactive moiety to chemically react with each other; and the first effector partial moiety and the second effector partial moiety form an active effector agent when in sufficient proximity.

In some embodiments, the first selectively-reactive moiety and the second selectively-reactive moiety are a chemically reactable pair of selectively-reactive moieties chosen from an azide, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, and a quadricyclane. In some embodiments, the first selectively-reactive moiety and the second selectively-reactive moiety are an azide-alkyne pair, an azide-phosphine pair, or a tetrazine-norbornene/trans-cyclooctene pair.

In the original TAPER process described above, reactive groups are brought into spatial proximity by virtue of their linkage with polynucleotides of predetermined sequence, which themselves co-hybridize in proximity on a nucleic acid template. Proximity of effective partial moieties greatly enhances active effector agent formation, and thus cell-type specific transcripts can direct the production of desired molecules in cells of interest. The general principle of TAPER can be altered to a two-level process, as described herein, by appending specific ligands to each haplomer instead of directly interactive functional groups. Thus, in the original configuration of TAPER (herein termed “conventional TAPER”), the process can be signified as occurring within a single reaction sequence, where the nucleic acid template can be considered functionally as a specific catalyst:

$$\begin{matrix}( 1 ) & \\ H 1 - A + H 2 - B {\to }^{Template} H 1 - [ A : B ] - H 2 ⟶ H 1 - [ P ] - H 2 & ( Equation 1 )\end{matrix}$$

where H1 and H2 represent haplomers bearing effector partial moieties A and B, respectively. Upon hybridization to a specific nucleic acid template, a proximity-driven reaction intermediate between effector partial moiety A and effector partial moiety B is formed [A:B], leading rapidly to the formation of an active effector agent product [P].

In a ligand-directed alternate of TAPER (i.e., LD-TAPER), the desired process occurs at two distinct levels:

$$\begin{matrix}( 1 ) & \\ H 1 - L 1 + H 2 - L 1 {\to }^{Template} H 1 - [ L 1 : L 1 ] - H 2 & ( Equations 2.1 and 2.2 )\end{matrix}$$

$$H 1 - [ L 1 : L 1 ] - H 2 + 2 D {\to }^{Template} H 1 - [ {L 1}_{D} : {L 1}_{D} ] - H 2 ⟶ H 1 - [ {L 1 : L 1}_{D - D} ] - H 2 \qquad ( 2 )$$

where H1 and H2 represent haplomers, L1 represents any form of ligand, and D indicates a protein binding domain or any other molecule capable of binding to the ligand in a specific manner. Here, the initial binding of two molecules of D to the two ligand molecules (displayed in spatial proximity via the haplomer template binding) is shown as a transitional state before the formation of a D-D dimer, which may or may not be thermodynamically reversible. In the second stage of LD-TAPER, the nucleic acid template is still required to enforce the ligand L1-L1 spatial proximity. This is the case for all variant embodiments of LD-TAPER except where a proximity-enhanced covalent interaction is designed to occur between two modified L1 molecules, or modified L1 and L2 molecules, such that they become covalently linked and, thus, stabilized as a pair. Provided of course that their interactions with cognate binding domains is not affected by the ligand-ligand covalent joining (as a necessary pre-condition for this embodiment), then the subsequent stage two of the LD-TAPER process becomes nucleic acid template-independent. The generalizable nature of the two-stage LD-TAPER process is schematically depicted in FIG. 1 of PCT Publication WO 18/94195. Referring to FIG. 1 of PCT Publication WO 18/94195, haplomers with specific appended ligands (L1 and L2) bind to nucleic acid template such that the ligands fall into spatial proximity with each other. Proteins or polypeptides (P1 and P2) fused with binding domains for the ligands of interest (R1 and R2) are directed to the templated haplomer site by the interaction between the ligand and the corresponding ligand binding domain. The P1 and P2 segments brought into enforced spatial proximity, promoting dimerization or protein folding.

Although LD-TAPER is a two-stage process, it is not essential in principle that the haplomer/template hybridization comprises the first step (as portrayed in FIG. 1 of PCT Publication WO 18/94195). In some embodiments, it may be desirable to initially pre-form each haplomer (bearing an appended ligand) with its designated cognate ligand binding domain, to form haplomer-fusion protein complexes. Subsequently, the hybridization step is performed, resulting in the desired molecular proximity of protein fragments fused with the ligand binding domains themselves. Various embodiments of LD-TAPER are possible, where H1 and H2 are appended with different ligands (L1 and L2), or where ligands can be bound by two separate binding domains (D1 and D2). If D1 and D2 are split-protein polypeptides, the second-level event can be comprised of mature protein folding between D1 and D2.

Evolution has provided important and very useful examples of natural small-molecule ligands, which can be exploited for biotechnological aims. For example, the interactions between: a) FK506, calcineurin, and FK506-binding protein (FKBP), and b) rapamycin, FKBP, and mTOR-FRB are depicted in FIG. 2 of PCT Publication WO 18/94195. To enhance the potential therapeutic utility of this interaction and minimize binding to endogenous FKBP protein, mutant derivatives of FKBP have been derived which preferentially bind altered FK506 analogs. Thus, the F36V FKBP mutant binds a specific FK506 derivative much more strongly than the wild-type molecule itself.

Although small molecule ligands are one type of ligand for use in LD-TAPER, they are not the only type of ligands that can be used. There exist relatively small mutually interactive protein domains (e.g., fragments of proteins) that are applicable in this context, an example of which are leucine zippers. Suitable examples of interactive protein domains are the c-jun and c-fos zipper domains, which generally are polypeptides of less than 50 amino acid residues, including helix-initiating and helix-terminating segments. While c-jun can form homodimers, c-fos cannot; and c-fos:c-jun heterodimers are significantly more stable than c-jun:c-jun homodimers. Appending such zipper sequences to polynucleotides for the purposes of creating LD-TAPER haplomers provides each haplomer, with the zipper acting as a ligand, to bind to fusion proteins of desired polypeptides with the complementary zipper as a ligand binding domain.

Whether the ligands used for LD-TAPER are small chemical entities or interactive protein domains, or any other structure for which a complementary binding element exists, the two-stage LD-TAPER process can be applied towards enforced dimerization of either the same partner protein fragments (homodimerization) or different partner protein fragments (heterodimerization). These multiple aspects of LD-TAPER are summarized in FIG. 3 of PCT Publication WO 18/94195.

For the purposes of small molecule LD-TAPER, a monovalent domain binding compound can be used, which is chemically appended to the 5′ or 3′ ends of short polynucleotide strands, which comprise a portion of the resulting haplomers. When such haplomers are hybridized to a complementary nucleic acid template, the appended monovalent compounds are brought into spatial proximity (see, FIG. 5 of PCT Publication WO 18/94195). Protein ligand binding domains which recognize and bind the monovalent compound (i.e., ligand) are likewise brought together close in spatial positioning, as are any other protein domains fused to the binding domains themselves (see, FIG. 5 of PCT Publication WO 18/94195). Such enforced dimerization of the fusion domains of the proteins of interest leads to functional activation with measurable biological consequences.

Where LD-TAPER is mediated via small molecule ligands, the ligand identity may correspond to other low molecular weight defined compounds, or natural or artificial peptides, peptidomimetics, or any other molecule with a defined binding partner. Small molecule LD-TAPER can be designed with a number of distinct template:haplomer architectures. In some embodiments comprising the simplest arrangement, the haplomers are mutually complementary to each other, such that the resulting duplex enforces the desired spatial proximity of the ligand-interacting protein fusions. This configuration is herein referred to as Architecture 1 (see, FIG. 9 of PCT Publication WO 18/94195). When the haplomers are not complementary to each other, but hybridize to spatially adjacent sites on a nucleic molecule template, Architecture 2 is achieved (see, FIG. 5 and FIG. 10 of PCT Publication WO 18/94195). Non-contiguous hybridization sites can still be LD-TAPER targets when suitable configurations exist. Thus, when LD-TAPER haplomers hybridize to the outer boundaries of a stem loop structure, spatial proximity is achieved, producing Architecture 3 (see, FIG. 11 of PCT Publication WO 18/94195). Conversely, the inner region of a stem loop can be potentially targeted if haplomers anneal with the appropriate sites relative to each other, thus producing Architecture 4 (see, FIG. 12 of PCT Publication WO 18/94195).

In some embodiments, the ligands for LD-TAPER are not small molecules in a conventional sense, but rather small interactive protein domains. These include, but are not limited to, interacting leucine zipper motifs, which themselves may be comprised of, but not limited to, parallel zippers such as c-jun:c-fos; mad:max; and c-myc:max, or antiparallel zippers, such as that from Thermus thermophilus seryl-tRNA synthetase.

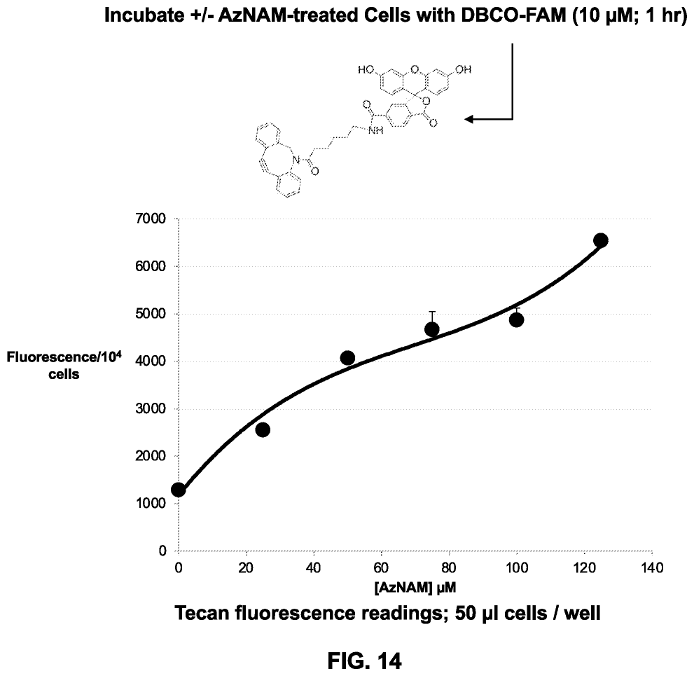

In some embodiments, when small interactive protein domains are used as ligands, the well-characterized c-jun:c-fos zipper pair is used, as depicted in FIG. 14 of PCT Publication WO 18/94195. Haplomers comprised of polynucleotide segments complementary to a nucleic acid template can be conjugated with c-jun domains, and then hybridized with the nucleic acid template. Subsequently, protein fragments of interest fused with c-fos domains are added, leading to complex formation and enforced dimerization of the protein fragment of interest. In the depiction of FIG. 14 of PCT Publication WO 18/94195, which corresponds to the two-stage strategy generalized with Equation 2.1 and 2.2 above, the initial duplex can be further stabilized by the formation of c-jun homodimers. However, since c-jun:c-fos heterodimers are significantly more stable, the introduction of the fos-fusion protein results in the preferential formation of the desired heterodimeric complex (c-fos itself cannot form homodimers). In an alternate version of this embodiment, the haplomers bearing c-jun conjugated tags are pre-assembled with the fos-fusion protein of interest, before adding to the target system containing the template of interest. This alternate arrangement corresponds to the two-stage strategy generalized with Equation 2.1 and 2.2 above.

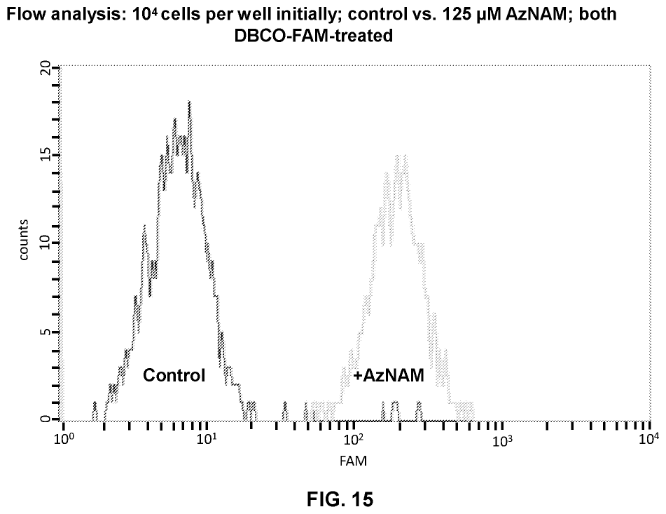

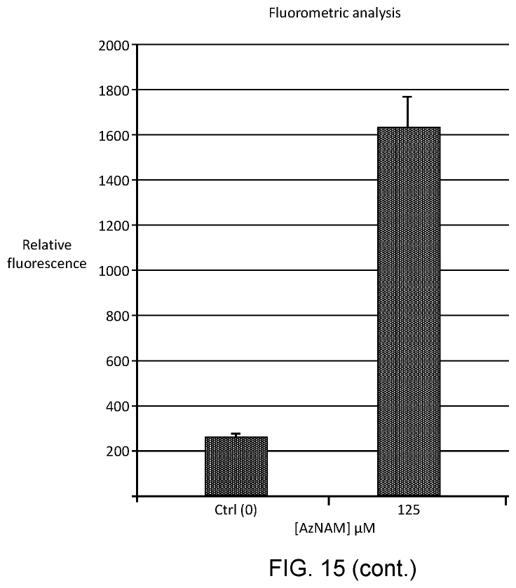

In embodiments using small interactive protein domains as ligands, the polarity of the conjugation of the small domain tag should be taken into account. This can be exemplified with the particular embodiments using fos-jun heterodimerization, where the leucine zipper interaction occurs with a parallel orientation. If haplomers have appended c-Jun tags such that their c-Jun helices are in a parallel orientation following hybridization (see, FIG. 15 of PCT Publication WO 18/94195), then subsequent complex formation with c-fos fusion proteins will orient the fusion in a parallel sense; the reverse situation may disfavor dimerization between the protein segments of interest (see, FIG. 15 of PCT Publication WO 18/94195). However, for certain other applications of LD-TAPER (most notably, for the assembly of split-protein fragments, as below), an antiparallel orientation can be beneficial. For this reason, it is advantageous if strategies exist for conjugating 5′ or 3′ polynucleotide ends with small protein tags by either their N- or C-termini.

In embodiments using small interactive protein domains as ligands, c-jun and c-fos have significant advantages. In both cases, their alpha-helical zippers are fully defined by relatively short polypeptides, neither of which possess internal cysteine residues. These sequences can be readily produced by expression systems within E. coli, and are short enough that complete synthesis is feasible. This is useful for the c-Jun segment, since it renders thiol-mediated conjugation with oligonucleotides a facile approach. For c-jun tags, the sequence used herein for making N-terminal conjugates is a 47-mer, where the N-terminal cysteine is shown, and the bold sequences denote helical boundaries: CSGGASLERI ARLEEKVKTLKAQNSELASTANMLREQVAQLKQKGAP (SEQ ID NO:2). For c-jun tags, the sequence used herein for making C-terminal conjugates is a 49-mer, where the C-terminal cysteine is shown, and the bold sequences denote helical boundaries: SGASLERIAR LEEKVKTLKAQNSELASTANMLREQVAQLKQKGAPSGGC (SEQ ID NO:3). The sequence of the fos zipper to be made as fusions with the protein fragment of interest is a 41-mer, where the bold sequences denote helical boundaries: ASRELTDTLQAETDQLEDEKS ALQTEIANLLKEKEKLEGAP (SEQ ID NO:4). Additional extended serine-glycine linkers can be inserted between the c-Fos sequence and the protein fragment of interest.

In some embodiments, mutants of c-Jun are used that cannot form homodimers, but which can still heterodimerize with c-Fos. Such modified sequences with N-terminal cysteine residues include, but are not limited to: CSGGASLERIARLEEKVKSFKAQNSENASTAN MLREQVAQLKQKGAP (SEQ ID NO:5), where bold residues denote changes from wild-type, and double-underlined sequences denote helical boundaries. Such modified sequences with C-terminal cysteine residues include, but are not limited to: SGASLERIARLEEKVKSF KAQNSENASTANMLREQVAQLKQKGAPSGGC (SEQ ID NO:6), where bold residues denote changes from wild-type, and double-underlined sequences denote helical boundaries. In some embodiments of LD-TAPER using either small molecule ligands or small interactive domain ligands, the application may be aimed towards the assembly of split-protein fragments. In other LD-TAPER embodiments, the protein fragments fused with ligand binding domains are self-folding into well-ordered and stable structures, but this is not the case with LD-TAPER applied towards split-protein assembly. In the latter, the protein sequences appended to the ligand-binding domains only attain their mature folds when they are placed in close spatial proximity in the correct orientation.

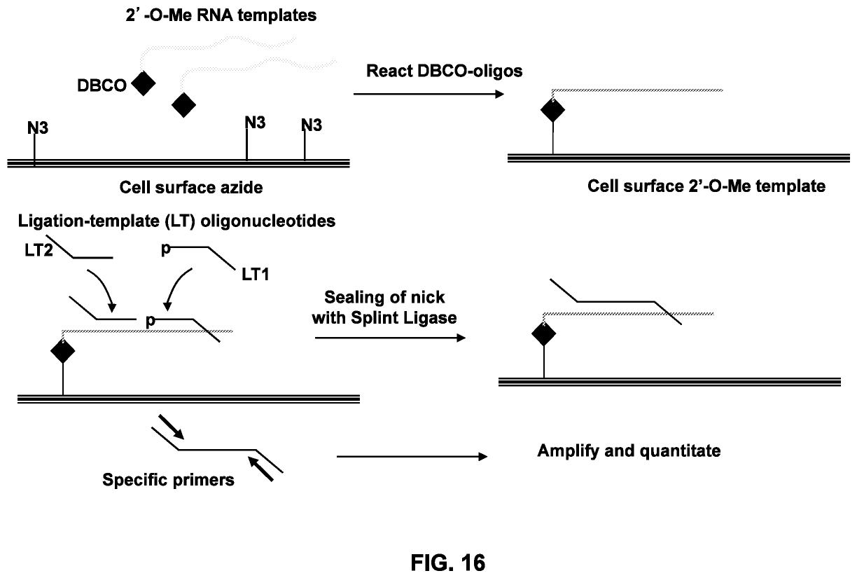

In some embodiments of LD-TAPER for split protein refolding that utilize small molecule ligands, the split protein polypeptides are separately expressed as fusions with the FKBP FK506-binding domain. The N-terminal split protein fragment is expressed with a C-terminal FKBP segment, while the C-terminal split protein fragment is expressed with an N-terminal FKBP segment (see, FIG. 16 of PCT Publication WO 18/94195). Upon binding of the FKBP domains to templated haplomeric conjugates bearing a monovalent FKBP-binder (see, FIGS. 6-8 of PCT Publication WO 18/94195), proximity-enabled folding of the mature polypeptide is elicited (see, FIG. 16, depicted for template Architecture 2, of PCT Publication WO 18/94195). Because the FKBP domains binding each haplomeric ligand are in the same (parallel) orientation, the split protein fragments are placed on opposite sides of the spatially proximal FKBP pair (see, FIG. 16 of PCT Publication WO 18/94195). However, split protein refolding can still occur if the FKBP domains and the split protein polypeptides are separated by sufficiently long serine-glycine linkers.

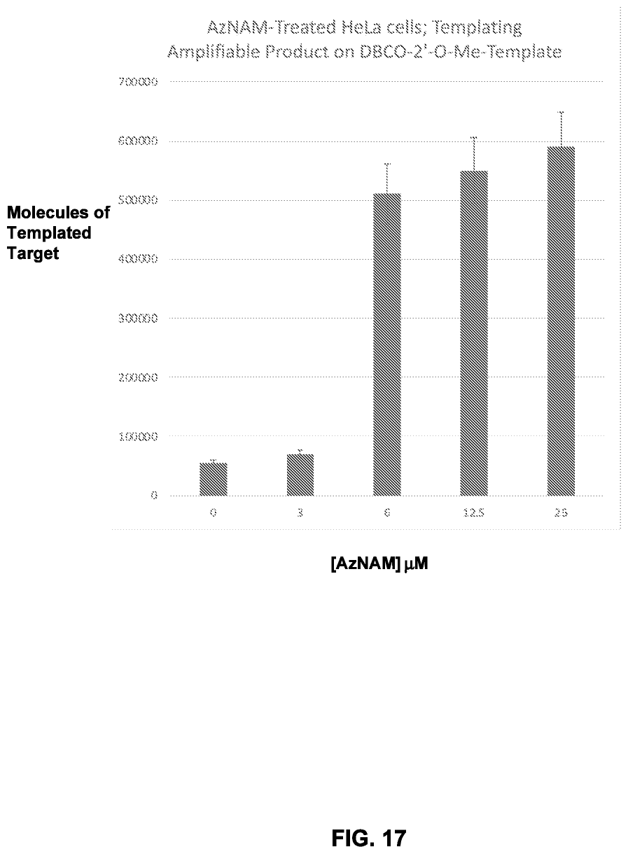

Similar principles apply for embodiments of LD-TAPER for split protein refolding that utilize small interactive protein domains as ligands. In some embodiments using c-Jun:c-Fos interactions, the N-terminal split protein fragment is expressed as a C-terminal fusion with c-Fos, while the C-terminal split protein fragment is expressed as an N-terminal with c-Fos (see, FIG. 17 of PCT Publication WO 18/94195). In these embodiments, an antiparallel arrangement of haplomers tagged with c-Jun can be readily accomplished, which is advantageous for placement of the protein fragments in juxtaposition on the same side of the c-Jun pair. Nevertheless, as for the split protein LD-TAPER mediated by small-molecule ligands (see, FIG. 16 of PCT Publication WO 18/94195), parallel c-Jun tags can still be used if a serine-glycine linker of sufficient length is employed (see, FIG. 17 of PCT Publication WO 18/94195). Although the depictions of split-protein embodiments of LD-TAPER use haplomer-template Architecture 2 (see, FIG. 10 of PCT Publication WO 18/94195), Architectures 3 and 4 (see, FIGS. 11 and 12 of PCT Publication WO 18/94195) are equally applicable. Architecture 1 (see, FIG. 9 of PCT Publication WO 18/94195) in this context corresponds to locked TAPER (see, FIG. 13 of PCT Publication WO 18/94195), also very compatible with split-protein embodiments of LD-TAPER.

In the above embodiments (as depicted in FIG. 5, and FIGS. 9-17 of PCT Publication WO 18/94195) both haplomers bear a common ligand tag, and proteins or polypeptide fragments of interest are tagged with a common ligand binding domain. This arrangement is well-suited to systems featuring homodimerization, but is not ideal for heterodimerization, by its nature as a two-stage process. If a protein heterodimer A-B is to be assembled on a template with haplomers H-A and H-B where the first stage is haplomer-template binding, and a monoligand/binding domain system is used, then the protein segments A and B can assort in three possible ways, only one of which is the correct A-B.

In some embodiments of LD-TAPER, it may nonetheless be advantageous to be able to use a heterocomponent system with full efficiency, without the need for pre-assembly of haplomers and protein fusion domains. This can be achieved by using two distinct ligands, each with a distinct and specific partner ligand binding domain.

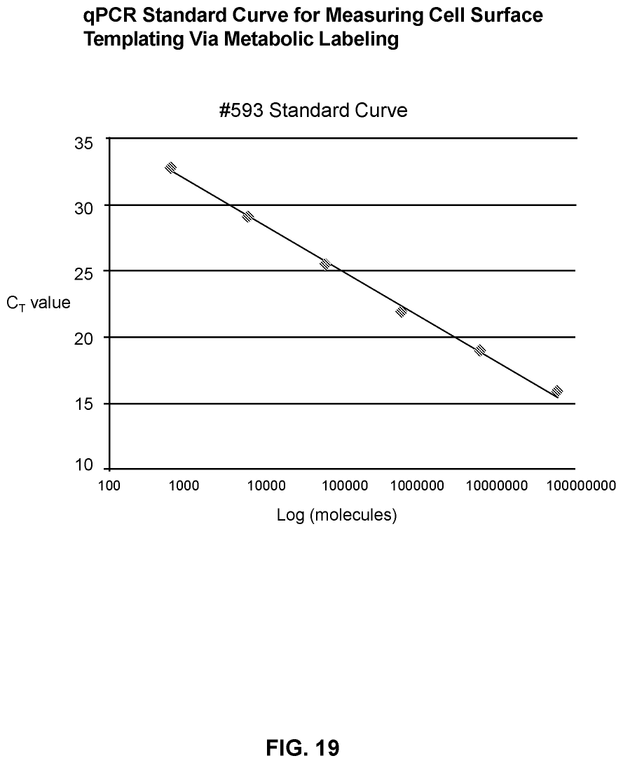

In some embodiments of heterocomponent LD-TAPER, the specificity of leucine zipper interactions is used. These include, but are not limited to, c-Jun:c-Fos, and c-Myc:Max heterodimer formation (see, FIG. 19 of PCT Publication WO 18/94195). Thus, haplomers can be prepared with terminal conjugations with c-Jun and c-Myc zippers, and protein domains of interest (or split-protein polypeptide fragments) can be expressed as fusions with c-Fos and Max. The templated jun/myc haplomers direct the forced proximity of the two domains of interest (see, FIG. 19 of PCT Publication WO 18/94195), for ensuing dimerization (pre-folded momomeric domains) or folding (split-protein polypeptide fragments).

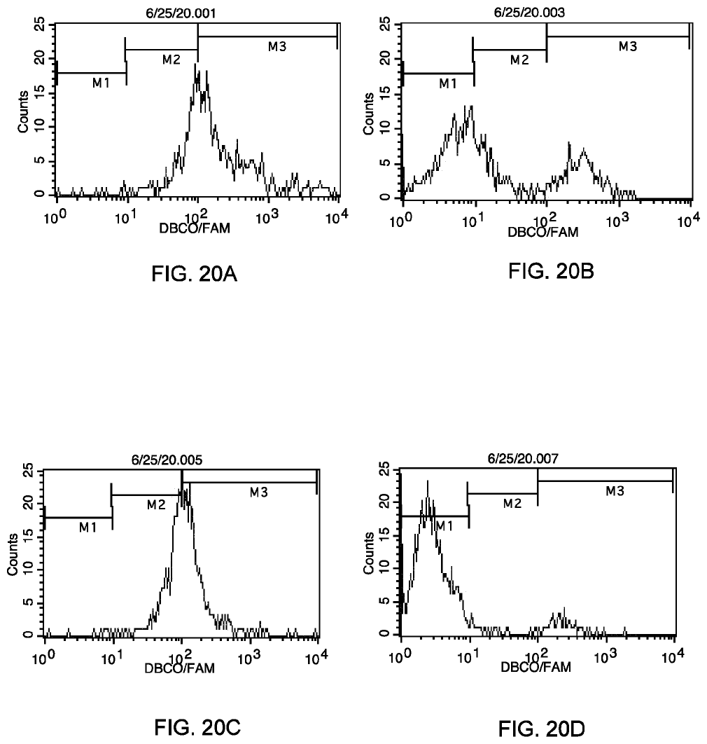

In some embodiments of LD-TAPER, it may be beneficial to stabilize small molecule monovalent ligands as a linked pair after the desired haplomeric templating has taken place. Although in many cases, enforced dimerization of polypeptide folding by small-molecule LD-TAPER is either irreversible or slowly reversed, the template-mediated conversion of the monovalent ligands to bivalency enables the subsequent dimerization to become template-independent, just as it is for conventional bivalent chemical dimerizers in isolation. Cross-linking of haplomeric small molecule ligands is affected by equipping the monovalent ligand compounds with side chains corresponding to selectively-reactive moieties, as schematically depicted in FIG. 20 of PCT Publication WO 18/94195.

Following the in situ templated click reaction, the initially monovalent ligands are converted in effect into a stable bivalent species, for subsequent interaction with appropriate ligand binding domains (see, FIG. 20 of PCT Publication WO 18/94195).

Some embodiments of LD-TAPER involve homo- or hetero-dimerization of pre-folded proteins that perform important biological functions. Such proteins may be natural, or artificial constructs. These include, but are not limited to, the iCasp9 fusion protein (an artificial construct with a modified Caspase-9 sequence fused with a mutant FKBP sequence), proapoptotic proteins, or dimeric transcription factors. The effects of enforced dimerization mediated by LD-TAPER can be gauged, in various embodiments, by apoptotic assays, activation of reporter genes, or generation of specific fluorescence.

The LD-TAPER processes and components thereof can be generally described by the following more specific embodiments.

In some embodiments, the LD-TAPER method comprises: a) contacting a nucleic acid template with a first haplomer-ligand complex comprising a small molecule ligand; b) contacting the nucleic acid template with a second haplomer-ligand complex comprising a small molecule ligand; c) contacting the first haplomer-ligand complex with a first fusion protein that comprises a ligand binding domain for a small molecule ligand; and d) contacting the second haplomer-ligand complex with a second fusion protein that comprises a ligand binding domain for a small molecule ligand; wherein: i) the ligand of the first haplomer-ligand complex is linked to the 5′ terminus of the polynucleotide of the first haplomer-ligand complex; ii) the ligand of the second haplomer-ligand complex is linked to the 3′ terminus of the polynucleotide of the second haplomer-ligand complex; iii) the polynucleotide of the first haplomer-ligand complex is substantially complementary to the nucleic acid template; iv) the polynucleotide of the second haplomer-ligand complex is substantially complementary to the nucleic acid template at a site in spatial proximity to the polynucleotide of the first haplomer-ligand complex; v) the ligand of the first haplomer-ligand complex and the ligand binding domain of the first fusion protein can interact; and vi) the ligand of the second haplomer-ligand complex and the ligand binding domain of the second fusion protein can interact; thereby resulting in the folding or dimerization of the fragment of the protein of interest of the first fusion protein with the fragment of the protein of interest of the second fusion protein.

In some embodiments, the LD-TAPER method comprises: a) contacting a nucleic acid template with a first haplomer-ligand complex comprising a ligand that is an interactive protein domain; b) contacting the nucleic acid template with a second haplomer-ligand complex comprising a ligand that is an interactive protein domain; c) contacting the first haplomer-ligand complex with a first fusion protein that comprises a fragment of a protein of interest fused to an interactive protein domain; and d) contacting the second haplomer-ligand complex with a second fusion protein that comprises a fragment of a protein of interest fused to an interactive protein domain; wherein: i) the ligand of the first haplomer-ligand complex is linked to the 5′ terminus of the polynucleotide of the first haplomer-ligand complex; ii) the ligand of the second haplomer-ligand complex is linked to the 3′ terminus of the polynucleotide of the second haplomer-ligand complex; iii) the polynucleotide of the first haplomer-ligand complex is substantially complementary to the nucleic acid template; iv) the polynucleotide of the second haplomer-ligand complex is substantially complementary to the nucleic acid template at a site in spatial proximity to the polynucleotide of the first haplomer-ligand complex; v) the ligand of the first haplomer-ligand complex and the ligand binding domain of the first fusion protein can interact; and vi) the ligand of the second haplomer-ligand complex and the ligand binding domain of the second fusion protein can interact; thereby resulting in the folding or dimerization of the fragment of the protein of interest of the first fusion protein with the fragment of the protein of interest of the second fusion protein.

In some embodiments, the polynucleotide of the first haplomer-ligand complex is complementary to the polynucleotide of the second haplomer-ligand complex. In some embodiments, the polynucleotide of the first haplomer-ligand complex binds to the nucleic acid template in spatial proximity to the binding of the polynucleotide of the second haplomer-ligand complex to the nucleic acid molecule template. In some embodiments, the ligand of the first haplomer-ligand complex is linked to the 5′ terminus of the polynucleotide of the first haplomer-ligand complex, and the polynucleotide of the first haplomer-ligand complex is complementary to a portion of the nucleic acid target 5′ adjacent to a stem-loop structure; and the ligand of the second haplomer-ligand complex is linked to the 3′ terminus of the polynucleotide of the second haplomer-ligand complex, and the polynucleotide of the second haplomer-ligand complex is complementary to a portion of the nucleic acid template 3′ adjacent to the stem-loop structure. In some embodiments, the ligand of the first haplomer-ligand complex is linked to the 3′ terminus of the polynucleotide of the first haplomer-ligand complex, and the polynucleotide of the first haplomer-ligand complex is complementary to a 5′ portion of a loop structure of a stem-loop structure of the nucleic acid template, wherein the 5′ portion of the loop structure is adjacent to the stem region of the stem-loop structure; and the ligand of the second haplomer-ligand complex is linked to the 5′ terminus of the polynucleotide of the second haplomer-ligand complex, and the polynucleotide of the second haplomer-ligand complex is complementary to a 3′ portion of the loop structure of the stem-loop structure of the nucleic acid template, wherein the 3′ portion of the loop structure is adjacent to the stem region of the stem-loop structure.

In some embodiments, the LD-TAPER method comprises: a) contacting a nucleic acid template with a complex formed by the interaction of a first haplomer-ligand complex comprising a small molecule ligand with a first fusion protein that comprises a ligand binding domain for a small molecule ligand, wherein the ligand of the first haplomer-ligand complex is linked to the 5′ terminus of the polynucleotide of the first haplomer-ligand complex, and wherein the ligand of the first haplomer-ligand complex interacts with the ligand binding domain of the first fusion protein; and b) contacting the nucleic acid template with a complex formed by the interaction of a second haplomer-ligand complex comprising a small molecule ligand with a second fusion protein that comprises a ligand binding domain for a small molecule ligand, wherein the ligand of the second haplomer-ligand complex is linked to the 5′ terminus of the polynucleotide of the second haplomer-ligand complex, and wherein the ligand of the second haplomer-ligand complex interacts with the ligand binding domain of the second fusion protein; thereby resulting in the folding or dimerization of the fragment of the protein of interest of the first fusion protein with the fragment of the protein of interest of the second fusion protein.

In some embodiments, the LD-TAPER method comprises: a) contacting a nucleic acid template with a complex formed by the interaction of a first haplomer-ligand complex comprising a ligand that is an interactive protein domain with a first fusion protein that comprises a fragment of a protein of interest fused to an interactive protein domain, wherein the ligand of the first haplomer-ligand complex is linked to the 5′ terminus of the polynucleotide of the first haplomer-ligand complex, and wherein the ligand of the first haplomer-ligand complex interacts with the ligand binding domain of the first fusion protein; and b) contacting the nucleic acid template with a complex formed by the interaction of a second haplomer-ligand complex comprising a ligand that is an interactive protein domain with a second fusion protein that comprises a fragment of a protein of interest fused to an interactive protein domain, wherein the ligand of the second haplomer-ligand complex is linked to the 5′ terminus of the polynucleotide of the second haplomer-ligand complex, and wherein the ligand of the second haplomer-ligand complex interacts with the ligand binding domain of the second fusion protein; thereby resulting in the folding or dimerization of the fragment of the protein of interest of the first fusion protein with the fragment of the protein of interest of the second fusion protein.

In some embodiments, the polynucleotide of the first haplomer-ligand complex is complementary to the polynucleotide of the second haplomer-ligand complex. In some embodiments, the polynucleotide of the first haplomer-ligand complex binds to the nucleic acid template in spatial proximity to the binding of the polynucleotide of the second haplomer-ligand complex to the nucleic acid template. In some embodiments, the ligand of the first haplomer-ligand complex is linked to the 5′ terminus of the polynucleotide of the first haplomer-ligand complex, and the polynucleotide of the first haplomer-ligand complex is complementary to a portion of the nucleic acid target 5′ adjacent to a stem-loop structure; and the ligand of the second haplomer-ligand complex is linked to the 3′ terminus of the polynucleotide of the second haplomer-ligand complex, and the polynucleotide of the second haplomer-ligand complex is complementary to a portion of the nucleic acid target 3′ adjacent to the stem-loop structure. In some embodiments, the ligand of the first haplomer-ligand complex is linked to the 3′ terminus of the polynucleotide of the first haplomer-ligand complex, and the polynucleotide of the first haplomer-ligand complex is complementary to a 5′ portion of a loop structure of a stem-loop structure of the nucleic acid target, wherein the 5′ portion of the loop structure is adjacent to the stem region of the stem-loop structure; and the ligand of the second haplomer-ligand complex is linked to the 5′ terminus of the polynucleotide of the second haplomer-ligand complex, and the polynucleotide of the second haplomer-ligand complex is complementary to a 3′ portion of the loop structure of the stem-loop structure of the nucleic acid target, wherein the 3′ portion of the loop structure is adjacent to the stem region of the stem-loop structure.

In some embodiments, the LD-TAPER method comprises: a) contacting a nucleic acid template with a bottle haplomer-ligand complex comprising a small molecule ligand; b) contacting the nucleic acid template with a second haplomer-ligand complex comprising a small molecule ligand, wherein the second haplomer-ligand complex comprises a nucleotide portion that is substantially complementary to the stem portion of the bottle haplomer-ligand complex that is linked to the ligand of the bottle haplomer-ligand complex; c) contacting the bottle haplomer-ligand complex with a first fusion protein that comprises a ligand binding domain for a small molecule ligand, wherein the ligand of the bottle haplomer-ligand complex and the ligand binding domain of the first fusion protein can interact; and d) contacting the second haplomer-ligand complex with a second fusion protein that comprises a ligand binding domain for a small molecule ligand, wherein the ligand of the second haplomer-ligand complex and the ligand binding domain of the second fusion protein can interact; thereby resulting in the folding or dimerization of the fragment of the protein of interest of the first fusion protein with the fragment of the protein of interest of the second fusion protein.

In some embodiments, the LD-TAPER method comprises: a) contacting a nucleic acid template with a bottle haplomer-ligand complex comprising a ligand that is an interactive protein domain; b) contacting the nucleic acid template with a second haplomer-ligand complex comprising a ligand that is an interactive protein domain, wherein the second haplomer-ligand complex comprises a nucleotide portion that is substantially complementary to the stem portion of the bottle haplomer-ligand complex that is linked to the ligand of the bottle haplomer-ligand complex; c) contacting the bottle haplomer-ligand complex with a first fusion protein that comprises a fragment of a protein of interest fused to an interactive protein domain, wherein the ligand of the bottle haplomer-ligand complex and the ligand binding domain of the first fusion protein can interact; and d) contacting the second haplomer-ligand complex with a second fusion protein that comprises a fragment of a protein of interest fused to an interactive protein domain, wherein the ligand of the second haplomer-ligand complex and the ligand binding domain of the second fusion protein can interact; thereby resulting in the folding or dimerization of the fragment of the protein of interest of the first fusion protein with the fragment of the protein of interest of the second fusion protein.

In some embodiments, the LD-TAPER method comprises: a) contacting a nucleic acid template with a bottle haplomer-ligand complex comprising a small molecule ligand; b) contacting the nucleic acid template with a second haplomer-ligand complex comprising a small molecule ligand, wherein the second haplomer-ligand complex comprises a nucleotide portion that is substantially complementary to the stem portion of the bottle haplomer-ligand complex that is linked to the ligand of the bottle haplomer-ligand complex; c) contacting the bottle haplomer-ligand complex with a first fusion protein that comprises a ligand binding domain for a small molecule ligand, wherein the ligand of the bottle haplomer-ligand complex and the ligand binding domain of the first fusion protein can interact; and d) contacting the second haplomer-ligand complex with a second fusion protein that comprises a ligand binding domain for a small molecule ligand, wherein the ligand of the second haplomer-ligand complex and the ligand binding domain of the second fusion protein can interact; thereby resulting in the folding or dimerization of the fragment of the protein of interest of the first fusion protein with the fragment of the protein of interest of the second fusion protein.

In some embodiments, the LD-TAPER method comprises: a) contacting a nucleic acid template with a bottle haplomer-ligand complex comprising a ligand that is an interactive protein domain; b) contacting the nucleic acid template with a second haplomer-ligand complex comprising a ligand that is an interactive protein domain, wherein the second haplomer-ligand complex comprises a nucleotide portion that is substantially complementary to the stem portion of the bottle haplomer-ligand complex that is linked to the ligand of the bottle haplomer-ligand complex; c) contacting the bottle haplomer-ligand complex with a first fusion protein that comprises a fragment of a protein of interest fused to an interactive protein domain, wherein the ligand of the bottle haplomer-ligand complex and the ligand binding domain of the first fusion protein can interact; and d) contacting the second haplomer-ligand complex with a second fusion protein that comprises a fragment of a protein of interest fused to an interactive protein domain, wherein the ligand of the second haplomer-ligand complex and the ligand binding domain of the second fusion protein can interact; thereby resulting in the folding or dimerization of the fragment of the protein of interest of the first fusion protein with the fragment of the protein of interest of the second fusion protein.

Administration of sets of corresponding haplomer-ligand complexes along with their corresponding fusion proteins to a mammal, or to a human, may vary according to the nature of the disease, disorder or condition sought to be treated. In some embodiments, the haplomer-ligand complexes, bottle haplomer-ligand complexes, and fusion proteins can be dispensed into a sample within a suitable vessel or chamber. In some embodiments, the sample may be dispensed into a vessel already containing the haplomer-ligand complexes, bottle haplomer-ligand complexes, and fusion proteins. In some embodiments, the haplomer-ligand complexes, bottle haplomer-ligand complexes, and fusion proteins can used in vitro or in situ. In some embodiment, the human will be in need of such treatment.

In some embodiments, the polynucleotide of the haplomer comprises from about 6 to about 20 nucleotide bases. In some embodiments, the polynucleotide of the haplomer comprises from about 8 to about 15 nucleotide bases.

In some embodiments, a pair of haplomer-ligand complexes works in tandem. In some embodiments, the ligand of the first haplomer-ligand complex is linked to the 5′ terminus of the polynucleotide of the first haplomer-ligand complex, and the ligand of the second haplomer-ligand complex is linked to the 3′ terminus of the polynucleotide of the second haplomer-ligand complex.

In some embodiments, the polynucleotide of the first haplomer-ligand complex is substantially complementary to the polynucleotide of the second haplomer-ligand complex. In some embodiments, the polynucleotide of the first haplomer-ligand complex is substantially complementary to a nucleic acid template, and the polynucleotide of the second haplomer-ligand complex is substantially complementary to the nucleic acid template at a site in spatial proximity to the polynucleotide of the first haplomer-ligand complex.

In any of the embodiments described herein, the haplomer-ligand complexes are in spatial proximity (when bound to a nucleic acid template) such that the ligands, and hence their respective ligand binding domains, can properly interact to induce the folding or dimerization of their respective fragments of the protein of interest. Thus, for any haplomer-ligand pairs, reactivity can occur where the gap N between the first and second haplomer-ligand complex binding to the nucleic acid template is 0 (i.e., the haplomer-ligand complexes are immediately juxtaposed), and progressively greater gaps (N>0) will progressively diminish activity. Thus, in some embodiments, there is 0 nucleotides between the binding of a first haplomer-ligand complex and second haplomer-ligand complex to the nucleic acid template. In some embodiments, there is less than 6 nucleotides between the binding of a first haplomer-ligand complex and second haplomer-ligand complex to the nucleic acid template. In some embodiments, there is less than 5 nucleotides between the binding of a first haplomer-ligand complex and second haplomer-ligand complex to the nucleic acid template. In some embodiments, there is less than 4 nucleotides between the binding of a first haplomer-ligand complex and second haplomer-ligand complex to the nucleic acid template. In some embodiments, there is less than 3 nucleotides between the binding of a first haplomer-ligand complex and second haplomer-ligand complex to the nucleic acid template. In some embodiments, there is less than 2 nucleotides between the binding of a first haplomer-ligand complex and second haplomer-ligand complex to the nucleic acid template.

In some embodiments, both ligands are small molecule ligands or both ligands are interactive protein domains. In some embodiments, the ligand of the first haplomer-ligand complex further comprises a selectively-reactive moiety, and the ligand of the second haplomer-ligand complex further comprises a selectively-reactive moiety, wherein the selectively-reactive moiety of the first haplomer-ligand complex is reactable with the selectively-reactive moiety of the second haplomer-ligand complex.

The present disclosure also provides LD-TAPER methods using bottle haplomer-ligand complexes comprising: a) a bottle haplomer, wherein the bottle haplomer comprises a polynucleotide, wherein the polynucleotide comprises: i) a first stem portion comprising from about 10 to about 20 nucleotide bases; ii) an anti-target loop portion comprising from about 16 to about 40 nucleotide bases and having a first end to which the first stem portion is linked, wherein the anti-target loop portion is substantially complementary to a nucleic acid template; and iii) a second stem portion comprising from about 10 to about 20 nucleotide bases linked to a second end of the anti-target loop portion, wherein the first stem portion is substantially complementary to the second stem portion; and b) a ligand linked to the terminal end of either the first stem portion or the second stem portion, wherein the ligand comprises a ligand partner binding site; wherein the Tₘ of the anti-target loop portion:nucleic acid template is greater than the Tₘ of the first stem portion:second stem portion.

In some embodiments, the first stem portion that comprises from about 10 to about 20 nucleotide bases. In some embodiments, the first stem portion comprises from about 12 to about 18 nucleotide bases.

In some embodiments, the anti-target loop portion comprises from about 16 to about 40 nucleotide bases. In some embodiments, the anti-target loop portion comprises from about 18 to about 35 nucleotide bases. The anti-target loop portion has a first end to which the first stem portion is linked. The anti-target loop portion is substantially complementary to a nucleic acid template. In some embodiments, a ligand is linked to the second stem portion.

In some embodiments, the anti-target loop portion can further comprise an internal hinge region, wherein the hinge region comprises one or more nucleotides that are not complementary to the nucleic acid template. In some embodiments, the hinge region comprises from about 1 nucleotide to about 6 nucleotides, from about 1 nucleotide to about 5 nucleotides, from about 1 nucleotide to about 4 nucleotides, from about 1 nucleotide to about 3 nucleotides, or 1 or 2 nucleotides.

In some embodiments, the second stem portion comprises from about 10 to about 20 nucleotide bases. In some embodiments, the second stem portion comprises from about 12 to about 18 nucleotide bases. The second stem portion is linked to a second end of the anti-target loop portion. The first stem portion is substantially complementary to the second stem portion. In some embodiments, a ligand is linked to the second stem portion.

In some embodiments, the bottle haplomer comprises the nucleotide sequence 5′-ACTCGAGACGTCTCCTTGTCTTTGCTTTTCTTCAGGACACAGTGGCGAGACGTCTC GAGT-3′ (SEQ ID NO:7) or 5′-ACTCGAGACGTCTCCTTCCTGCCCCTCCTCCTGCTC CGAGACGTCTCGAGT-3′ (SEQ ID NO:8).

For the polynucleotides of the bottle haplomers described herein, the length of the particular polynucleotide or portion thereof is less important than the Tₘ of the duplex formed by the interaction of the polynucleotide, or portion thereof, with another nucleic acid molecule, or portion thereof. For example, the Tₘ of the duplex formed by the interaction of the anti-target loop portion with the nucleic acid template (e.g., anti-target loop portion:nucleic acid template) is greater than the Tₘ of the duplex formed by the interaction of the first stem portion with the second stem portion (e.g., first stem portion:second stem portion). In some embodiments, the Tₘ of the first stem portion:second stem portion subtracted from the Tₘ of the anti-target loop portion:nucleic acid template is from about 10° C. to about 40° C. In some embodiments, the Tₘ of the first stem portion:second stem portion subtracted from the Tₘ of the anti-target loop portion:nucleic acid template is from about 10° C. to about 20° C. In some embodiments, the Tₘ of the first stem portion:second stem portion is from about 40° C. to about 50° C. In some embodiments, the Tₘ of the anti-target loop portion:nucleic acid template is from about 60° C. to about 80° C.

In addition, translating the Tₘ information above into specific lengths of the nucleic acid molecules described herein can also depend on the GC content of each nucleic acid molecule. For example, the length of a suitable HPV model nucleic acid template is 30 bases (having a Tₘ of 70° C.), while that for the EBV model nucleic acid template is only 21 bases (having a Tₘ of 69° C.), owing to its greater % GC.

In some embodiments, a bottle haplomer-ligand complex works in tandem with a second haplomer-ligand complex. In some embodiments, the bottle haplomer-ligand complex is any bottle haplomer-ligand complex described herein, and the second haplomer-ligand complex is any of the haplomer-ligand complexes described herein. In some embodiments, the second haplomer-ligand complex comprises: a) a nucleotide portion comprising from about 6 to about 20 nucleotide bases that is substantially complementary to the stem portion of the bottle haplomer-ligand complex that is linked to the ligand of the bottle haplomer-ligand complex; and b) a ligand linked to the 5′ or 3′ terminus of the nucleotide portion of the second haplomer-ligand complex, wherein the ligand comprises a ligand partner binding site; wherein the Tₘ of the second haplomer-ligand complex:first or second stem portion linked to the ligand of the bottle haplomer-ligand complex is less than or equal to the Tₘ of the first stem portion:second stem portion of the bottle haplomer-ligand complex.

In some embodiments, the Tₘ of the duplex formed by the interaction of the second haplomer-ligand complex with either the first stem portion or the second stem portion, whichever stem portion is linked to the ligand (e.g., second haplomer-ligand complex:first or second stem portion linked to the ligand), is less than or equal to the Tₘ of the first stem portion: second stem portion. In some embodiments, the Tₘ of the duplex formed by the second haplomer-ligand complex and the first or second stem portion linked to the ligand subtracted from the Tₘ of the first stem portion:second stem portion is from about 0° C. to about 20° C. In some embodiments, the Tₘ of the duplex formed by the second haplomer-ligand complex and the first or second stem portion linked to the ligand subtracted from the Tₘ of the first stem portion:second stem portion is from about 5° C. to about 10° C. In some embodiments, the Tₘ of the duplex formed by the second haplomer-ligand complex and the first or second stem portion linked to the ligand is from about 30° C. to about 40° C.

This structural arrangement is designed such that in the absence of nucleic acid template, the locked first haplomer bottle does not significantly hybridize to its complementary second haplomer and, thus, template-directed product assembly is not promoted under such conditions. When the specific nucleic acid template is present, on the other hand, the bottle haplomer-ligand complex is “unlocked” by the formation of a more stable hybrid between the anti-target loop region of the bottle haplomer and the nucleic acid template itself. Once this occurs, the first stem portion of the bottle haplomer that is linked to the ligand is free to hybridize to the available second haplomer-ligand complex, with resulting proximity between the ligands on both.

In any of the haplomer polynucleotides described herein, or any portion thereof, the nucleotide bases are selected from the group consisting of DNA nucleotides, RNA nucleotides, phosphorothioate-modified nucleotides, 2-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, DNA analogs with L-ribose (L-DNA), Xeno nucleic acid (XNA) analogues, or other nucleic acid analogues capable of base-pair formation, or artificial nucleic acid analogues with altered backbones, or any combination or mixture thereof.

For any of the any of the haplomer polynucleotides described herein, the complementarity with another nucleic acid molecule can be 100%. In some embodiments, one particular nucleic acid molecule can be substantially complementary to another nucleic acid molecule. As used herein, the phrase “substantially complementary” means from 1 to 10 mismatched base positions, from 1 to 9 mismatched base positions, from 1 to 8 mismatched base positions, from 1 to 7 mismatched base positions, from 1 to 6 mismatched base positions, from 1 to 5 mismatched base positions, from 1 to 4 mismatched base positions, from 1 to 3 mismatched base positions, and 1 or 2 mismatched base positions. In some embodiments, it is desirable to avoid reducing the Tₘ of the anti-target loop portion:nucleic acid template by more than 10% via mismatched base positions. The bottle haplomer stem is designed with respect to a second haplomer, and its structure is deliberately arranged to be somewhat more stable than the formation of the second haplomer duplex.

In some embodiments, the portion of the bottle haplomer-ligand complex that is not linked to a ligand can have additional nucleotide bases that overhang and do not form a part of the stem structure. In some embodiments, the end of the second haplomer-ligand complex that is not linked to a ligand can have additional nucleotide bases that overhang and do not form a complementary part of the structure with the stem portion of the bottle haplomer-ligand complex. In addition, in some embodiments, the portion of the stem that is linked to the ligand can also have nucleotide bases that are not base paired with the first stem portion. Such an extension of the stem with a non-hybridized “arm” places the two ligands at a greater spatial distance, thus, tending to reduce their mutual reactivity. So, for a few nucleotide bases (less than 10 or less than 5), enforced reactivity is still likely to occur, but will tend to diminish as the non-base paired segment grows in length.

In some embodiments, both ligands are small molecule ligands or both ligands are interactive protein domains. In some embodiments, the N-terminus of the interactive protein domain of the bottle haplomer-ligand complex is linked to the 5′ terminus of the polynucleotide of the bottle haplomer-ligand complex, and the N-terminus of the interactive protein domain of the second haplomer-ligand complex is linked to the 3′ terminus of the polynucleotide of the second haplomer-ligand complex. In some embodiments, the C-terminus of the interactive protein domain of the bottle haplomer-ligand complex is linked to the 5′ terminus of the polynucleotide of the bottle haplomer-ligand complex, and the C-terminus of the interactive protein domain of the second haplomer-ligand complex is linked to the 3′ terminus of the polynucleotide of the second haplomer-ligand complex.

In some embodiments, the C-terminus of the interactive protein domain of the bottle haplomer-ligand complex is linked to the 5′ terminus of the polynucleotide of the bottle haplomer-ligand complex, and the N-terminus of the interactive protein domain of the second haplomer-ligand complex is linked to the 3′ terminus of the polynucleotide of the second haplomer-ligand complex; or the N-terminus of the interactive protein domain of the bottle haplomer-ligand complex is linked to the 5′ terminus of the polynucleotide of the bottle haplomer-ligand complex, and the C-terminus of the interactive protein domain of the second haplomer-ligand complex is linked to the 3′ terminus of the polynucleotide of the second haplomer-ligand complex.

Any of the bottle haplomers described herein, or any portion thereof, can further comprise a linker between any one or more of the first stem portion and the anti-target loop portion, between the anti-target loop portion and the second stem portion, between the second stem portion and the ligand, between the first stem portion and the ligand, or between the second haplomer and its ligand. In some embodiments, the linker is selected from the group consisting of an alkyl group, an alkenyl group, an amide, an ester, a thioester, a ketone, an ether, a thioether, a disulfide, an ethylene glycol, a cycloalkyl group, a benzyl group, a heterocyclic group, a maleimidyl group, a hydrazone, a urethane, azoles, an imine, a haloalkyl, and a carbamate, or any combination thereof.

In some embodiments, the second haplomer-ligand complex comprises the nucleotide sequence 5′-AGCTCTCGAGT-3′ (SEQ ID NO:9), or 5′-GACGTCTCGA GT-3′ (SEQ ID NO:10).

In any of the embodiments described herein, the ligand is a small molecule ligand or an interactive protein domain.

In some embodiments, the ligand is a small molecule ligand. In some embodiments, the small molecule ligand is less than about 2500 Daltons. In some embodiments, the small molecule ligand is a small molecule, a peptide having less than about 20 amino acid residues, a naturally- or artificially-modified peptide, a peptidomimetic, a glycan, an organic enzyme cofactor, or an artificially-derived small molecular ligand. In some embodiments, the small molecule ligand is derived from compounds designed to target FKBP. In some embodiments, the small molecule ligand is an FKM monovalent ligand.

In some embodiments, the small molecule ligand is derived from compounds designed to target mutant versions of FKBP (Clackson et al., Proc. Natl. Acad. Sci. USA, 1998, 95, 103437-10432). One such modification is FKM-NHS, which comprises a modified monovalent mutant FKBP binding moiety with an appended amide, 3-carbon spacer, and a carboxylic acid group, esterified with N-hydroxysuccinimide (NHS), which can be used, for example, for coupling to amino-labeled oligonucleotides. The spacer moiety of FKM-NHS can comprise 4, 5 or 6 carbon atoms in length. FKM-NHS can also be modified to enhance solubility.

In some embodiments, the small molecule ligand is FKM-sulfo-NHS, which can be used, for example, for coupling to amino-labeled oligonucleotides, and with an additional sulfo-group for solubility enhancement. The NHS moeity carries a sulfo-group. The spacer moiety of FKM-sulfo-NHS can comprise 4, 5 or 6 carbon atoms in length.

In some embodiments, the small molecule ligand comprises another solubility-enhancing modification of FKM-NHS, where the spacer arm is converted into a short segment of polyethylene glycol (PEG) to provide FKM-PEG3-NHS, which can be used, for example, for coupling to amino-labeled oligonucleotide, and with a PEG spacer for solubility enhancement. The spacer arm of FKM-PEG3-NHS can comprise 1, 2, 4, 5, or 6 copies of the monomer ethylene glycol. FKM-NHS and all derivatives of it can be readily and directly coupled to amino-labeled oligonucleotides, at either 5′ or 3′ ends. In addition, the site of appending the reactive group to the PEG chain can be varied. Thus, if the carbon atoms in the PEG chain are numbered, the reactive group could be positioned at any of these sites.

In some embodiments, the FKM monovalent ligand is FKM-NHS, FKM-sulfo-NHS, FKM-PEG3-NHS, or monovalent FKBP Ligand-2 (MFL2), wherein: FKM-NHS is

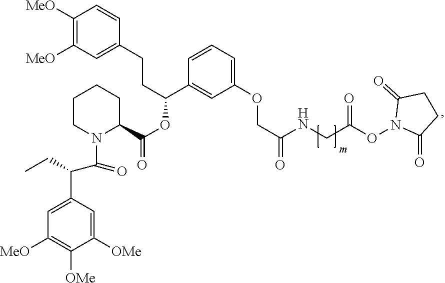

where m is from 3 to 6; FKM-sulfo-NHS is

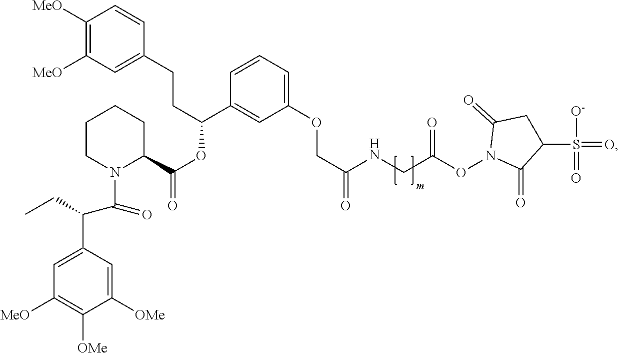

where m is
from 3 to 6; FKM-PEG3-NHS is

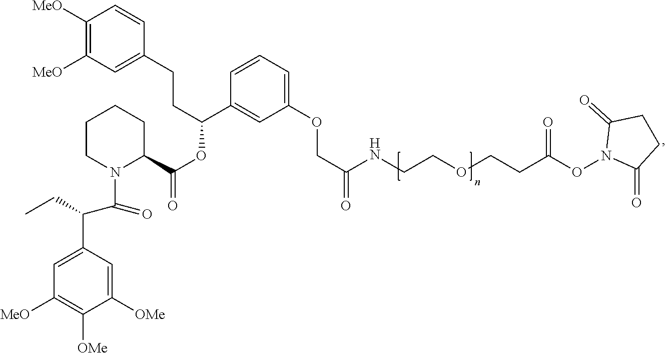

where n is from 1 to 6; and MFL2 is

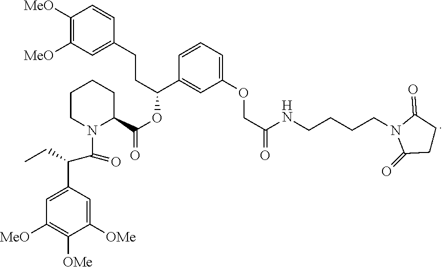 [SMILES: CC[C@H](C(=O)N1CCCC[C@H]1C(=O)O[C@H](CCc1ccc(OC)c(OC)c1)c1cccc(OCC(=O)NCCCCN2C(=O)CCC2=O)c1)c1cc(OC)c(OC)c(OC)c1]

In some embodiments, the ligand is an interactive protein domain. In some embodiments, the interactive protein domain comprises less than 100 amino acid residues. In some embodiments, the interactive protein domain is a leucine zipper domain. In some embodiments, the interactive protein domain is a c-jun domain, a c-fos domain, a c-myc domain, a c-max domain, an NZ domain, or a CZ domain.

In some embodiments, the NZ domain comprises the amino acid sequence ALKKEL QANKKELAQLKWELQALKKELAQ (SEQ ID NO:11), and the CZ domain comprises the amino acid sequence EQLEKKLQALEKKLAQLEWKNQALEKKLAQ (SEQ ID NO:12).

In some embodiments, the N-terminus of the c-jun domain is linked to the 5′ or 3′ terminus of the polynucleotide of the haplomer. In some embodiments, the c-jun domain comprises the amino acid sequence CSGGASLERIARLEEKVKTLKAQNSELASTANMLR EQVAQLKQKGAP (SEQ ID NO:2), CSGGASLERIARLEEKVKSFKAQNSENASTANM LREQVAQLKQKGAP (SEQ ID NO:5), or CSGASLERIARLEEKVKSFKAQNSENASTA NMLREQVAQLKQKGAP (SEQ ID NO:13). In some embodiments, the C-terminus of the c-jun domain is linked to the 5′ or 3′ terminus of the polynucleotide of the haplomer. In some embodiments, the c-jun domain comprises the amino acid sequence GASLERIARLEEKVKT LKAQNSELASTANMLREQVAQLKQKGAPSGGC (SEQ ID NO:3), or SGASLERIARLE EKVKSFKAQNSENASTANMLREQVAQLKQKGAPSGGC (SEQ ID NO:6).

In some embodiments, the c-Fos domain comprises the amino acid sequence ASRE TDTLQAETDQLEDEKSALQTEIANLLKEKEKLEGAP (SEQ ID NO:4) or SGASRELTD TLQAETDQLEDEKSALQTEIANLLKEKEKLEGAP (SEQ ID NO:14).

In some embodiments where two haplomer-ligand complexes work in tandem, or where a bottle haplomer-ligand complexes work in tandem with a second haplomer-ligand complex, both ligands are either small molecule ligands or both ligands are interactive protein domains. In some embodiments, both interactive protein domains are leucine zipper domains.

In some embodiments, the N-terminus of the interactive protein domain of the first haplomer-ligand complex is linked to the 5′ terminus of the polynucleotide of the first haplomer-ligand complex, and the N-terminus of the interactive protein domain of the second haplomer-ligand complex is linked to the 3′ terminus of the polynucleotide of the second haplomer-ligand complex. In some embodiments, the C-terminus of the interactive protein domain of the first haplomer-ligand complex is linked to the 5′ terminus of the polynucleotide of the first haplomer-ligand complex, and the C-terminus of the interactive protein domain of the second haplomer-ligand complex is linked to the 3′ terminus of the polynucleotide of the second haplomer-ligand complex.

In some embodiments, the C-terminus of the interactive protein domain of the first haplomer-ligand complex is linked to the 5′ terminus of the polynucleotide of the first haplomer-ligand complex, and the N-terminus of the interactive protein domain of the second haplomer-ligand complex is linked to the 3′ terminus of the polynucleotide of the second haplomer-ligand complex; or the N-terminus of the interactive protein domain of the first haplomer-ligand complex is linked to the 5′ terminus of the polynucleotide of the first haplomer-ligand complex, and the C-terminus of the interactive protein domain of the second haplomer-ligand complex is linked to the 3′ terminus of the polynucleotide of the second haplomer-ligand complex.

In some embodiments, the C-terminus of the c-jun domain is linked to the 5′ or 3′ terminus of the polynucleotides of either or both of the first haplomer-ligand complex and second haplomer-ligand complex.

In some embodiments, the ligand linked to the polynucleotide of the first haplomer-ligand complex is a c-jun domain or a c-myc domain, and the ligand linked to the polynucleotide of the second haplomer-ligand complex is a c-jun domain or a c-myc domain. In some embodiments, the ligand linked to the polynucleotide of the first haplomer-ligand complex or second haplomer-ligand complex is a c-jun domain, and the ligand linked to the polynucleotide of the other of the first haplomer-ligand complex and second haplomer-ligand complex is a c-myc domain. In some embodiments, the ligand linked to the polynucleotide of the first haplomer-ligand complex is a c-jun domain, and the ligand linked to the polynucleotide of the second haplomer-ligand complex is a c-jun domain.

In some embodiments, the ligand linked to the polynucleotide of the bottle haplomer-ligand complex is a c-jun domain, and the ligand linked to the polynucleotide of the second haplomer-ligand complex is a c-jun domain. In some embodiments, the ligand linked to the polynucleotide of one of the bottle haplomer-ligand complex and second haplomer-ligand complex is a c-jun domain, and the ligand linked to the polynucleotide of the other of the bottle haplomer-ligand complex and second haplomer-ligand complex is a c-myc domain.

In some embodiments, the polynucleotide of the bottle haplomer-ligand complex comprises the nucleotide sequence of 5′-ACTCGAGACGTCTCCTTGTCTTTGCTTTTCTT CAGGACACAGTGGCGAGACGTCTCGAGT-3′ (SEQ ID NO:7), and the polynucleotide of the second haplomer-ligand complex comprises the nucleotide sequence of 5′-AGCTCTCGAGT-3′ (SEQ ID NO:9); or the polynucleotide of the bottle haplomer-ligand complex comprises the nucleotide sequence of 5′-ACTCGAGACGTCTCCTTCCTGCCCC TCCTCCTGCTCCGAGACGTCTCGAGT-3′ (SEQ ID NO:8), and the polynucleotide of the second haplomer-ligand complex comprises the nucleotide sequence 5′-GACGTCTCGAGT-3′ (SEQ ID NO:10).

The nucleic acid templates that serve as templates in the embodiments described herein can be comprised of any desired nucleic acid sequence capable of hybridizing with the polynucleotides of the haplomers or the anti-target loop portion of a bottle haplomer. Any single-stranded nucleic acid molecule with an accessible sequence is potentially targetable. These include, but are not limited to, cellular RNAs, mRNA, genomic or organellar DNA, episomal or plasmid DNA, viral DNA or RNA, miRNA, rRNA, snRNA, tRNA, short and long non-coding RNAs, and any artificial sequences used for templating purposes, or any other biological or artificial nucleic acid sequence. Artificial sequences include, but are not limited to, aptamers and macromolecular-nucleic acid conjugates. Aptamer templates are also included, where these are designed to convert a non-nucleic acid cellular product into a targetable sequence for any form of TAPER, including locked TAPER. In some embodiments, the nucleic acid template hybridization site is kept as short as possible while: 1) maintaining specificity within a complex transcriptome or other complex targets; and 2) maintaining the locked TAPER design guidelines described herein.

In some embodiments, when the ligand is a small molecule ligand, the small molecule ligand can further comprise a selectively-reactive moiety. In some embodiments where two haplomer-ligand complexes work in tandem, the ligand of the first haplomer-ligand complex further comprises a bio-orthogonal moiety, and the ligand of the second haplomer-ligand complex further comprises a selectively-reactive moiety, wherein the selectively-reactive moiety of the first haplomer-ligand complex is reactable with the selectively-reactive moiety of the second haplomer-ligand complex. In some embodiments where a bottle haplomer-ligand complex work in tandem with a second haplomer-ligand complex, the ligand of the bottle haplomer-ligand complex further comprises a selectively-reactive moiety, and the ligand of the second haplomer-ligand complex further comprises a selectively-reactive moiety, wherein the selectively-reactive moiety of the bottle haplomer-ligand complex is reactable with the selectively-reactive moiety of the second haplomer-ligand complex.

The configurations involving the ligands described herein can be reversed. In other words, the ligand could be linked to the 3′ end of the bottle haplomer-ligand complex, as long as the second haplomer-ligand complex accordingly had its ligand linked to its 5′ end. Likewise, in this system, the selectively-reactive moieties can be switched around. For example, instead of using the bottle haplomer-ligand complex with a 5′-hexynyl and the second haplomer-ligand complex with a 3′-azide, the bottle haplomer-ligand complex could bear the azide, and the second haplomer-ligand complex the hexynyl group.

In some embodiments, the selectively-reactive moiety is chosen from an azide, an alkyne, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, or a quadricyclane, or any derivative thereof. In some embodiments, the selectively-reactive moiety of the first haplomer is hexynyl and the selectively-reactive moiety of the second haplomer is azide. In some embodiments, the selectively-reactive moiety of the first haplomer is azide and the selectively-reactive moiety of the second haplomer is hexynyl.

For example, some embodiments use modifications of the FKM-NHS series of compounds (see, FIGS. 6-8 of PCT Publication WO 18/94195) with selectively-reactive moiety side chains. In some embodiments, FKM-PEG3-NHS is modified at the C₂ position of the PEG chain with a methyltetrazine group (FKM-PEG3-MTZ-NHS), or a trans-cyclooctene group (FKM-PEG3-TCO-NHS). When these compounds are appended to amino-labeled polynucleotides via standard NHS chemistry to form click-modified ligand haplomers, they can react with each other in a bio-orthogonal fashion after templating places them in close spatial proximity. FKM-PEG3-MTZ-NHS is:

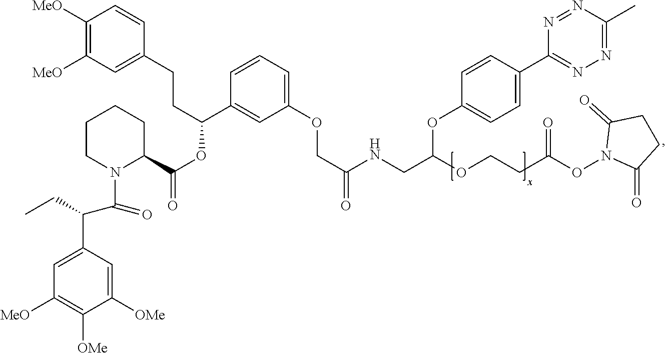

where x is from 1 to 6; and FKM-PEG3-TCO-NHS is

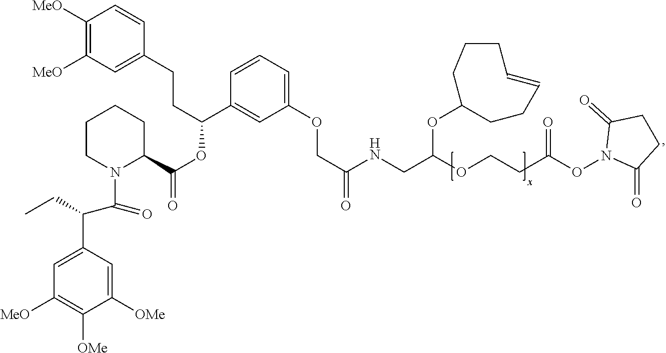

where x is from 1 to 6.

In some embodiments, the ligand of one of the first haplomer-ligand complex or second haplomer-ligand complex is an FKM monovalent ligand that is FKM-PEG3-MTZ-NHS and the ligand of the other of the first haplomer-ligand complex or second haplomer-ligand complex is an FKM monovalent ligand that is FKM-PEG3-TCO-NHS. In some embodiments, the ligand of one of the bottle haplomer-ligand complex and second haplomer-ligand complex is an FKM monovalent ligand that is FKM-PEG3-MTZ-NHS and the ligand of the other of the bottle haplomer-ligand complex and second haplomer-ligand complex is an FKM monovalent ligand that is FKM-PEG3-TCO-NHS.

In some embodiments, the ligand binding domain is a ligand binding domain for small molecule ligands. In some embodiments, the ligand binding domain is an FKBP domain or an FRB domain. In some embodiments, the FKBP domain is a mutant FKBP domain. In some embodiments, the mutant FKBP domain is the F36V FKBP mutant domain comprising the amino acid sequence GVQVETISPGDGRTFPKRGQTCVVHYTGMLEDGK KVDSSRDRNKPFKFMLGKQEVIRGWEEGVAQMCPDYAYGATGHPGIIPPHATLVFD VELLKLE (SEQ ID NO:15) or MGVQVETISPGDGRTFPKRGQTCVVHYTGMLEDGKK VDSSRDRNKPFKFMLGKQEVIRGWEEGVAQMSVGQRAKLTISPDYAYGATGHPGIIP PHATLVFDVELLKLE (SEQ ID NO:16).

In some embodiments, the ligand binding domain is an interactive protein domain. In some embodiments, the interactive protein domain comprises less than 100 amino acid residues. In some embodiments, the interactive protein domain is a leucine zipper domain. In some embodiments, the interactive protein domain is a c-jun domain, a c-fos domain, a c-myc domain, a c-max domain, an NZ domain, or a CZ domain.

In some embodiments, the interactive protein domain is fused to the N-terminus of the protein of interest. In some embodiments, the interactive protein domain is fused to the C-terminus of the protein of interest.

In some embodiments, the fusion protein comprises a linker between the protein of interest and the ligand binding domain. In some embodiments, the linker is a Ser/Gly linker, a Poly-Asparagine linker, or a linker comprising the amino acid sequence AGSSAAGSGS (SEQ ID NO:17). In some embodiments, the Poly-Asparagine linker comprises from about 8 to about 16 asparagine residues. In some embodiments, the Ser/Gly linker comprises GGSG GGSGGGSGGGSGGG (SEQ ID NO:18), GGSGGGSGGGSGGGSGGGSGGG (SEQ ID NO:19), GGSGGGSGGGSGGGSGGGSGGGSGGG (SEQ ID NO:20), SGGGGSGGGG SGGGG (SEQ ID NO:21), SGGGGSGGGGSGGGGSGGGG (SEQ ID NO:22), SGGGGS GGGGSGGGGSGGGGSGGGG (SEQ ID NO:23), SGGGS (SEQ ID NO:24), SGSG (SEQ ID NO:25), SGGGGS (SEQ ID NO:26), or SGSGG (SEQ ID NO:27).

In some embodiments, the protein of interest is a fragment of: superfolder GFP (sfGFP), Renilla luciferase, murine dihydrofolate reductase (DHFR), S. cerevisiae ubiquitin, β-lactamase, or Herpes simplex virus type 1 thymidine kinase, wherein one fragment of the protein of interest dimerizes or folds together with the other fragment of the protein of interest.

In some embodiments, the fragment of superfolder GFP (sfGFP) comprises MRKG ELFTGVVPILVELDGDVNGHKFSVRGEGEGDATNGKLTLKFICTTGKLPVPWPTLVT TLTYGVQCFARYPDHMKQHDFFKSAMPEGYVQERTISFKDDGTYK 1′RAEVKFEGDT LVNRIELKGIDFKEDGNILGHKLEYNFNSHNVYITADKQ (SEQ ID NO:28) or KNGIK ANFKIRHNVEDGSVQLADHYQQNTPIGDGPVLLPDNHYLSTQSVLSKDPNEKRDHM VLLEFVTAAGITHGMDELYK (SEQ ID NO:29), wherein one fragment dimerizes or folds together with the other fragment.

In some embodiments, the fragment of Renilla luciferase comprises MASKVYDPE QRKRMITGPQWWARCKQMNVLDSFINYYDSEKHAENAVIFLHGNAAS SYLWRHVV PHIEPVARCIIPDLIGMGKSGKSGNGSYRLLDHYKYLTAWFELLNLPKKIIFVGHDWG ACLAFHYSYEHQDKIKAIVHAESVVDVIESWDEWPDIEEDIALIKSEEGEKMVLENNF FVETMLPSKIMRKLEPEEFAAYLEPFKEKGEVRRPTLSWPREIPLVKGG (SEQ ID NO:30) or KPDVVQIVRNYNAYLRASDDLPKMFIESDPGFFSNAIVEGAKKFPNTEFV KVKGLHFSQEDAPDEMGKYIKSFVERVLKNEQ (SEQ ID NO:31), wherein one fragment dimerizes or folds together with the other fragment.

In some embodiments, the fragment of murine dihydrofolate reductase (DHFR) comprises amino acids 1-105 or 106-186 thereof, wherein one fragment dimerizes or folds together with the other fragment.

In some embodiments, the fragment of S. cerevisiae ubiquitin comprises amino acids 1-34 (MQIFVKTLTGKTITLEVESSDTIDNVKSKIQDKE; SEQ ID NO:63) or 35-76 (GIPPDQQRLIFAGKQLEDGRTLSDYNIQKESTLHLVLRLRGG; SEQ ID NO:64) thereof, wherein one fragment dimerizes or folds together with the other fragment.

In some embodiments, the fragment of β-lactamase comprises amino acids 25-197 or 198-286 thereof, wherein one fragment dimerizes or folds together with the other fragment.

In some embodiments, the fragment of Herpes simplex virus type 1 thymidine kinase comprises amino acids 1-265 or 266-376 thereof, wherein one fragment dimerizes or folds together with the other fragment.

In some embodiments, the fragment of the protein of interest of a first fusion protein and the fragment of the protein of interest of a second fusion protein can dimerize or fold together. In some embodiments, a first fusion protein comprises a fragment of a protein of interest fused to a ligand binding domain for a small molecule ligand, and a second fusion protein comprises a fragment of a protein of interest fused to a ligand binding domain for a small molecule ligand. In some embodiments, a first fusion protein comprises a fragment of a protein of interest fused to an interactive protein domain, and a second fusion protein comprises a fragment of a protein of interest fused to an interactive protein domain.

In some embodiments, the interactive protein domain of a first fusion protein is fused to the N-terminus of the fragment of the protein of interest, and the interactive protein domain of a second fusion protein is fused to the N-terminus of the fragment of the protein of interest; or the interactive protein domain of a first fusion protein is fused to the C-terminus of the fragment of the protein of interest, and the interactive protein domain of a second fusion protein is fused to the C-terminus of the fragment of the protein of interest; or the interactive protein domain of one of the first fusion protein and second fusion protein is fused to the N-terminus of the fragment of the protein of interest, and the interactive protein domain of the other of the first fusion protein and second fusion protein is fused to the C-terminus of the fragment of the protein of interest.

In some embodiments, both the first fusion protein and second fusion protein comprise a linker between the protein of interest and the ligand binding domain. In some embodiments, each linker is, independently, a Ser/Gly linker, a Poly-Asparagine linker, or a linker comprising the amino acid sequence AGSSAAGSGS (SEQ ID NO:17), as described herein.

Thus, the present disclosure provides any of the metabolic labeling methods described herein, further comprising performing a Ligand Directed TAPER (LD-TAPER) reaction using the nucleic acid template as a target nucleic acid sequence for the LD-TAPER reaction.

In some embodiments, the LD-TAPER reaction comprises: contacting the nucleic acid template with a first haplomer-ligand complex, wherein the first haplomer-ligand complex comprises a first haplomer, wherein the first haplomer comprises a first polynucleotide, and a first small molecule ligand linked to the 5′ or 3′ terminus of the first haplomer, wherein the first small molecule ligand comprises a first small molecule ligand partner binding site; contacting the nucleic acid template with a second haplomer-ligand complex, wherein the second haplomer-ligand complex comprises a second haplomer, wherein the second haplomer comprises a second polynucleotide, and a second small molecule ligand linked to the 5′ or 3′ terminus of the second haplomer, wherein the second small molecule ligand comprises a second small molecule ligand partner binding site; contacting the first haplomer-ligand complex with a first fusion protein, wherein the first fusion protein comprises a first fragment of a protein of interest fused to a first ligand binding domain for a small molecule ligand; contacting the second haplomer-ligand complex with a second fusion protein, wherein the second fusion protein comprises a second fragment of the protein of interest fused to a second ligand binding domain for a small molecule ligand; wherein the first ligand of the first haplomer-ligand complex is linked to the 5′ terminus of the first polynucleotide of the first haplomer-ligand complex; wherein the second ligand of the second haplomer-ligand complex is linked to the 3′ terminus of the second polynucleotide of the second haplomer-ligand complex; wherein the first polynucleotide of the first haplomer-ligand complex is substantially complementary to the nucleic acid template; wherein the second polynucleotide of the second haplomer-ligand complex is substantially complementary to the nucleic acid template at a site in spatial proximity to the first polynucleotide of the first haplomer-ligand complex; wherein the first small molecule ligand of the first haplomer-ligand complex and the first ligand binding domain of the first fusion protein can interact; and wherein the second small molecule ligand of the second haplomer-ligand complex and the second ligand binding domain of the second fusion protein can interact; thereby resulting in the folding or dimerization of the first fragment of the protein of interest of the first fusion protein with the second fragment of the protein of interest of the second fusion protein. In some embodiments, the first small molecule ligand is an FKBP binding compound, and the first ligand binding domain for the first small molecule ligand is an FKBP domain or a FRB domain; and the second small molecule ligand is an FKBP binding compound, and the second ligand binding domain for the second small molecule ligand is an FKBP domain or a FRB domain.

In some embodiments, the LD-TAPER reaction comprises: contacting the nucleic acid template with a first haplomer-ligand complex, wherein the first haplomer-ligand complex comprises a first haplomer, wherein the first haplomer comprises a first polynucleotide, and a first ligand linked to the 5′ or 3′ terminus of the first haplomer, wherein the first ligand is a first interactive protein domain and comprises a first ligand partner binding site; contacting the nucleic acid template with a second haplomer-ligand complex, wherein the second haplomer-ligand complex comprises a second haplomer, wherein the second haplomer comprises a second polynucleotide, and a second ligand linked to the 5′ or 3′ terminus of the second haplomer, wherein the second ligand is a second interactive protein domain and comprises a second ligand partner binding site; contacting the first haplomer-ligand complex with a first fusion protein, wherein the first fusion protein comprises a first fragment of a protein of interest fused to a third interactive protein domain; contacting the second haplomer-ligand complex with a second fusion protein, wherein the second fusion protein comprises a second fragment of the protein of interest fused to a fourth interactive protein domain; wherein the first ligand of the first haplomer-ligand complex is linked to the 5′ terminus of the first polynucleotide of the first haplomer-ligand complex; wherein the second ligand of the second haplomer-ligand complex is linked to the 3′ terminus of the second polynucleotide of the second haplomer-ligand complex; wherein the first polynucleotide of the first haplomer-ligand complex is substantially complementary to the nucleic acid template; wherein the second polynucleotide of the second haplomer-ligand complex is substantially complementary to the nucleic acid template at a site in spatial proximity to the first polynucleotide of the first haplomer-ligand complex; wherein the first interactive protein domain of the first haplomer-ligand complex and the third interactive protein domain of the first fusion protein can interact; and wherein the second interactive protein domain of the second haplomer-ligand complex and the fourth interactive protein domain of the second fusion protein can interact; and thereby resulting in the folding or dimerization of the first fragment of the protein of interest of the first fusion protein with the second fragment of the protein of interest of the second fusion protein. In some embodiments, the first interactive protein domain and the third interactive protein domain are jun/fos, mad/max, myc/max, or NZ/CZ interacting zipper motifs; and the second interactive protein domain and the fourth interactive protein domain are jun/fos, mad/max, myc/max, or NZ/CZ interacting zipper motifs.

In some embodiments, the LD-TAPER reaction comprises: contacting the nucleic acid template with a complex formed by the interaction of a first haplomer-ligand complex with a first fusion protein, wherein: the first haplomer-ligand complex comprises a first haplomer, wherein the first haplomer comprises a first polynucleotide, and a first small molecule ligand linked to the 5′ or 3′ terminus of the first polynucleotide, wherein the first small molecule ligand comprises a first ligand partner binding site; the first fusion protein comprises a first fragment of a protein of interest fused to a first ligand binding domain for the first small molecule ligand; and the first small molecule ligand of the first haplomer-ligand complex interacts with the first ligand binding domain for the first small molecule ligand of the first fusion protein; and contacting the nucleic acid template with a complex formed by the interaction of a second haplomer-ligand complex with a second fusion protein, wherein: the second haplomer-ligand complex comprises a second haplomer, wherein the second haplomer comprises a second polynucleotide, and a second small molecule ligand linked to the 5′ or 3′ terminus of the second polynucleotide, wherein the second small molecule ligand comprises a second ligand partner binding site; the second fusion protein comprises a second fragment of the protein of interest fused to a second ligand binding domain for the second small molecule ligand; the second small molecule ligand of the second haplomer-ligand complex interacts with the second ligand binding domain for the second small molecule ligand of the second fusion protein; thereby resulting in the folding or dimerization of the fragment of the protein of interest of the first fusion protein with the fragment of the protein of interest of the second fusion protein. In some embodiments, the first small molecule ligand is an FKBP binding compound, and the first ligand binding domain for the first small molecule ligand is an FKBP domain or a FRB domain; and the second small molecule ligand is an FKBP binding compound, and the second ligand binding domain for the second small molecule ligand is an FKBP domain or a FRB domain.

In some embodiments, the LD-TAPER reaction comprises: contacting the nucleic acid template with a complex formed by the interaction of a first haplomer-ligand complex with a first fusion protein, wherein: the first haplomer-ligand complex comprises a first haplomer, wherein the first haplomer comprises a first polynucleotide, and a first ligand linked to the 5′ or 3′ terminus of the first polynucleotide, wherein the first ligand is a first interactive protein domain and comprises a first ligand partner binding site; the first fusion protein comprises a first fragment of a protein of interest fused to a third interactive protein domain; the first interactive protein domain of the first haplomer-ligand complex and the third interactive protein domain of the first fusion protein can interact; and contacting the nucleic acid template with a complex formed by the interaction of a second haplomer-ligand complex with a second fusion protein, wherein: the second haplomer-ligand complex comprises a second haplomer, wherein the second haplomer comprises a second polynucleotide, and a second ligand linked to the 5′ or 3′ terminus of the second polynucleotide, wherein the second ligand is a second interactive protein domain and comprises a second ligand partner binding site; the second fusion protein comprises a second fragment of a protein of interest fused to a fourth interactive protein domain; the second interactive protein domain of the second haplomer-ligand complex and the fourth interactive protein domain of the second fusion protein can interact; thereby resulting in the folding or dimerization of the first fragment of the protein of interest of the first fusion protein with the second fragment of the protein of interest of the second fusion protein. In some embodiments, the first interactive protein domain and the third interactive protein domain are jun/fos, mad/max, myc/max, or NZ/CZ interacting zipper motifs; and the second interactive protein domain and the fourth interactive protein domain are jun/fos, mad/max, myc/max, or NZ/CZ interacting zipper motifs.

In some embodiments, the LD-TAPER reaction comprises: contacting the nucleic acid template with a bottle haplomer-ligand complex, wherein the bottle haplomer-ligand complex comprises: a) a bottle haplomer, wherein the bottle haplomer comprises a first polynucleotide, wherein the first polynucleotide comprises: i) a first stem portion comprising from about 10 to about 20 nucleotide bases; ii) an anti-target loop portion comprising from about 16 to about 40 nucleotide bases and having a first end to which the first stem portion is linked, wherein the anti-target loop portion is substantially complementary to the nucleic acid template; and iii) a second stem portion comprising from about 10 to about 20 nucleotide bases linked to a second end of the anti-target loop portion, wherein the first stem portion is substantially complementary to the second stem portion; and b) a first small molecule ligand linked to the terminal end of either the first stem portion or the second stem portion, wherein the first small molecule ligand comprises a first small molecule ligand partner binding site; wherein the Tₘ of the anti-target loop portion:nucleic acid template is greater than the Tₘ of the first stem portion:second stem portion; contacting the nucleic acid template with a second haplomer-ligand complex, wherein the second haplomer-ligand complex comprises a second haplomer, wherein the second haplomer comprises a second polynucleotide, and a second small molecule ligand linked to the 5′ or 3′ terminus of the second polynucleotide, wherein the second small molecule ligand comprises a second small molecule ligand partner binding site; wherein the second haplomer-ligand complex comprises a nucleotide portion that is substantially complementary to the stem portion of the bottle haplomer-ligand complex that is linked to the first small molecule ligand of the bottle haplomer-ligand complex; contacting the bottle haplomer-ligand complex with a first fusion protein, wherein the first fusion protein comprises a first fragment of a protein of interest fused to a first ligand binding domain for the first small molecule ligand, wherein the first small molecule ligand of the bottle haplomer-ligand complex and the first small molecule ligand binding domain of the first fusion protein can interact; and contacting the second haplomer-ligand complex with a second fusion protein, wherein the second fusion protein comprises a second fragment of the protein of interest fused to a second small molecule ligand binding domain for the second small molecule ligand, wherein the second small molecule ligand of the second haplomer-ligand complex and the second small molecule ligand binding domain of the second fusion protein can interact; thereby resulting in the folding or dimerization of the first fragment of the protein of interest of the first fusion protein with the second fragment of the protein of interest of the second fusion protein. In some embodiments, the first small molecule ligand is an FKBP binding compound, and the first ligand binding domain for the first small molecule ligand is an FKBP domain or a FRB domain; and the second small molecule ligand is an FKBP binding compound, and the second ligand binding domain for the second small molecule ligand is an FKBP domain or a FRB domain.

In some embodiments, the LD-TAPER reaction comprises: contacting the nucleic acid template with a bottle haplomer-ligand complex, wherein the bottle haplomer-ligand complex comprises: a) a bottle haplomer, wherein the bottle haplomer comprises a first polynucleotide, wherein the first polynucleotide comprises: i) a first stem portion comprising from about 10 to about 20 nucleotide bases; ii) an anti-target loop portion comprising from about 16 to about 40 nucleotide bases and having a first end to which the first stem portion is linked, wherein the anti-target loop portion is substantially complementary to the nucleic acid template; and iii) a second stem portion comprising from about 10 to about 20 nucleotide bases linked to a second end of the anti-target loop portion, wherein the first stem portion is substantially complementary to the second stem portion; and b) a first ligand linked to the terminal end of either the first stem portion or the second stem portion, wherein the first ligand is a first interactive protein domain and comprises a first ligand partner binding site; wherein the Tₘ of the anti-target loop portion:nucleic acid template is greater than the Tₘ of the first stem portion:second stem portion; contacting the nucleic acid template with a second haplomer-ligand complex, wherein the second haplomer-ligand complex comprises a second haplomer, wherein the second haplomer comprises a second polynucleotide, and a second ligand linked to the 5′ or 3′ terminus of the second polynucleotide, wherein the second ligand is a second interactive protein domain and comprises a second ligand partner binding site; wherein the second haplomer-ligand complex comprises a nucleotide portion that is substantially complementary to the stem portion of the bottle haplomer-ligand complex that is linked to the first ligand of the bottle haplomer-ligand complex; contacting the bottle haplomer-ligand complex with a first fusion protein, wherein the first fusion protein comprises a first fragment of a protein of interest fused to a third interactive protein domain; wherein the first interactive protein domain of the bottle haplomer-ligand complex and the third interactive protein domain of the first fusion protein can interact; and contacting the second haplomer-ligand complex with a second fusion protein, wherein the second fusion protein comprises a second fragment of the protein of interest fused to a fourth interactive protein domain; wherein the second interactive protein domain of the second haplomer-ligand complex and the fourth interactive protein domain of the second fusion protein can interact; thereby resulting in the folding or dimerization of the first fragment of the protein of interest of the first fusion protein with the second fragment of the protein of interest of the second fusion protein. In some embodiments, the first interactive protein domain and the third interactive protein domain are jun/fos, mad/max, myc/max, or NZ/CZ interacting zipper motifs; and the second interactive protein domain and the fourth interactive protein domain are jun/fos, mad/max, myc/max, or NZ/CZ interacting zipper motifs.

In any of the methods described herein, the first polynucleotide and the second polynucleotide can comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

In any of the methods described herein, the first fragment of the protein of interest and the second fragment of the protein of interest are fragments of an active effector agent chosen from a peptide, protein, or a therapeutic agent. In some embodiments, the peptide or protein is a toxin, a pro-apoptotic agent, or a ligand for an antibody or antibody fragment.

The Split-Protein TAPER (SP-TAPER) processes and components thereof can be generally described by the following general representations. Numerous proteins can be divided into two separate polypeptide fragments that are disordered in isolation, but which can undergo accurate co-folding when held together in the correct orientation in spatial proximity. Such spatially enforced folding can result in the formation of the mature protein, including reconstitution of its original functional properties. One means for eliciting spatial proximity between such protein fragments has been to append each to independently folding and mutually interactive small protein domains, such as leucine zippers. This process has commonly been called the Protein Complementation Assay (PCA), or split-protein technology (see, FIGS. 1 and 2 of PCT Publication WO 18/93978). The specific choice of a site within a primary amino acid sequence for division of a protein of interest can be rationally guided when the protein three-dimensional structure is available. Loops or other structural features which can be modified without compromising general protein folding or function are accordingly favored for split-protein procedures. The spatial orientation of N and C-termini of proteins of interest may also be significant. For example, where the N- and C-termini are packed in spatial proximity in the mature folded protein (see, FIG. 1 of PCT Publication WO 18/93978), a parallel orientation of these termini in split-protein complementation may be more compatible with the required folding pathway than an anti-parallel orientation. Nevertheless, such potential constraints may be reduced or eliminated if each fragment is equipped with a flexible linker sequence of sufficient length to allow spatial positioning. Where no other information exists regarding the utility of a chosen fragmentation point for split-protein analyses, the system may be empirically tested by separately expressing fragments as appropriate fusions with self-folding and interactive protein domains, and testing reconstitution of functional activity upon mixing of fragments in vitro, or co-expressing the fusion products intracellularly. As an example of one such arrangement, a protein rendered as two fragments A and B is engineered to be expressed separately as A-(C-terminal)-Jun and Fos-(N-terminal)-B, where Jun and Fos are derived from c-Jun and c-Fos mutually interactive leucine zippers, and where long serine-glycine linkers separate the Jun/Fos segments from the desired polypeptide.

To adapt conventional TAPER to SP-TAPER, protein fragments are coupled with nucleic acid haplomers, whereby the haplomers enable hybridization-mediated molecular proximity between the two protein fragments. These haplomers are appended to the new N- or C-termini generated by expression of the protein of choice as two separate fragments (herein, these new termini are referred to as N*- and C*- respectively). The 5′ or 3′ ends of an oligonucleotide can be appended to either the N*- and C*-termini of split-protein fragments (see, FIG. 3 of PCT Publication WO 18/93978) by various chemistries (panel A vs. panel B).

Prior to nucleic acid conjugations, protein fragments of interest are expressed in bacterial systems and purified. In some embodiments, expression systems include, but are not limited to, affinity fusions with maltose-binding protein, or hexahistidine tags. In some embodiments, intein fusions are expressed such that the desired protein fragments are cleaved off in vitro under appropriate conditions.

In some embodiments, the coupling between haplomers and protein fragments is mediated by bridging terminal —SH groups. For oligonucleotides, 5′ or 3′ —SH groups are readily created by syntheses, where the sulfhydryl group is typically generated from a terminal precursor disulfide immediately prior to use, by treatment with reducing agents such as dithiothreitol (DTT) or TCEP. For polypeptides, N- or C-terminal —SH groups are most simply generated by placing a terminal cysteine residue at the appropriate site. Joining of —SH tagged oligonucleotides can be affected by means of bifunctional maleimide reagents including, but not limited to, 1,8-bis(maleimido)diethylene glycol and 1,11-bis(maleimido)triethylene glycol. The presence of internal cysteines within the polypeptide fragments of interest is a potential hurdle of this approach, but in practice it has been found that terminal cysteines are much more efficiently modified than those embedded within a longer sequence.

In some embodiments, the coupling between haplomers and protein fragments is mediated by alternative chemistries. For N-terminal polypeptide conjugations with haplomers, these approaches include, but are not limited to, ketene chemistry, thioazolidines, or isocyanato chelates. For C-terminal polypeptide conjugations with haplomers, these approaches include, but are not limited to, iodinylation of engineered C-terminal selenocysteines, and methods where labeling is coupled with intein cleavage. In the latter circumstances, cleavage of an N-terminal protein fragment of interest from a fused intein sequence can be effected by means of a hydrazino compound bearing an azido group (Kalia, et al., Chem. BioChem., 2006, 7, 1375-1383). Subsequent to this, an oligonucleotide carrying a 5′ or 3′ cyclooctyne group can be readily joined to the azide moiety through copper-free click chemistry. Alternately, an N-terminal protein fragment of interest can be cleaved from a fused intein sequence by conventional treatment with 2-mercaptoethane sulfonic acid, while co-reacted with a novel modified cysteine bearing a methyltetrazine group ((R)-2-amino-3-mercapto-N-(3-(4-(6-methyl-1,2,4,5-tetrazin-3-yl)phenoxy)propyl)propanamide):

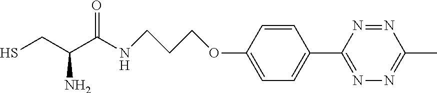 [SMILES: Cc1nnc(-c2ccc(OCCCNC(=O)[C@@H](N)CS)cc2)nn1]

This combines release of the desired N-terminal protein fragment with conjugation of the cysteine-methytetrazine. In turn, an oligonucleotide carrying a 5′ or 3′ trans-cyclooctene group reacts rapidly and specifically with the appended methytetrazine moiety.

In some embodiments, the coupling between haplomers and protein fragments is mediated through an extended genetic code. To implement this, a TAG stop codon (at the DNA level) is engineered at an N- or C-terminal position, and the bacterial strain used for expression purposes is co-transfected with plasmids encoding a bio-orthogonal aminoacyl tRNA synthase/tRNA pair, derived from an archaeal source with specific sequence modifications. In such circumstances, the aminoacyl tRNA synthase has been engineered and selected to bio-orthogonally charge its cognate tRNA with the desired unnatural amino acid, which is incorporated into proteins in a site-specific manner by virtue of the recognition of UAG codons by the tRNA anticodon triplet. In some instantiations of the extended genetic code approach, the unnatural amino bears a selectively-reactive moiety, including, but not limited to, trans-cyclooctene. When an unnatural amino acid residue with a side-chain bearing a specific selectively-reactive moiety group is incorporated at or near the N- or C-terminus of a polypeptide of choice, an oligonucleotide bearing a reaction-complementary selectively-reactive moiety can be chemically ligated to the polypeptide via the particular click reaction itself. In the embodiment where the incorporated unnatural amino acid carries a trans-cyclooctene, bio-orthogonally reactive oligonucleotides are appended with a 5′ or 3′ methyltetrazine group.

In some embodiments, where the haplomers appended to polypeptides are composed of DNA, conjugates may be purified by hybridization with a biotinylated complementary RNA strand and subsequent immobilization on solid-phase streptavidin. Components of the initial mixture which lack DNA oligonucleotide haplomers are not bound to the solid-phase streptavidin, and are therefore removed by washing steps. Bound conjugates are then released by treatment with RNaseH, which specifically digests the RNA strand in RNA:DNA hybrids.

SP-TAPER can be instituted where the hybridization-mediated polypeptide juxtaposition (that enables folding and functional activity reconstitution) occurs by means of a number of distinct molecular architectures. In the simplest arrangement, the haplomers on each split-protein fragments are substantially complementary to each other. Direct hybridization between such haplomers promotes spatial proximity of the appended protein fragments, and in turn their co-interaction via the native folding pathway (see, FIG. 4 of PCT Publication WO 18/93978). Herein, this configuration is referred to as “Architecture 1.” To closely parallel conventional TAPER, a pair of SP-haplomers can also co-hybridize in spatial proximity to a third-party linear target template, rather than being complementary to each other. By so doing, the appended polypeptide sequences are arranged in spatial juxtaposition in the desired orientation, such that the mature folded protein product can form (see, FIG. 5 of PCT Publication WO 18/93978). Herein, this configuration is referred to as “Architecture 2.” Within this architecture, the gap between the two hybridizing SP-haplomers on a complementary template (i.e., nucleic acid template) may be zero (that is, when the SP-haplomers are precisely juxtaposed) or with N>0, where N=the number of template nucleotides between the 5′ and 3′ ends of the SP-haplomer pair. (In practice, as N increases, the efficiency of interaction between haplomer polypeptides will tend to diminish).

Additional architectures are possible for SP-TAPER, where the sites of hybridization of SP-haplomers are non-contiguous in terms of the primary sequence of the nucleic acid template. Where discontinous recognition sites for the pair of SP-haplomers are brought into spatial proximity by a stem-loop structure (herein, termed “Architecture 3”), the appended polypeptide sequences can co-fold into the mature protein structure (see, FIG. 6 of PCT Publication WO 18/93978).

In the template-based Architectures 2 and 3, the 5′ and 3′ of the SP-haplomers are directed towards each other in terms of the coordinates of the template strand. This has previously been termed an “endo” configuration. Where the template strand can form a sizeable loop structure, opposite haplomer arrangement (“exo” configuration, with the 5′ and 3′ of the SP-haplomers directed away each other in terms of the coordinates of the template strand (see FIG. 7 of PCT Publication WO 18/93978) can also result in spatial proximity of the appended polypeptide segments, along with functional co-folding Herein, this configuration is referred to as “Architecture 4.”

Proteins that can be applied as split polypeptides towards templated reassembly directed by SP-TAPER include all those capable of delivering a reporter signal. A non-limiting set of reporter protein examples includes fluorescent proteins (such as GFP and derivatives, YFP, mCherry, dsRed, VENUS, and CFP), and luciferases (firefly luciferase, Renilla luciferase). Other classes of proteins encompassed by SP-TAPER applications include, but are not limited to, transcription factors, signal transduction pathway factors, and gene editing proteins.

In some embodiments, SP-TAPER is targeted towards the templated assembly of single-chain immunoglobulin variable region (scFv) proteins. These typically contain extended serine-glycine linker sequences which enable the association of the variable region heavy and light chain segments. This linker sequence is a convenient site for split protein generation, where the two immunoglobulin variable region segments are appended with nucleic acid tags according to the desired architecture, after which their assembly and resulting antigen-binding properties are mediated by the presence of specific template. This enables in situ generation of a desired antigen-binding specificity in a cell target of interest, as defined by a cell-specific nucleic acid sequence. Applications of scFv-targeted SP-TAPER include, but are not limited to, the use of fluorescence-activating proteins (FAPs, scFv molecules generating fluorescence in target ligands).

In some embodiments, SP-TAPER is applied towards the templated assembly of small highly toxic proteins, or ribotoxins, which function by enzymatically disabling eukaryotic ribosomes. Such proteins include, but are not limited to, ricin A chain, Aspf1, α-sarcin, mitogillin, and hirsutellin A. These proteins are attractive for SP-TAPER through their small sizes and high toxicities. Hirsutellin A, as a non-limiting example, has a number of potential split-protein fragmentation sites, including a diglycine turn (see, FIG. 10 of PCT Publication WO 18/93978). While extreme toxicity can be a significant restriction on the deployment of ribotoxins as direct immunoconjugates (through unacceptable by-stander effects) this is effectively circumvented by SP-TAPER. Where ribotoxin split-protein fragments lack the toxic activity of their parental protein, their circulating fragments are innocuous. With SP-TAPER, such fragments only assemble into fully active proteins in the presence of specific templates associated with a pathological cell target.

By the same principles as noted for ribotoxins, SP-TAPER is also applicable to the template-directed assembly of other small and highly toxic proteins, including, but not limited to, diphtheria toxin and cholera toxin. Further examples are provided below.

Nucleic acid templates that serve as templates for SP-TAPER include any nucleic acid sequence which distinguishes a target of choice, whether the sequence corresponds to a cellular RNA molecule of any description, or derives from an aptamer-mediated adaptation process (see, PCT Publication WO 17/205277), or from any other process whereby a suitable template sequence is affixed at a desired cellular site.

Nucleic acid templates that serve as templates for SP-TAPER can be produced on cell surfaces, where the template-promoted assembly of SP-haplomers is also a surface effect. In some embodiments, the specific desired templates (and desired split-protein assembly sites) are internally situated within a target cell type, whether of tumor origin, arising through aberrant immune pathways, or originating by means of any other type of pathological process. In such cases, the SP-haplomers are dispatched to the intracellular environment by various delivery technologies including, but not limited to, gymnotic approaches, and a wide variety of nanoparticles. The latter category includes, but is not limited to, simple and multi-layered liposomes, dendrimers, extracellular vesicles, DNA or other nucleic acid origami cages, engineered bacterial vehicles, engineered mitochondria, virally-derived structures, ribonucleoprotein vaults, and protein or PEGylated protein self-assembling compartments. As with conventional TAPER, while target precision of delivery is useful, it is not essential, since in the absence of the pathologically-defined target sequence, no split-protein assembly will take place. In other words, delivery to a normal “off-target” cell does not have deleterious side-effects for the implementation of SP-TAPER.

In some embodiments, the folding pathway of the split polypeptide fragments in SP-TAPER may be assisted by the provision of protein chaperones (including, but not limited to, members of diverse heat-shock protein families), or low-molecular weight chemical chaperones. Small-molecule chaperones in the latter category with non-specific chaperoning function include, but are not limited to, 4-phenyl butyrate, deoxycholic acid, ursodeoxycholic acid, or taurourso-deoxycholic acid. In some embodiments, SP-TAPER may utilize small molecules that have beneficial folding enhancement towards specific target polypeptide fragments of interest, where such low-molecular weight compounds are defined as pharmacological chaperones, or pharmacoperones.

The haplomers for SP-TAPER comprise: a) a polynucleotide that is substantially complementary to a nucleic acid template; and b) an N-terminal protein fragment or a C-terminal protein fragment, wherein the 3′ or 5′ terminus of the polynucleotide is linked to the N-terminus of the C-terminal protein fragment or the C-terminus of the N-terminal protein fragment. In some embodiments, the polynucleotide of the haplomer comprises from about 6 to about 20 nucleotide bases. In some embodiments, the the polynucleotide of the haplomer comprises from about 8 to about 15 nucleotide bases.

In some embodiments, a pair of haplomers works in tandem. In some embodiments, the protein fragment of the first haplomer is linked to the 5′ terminus of the polynucleotide of the first haplomer, and the protein fragment of the second haplomer is linked to the 3′ terminus of the polynucleotide of the second haplomer.

In some embodiments, the polynucleotide of the first haplomer is substantially complementary to the polynucleotide of the second haplomer. In some embodiments, the polynucleotide of the first haplomer is substantially complementary to a nucleic acid template, and the polynucleotide of the second haplomer is substantially complementary to the nucleic acid template at a site in spatial proximity to the polynucleotide of the first haplomer.

In any of the embodiments described herein, the haplomers are in spatial proximity (when bound to a nucleic acid template) such that the protein fragments can properly interact to induce the interaction of their respective fragments of the protein of interest. Thus, for any haplomer pairs, reactivity can occur where the gap N between the first and second haplomer binding to the nucleic acid template is 0 (i.e., the haplomers are immediately juxtaposed), and progressively greater gaps (N>0) will progressively diminish activity. Thus, in some embodiments, there is 0 nucleotides between the binding of a first haplomer and second haplomer to the nucleic acid template. In some embodiments, there is less than 6 nucleotides between the binding of a first haplomer and second haplomer to the nucleic acid template. In some embodiments, there is less than 5 nucleotides between the binding of a first haplomer and second haplomer to the nucleic acid template. In some embodiments, there is less than 4 nucleotides between the binding of a first haplomer and second haplomer to the nucleic acid template. In some embodiments, there is less than 3 nucleotides between the binding of a first haplomer and second haplomer to the nucleic acid template. In some embodiments, there is less than 2 nucleotides between the binding of a first haplomer and second haplomer to the nucleic acid template.

In some embodiments, the protein fragment and polynucleotide of the first haplomer both comprises reactive bio-orthogonal moieties, and/or the protein fragment and polynucleotide of the second haplomer both comprises selectively-reactive moieties, wherein the selectively-reactive moiety of the first haplomer is reactable with the selectively-reactive moiety of the second haplomer.

In some embodiments, the N-terminal fragment comprises the amino acid sequence of APIVTCRKLDGREKPFKVDVATAQAQARKAGLTTGKSGDPHRYFAGDHIRWGVN NCDKADAILWEYPIYWVGKNAEWAKDVKTSQQKG (SEQ ID NO:34), and the C-terminal fragment comprises the amino acid sequence of GPTPIRVVYANSRGAVQYCGV MTHSKVDKNNQGKEFFEKCD (SEQ ID NO:35).

In some embodiments, the N-terminal fragment comprises the amino acid sequence of APIVTCRPKLDG (SEQ ID NO:36), and the C-terminal fragment comprises the amino acid sequence of REKPFKVDVATAQAQARKAGLTTGKSGDPHRYFAGDHIRWGVNN CDKADAILWEYPIYWVGKNAEWAKDVKTSQQKGGPTPIRVVYANSRGAVQYCGV MTHSKVDKNNQGKEFFEKCD (SEQ ID NO:37).

In some embodiments, the N-terminal fragment comprises the amino acid sequence of APIVTCRPKLDGREKPFKVDVATAQAQARKAGLTTGK (SEQ ID NO:38), and the C-terminal fragment comprises the amino acid sequence of SGDPHRYFAGDHIRWGVNNC DKADAILWEYPIYWVGKNAEWAKDVKTSQQKGGPTPIRVVYANSRGAVQYCGVM THSKVDKNNQGKEFFEKCD (SEQ ID NO:39).

In some embodiments, the N-terminal fragment comprises the amino acid sequence of APIVTCRPKLDGREKPFKVDVATAQAQARKAGLTTGKSGDPHRYFAGDHIRWGV NNCDKAD (SEQ ID NO:40), and the C-terminal fragment comprises the amino acid sequence of AILWEYPIYWVGKNAEWAKDVKTSQQKGGPTPIRVVYANSRGAVQYC GVMTHSKVDKNNQGKEFFEKCD (SEQ ID NO:41).

In some embodiments, the N-terminal fragment comprises the amino acid sequence of APIVTCRPKLDGREKPFKVDVATAQAQARKAGLTTGKSGDPHRYFAGDHIRWGV NNCDKADAILWEYPIYWVG (SEQ ID NO:42), and the C-terminal fragment comprises the amino acid sequence of KNAEWAKDVKTSQQKGGPTPIRVVYANSRGAVQYCGVM THSKVDKNNQGKEFFEKCD (SEQ ID NO:43).

In some embodiments, the N-terminal fragment comprises the amino acid sequence of APIVTCRPKLDGREKPFKVDVATAQAQARKAGLTTGKSGDPHRYFAGDHIRWGV NNCDKADAILWEYPIYWVGKNAEWAKD (SEQ ID NO:44), and the C-terminal fragment comprises the amino acid sequence of VKTSQQKGGPTPIRVVYANSRGAVQYC GVMTHSKVDKNNQGKEFFEKCD (SEQ ID NO:45).

In some embodiments, the N-terminal fragment comprises the amino acid sequence of APIVTCRPKLDGREKPFKVDVATAQAQARKAGLTTGKSGDPHRYFAGDHIRWGV NNCDKADAILWEYPIYWVGKNAEWAKDVKTSQ (SEQ ID NO:46), and the C-terminal fragment comprises the amino acid sequence of QKGGPTPIRVVYANSRGAVQYCGVMT HSKVDKNNQGKEFFEKCD (SEQ ID NO:47).

In some embodiments, the N-terminal fragment comprises the amino acid sequence of APIVTCRPKLDGREKPFKVDVATAQAQARKAGLTTGKSGDPHRYFAGDHIRWGV NNCDKADAILWEYPIYWVGKNAEWAKDVKTSQQKGGPTPIRVVYANSRG (SEQ ID NO:48), and the C-terminal fragment comprises the amino acid sequence of AVQYCGVMT HSKVDKNNQGKEFFEKCD (SEQ ID NO:49).

In some embodiments, the N-terminal fragment comprises the amino acid sequence of APIVTCRPKLDGREKPFKVDVATAQAQARKAGLTTGKSGDPHRYFAGDHIRWGV NNCDKADAILWEYPIYWVGKNAEWAKDVKTSQQKGGPTPIRVVYANSRGAVQYC GVMTHSKVDKN (SEQ ID NO:50), and the C-terminal fragment comprises the amino acid sequence of NQGKEFFEKCD (SEQ ID NO:51).

In some embodiments, the N-terminal fragment comprises the amino acid sequence of APIVTCRPKLDGREKPFKVDVATAQAQARKAGLT (SEQ ID NO:52), and the C-terminal fragment comprises the amino acid sequence of TGKSGDPHRYFAGDHIRWGVN NCDKADAILWEYPIYWVGKNAEWAKDVKTSQQKGGPTPIRVVYANSRGAVQYCG VMTHSKVDKNNQGKEFFEKCD (SEQ ID NO:53).

In the SP-TAPER methods described herein, the bottle haplomers can comprise a polynucleotide, wherein the polynucleotide comprises: a) a first 3′ stem portion comprising from about 10 to about 20 nucleotide bases; b) an anti-target loop portion comprising from about 16 to about 40 nucleotide bases linked to the first 3′ stem portion, wherein the anti-target loop portion is substantially complementary to a nucleic acid template; and c) a second 5′ stem portion comprising from about 10 to about 20 nucleotide bases linked to the anti-target loop portion, wherein the first 3′ stem portion is substantially complementary to the second 5′ stem portion; wherein: i) the 5′ terminus of the polynucleotide comprises a —SH moiety; and ii) the Tₘ of the anti-target loop portion:nucleic acid template is greater than the Tₘ of the first stem portion: second stem portion.

In the SP-TAPER methods described herein, the bottle haplomers can comprise a polynucleotide, wherein the polynucleotide comprises: a) a first 3′ stem portion comprising from about 10 to about 20 nucleotide bases; b) an anti-target loop portion comprising from about 16 to about 40 nucleotide bases linked to the first 3′ stem portion, wherein the anti-target loop portion is substantially complementary to a nucleic acid template; and c) a second 5′ stem portion comprising from about 10 to about 20 nucleotide bases linked to the anti-target loop portion, wherein the first 3′ stem portion is substantially complementary to the second 5′ stem portion; wherein: i) the Tₘ of the anti-target loop portion:nucleic acid template is greater than the Tₘ of the first stem portion: second stem portion; and ii) the 5′ terminus or 3′ terminus of the polynucleotide is linked to the C-terminus of an N-terminal protein fragment or the N-terminus of a C-terminal protein fragment, wherein the terminus of the protein fragment lined to the polynucleotide comprises a cysteine or selenocysteine.

In some embodiments, the first stem portion comprises from about 12 to about 18 nucleotide bases. In some embodiments, the anti-target loop portion comprises from about 18 to about 35 nucleotide bases. In some embodiments, the second stem portion comprises from about 12 to about 18 nucleotide bases. The anti-target loop portion has a first end to which the first stem portion is linked. The anti-target loop portion is substantially complementary to a nucleic acid template. The second stem portion is linked to a second end of the anti-target loop portion. The first stem portion is substantially complementary to the second stem portion.

In some embodiments, the anti-target loop portion can further comprise an internal hinge region, wherein the hinge region comprises one or more nucleotides that are not complementary to the nucleic acid template. In some embodiments, the hinge region comprises from about 1 nucleotide to about 6 nucleotides, from about 1 nucleotide to about 5 nucleotides, from about 1 nucleotide to about 4 nucleotides, from about 1 nucleotide to about 3 nucleotides, or 1 or 2 nucleotides.

For the polynucleotides of the bottle haplomers described herein, the length of the particular polynucleotide or portion thereof is less important than the Tₘ of the duplex formed by the interaction of the polynucleotide, or portion thereof, with another nucleic acid molecule, or portion thereof. For example, the Tₘ of the duplex formed by the interaction of the anti-target loop portion with the nucleic acid template (e.g., anti-target loop portion:nucleic acid template) is greater than the Tₘ of the duplex formed by the interaction of the first stem portion with the second stem portion (e.g., first stem portion:second stem portion). In some embodiments, the Tₘ of the first stem portion:second stem portion subtracted from the Tₘ of the anti-target loop portion:nucleic acid template is from about 10° C. to about 40° C. In some embodiments, the Tₘ of the first stem portion:second stem portion subtracted from the Tₘ of the anti-target loop portion:nucleic acid template is from about 10° C. to about 20° C. In some embodiments, the Tₘ of the first stem portion:second stem portion is from about 40° C. to about 50° C. In some embodiments, the Tₘ of the anti-target loop portion:nucleic acid template is from about 60° C. to about 80° C.

In addition, translating the Tₘ information above into specific lengths of the nucleic acid molecules described herein can also depend on the GC content of each nucleic acid molecule. For example, the length of a suitable HPV model nucleic acid template is 30 bases (having a Tₘ of 70° C.), while that for the EBV model nucleic acid template is only 21 bases (having a Tₘ of 69° C.), owing to its greater % GC.

In some embodiments, a bottle haplomer works in tandem with a second haplomer.

In some embodiments, the bottle haplomer is any bottle haplomer described herein, and the second haplomer is any of the haplomers described herein. In some embodiments, the second haplomer comprises: a) a nucleotide portion comprising from about 6 to about 20 nucleotide bases that is substantially complementary to the stem portion of the bottle haplomer that is linked to the protein fragment of the bottle haplomer; and b) a protein fragment linked to the 5′ or 3′ terminus of the nucleotide portion of the second haplomer; wherein the Tₘ of the second haplomer:first or second stem portion linked to the protein fragment of the bottle haplomer is less than or equal to the Tₘ of the first stem portion:second stem portion of the bottle haplomer.

In some embodiments, the Tₘ of the duplex formed by the interaction of the second haplomer with either the first stem portion or the second stem portion, whichever stem portion is linked to the protein fragment (e.g., second haplomer:first or second stem portion linked to the protein fragment), is less than or equal to the Tₘ of the first stem portion:second stem portion. In some embodiments, the Tₘ of the duplex formed by the second haplomer and the first or second stem portion linked to the protein fragment subtracted from the Tₘ of the first stem portion:second stem portion is from about 0° C. to about 20° C. In some embodiments, the Tₘ of the duplex formed by the second haplomer and the first or second stem portion linked to the protein fragment subtracted from the Tₘ of the first stem portion:second stem portion is from about 5° C. to about 10° C. In some embodiments, the Tₘ of the duplex formed by the second haplomer and the first or second stem portion linked to the protein fragment is from about 30° C. to about 40° C.

This structural arrangement is designed such that in the absence of nucleic acid template, the locked first haplomer bottle does not significantly hybridize to its complementary second haplomer and, thus, template-directed product assembly is not promoted under such conditions. When the specific nucleic acid template is present, on the other hand, the bottle haplomer is “unlocked” by the formation of a more stable hybrid between the anti-target loop portion of the bottle haplomer and the nucleic acid template itself. Once this occurs, the first stem portion of the bottle haplomer that is linked to the protein fragment is free to hybridize to the available second haplomer, with resulting proximity between the protein fragments on both.

In the SP-TAPER methods described herein, the template polynucleotide can further be linked to a peptide; wherein: i) the 5′ terminus of the polynucleotide is coupled to the N-terminus or C-terminus of the peptide, or the 3′ terminus of the polynucleotide is coupled to the N-terminus or C-terminus of the peptide; and ii) the peptide is a ligand for a cell-surface molecule.

In some embodiments, the ligand is a peptide hormone or a neuropeptide. Examples of peptide hormones include, but are not limited to, alpha-MSH, amylin, anti-Müllerian hormone, adiponectin, atriopeptide, human growth hormone, gonadotropin releasing hormone, inhibin, somatostatin, adrenocorticotropic hormone, vasopressin, vasoactive intestinal peptide, gastrin, secretin, gastric inhibitory polypeptide, motilin, hepcidin, renin, relaxin, ghrelin, leptin, lipotropin, angiotensin I, angiotensin II, bradykinin, calcitonin, insulin, glucagon, insulin-like growth factor I, insulin-like growth factor II, glucagon-like peptide I, pancreatic polypeptide, betatrophin, cholecystokinin, endothelin, erythropoietin, thrombopoietin, follicle-stimulating hormone, human chorionic gonadotropin, human placental lactogen, prolactin, prolactin releasing hormone, luteinizing hormone, thyroid-stimulating hormone, thyrotropin-releasing hormone, parathyroid hormone, and pituitary adenylate cyclase-activating peptide.

Examples of neuropeptides include, but are not limited to, neuropeptide Y, an endorphin, an encephalin, brain natriuretic peptide, tachykinin, cortistatin, galanin, orexin, and oxytocin.

In some embodiments, the polynucleotide comprises the nucleotide sequence AAGC CACTGTGTCCTGAAGAAAAGCAAAGACATC (SEQ ID NO:54), and the peptide comprises the amino acid sequence SYSMEHFRWGKPVGGGSSGGGC (SEQ ID NO:55), SYSXEHFRWGKPVGGGSSGGGC (SEQ ID NO:56), CSGGGSSGGGSYSMEHFRWG KPV-NH₂ (SEQ ID NO:57), or CSGGGSSGGGSYSXEHFRWGKPV-NH₂ (SEQ ID NO:58), wherein X is norleucine and the F residue is D-phenylalanine.

In some embodiments, the fusion proteins described herein can comprise: an N-terminal protein fragment, a fusion partner protein, and a purification domain, wherein the C-terminus of the N-terminal protein fragment is coupled to the N-terminus of the fusion partner protein, and the C-terminus of the fusion partner protein is coupled to the N-terminus of the purification domain; or an N-terminal protein fragment, a fusion partner protein, and a cleavage site, wherein the C-terminus of the fusion partner protein is coupled to the N-terminus of the cleavage site, and the C-terminus of the cleavage site is coupled to the N-terminus of the N-terminal protein fragment, wherein the N-terminal protein fragment comprises an N-terminal methionine and a C-terminal cysteine; or a C-terminal protein fragment, a fusion partner protein, and a cleavage site, wherein the C-terminus of the fusion partner protein is coupled to the N-terminus of the cleavage site, and the C-terminus of the cleavage site is coupled to the N-terminus of the C-terminal protein fragment, wherein the C-terminal protein fragment comprises an N-terminal cysteine.

In some embodiments, the fusion protein comprises an N-terminal protein fragment, intein, and a chitin-binding domain, wherein the C-terminus of the N-terminal protein fragment is coupled to the N-terminus of intein, and the C-terminus of intein is coupled to the N-terminus of the chitin-binding domain. In some embodiments, the fusion protein comprises an N-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the N-terminal protein fragment, wherein the N-terminal protein fragment comprises an N-terminal methionine and a C-terminal cysteine. In some embodiments, the fusion protein comprises a C-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the C-terminal protein fragment, wherein the C-terminal protein fragment comprises an N-terminal cysteine.

In some embodiments, the fusion protein comprises an N-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the N-terminal protein fragment, wherein the N-terminal protein fragment comprises the amino acid sequence APIVTCRPKLDGREKPFKVDVATAQAQARKAGLTTGKSGDPHRYFAGDHI RWGVNNCDKADAILWEYPIYWVGKNAEWAKDVKTSQQKGC (SEQ ID NO:59).

In some embodiments, the fusion protein comprises a C-terminal protein fragment, a maltose-binding protein, and an enterokinase cleavage site, wherein the C-terminus of the maltose-binding protein is coupled to the N-terminus of the enterokinase cleavage site, and the C-terminus of the enterokinase cleavage site is coupled to the N-terminus of the C-terminal protein fragment, wherein the C-terminal protein fragment comprises the amino acid sequence CGPTPIRVVYANSRGAVQYCGVMTHSKVDKNNQGKEFFEKCD (SEQ ID NO:60).

In some embodiments, the fusion partner protein is intein, a maltose-binding protein, glutathione-S-transferase, β-galactosidase, or Omp F.

In some embodiments, the cleavage site is an enterokinase cleavage site or a Factor Xa protease cleavage site. In some embodiments, the Factor Xa protease cleavage site is IEGR (SEQ ID NO:61).

In some embodiments, the purification domain is a chitin-binding domain or a hexahistidine tag.

In some embodiments, coupling of oligonucleotides for SP-TAPER is affected by covalently modifying nucleic acid 5′ or 3′ termini with a chelating agent to enable oligonucleotide binding to hexahistidine split-protein fragment fusions. Oligonucleotides with 5′ or 3′ disulfide modifications are initially reduced with a molar excess of TCEP, and then run through desalting columns to purify the resulting thiol-terminal oligonucleotides from TCEP and low-molecular weight products. Following this, the free-thiol oligonucleotides are reacted with maleimido-C3-nitrilotriacetic acid (MNTA; Dojindo Molecular Technologies), such that the maleimide moiety of MNTA reacts with the available thiols to form a conjugate. This product is again purified from low-molecular species by desalting, and then is loaded with nickel ions by incubating with a molar excess of NiCl₂, and re-desalted to remove nickel excess. The resulting chelation conjugate can then be used to form a complex with split-protein fragments bearing either a C-terminal or N-terminal hexahistidine tag, produced by expression of appropriate coding sequences. The conjugation process is depicted in FIG. 19 of PCT Publication WO 18/93978.

In some embodiments, excess NTA::Ni oligonucleotides can be removed from reactions forming complexes with hexahistidine, by means of a biotinylated oligonucleotide with a tetrahistidine sequence (biotin-GSGSGHHHH; SEQ ID NO:62). Since nickel chelates can still bind tetrahistidine but with reduced affinity relative to hexahistidine (Knecht et al., J. Molec. Recognition, 2009, 22, 270-279), excess tetrahistidine peptide can deplete unconjugated NTA::Ni oligonucleotides without competitively stripping oligonucleotides from the protein fragment histidine tag. The biotinylated peptide/oligonucleotide excess are then removed on solid-phase streptavidin preparations (see, FIG. 20 of PCT Publication WO 18/93978). If necessary, the depletion step with the biotinylated tetrahistidine peptide can be repeated to remove residual unconjugated NTA::Ni oligonucleotide chelates.

Conjugates formed by complexing between NTA::Ni chelate and hexahistidine tags can be used in SP-TAPER in the same manner as for other chemical conjugation pathways, using any of the Architectures 1-4, and locked TAPER configurations (see, FIGS. 4-9 of PCT Publication WO 18/93978).

Compounds having the following formula

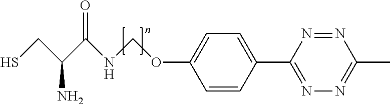

wherein n is from about 3 to about 6. In some embodiments, n is from about 4 to about 6 or from 5 to 6 can also be used in SP-TAPER reactions. In some embodiments, n is 3. In some embodiments, n is 4. In some embodiments, n is 5. In some embodiments, n is 6. In some embodiments, the compound is modified by replacing one or more hydrogens with various substituents including, for example, —OH, —C₁-C₆alkyl, —C₁-C₆alkenyl, and a halogen, and the like.

For any of the any of the haplomer polynucleotides described herein, the complementarity with another nucleic acid molecule can be 100%. In some embodiments, one particular nucleic acid molecule can be substantially complementary to another nucleic acid molecule. As used herein, the phrase “substantially complementary” means from 1 to 10 mismatched base positions, from 1 to 9 mismatched base positions, from 1 to 8 mismatched base positions, from 1 to 7 mismatched base positions, from 1 to 6 mismatched base positions, from 1 to 5 mismatched base positions, from 1 to 4 mismatched base positions, from 1 to 3 mismatched base positions, and 1 or 2 mismatched base positions. In some embodiments, it is desirable to avoid reducing the Tₘ of the anti-target loop portion:nucleic acid template by more than 10% via mismatched base positions. The bottle haplomer stem is designed with respect to a second haplomer, and its structure is deliberately arranged to be somewhat more stable than the formation of the second haplomer duplex.

In some embodiments, the portion of the bottle haplomer that is not linked to a protein fragment can have additional nucleotide bases that overhang and do not form a part of the stem structure. In some embodiments, the end of the second haplomer that is not linked to a protein fragment can have additional nucleotide bases that overhang and do not form a complementary part of the structure with the stem portion of the bottle haplomer. In addition, in some embodiments, the portion of the stem that is linked to the protein fragment can also have nucleotide bases that are not base paired with the first stem portion. Such an extension of the stem with a non-hybridized “arm” places the two protein fragments at a greater spatial distance, thus, tending to reduce their mutual reactivity. So, for a few nucleotide bases (less than 10 or less than 5), enforced reactivity is still likely to occur, but will tend to diminish as the non-base paired segment grows in length.

In some embodiments, added nucleotide bases can be of indefinite length, as long as they did not: 1) have significant homologies with any of the other regions of the locked TAPER oligonucleotides, and thus tend to cross-hybridize and interfere; or 2) interfere non-specifically with any other features of the system. For example, a long appended sequence might reduce transformation efficiencies of locked TAPER oligonucleotides used in a therapeutic context. Also, appended sequences should be designed to avoid spurious hybridizations with other cellular transcripts. Appended non-homologous sequences of 20-30 nucleotide bases are suitable. The appended nucleic acid sequences may contain primer sequences commonly used in the art. Such examples may include, but are not limited to, M13, T3, T7, SP6, VF2, VR, modified versions thereof, complementary sequences thereof, and reverse sequences thereof. In addition, custom primer sequences are also included. Such primer sequences can be used, for example, the possible application of chemically-ligated oligonucleotides spatially elicited (CLOSE) to the locked TAPER strategy, (see, PCT Publication WO 16/89958).

Any of the haplomers and bottle haplomers described herein, or any portion thereof, can further comprise a linker between any one or more of the first stem portion and the anti-target loop portion, between the anti-target loop portion and the second stem portion, between the second stem portion and the protein fragment, between the first stem portion and the ligand, or between the second haplomer and its protein fragment. In some embodiments, the linker is selected from the group consisting of an alkyl group, an alkenyl group, an amide, an ester, a thioester, a ketone, an ether, a thioether, a disulfide, an ethylene glycol, a cycloalkyl group, a benzyl group, a heterocyclic group, a maleimidyl group, a hydrazone, a urethane, azoles, an imine, a haloalkyl, nitrilotriacetic acid, nickel, cobalt, copper, and a carbamate, or any combination thereof.

In some embodiments, the bottle haplomer comprises the nucleotide sequence 5′-A CTCGAGACGTCTCCTTGTCTTTGCTTTTCTTCAGGACACAGTGGCGAGACGTCTCG AGT-3′ (SEQ ID NO:7) or 5′-ACTCGAGACGTCTCCTTCCTGCCCCTCCTCCTGCTCC GAGACGTCTCGAGT-3′ (SEQ ID NO:8).

In some embodiments, the second haplomer comprises the nucleotide sequence 5′-AGCTCTCGAGT-3′ (SEQ ID NO:9), or 5′-GACGTCTCGAGT-3′ (SEQ ID NO:10).

In some embodiments, the polynucleotide of the bottle haplomer comprises the nucleotide sequence of 5′-ACTCGAGACGTCTCCTTGTCTTTGCTTTTCTTCAGGACAC AGTGGCGAGACGTCTCGAGT-3′ (SEQ ID NO:7), and the polynucleotide of the second haplomer comprises the nucleotide sequence of 5′-AGCTCTCGAGT-3′ (SEQ ID NO:9); or the polynucleotide of the bottle haplomer comprises the nucleotide sequence of 5′-ACTCGA GACGTCTCCTTCCTGCCCCTCCTCCTGCTCCGAGACGTCTCGAGT-3′ (SEQ ID NO:8), and the polynucleotide of the second haplomer comprises the nucleotide sequence 5′-GACGTCTCGAGT-3′ (SEQ ID NO:10).

In some embodiments, the reporter protein is a fluorescent protein, a luciferase, a choramphenicol acetyl transferase, a β-galactosidase, or a β-glucuronidase.

In some embodiments, the fluorescent protein is GFP, YFP, mCherry, dsRed, VENUS, or CFP, a blue fluorescent protein, or any analog thereof. In some embodiments, the fluorescent protein is superfolder GFP. In some embodiments, the N-terminal fragment of the superfolder GFP comprises the amino acid sequence of MSKGEELFTGVVPILVELDGDVN GHKFSVRGEGEGDATNGKLTLKFICTTGKLPVPWPTLVTTLTYGVQCFSRYPDHMK RHDFFKSAMPEGYVQERTISFKDDGTYKTRAEVKFEGDTLVNRIELKGIDFKEDGNIL GHKLEYNFNSHNVYITADKQ (SEQ ID NO:65). In some embodiments, the C-terminal fragment of the superfolder GFP comprises the amino acid sequence of KNGIKANFKIRHN VEDGSVQLADHYQQNTPIGDGPVLLPDNHYLSTQSVLSKDPNEKRDHMVLLEFVTA AGITHGMDELYK (SEQ ID NO:29). In some embodiments, the fragment of superfolder GFP (sfGFP) comprises MRKGEELFTGVVPILVELDGDVNGHKFSVRGEGEGDATNGK LTLKFICTTGKLPVPWPTLVTTLTYGVQCFARYPDHMKQHDFFKSAMPEGYVQERTI SFKDDGTYKTRAEVKFEGDTLVNRIELKGIDFKEDGNILGHKLEYNFNSHNVYITAD KQ (SEQ ID NO:28) or KNGIKANFKIRHNVEDGSVQLADHYQQNTPIGDGPVLLPDN HYLSTQSVLSKDPNEKRDHMVLLEFVTAAGITHGMDELYK (SEQ ID NO:29), wherein one fragment interacts with the other fragment.

In some embodiments, the luciferase is firefly luciferase, Renilla luciferase, or Gaussia princeps luciferase. In some embodiments, the luciferase is Renilla luciferase. In some embodiments, the N-terminal fragment of the Renilla luciferase comprises the amino acid sequence of MASKVYDPEQRKRMITGPQWWARCKQMNVLDSFINYYDSEKHAE NAVIFLHGNAASSYLWRHVVPHIEPVARCIIPDLIGMGKSGKSGNGSYRLLDHYKYL TAWFELLNLPKKIIFVGHDWGACLAFHYSYEHQDKIKAIVHAESVVDVIESWDEWP DIEEDIALIKSEEGEKMVLENNFFVETMLPSKIMRKLEPEEFAAYLEPFKEKGEVRRPT LSWPREIPLVKGG (SEQ ID NO:30). In some embodiments, the C-terminal fragment of the Renilla luciferase comprises the amino acid sequence of KPDVVQIVRNYNAYLRASDDLP KMFIESDPGFFSNAIVEGAKKFPNTEFVKVKGLHFSQEDAPDEMGKYIKSFVERVLK NEQZ (SEQ ID NO:31). In some embodiments, the fragment of Renilla luciferase comprises MASKVYDPEQRKRMITGPQWWARCKQMNVLDSFINYYDSEKHAENAVIFLHGNAA SSYLWRHVVPHIEPVARCIIPDLIGMGKSGKSGNGSYRLLDHYKYLTAWFELLNLPK KIIFVGHDWGACLAFHYSYEHQDKIKAIVHAESVVDVIESWDEWPDIEEDIALIKSEE GEKMVLENNFFVETMLPSKIMRKLEPEEFAAYLEPFKEKGEVRRPTLSWPREIPLVK GG (SEQ ID NO:30) or KPDVVQIVRNYNAYLRASDDLPKMFIESDPGFFSNAIVEGAK KFPNTEFVKVKGLHFSQEDAPDEMGKYIKSFVERVLKNEQ (SEQ ID NO:31), wherein one fragment interacts with the other fragment. In some embodiments, the luciferase is Gaussia princeps luciferase. In some embodiments, the N-terminal fragment of the Gaussia princeps luciferase comprises the amino acid sequence of MKPTENNEDFNIVAVASNFAT TDLDADRGKLPGKKLPLEVLKEMEANARKAGCTRGCLICLSHIKCTPKMKKFIPGRC HTYEGDKESAQGGIG (SEQ ID NO:32). In some embodiments, the C-terminal fragment of the Gaussia princeps luciferase comprises the amino acid sequence of EAIVDIPEIPGFK DLEPMEQFIAQVDLCVDCTTGCLKGLANVQCSDLLKKWLPQRCATFASKIQGQVDK IKGAGGD (SEQ ID NO:33).

In some embodiments, killing or growth inhibition of target cells can be induced by direct interaction with cytotoxic, microbicidal, or virucidal effector agents. Numerous toxic molecules known in the art can be produced. In some embodiments, the protein of interest is a toxic peptide or toxic protein. Examples of toxic peptides include, but are not limited to, bee melittin, conotoxins, cathelicidins, defensins, protegrins, and NK-lysin. Examples of toxic proteins include, but are not limited to, ricin A chain, Aspf1, α-sarcin, mitogillin, hirsutellin A, diphtheria toxin, botulinum A toxin, and cholera toxin. In some embodiments, the toxic protein is a ribotoxin that cleaves the large 28S ribosomal RNA.

In some embodiments, the Tₘ of the first stem portion:second stem portion subtracted from the Tₘ of the anti-target loop portion:nucleic acid template is from about 10° C. to about 40° C. In some embodiments, the Tₘ of the first stem portion:second stem portion subtracted from the Tₘ of the anti-target loop portion:nucleic acid template is from about 10° C. to about 20° C. In some embodiments, the Tₘ of the first stem portion:second stem portion is from about 40° C. to about 50° C. In some embodiments, the Tₘ of the anti-target loop portion:nucleic acid template is from about 60° C. to about 80° C. In some embodiments, the Tₘ of the duplex formed by the second haplomer and the first or second stem portion of the bottle haplomer subtracted from the Tₘ of the first stem portion: second stem portion is from about 0° C. to about 20° C. In some embodiments, the Tₘ of the duplex formed by the second haplomer and the first or second stem portion of the bottle haplomer subtracted from the Tₘ of the first stem portion:second stem portion is from about 5° C. to about 10° C. In some embodiments, the Tₘ of the duplex formed by the second haplomer and the first or second stem portion of the bottle haplomer is from about 30° C. to about 40° C.

In some embodiments, the first stem portion comprises from about 12 to about 18 nucleotide bases. In some embodiments, the anti-target loop portion comprises from about 18 to about 35 nucleotide bases. In some embodiments, the second stem portion comprises from about 12 to about 18 nucleotide bases.

In some embodiments, the SP-TAPER methods comprise: a) contacting a cell with a first haplomer comprising a polynucleotide linked to the C-terminus of an N-terminal protein fragment; and b) contacting the cell with a second haplomer comprising a polynucleotide linked to the N-terminus of a C-terminal protein fragment; wherein: i) the polynucleotide of one of the first or second haplomers is linked at its 5′ terminus to the protein fragment, and the other of the first and second haplomers is linked at its 3′ terminus to the protein fragment; ii) the N-terminal protein fragment and the C-terminal protein fragment are derived from a single protein; and iii) wherein: the polynucleotide of the first haplomer is substantially complementary to the polynucleotide of the second haplomer; or the polynucleotide of the first haplomer is substantially complementary to a nucleic acid template, and the polynucleotide of the second haplomer is substantially complementary to the nucleic acid template at a site in spatial proximity to the polynucleotide of the first haplomer; or the polynucleotide of the first haplomer is substantially complementary to a portion of a nucleic acid template 5′ adjacent to a stem-loop structure, and the polynucleotide of the second haplomer is substantially complementary to a portion of the nucleic acid template 3′ adjacent to the stem-loop structure; or the polynucleotide of the first haplomer is substantially complementary to a 5′ portion of a loop of a stem-loop structure of a nucleic acid template, and the polynucleotide of the second haplomer is substantially complementary to a 3′ portion of the loop of the stem-loop structure of the nucleic acid template; thereby resulting in the assembly of the protein from the N-terminal protein fragment and the C-terminal protein fragment.

In some embodiments, the polynucleotide of the first haplomer is substantially complementary to the polynucleotide of the second haplomer. In some embodiments, the polynucleotide of the first haplomer binds to the nucleic acid template in spatial proximity to the binding of the polynucleotide of the second haplomer to the nucleic acid template.

In some embodiments, the protein fragment of the first haplomer is linked to the 5′ terminus of the polynucleotide of the first haplomer, and the polynucleotide of the first haplomer is substantially complementary to a portion of the nucleic acid target 5′ adjacent to a stem-loop structure; and the protein fragment of the second haplomer is linked to the 3′ terminus of the polynucleotide of the second haplomer, and the polynucleotide of the second haplomer is substantially complementary to a portion of the nucleic acid target 3′ adjacent to the stem-loop structure.

In some embodiments, the protein fragment of the first haplomer is linked to the 3′ terminus of the polynucleotide of the first haplomer, and the polynucleotide of the first haplomer is substantially complementary to a 5′ portion of a loop structure of a stem-loop structure of the nucleic acid template, wherein the 5′ portion of the loop structure is adjacent to the stem region of the stem-loop structure; and the protein fragment of the second haplomer is linked to the 5′ terminus of the polynucleotide of the second haplomer, and the polynucleotide of the second haplomer is substantially complementary to a 3′ portion of the loop structure of the stem-loop structure of the nucleic acid template, wherein the 3′ portion of the loop structure is adjacent to the stem region of the stem-loop structure.

In some embodiments, the SP-TAPER methods comprise: a) contacting a nucleic acid template with a bottle haplomer comprising: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to a nucleic acid template; and iii) a second 5′ stem portion comprising from about 10 to about 20 nucleotide bases linked to the anti-target loop portion, wherein the first 3′ stem portion is substantially complementary to the second 5′ stem portion; wherein the 5′ terminus of the polynucleotide is linked to the C-terminus of an N-terminal protein fragment, wherein the C-terminus comprises a cysteine; and b) contacting the bottle haplomer with a second haplomer comprising a polynucleotide linked to the N-terminus of a C-terminal protein fragment, wherein the polynucleotide of the second haplomer is substantially complementary to the second 5′ stem portion of the polynucleotide of the bottle haplomer; wherein: i) the N-terminal protein fragment and the C-terminal protein fragment are derived from a single protein; ii) the Tₘ of the anti-target loop portion:nucleic acid template is greater than the Tₘ of the first stem portion:second stem portion; and iii) the Tₘ of the duplex formed by the second haplomer and the second stem portion of the bottle haplomer subtracted from the Tₘ of the first stem portion:second stem portion is from about 0° C. to about 20° C.; thereby resulting in the assembly of the protein from the N-terminal protein fragment and the C-terminal protein fragment.

In some embodiments, the SP-TAPER methods comprise: a) contacting a cell with a surface target compound comprising: i) a template polynucleotide; and ii) a peptide; wherein: i) the 5′ terminus of the polynucleotide is coupled to the N-terminus or C-terminus of the peptide, or the 3′ terminus of the polynucleotide is coupled to the N-terminus or C-terminus of the peptide; and ii) the peptide is a ligand for a cell-surface molecule; b) contacting the cell with a first haplomer comprising a polynucleotide linked to the C-terminus of an N-terminal protein fragment; and c) contacting the cell with a second haplomer comprising a polynucleotide linked to the N-terminus of a C-terminal protein fragment; wherein: i) the polynucleotide of one of the first or second haplomers is linked at its 5′ terminus to the protein fragment, and the other of the first and second haplomers is linked at its 3′ terminus to the protein fragment; ii) the N-terminal protein fragment and the C-terminal protein fragment are derived from a single protein; and iii) the polynucleotide of the first haplomer is substantially complementary to the template polynucleotide of the surface target compound, and the polynucleotide of the second haplomer is substantially complementary to the template polynucleotide of the surface target compound at a site in spatial proximity to the polynucleotide of the first haplomer; thereby resulting in the assembly of the protein from the N-terminal protein fragment and the C-terminal protein fragment.

In some embodiments, the SP-TAPER methods comprise: a) contacting a cell with a surface target compound comprising: i) a template polynucleotide; and ii) a peptide; wherein: i) the 5′ terminus of the polynucleotide is coupled to the N-terminus or C-terminus of the peptide, or the 3′ terminus of the polynucleotide is coupled to the N-terminus or C-terminus of the peptide; and ii) the peptide is a ligand for a cell-surface molecule; b) contacting a nucleic acid template with a bottle haplomer comprising: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to the template polynucleotide of the surface target compound; and iii) a second 5′ stem portion comprising from about 10 to about 20 nucleotide bases linked to the anti-target loop portion, wherein the first 3′ stem portion is substantially complementary to the second 5′ stem portion; wherein the 5′ terminus of the polynucleotide is linked to the C-terminus of an N-terminal protein fragment, wherein the C-terminus comprises a cysteine; and c) contacting the bottle haplomer with a second haplomer comprising a polynucleotide linked to the N-terminus of a C-terminal protein fragment, wherein the polynucleotide of the second haplomer is substantially complementary to the second 5′ stem portion of the polynucleotide of the bottle haplomer; wherein: i) the N-terminal protein fragment and the C-terminal protein fragment are derived from a single protein; ii) the Tₘ of the anti-target loop portion:nucleic acid template is greater than the Tₘ of the first stem portion:second stem portion; and iii) the Tₘ of the duplex formed by the second haplomer and the second stem portion of the bottle haplomer subtracted from the Tₘ of the first stem portion:second stem portion is from about 0° C. to about 20° C.; thereby resulting in the assembly of the protein from the N-terminal protein fragment and the C-terminal protein fragment.

The present disclosure also provides any of the metabolic labeling methods described herein, further comprising performing an SP-TAPER reaction using the nucleic acid template as a target nucleic acid sequence for the TAPER reaction.

In some embodiments, the SP-TAPER reaction comprises: contacting the cell with a first haplomer comprising a first polynucleotide linked to the C-terminus of an N-terminal protein fragment; and contacting the cell with a second haplomer comprising a second polynucleotide linked to the N-terminus of a C-terminal protein fragment; wherein: the polynucleotide of one of the first or second haplomers is linked at its 5′ terminus to the protein fragment, and the other of the first and second haplomers is linked at its 3′ terminus to the protein fragment; the N-terminal protein fragment and the C-terminal protein fragment are derived from a single active effector agent; and wherein: the first polynucleotide of the first haplomer is substantially complementary to the nucleic acid template, and the second polynucleotide of the second haplomer is substantially complementary to the nucleic acid template at a site in spatial proximity to the first polynucleotide of the first haplomer; or the first polynucleotide of the first haplomer is substantially complementary to a portion of the nucleic acid template 5′ adjacent to a stem-loop structure, and the second polynucleotide of the second haplomer is substantially complementary to a portion of the nucleic acid template 3′ adjacent to the stem-loop structure; or the first polynucleotide of the first haplomer is substantially complementary to a 5′ portion of a loop of a stem-loop structure of the nucleic acid template, and the second polynucleotide of the second haplomer is substantially complementary to a 3′ portion of the loop of the stem-loop structure of the nucleic acid template; thereby resulting in the assembly of the protein from the N-terminal protein fragment and the C-terminal protein fragment.

In some embodiments, the N-terminal fragment comprises the amino acid sequence of APIVTCRKLDGREKPFKVDVATAQAQARKAGLTTGKSGDPHRYFAGDHIRWGVNN CDKADAILWEYPIYWVGKNAEWAKDVKTSQQKG (SEQ ID NO:34), and the C-terminal fragment comprises the amino acid sequence of GPTPIRVVYANSRGAVQYCGV MTHSKVDKNNQGKEFFEKCD (SEQ ID NO:35); the N-terminal fragment comprises the amino acid sequence of APIVTCRPKLDG (SEQ ID NO:36), and the C-terminal fragment comprises the amino acid sequence of REKPFKVDVATAQAQARKAGLTTGKSGDPHR YFAGDHIRWGVNNCDKADAILWEYPIYWVGKNAEWAKDVKTSQQKGGPTPIRVVY ANSRGAVQYCGVMTHSKVDKNNQGKEFFEKCD (SEQ ID NO:37); the N-terminal fragment comprises the amino acid sequence of APIVTCRPKLDGREKPFKVDVATAQAQ ARKAGLTTGK (SEQ ID NO:38), and the C-terminal fragment comprises the amino acid sequence of SGDPHRYFAGDHIRWGVNNCDKADAILWEYPIYWVGKNAEWAKDVK TSQQKGGPTPIRVVYANSRGAVQYCGVMTHSKVDKNNQGKEFFEKCD (SEQ ID NO:39); the N-terminal fragment comprises the amino acid sequence of APIVTCRPKLDGR EKPFKVDVATAQAQARKAGLTTGKSGDPHRYFAGDHIRWGVNNCDKAD (SEQ ID NO:40), and the C-terminal fragment comprises the amino acid sequence of AILWEYPIYW VGKNAEWAKDVKTSQQKGGPTPIRVVYANSRGAVQYCGVMTHSKVDKNNQGKEF FEKCD (SEQ ID NO:41); the N-terminal fragment comprises the amino acid sequence of APIVTCRPKLDGREKPFKVDVATAQAQARKAGLTTGKSGDPHRYFAGDHIRWGVN NCDKADAILWEYPIYWVG (SEQ ID NO:42), and the C-terminal fragment comprises the amino acid sequence of KNAEWAKDVKTSQQKGGPTPIRVVYANSRGAVQYCGVMTH SKVDKNNQGKEFFEKCD (SEQ ID NO:43); the N-terminal fragment comprises the amino acid sequence of APIVTCRPKLDGREKPFKVDVATAQAQARKAGLTTGKSGDPHRYFA GDHIRWGVNNCDKADAILWEYPIYWVGKNAEWAKD (SEQ ID NO:44), and the C-terminal fragment comprises the amino acid sequence of VKTSQQKGGPTPIRVVYANSRG AVQYCGVMTHSKVDKNNQGKEFFEKCD (SEQ ID NO:45); the N-terminal fragment comprises the amino acid sequence of APIVTCRPKLDGREKPFKVDVATAQAQARKAG LTTGKSGDPHRYFAGDHIRWGVNNCDKADAILWEYPIYWVGKNAEWAKDVKTSQ (SEQ ID NO:46), and the C-terminal fragment comprises the amino acid sequence of QKGG PTPIRVVYANSRGAVQYCGVMTHSKVDKNNQGKEFFEKCD (SEQ ID NO:47); the N-terminal fragment comprises the amino acid sequence of APIVTCRPKLDGREKPFKVDVA TAQAQARKAGLTTGKSGDPHRYFAGDHIRWGVNNCDKADAILWEYPIYWVGKNAE WAKDVKTSQQKGGPTPIRVVYANSRG (SEQ ID NO:48), and the C-terminal fragment comprises the amino acid sequence of AVQYCGVMTHSKVDKNNQGKEFFEKCD (SEQ ID NO:49); the N-terminal fragment comprises the amino acid sequence of APIVTCRPKLD GREKPFKVDVATAQAQARKAGLTTGKSGDPHRYFAGDHIRWGVNNCDKADAILWE YPIYWVGKNAEWAKDVKTSQQKGGPTPIRVVYANSRGAVQYCGVMTHSKVDKN (SEQ ID NO:50), and the C-terminal fragment comprises the amino acid sequence of NQGK EFFEKCD (SEQ ID NO:51); or the N-terminal fragment comprises the amino acid sequence of APIVTCRPKLDGREKPFKVDVATAQAQARKAGLT; (SEQ ID NO:52), and the C-terminal fragment comprises the amino acid sequence of TGKSGDPHRYFAGDHIRWGVN NCDKADAILWEYPIYWVGKNAEWAKDVKTSQQKGGPTPIRVVYANSRGAVQYCG VMTHSKVDKNNQGKEFFEKCD (SEQ ID NO:53).

In some embodiments, the SP-TAPER reaction comprises: a) contacting the nucleic acid template with a bottle haplomer, wherein the bottle haplomer comprises a first polynucleotide comprising: i) a first 3′ stem portion comprising from about 10 to about 20 nucleotide bases; ii) an anti-target loop portion comprising from about 16 to about 40 nucleotide bases linked to the first 3′ stem portion, wherein the anti-target loop portion is substantially complementary to the nucleic acid template; and iii) a second 5′ stem portion comprising from about 10 to about 20 nucleotide bases linked to the anti-target loop portion, wherein the first 3′ stem portion is substantially complementary to the second 5′ stem portion; wherein the 5′ terminus of the first polynucleotide comprises an —SH moiety and is linked to the C-terminus of an N-terminal protein fragment, wherein the C-terminus comprises a cysteine or a selenocysteine; and b) contacting the bottle haplomer with a second haplomer comprising a second polynucleotide linked to the N-terminus of a C-terminal protein fragment, wherein the second polynucleotide of the second haplomer is substantially complementary to the second 5′ stem portion of the first polynucleotide of the bottle haplomer; wherein: the N-terminal protein fragment and the C-terminal protein fragment are derived from a single active effector agent; the Tₘ of the anti-target loop portion:nucleic acid template is greater than the Tₘ of the first stem portion: second stem portion; and the Tₘ of the duplex formed by the second haplomer and the second stem portion of the bottle haplomer subtracted from the Tₘ of the first stem portion:second stem portion is from about 0° C. to about 20° C.; thereby resulting in the assembly of the protein from the N-terminal protein fragment and the C-terminal protein fragment.

In some embodiments, the Tₘ of the first stem portion:second stem portion subtracted from the Tₘ of the anti-target loop portion:nucleic acid template is from about 10° C. to about 40° C.; the Tₘ of the first stem portion: second stem portion is from about 40° C. to about 50° C.; the Tₘ of the anti-target loop portion:nucleic acid template is from about 60° C. to about 80° C.; and/or the Tₘ of the first stem portion:second stem portion subtracted from the T1 of the anti-target loop portion:nucleic acid template is from about 10° C. to about 20° C.

In some embodiments, the first stem portion comprises from about 12 to about 18 nucleotide bases; the anti-target loop portion comprises from about 18 to about 35 nucleotide bases; and/or the second stem portion comprises from about 12 to about 18 nucleotide bases. In some embodiments, the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

In any of the SP-TAPER methods described herein, the N-terminal protein fragment and the C-terminal protein fragment are fragments of an active effector agent chosen from a peptide, protein, or a therapeutic agent. In some embodiments, the peptide or protein is a toxin, a pro-apoptotic agent, or a ligand for an antibody or antibody fragment.

A distinction in Locked TAPER from conventional TAPER is that the nucleic acid template for haplomer binding does not correspond to a cellular nucleic acid. In locked TAPER, the haplomer sequences are fixed and complementary to each other, where the first haplomer is “locked” by hybridization to a complementary sequence within the same longer oligonucleotide (see, FIG. 4 of PCT Publication WO 18/94070). An internal anti-target loop portion sequence within this oligonucleotide structure corresponds to the target-complementary sequence. The structure comprising the first haplomer constrained by internal self-hybridization and an anti-target loop portion sequence that is complementary to the nucleic acid template is the “first haplomer” or “first haplomer bottle.”

In locked TAPER, the nucleic acid molecules comprise: a) a first stem portion comprising from about 10 to about 20 nucleotide bases; b) an anti-target loop portion comprising from about 16 to about 40 nucleotide bases and having a first end to which the first stem portion is linked, wherein the anti-target loop portion is substantially complementary to a nucleic acid template; c) a second stem portion comprising from about 10 to about 20 nucleotide bases linked to a second end of the anti-target loop portion, wherein the first stem portion is substantially complementary to the second stem portion; and d) a reactive effector moiety linked to either the first stem portion or the second stem portion; wherein the Tₘ of the anti-target loop portion:nucleic acid template is greater than the Tₘ of the first stem portion:second stem portion. A nucleic acid molecule comprising these features is referred to herein as: “first nucleic acid molecule”, “first haplomer bottle”, “first haplomer” and “bottle haplomer.”

In some embodiments, the first nucleic acid molecule comprises a first stem portion that comprises from about 10 to about 20 nucleotide bases. In some embodiments, the first stem portion comprises from about 12 to about 18 nucleotide bases. In some embodiments, a reactive effector moiety is linked to the first stem portion.

In some embodiments, the first nucleic acid molecule comprises an anti-target loop portion that comprises from about 16 to about 40 nucleotide bases. In some embodiments, the anti-target loop portion comprises from about 18 to about 35 nucleotide bases. The anti-target loop portion has a first end to which the first stem portion is linked. The anti-target loop portion is substantially complementary to a nucleic acid template.

In some embodiments, the anti-target loop portion can further comprise an internal hinge region, wherein the hinge region comprises one or more nucleotides that are not complementary to the nucleic acid template. In some embodiments, the hinge region comprises from about 1 nucleotide to about 6 nucleotides, from about 1 nucleotide to about 5 nucleotides, from about 1 nucleotide to about 4 nucleotides, from about 1 nucleotide to about 3 nucleotides, or 1 or 2 nucleotides.

The nucleic acid template (complementary to the anti-target loop portion of a general first haplomer bottle) for all locked TAPER embodiments can comprise any desired nucleic acid sequence capable of hybridizing with the specific anti-loop region portion itself. Any single-stranded nucleic acid molecule with an accessible sequence is potentially targetable. These include, but are not limited to, cellular RNAs, mRNA, genomic or organellar DNA, episomal or plasmid DNA, viral DNA or RNA, miRNA, rRNA, snRNA, tRNA, short and long non-coding RNAs, and any artificial sequences used for templating purposes, or any other biological or artificial nucleic acid sequence. Artificial sequences include, but are not limited to, aptamers and macromolecular-nucleic acid conjugates. Aptamer templates are also included, where these are designed to convert a non-nucleic acid cellular product into a targetable sequence for any form of TAPER, including locked TAPER. In some embodiments, the nucleic acid template hybridization site is kept as short as possible while: 1) maintaining specificity within a complex transcriptome or other complex targets; and 2) maintaining the locked TAPER design guidelines described herein.

In some embodiments, the first nucleic acid molecule comprises a second stem portion that comprises from about 10 to about 20 nucleotide bases. In some embodiments, the second stem portion comprises from about 12 to about 18 nucleotide bases. The second stem portion is linked to a second end of the anti-target loop portion. The first stem portion is substantially complementary to the second stem portion. In some embodiments, a reactive effector moiety is linked to the second stem portion.

In some embodiments, the first nucleic acid molecule comprises the nucleotide sequence 5′-ACTCGAGACGTCTCCTTGTCTTTGCTTTTCTTCAGGACACAGTGGCG AGACGTCTCGAGT-3′ (SEQ ID NO:7) or 5′-ACTCGAGACGTCTCCTTCCTGCCCCT CCTCCTGCTCCGAGACGTCTCGAGT-3′ (SEQ ID NO:8).

In locked TAPER, nucleic acid molecules comprise from about 6 nucleotide bases to about 20 nucleotide bases, which comprises: a nucleotide portion that is substantially complementary to the stem portion (either the first stem portion or the second stem portion) of the first nucleic acid molecule that is linked to the reactive effector moiety; and a reactive effector moiety which can chemically interact with the reactive effector molecule of the first nucleic acid molecule; wherein the Tₘ of the second nucleic acid molecule:first or second stem portion linked to the reactive effector moiety is less than or equal to the Tₘ of the first stem portion:second stem portion. A nucleic acid molecule comprising these features is referred to herein as: “second nucleic acid molecule” and “second haplomer.”

In some embodiments, the second nucleic acid molecule comprises from about 6 to about 20 nucleotide bases. In some embodiments, the second nucleic acid molecule comprises from about 8 to about 15 nucleotide bases.

In some embodiments, the second nucleic acid molecule comprises the nucleotide sequence 5′-AGCTCTCGAGT-3′ (SEQ ID NO:9), or 5′-GACGTCTCGAGT-3′ (SEQ ID NO:10).

In some embodiments, the first nucleic acid molecule comprises the nucleotide sequence of SEQ ID NO:7, and the second nucleic acid molecule comprises the nucleotide sequence of SEQ ID NO:9; or the first nucleic acid molecule comprises the nucleotide sequence of SEQ ID NO:8, and the second nucleic acid molecule comprises the nucleotide sequence of SEQ ID NO:10.

For the nucleic acid molecules described herein, the length of the particular nucleic acid molecule is less important than the Tₘ of the duplex formed by the interaction of the nucleic acid molecule, or portion thereof, with another nucleic acid molecule, or portion thereof. For example, the Tₘ of the duplex formed by the interaction of the anti-target loop portion with the nucleic acid template (e.g., anti-target loop portion:nucleic acid template) is greater than the Tₘ of the duplex formed by the interaction of the first stem portion of the first nucleic acid molecule with the second stem portion of the first nucleic acid molecule (e.g., first stem portion:second stem portion). In some embodiments, the Tₘ of the first stem portion:second stem portion subtracted from the Tₘ of the anti-target loop portion:nucleic acid template is from about 10° C. to about 40° C. In some embodiments, the Tₘ of the first stem portion:second stem portion subtracted from the Tₘ of the anti-target loop portion:nucleic acid template is from about 10° C. to about 20° C. In some embodiments, the Tₘ of the first stem portion:second stem portion is from about 40° C. to about 50° C. In some embodiments, the Tₘ of the anti-target loop portion:nucleic acid template is from about 60° C. to about 80° C. In some embodiments, the Tₘ of the duplex formed by the interaction of the second nucleic acid molecule with either the first stem portion or the second stem portion, whichever stem portion is linked to the reactive effector moiety (e.g., second nucleic acid molecule:first or second stem portion linked to the reactive effector moiety), is less than or equal to the Tₘ of the first stem portion:second stem portion. In some embodiments, the Tₘ of the duplex formed by the second nucleic acid molecule and the first or second stem portion linked to the reactive effector moiety subtracted from the Tₘ of the first stem portion:second stem portion is from about 0° C. to about 20° C. In some embodiments, the Tₘ of the duplex formed by the nucleic acid molecule and the first or second stem portion linked to the reactive effector moiety subtracted from the Tₘ of the first stem portion:second stem portion is from about 5° C. to about 10° C. In some embodiments, the Tₘ of the duplex formed by the second nucleic acid molecule and the first or second stem portion linked to the reactive effector moiety is from about 30° C. to about 40° C.

In addition, translating the Tₘ information above into specific lengths of the nucleic acid molecules described herein also depends on the GC content of each nucleic acid molecule. For example, the length of a suitable HPV model nucleic acid template is 30 bases (having a Tₘ of 70° C.), while that for the EBV model nucleic acid template is only 21 bases (having a Tₘ of 69° C.), owing to its greater % GC.

This structural arrangement is designed such that in the absence of nucleic acid template, the locked first haplomer bottle (e.g., the first nucleic acid molecule) does not significantly hybridize to its complementary second haplomer (e.g., the second nucleic acid molecule), and thus template-directed product assembly is not promoted under such conditions. When the specific nucleic acid template is present, on the other hand, the first haplomer bottle is “unlocked” by the formation of a more stable hybrid between the anti-target loop region of the bottle haplomer and the nucleic acid template itself (see, FIG. 4 of PCT Publication WO 18/94070). Once this occurs, the first stem portion of the bottle haplomer that is linked to the reactive moiety is free to hybridize to the available second haplomer, with resulting proximity between the mutually reactive effector moieties on both, and generation of a specific assembly reaction (see, FIG. 4 of PCT Publication WO 18/94070). It is the exposure of the accessible first haplomer bottle that renders the process resistant to the template titration effect, since there is a 1:1 correspondence between the binding of anti-target loop potion to the corresponding nucleic acid template and generation of first haplomer accessibility for the second haplomer. This can be expressed as:

B[L]cH1::H1+T→cH1-T::L-H1 (1)

cH1-T::L-H1+H2→cH1-T::L-H1::H2 (2),

where B[L]cH1::H1 is the first haplomer bottle with the anti-target loop region (L); T is the nucleic acid template; cH1 is the complement to the first haplomer sequence H1; T::L-H1 is the anti-target loop region duplex with the exposed first haplomer sequence; and H2 is the second haplomer. Since the second haplomer (H2) can only hybridize to the first haplomer (H1) after the latter has been exposed through the presence of the specific nucleic acid molecule template, a template excess cannot have a titration effect, and indeed is beneficial through shifting equation (1) further to the right, thus providing more available H1. The unlocking of a single copy of the first haplomer bottle in the presence of excess nucleic acid template is depicted schematically in FIG. 5 of PCT Publication WO 18/94070. Since the exposed first haplomer sequence H1 is unique and designed to be absent from the target expressed genome, spurious hybridization between H1 and an off-target sequence is minimal. This applies also to the designed complement to H1, the second haplomer H2.

As described above, the specificity of the H1::H2 interaction can be enhanced by rendering the hybridization bio-orthogonal. This can be achieved by, for example, synthesizing a hybrid first haplomer bottle where the H1 and H1-complementary (cH1) sequences are comprised of DNA bearing L-ribose, or L-DNA (see, FIG. 6 of PCT Publication WO 18/94070). Since DNA can only form duplexes between homochiral complementary single strands, when the H1 sequence is exposed following hybridization of the (normal) anti-target loop sequence with cellular target, it follows that H1 can only form a duplex with a corresponding H2 L-DNA sequence.

In all locked TAPER embodiments, one can modulate the hybridization Tₘ values of each component in line with the desired differential hybridization effects. Thus, the designed thermal stabilities of relevant components should be: T::L>>cH1::H1>H1::H2. It is notable that the sequences of cH1::H1 and H1::H2 are similar but not identical, in order to ensure that the sequestration of the H1 haplomer sequence within the first haplomer bottle is marginally more stable than the H1::H2 inter-haplomer duplex. By this means, mixtures of the first haplomer bottle and the second haplomer H2 in the absence of target will favor retention of the cH1::H1 configuration rather than formation of H1::H2.

Within a locked TAPER system, when the two haplomers bearing selectively-reactive moieties (e.g., reactive effector moiety linked to a bio-orthogonal reactive molecule) or other modifications are in hybridization-mediated spatial proximity (see, FIG. 4 of PCT Publication WO 18/94070), it is by virtue of their possessing mutual complementarity. This is quite distinct from conventional TAPER, where the spatial proximity is achieved by haplomer complementarity to a third-party template strand. Nevertheless, since the anti-target loop portion of a locked-TAPER first haplomer bottle hybridizes to a nucleic acid template to expose the recognition site for the second haplomer, the anti-target loop-target binding itself can occur via different architectures. These alternative structural arrangements can include hybridizations to discontinuous sites. Thus, the target hybridization of the locked TAPER oligonucleotide schematically depicted in FIG. 7 of PCT Publication WO 18/94070 is achieved with discontinuous sites brought into spatial proximity in the exterior arms of a stem loop structure. Alternately, equivalent spatial proximity can be engendered by hybridization sites juxtaposed within a loop formed by a template secondary structure (see, FIG. 8 of PCT Publication WO 18/94070). In both of these embodiments, the regions within the first haplomer loops that hybridize to discontinuous targets may be separated by an additional “hinge” sequence of d(T)N, where N is from 1 to about 6 bases. The provision of such a hinge sequence is designed to confer flexibility between the two hybridizing segments, and minimize torsional strain on these regions.

In these alternative architectures for Locked TAPER, it is important to maintain the differential rules of hybridization stabilities for T::L>>cH1::H1>H1::H2 as described above. Locked TAPER accordingly has the unique feature whereby the TAPER assembly is always constant through haplomer mutual complementarity, but target hybridization can assume variable architectures. In other words, for conventional TAPER, the target hybridization and assembly-directing hybridizations coincide, but for locked TAPER they are distinct and separable.

In these embodiments, locked TAPER affords considerable advantages compared to conventional TAPER. These advantages include, for example, evasion of template titration, boosting of signal strength with high copy-number template, provision of bio-orthogonal hybridization, and the use of fixed haplomeric sequences. In the latter case, a single pair of specific haplomers (bearing bio-orthogonal reactive molecules) can be used for an indefinite number of targets, where the loop region of first haplomer bottles can be varied according to the desired target sequence complement. In addition, solving the template titration problem enables the targeting of repeat sequence motifs. Where N copies of a specific motif occurs in M steady-state copies of a transcript of interest, the total number of theoretically targetable motifs is N×M. In practice, not all such motifs may be accessible, and the targetable motif number per cell becomes <N×M, owing to secondary structural constraints. Nevertheless, attempting to target a repeated motif with conventional TAPER is very likely to suffer restrictions from template titration, when accessible N×M copy number becomes greater than the molar quantities of each separate haplomer achievable after delivery into a target cellular environment. No such restriction exists for locked-TAPER, and indeed, increased copy number from a repeat motif is an advantage in terms of the potential increase in read out product assembly levels. In addition, the above observation that repeat motifs within a single transcript may vary in their accessibilities for TAPER purposes may be another inherent advantage of repeat motifs. In a dynamic cellular environment, where some single-copy mRNA motifs may have variable accessibility, multiple repeat motifs may increase the likelihood of access.

The locked TAPER process also uses a single segment for the hybridization that enables specific RNA targeting, in contrast to conventional TAPER, where two such sequences are used for each haplomer. Clearly, the length of these segment is a significant issue in terms of achieving the necessary specificity towards a target template. In the locked TAPER strategy, the length of the target-complementary loop sequence can be varied as desired, subject to the requirement that the Tₘ of loop::target is >>the bottle stem Tₘ But in specific targeting circumstances, the length of the target sequence with locked TAPER could achieve the necessary specificity and still be less than the total sequence required for conventional bimolecular effector partials. Thus, targeting by locked TAPER approach may be less demanding than the conventional TAPER strategy.

For any of the nucleic acid molecules described herein for locked TAPER, the complementarity with another nucleic acid molecule can be 100%. In some embodiments, one particular nucleic acid molecule can be substantially complementary to another nucleic acid molecule. As used herein, the phrase “substantially complementary” means from 1 to 10 mismatched base positions, from 1 to 9 mismatched base positions, from 1 to 8 mismatched base positions, from 1 to 7 mismatched base positions, from 1 to 6 mismatched base positions, from 1 to 5 mismatched base positions, from 1 to 4 mismatched base positions, from 1 to 3 mismatched base positions, and 1 or 2 mismatched base positions. In some embodiments, it is desirable to avoid reducing the Tₘ of the anti-target loop portion:nucleic acid template by more than 10% via mismatched base positions. The first haplomer bottle stem is designed with respect to second haplomer, and its structure is deliberately arranged to be somewhat more stable than the formation of the second haplomer duplex.

Any of the nucleic acid molecules described herein, or any portion thereof, can further comprise a linker between any one or more of the first stem portion and the anti-target loop portion, between the anti-target loop portion and the second stem portion, and between the second stem portion and the reactive effector moiety of the first nucleic acid molecule or between the second nucleic acid molecule and its reactive effector moiety. In some embodiments, the linker is selected from the group consisting of an alkyl group, an alkenyl group, an amide, an ester, a thioester, a ketone, an ether, a thioether, a disulfide, an ethylene glycol, a cycloalkyl group, a benzyl group, a heterocyclic group, a maleimidyl group, a hydrazone, a urethane, azoles, an imine, a haloalkyl, and a carbamate, or any combination thereof.

In some embodiments, the portion of the first nucleic acid molecule that is not linked to a reactive effector moiety can have additional nucleotide bases that overhang and do not form a part of the stem structure. In some embodiments, the end of the second nucleic acid molecule that is not linked to a reactive effector moiety can have additional nucleotide bases that overhang and do not form a complementary part of the structure with the stem portion of the first nucleic acid molecule. In addition, in some embodiments, the portion of the stem that is linked to the reactive effector moiety can also have nucleotide bases that are not base paired with the first stem portion. Such an extension of the stem with a non-hybridized “arm” places the two reactive effectors at a greater spatial distance, thus, tending to reduce their mutual reactivity. So, for a few nucleotide bases (less than 10 or less than 5), enforced reactivity is still likely to occur, but will tend to diminish as the non-base paired segment grows in length.

The present disclosure thus provides any of the metabolic labeling methods described herein, further comprising performing a Locked TAPER reaction using the nucleic acid template as a target nucleic acid sequence for the TAPER reaction.

In some embodiments, the Locked TAPER reaction comprises: contacting the nucleic acid template with a first haplomer, wherein the first haplomer comprises: a) a first polynucleotide comprising: i) a first stem portion comprising from about 10 to about 20 nucleotide bases; ii) an anti-target loop portion comprising from about 16 to about 40 nucleotide bases and having a first end to which the first stem portion is linked, wherein the anti-target loop portion is substantially complementary to the nucleic acid template; and iii) a second stem portion comprising from about 10 to about 20 nucleotide bases linked to a second end of the anti-target loop portion, wherein the first stem portion is substantially complementary to the second stem portion; and b) a first effector partial moiety linked to either the first stem portion or the second stem portion; wherein the Tₘ of the anti-target loop portion:nucleic acid template is greater than the Tₘ of the first stem portion:second stem portion; and contacting the first haplomer with a second haplomer, wherein the second haplomer comprises: a) a second polynucleotide comprising a nucleotide portion that is substantially complementary to the stem portion of the first polynucleotide that is linked to the first effector partial moiety; and b) a second effector partial moiety linked to the second polynucleotide, wherein the second effector partial moiety can chemically interact with the first effector partial moiety of the first haplomer; wherein the Tₘ of the second polynucleotide:first or second stem portion linked to the first effector partial moiety is less than or equal to the Tₘ of the first stem portion:second stem portion; and wherein the first effector partial moiety and the second effector partial moiety form an active effector agent when in sufficient proximity.

In some embodiments, the Tₘ of the first stem portion:second stem portion subtracted from the Tₘ of the anti-target loop portion:nucleic acid template is from about 10° C. to about 40° C.; and/or the Tₘ of the first stem portion:second stem portion is from about 40° C. to about 50° C.; and/or the Tₘ of the anti-target loop portion:nucleic acid template is from about 60° C. to about 80° C.; and/or the Tₘ of the first stem portion:second stem portion subtracted from the Tₘ of the anti-target loop portion:nucleic acid template is from about 10° C. to about 20° C.

In some embodiments, the first stem portion comprises from about 12 to about 18 nucleotide bases; and/or the anti-target loop portion comprises from about 18 to about 35 nucleotide bases; and/or the second stem portion comprises from about 12 to about 18 nucleotide bases.

In some embodiments, the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

In some embodiments, the first effector partial moiety and the second effector partial moiety are fragments of an active effector agent chosen from a peptide, protein, or a therapeutic agent. In some embodiments, the peptide or protein is a toxin, a pro-apoptotic agent, or a ligand for an antibody or antibody fragment.

In some embodiments, the first effector partial moiety and the second effector partial moiety each further comprise a selectively-reactive moiety. In some embodiments, the first selectively-reactive moiety and the second selectively-reactive moiety are a chemically reactable pair of selectively-reactive moieties chosen from an azide, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, and a quadricyclane. In some embodiments, the first selectively-reactive moiety and the second selectively-reactive moiety are an azide-alkyne pair, an azide-phosphine pair, or a tetrazine-norbornene/trans-cyclooctene pair.

In some embodiments, the anti-target loop portion further comprises an internal hinge region, wherein the hinge region comprises one or more nucleotides that are not complementary to the nucleic acid template.

In some embodiments, the hinge region comprises from about 1 nucleotide to about 6 nucleotides.

In any of the Locked TAPER methods described herein, the first polynucleotide can comprise the nucleotide sequence 5′-ACTCGAGACGTCTCCTTGTCTTTGCTTTTCTTCA GGACACAGTGGCGAGACGTCTCGAGT-3′ (SEQ ID NO:7), and the second polynucleotide comprises the nucleotide sequence 5′-AGCTCTCGAGT-3′ (SEQ ID NO:9); or the first polynucleotide comprises the nucleotide sequence 5′-ACTCGAGACGTCTCCTT CCTGCCCCTCCTCCTGCTCCGAGACGTCTCGAGT-3′ (SEQ ID NO:8), and the second polynucleotide comprises the nucleotide sequence 5′-GACGTCTCGAGT-3′ (SEQ ID NO:10).

In any of the TAPER reactions described herein, the haplomer comprises at least one selectively-reactive moiety. The selectively-reactive moiety allows the formation of an active effector agent, such as through a chemical reaction or physical interaction with a corresponding selectively-reactive moiety. The selectively-reactive moiety can interact with or bind to the nucleic acid recognition moiety. The selectively-reactive moiety can also interact with or bind to the effector partial moiety.

A selectively-reactive moiety can be biologically inert. In particular, the selectively-reactive moiety can interact readily with a corresponding selectively-reactive moiety, but will not readily interact with natural biomolecules. This is to ensure that the nucleic acid templated assembly product is formed when corresponding haplomers are assembled on a nucleic acid template. It also safeguards the haplomers from reacting with functional groups on other molecules present in the environment in which the assembly occurs, preventing the formation of intended product.

The selectively-reactive moiety provides a mechanism for templated reactions to occur in complex target compartments, such as a cell, virus, tissue, tumor, lysate, other biological structure, or spatial region within a sample that contains the nucleic acid template, or that contains a different amount of nucleic acid template than a non-target compartment. A selectively-reactive moiety can react with a corresponding selectively-reactive moiety, but does not react with common biochemical molecules under typical conditions. Unlike other reactive entities, the selectivity of selectively-reactive moiety prevents ablation of the reactive group prior to assembly of the product or reactant.

An example of selectively-reactive moiety can include a bio-orthogonal reactive moiety. The bio-orthogonal reactive moiety includes those groups that can undergo “click” reactions between azides and alkynes, traceless or non-traceless Staudinger reactions between azides and phosphines, and native chemical ligation reactions between thioesters and thiols. Additionally, the bio-orthogonal moiety can be any of an azide, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, a quadricyclane, and derivatives thereof. Selectively reactive moieties of members of a set of corresponding haplomers are selected such that they will react with each other to produce an active effector agent.

Numerous selectively-reactive moieties can be used with the methods and compositions disclosed herein. Examples include, but are not limited to, azide-alkyne pairs, azide-activated alkyne pairs (e.g., cyclooctynes), azide-phosphine Staudinger/non-traceless Staudinger/traceless Staudinger chemistry pairs, traceless phosphinophenol Staudinger ligation, traceless phosphinomethanethiol Staudinger ligation, and native chemical ligation pairs, such as those described in, for example, PCT Publication WO 14/197547.

When any of the compounds described herein are contacted with a cell in vivo, the compounds can be delivered to a mammal, such as a human, by numerous routes of administration. Suitable routes of administration include, but are not limited to, oral, sublingual, buccal, rectal, intranasal, inhalation, eye drops, ear drops, epidural, intracerebral, intracerebroventricular, intrathecal, epicutaneous, transdermal, subcutaneous, intradermal, intravenous, intraarterial, intraosseous infusion, intramuscular, intracardiac, intraperitoneal, intravesical infusion, and intravitreal. In some embodiments, the administration is oral, sublingual, buccal, rectal, intranasal, inhalation, eye drops, or ear drops. In some embodiments, the administration is oral, sublingual, buccal, rectal, intranasal, or inhalation. In some embodiments, the administration is epidural, intracerebral, intracerebroventricular, or intrathecal. In some embodiments, the administration is epicutaneous, transdermal, subcutaneous, or intradermal. In some embodiments, the administration is intravenous, intraarterial, intraosseous infusion, intramuscular, intracardiac, intraperitoneal, intravesical infusion, or intravitreal. In some embodiments, the administration is intravenous, intramuscular, or intraperitoneal. The route of administration can depend on the particular disease, disorder, or condition being treated and can be selected or adjusted by the clinician according to methods known to the clinician to obtain desired clinical responses. Methods for administration are known in the art and one skilled in the art can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

In some embodiments, it may be desirable to administer one or more compounds, or a pharmaceutically acceptable salt thereof, to a particular area in need of treatment. This may be achieved, for example, by local infusion (for example, during surgery), topical application (for example, with a wound dressing after surgery), by injection (for example, by depot injection), catheterization, by suppository, or by an implant (for example, where the implant is of a porous, non-porous, or gelatinous material, including membranes, such as sialastic membranes, or fibers). Formulations for injection can be presented in unit dosage form, such as in ampoules or in multi-dose containers, with an added preservative.

The compounds described herein can be formulated for parenteral administration by injection, such as by bolus injection or continuous infusion. The compounds can be administered by continuous infusion subcutaneously over a period of about 15 minutes to about 24 hours. The compositions can take such forms as suspensions, solutions or emulsions in oily or aqueous vehicles, and can contain formulatory agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a solution, suspension, emulsion, or dry powder.

For oral administration, the compounds described herein can be formulated by combining the compounds with pharmaceutically acceptable carriers well known in the art. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations including, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, including, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as, for example, mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

For buccal administration, the compositions can take the form of, such as, tablets or lozenges formulated in a conventional manner.

For administration by inhalation, the compounds described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

In transdermal administration, the compounds can be applied to a plaster, or can be applied by transdermal, therapeutic systems that are consequently supplied to the organism. In some embodiments, the compounds are present in creams, solutions, powders, fluid emulsions, fluid suspensions, semi-solids, ointments, pastes, gels, jellies, and foams, or in patches containing any of the same.

The compounds described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compounds described herein can be contained in formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol). Pharmaceutical carriers can be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

The amount of compound to be administered may be that amount which is effective to produce sufficient cell labeling. The dosage to be administered may depend on the characteristics of the subject being treated, e.g., the particular animal treated, age, weight, health, types of concurrent treatment, if any, and frequency of treatments, and on the nature and extent of the disease, condition, or disorder, and can be easily determined by one skilled in the art (e.g., by the clinician). The selection of the specific dose regimen can be selected or adjusted or titrated by the clinician according to methods known to the clinician to obtain the desired clinical response. In addition, in vitro or in vivo assays may optionally be employed to help identify optimal dosage ranges. The precise dose to be employed in the compositions may also depend on the route of administration, and should be decided according to the judgment of the practitioner and each patient's circumstances.

Suitable dosage ranges for oral administration include, but are not limited to, from about 0.001 mg to about 200 mg, from about 0.01 mg to about 100 mg, from about 0.01 mg to about 70 mg, from about 0.1 mg to about 50 mg, from 0.5 mg to about 20 mg, or from about 1 mg to about 10 mg. In some embodiments, the oral dose is about 5 mg.

Suitable dosage ranges for intravenous administration include, but are not limited to, from about 0.01 mg to about 500 mg, from about 0.1 mg to about 100 mg, from about 1 mg to about 50 mg, or from about 10 mg to about 35 mg.

Suitable dosage ranges for other routes of administration can be calculated based on the forgoing dosages as known by one skilled in the art. For example, recommended dosages for intradermal, intramuscular, intraperitoneal, subcutaneous, epidural, sublingual, intracerebral, transdermal, or inhalation are in the range from about 0.001 mg to about 200 mg, from about 0.01 mg to about 100 mg, from about 0.1 mg to about 50 mg, or from about 1 mg to about 20 mg. Effective doses may be extrapolated from dose-response curves derived from in vitro or animal model test systems.

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, and 1.2% saline, and any combination thereof. In some embodiments, excipient is chosen from propylene glycol, purified water, and glycerin.

In some embodiments, the formulation can be lyophilized to a solid and reconstituted with, for example, water prior to use.

When administered to a mammal (e.g., to an animal for veterinary use or to a human for clinical use) the compounds can be administered in isolated form.

When administered to a human, the compounds can be sterile. Water is a suitable carrier when the compound is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences, A. R. Gennaro (Editor) Mack Publishing Co.

In some embodiments, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, the composition is in the form of a liquid wherein the active agent (i.e., one of the facially amphiphilic polymers or oligomers disclosed herein) is present in solution, in suspension, as an emulsion, or as a solution/suspension. In some embodiments, the liquid composition is in the form of a gel. In other embodiments, the liquid composition is aqueous. In other embodiments, the composition is in the form of an ointment. In some embodiments, the composition is an in situ gellable aqueous solution, suspension or solution/suspension, comprising about from 0.2% to about 3% or from about 0.5% to about 1% by weight of a gelling polysaccharide, chosen from gellan gum, alginate gum and chitosan, and about 1% to about 50% of a water-soluble film-forming polymer, preferably selected from alkylcelluloses (e.g., methylcellulose, ethylcellulose), hydroxyalkylcelluloses (e.g., hydroxyethylcellulose, hydroxypropyl methylcellulose), hyaluronic acid and salts thereof, chondroitin sulfate and salts thereof, polymers of acrylamide, acrylic acid and polycyanoacrylates, polymers of methyl methacrylate and 2-hydroxyethyl methacrylate, polydextrose, cyclodextrins, polydextrin, maltodextrin, dextran, polydextrose, gelatin, collagen, natural gums (e.g., xanthan, locust bean, acacia, tragacanth and carrageenan gums and agar), polygalacturonic acid derivatives (e.g., pectin), polyvinyl alcohol, polyvinylpyrrolidone and polyethylene glycol. The composition can optionally contain a gel-promoting counterion such as calcium in latent form, for example encapsulated in gelatin.

Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof phenoxyethanol; chlorophenoxyethanol; phenoxypropanol; chlorobutanol; chlorocresol; phenylethyl alcohol; disodium EDTA; and sorbic acid and salts thereof.

Optionally one or more stabilizers can be included in the compositions to enhance chemical stability where required. Suitable stabilizers include, but are not limited to, chelating agents or complexing agents, such as, for example, the calcium complexing agent ethylene diamine tetraacetic acid (EDTA). For example, an appropriate amount of EDTA or a salt thereof, e.g., the disodium salt, can be included in the composition to complex excess calcium ions and prevent gel formation during storage. EDTA or a salt thereof can suitably be included in an amount of about 0.01% to about 0.5%. In those embodiments containing a preservative other than EDTA, the EDTA or a salt thereof, more particularly disodium EDTA, can be present in an amount of about 0.025% to about 0.1% by weight.

One or more antioxidants can also be included in the compositions. Suitable antioxidants include, but are not limited to, ascorbic acid, sodium metabisulfite, sodium bisulfite, acetylcysteine, polyquaternium-1, benzalkonium chloride, thimerosal, chlorobutanol, methyl paraben, propyl paraben, phenylethyl alcohol, edetate disodium, sorbic acid, or other agents know to those of skill in the art. Such preservatives are typically employed at a level of from about 0.001% to about 1.0% by weight.

Suitable solubilizing agents for solution and solution/suspension compositions are cyclodextrins. Suitable cyclodextrins can be chosen from α-cyclodextrin, O-cyclodextrin, γ-cyclodextrin, alkylcyclodextrins (e.g., methyl-β-cyclodextrin, dimethyl-β-cyclodextrin, diethyl-β-cyclodextrin), hydroxyalkylcyclodextrins (e.g., hydroxyethyl-β-cyclodextrin, hydroxypropyl-β-cyclodextrin), carboxy-alkylcyclodextrins (e.g., carboxymethyl-O-cyclodextrin), sulfoalkylether cyclodextrins (e.g., sulfobutylether-β-cyclodextrin), and the like. Applications of cyclodextrins have been reviewed in Rajewski et al., J. Pharm. Sci., 1996, 85, 1155-1159.

In some embodiments, the composition optionally contains a suspending agent. For example, in those embodiments in which the composition is an aqueous suspension or solution/suspension, the composition can contain one or more polymers as suspending agents. Useful polymers include, but are not limited to, water-soluble polymers such as cellulosic polymers, for example, hydroxypropyl methylcellulose, and water-insoluble polymers such as cross-linked carboxyl-containing polymers. However, in some embodiments, the compositions do not contain substantial amounts of solid particulate matter, whether of the anti-microbial polymer or oligomer active agent, an excipient, or both. One or more acceptable pH adjusting agents and/or buffering agents can be included in the compositions, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

One or more acceptable salts can be included in the compositions in an amount required to bring osmolality of the composition into an acceptable range. Such salts include, but are not limited to, those having sodium, potassium or ammonium cations and chloride, citrate, ascorbate, borate, phosphate, bicarbonate, sulfate, thiosulfate or bisulfite anions. In some embodiments, salts include sodium chloride, potassium chloride, sodium thiosulfate, sodium bisulfite and ammonium sulfate. In some embodiments, the salt is sodium chloride.

Optionally one or more acceptable surfactants, preferably nonionic surfactants, or co-solvents can be included in the compositions to enhance solubility of the components of the compositions or to impart physical stability, or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40; polysorbate 20, 60 and 80; polyoxyethylene/polyoxypropylene surfactants (e.g., Pluronic® F-68, F84 and P-103); cyclodextrin; or other agents known to those of skill in the art. Typically, such co-solvents or surfactants are employed in the compositions at a level of from about 0.01% to about 2% by weight.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### Examples

### Example 1: Labeling Cells with Azide-Modified Sugars

Demonstration of the presence of azides on a cell surface was achieved by treating a cell preparation with a fluorescent azide reactive molecule that reacts only with the azide group, without chemical reactivity with normal biological molecules. Initially, cells were cultured in a suitable culture vessel such that their level of confluency at the time of addition of the azide-modified sugar, AzNAM, was not more than 80%.

In particular, HeLa cells were plated in 6-well plates (2.5×10⁴ cells/well) and incubated for 48 hours in DMEM-10% FBS medium in a standard 5% CO₂ atmosphere. Medium from each well was removed, and each well was washed with 2 ml of phosphate buffered saline (PBS), and fresh DMEM-FBS (1 ml) added. Subsequently, varying amounts of AzNAM (solubilized in DMSO) were added to the wells in small (10 μl) volumes to produce the desired final concentrations in the range 10-125 μM (see, FIG. 1). After an additional 20 hours incubation, the medium in each well was removed, and the wells were washed with 2 ml of PBS, followed by addition of 1 ml of PBS. Then, a fluorescent azide reactive molecule, DBCO-FAM (Broadpharm), was directly added to each well to produce a final concentration of 10 μM.

Plates with added DBCO-FAM were protected from bright light and incubated for 1 hour at room temperature. Supernatants in each well were removed, and each well was washed twice with 2 ml of PBS. Cells were then taken up with CellStripper (Thermo) reagent and transferred into 1.5 ml tubes. After pelleting and washing in PBS, cells were resuspended in 150 μl of PBS and counted. Defined numbers in 50 μl volumes in a 96-well Blackwell plate (Corning) were assayed for fluorescence in a fluorescent plate reader (Tecan).

Fluorescence arising from the reaction between DBCO-FAM and cell-surface azide label was also demonstrated by flow cytometry. In separate cultures, HeLa cells were added to wells of a 12-well plate at 10,000 cells/well, and cultured for 72 hours under normal conditions. The wells were washed with 1 ml of PBS, and 0.5 ml of DMEM-10% FBS was added. AzNAM was added to a final concentration of 125 μM, with cells receiving no azide-modified sugar as controls. After 20 hours, cells were harvested with CellStripper (250 μl/well), washed with PBS, and subjected to flow analysis.

By fluorescence readings in a 96-well plate format, it was demonstrated that cell-associated fluorescence increased as a function of the levels of AzNAM used in the initial labeling test (see, FIG. 14). With flow analyses, marked and well-demarcated peak shifts were observed with the AzNAM-treated cells, which was corroborated by measuring fluorescence from equal numbers of cells in a fluorescent plate reader (see, FIG. 15).

### Example 2: Labeling Cells Having Surface Azide-Modified Sugars with Bifunctional Compounds (Prophetic)

Cells that have been metabolically labeled with surface azide-modified sugars (see, Example 1) can be subsequently reacted with the bifunctional compounds described herein (see, for example, FIG. 5). After such reactions have occurred and excess compound is removed, the portion of the bifunctional molecule that is a small molecule ligand that binds to an FKBP binding site can be displayed on the surface of the cell and can be available for subsequent reactions with a complex comprising an FKBP binding site linked to a polynucleotide, peptide, or small molecule (see, FIG. 6). Alternately, the portion of the bifunctional molecule that is a small molecule ligand that binds to an FKBP binding site can first be allowed to react with the complex comprising an FKBP binding site linked to a polynucleotide, peptide, or small molecule, after which the exposed azide reactive molecule of the bifunctional compound can be used for targeting the complex to the cell surface labeled with azide-modified sugars.

In particular, any of the bifunctional compounds described herein can be used for the purposes of cell surface positioning of any of the complexes described herein. Cells displaying azide moieties on surface glycan molecules (as in Example 1) can be treated with 1 mM of the bifunctional compound (initially solubilized in DMSO as a 100 mM stock solution and diluted accordingly to the final desired concentration) in serum-free RPMI medium for 2 hours at room temperature in the presence of 1 mg/ml bovine serum albumin (BSA) (Sigma) and 500 μg/ml salmon sperm DNA. This treatment is followed by centrifugation (5 minutes at 2000 rpm in an Eppendorf centrifuge), followed by two washes with serum-free RPMI medium, with resuspension in 100 μl of the same medium. Following this, a complex comprising an FKBP binding site linked to a polynucleotide, peptide, or small molecule can be added to the bifunctional compound-modified cells at a concentration of 1 pmol/μl, for a one hour incubation at room temperature. The cell preparations can be repelleted, washed twice with serum-free RPMI medium and once with PBS, with a final resuspension in 100 μl of PBS.

In an alternate embodiment, the complex comprising an FKBP binding site linked to a polynucleotide, peptide, or small molecule can be pre-incubated with excess bifunctional compound prior to exposure to the target cells displaying surface azide. The complex in PBS (100 pmol) can be incubated with a 10-fold molar excess of the bifunctional compound for one hour at room temperature, followed by passage through a PBS-equilibrated P6 desalting column (Bio-Rad) to remove excess bifunctional compound. The resulting complex-bifunctional compound can be used to treat cells having surface azide, followed by washing steps as above.

### Example 3: Surface Templating Via Metabolically Placed Azides and Detection by Ligation-Template PCR

The efficacy of templates placed on target cell surfaces can be demonstrated by means of a quantifiable assay that recapitulates the general haplomer assembly approach. For example, a nuclease-resistant template (with a 2′-O-methyl RNA backbone) can be modified with a 5′ or 3′ DBCO group, allowing it to specifically react with azides on the surface of metabolically labeled cells. Following this, the template-bearing cells can be treated with two shorter “ligation template” (LT) oligonucleotides each complementary to contiguous regions of the template, and where one of the LT oligonucleotides bears a 5′-phosphate (LT1). After annealing, the 5′-phosphate of LT1 can be ligated with the 3′-hydroxyl on its partner LT (LT2) adjacent on the same template (see, FIG. 16). For this ligation, a ligase enzyme can be used that is compatible with modified nucleic acid backbones, such as Splint® Ligase (New England Biolabs). Since each LT oligonucleotide is also equipped with an additional priming site, the joined LT (1+2) sequence can be amplified by PCR, and can also easily be amenable to quantitation by qPCR. In the latter case, standard curves can be set up using a single contiguous synthetic oligonucleotide that corresponds in sequence to the ligated LT(1+2) sequence.

To apply the surface template/LT assay to azide-labeled HeLa cells, cells were treated with several concentrations of AzNAM in 6-well plates at about 80% confluency. After 20 hours, cells were removed from the plate with CellStripper, washed with serum-RPMI 1640 medium (SF-RPMI), and counted. Tubes with 5×10⁵ cells in 0.5 ml SF-RPMI were then treated with 250 pmol of a template 40-mer with the sequence: (DBCO)-C6- Um UmUmUmUmCmCmUmGmUmGmUmCmCmUmGmAmAmGmAmAmAmGmCmAmAm AmGmAmCmAmUmCmUmGmGmAmCmAmA (SEQ ID NO:66), where ‘m’ denotes a 2′ O-methyl-modified ribose. Cell preparations incubated with the above template in SF-RPMI were co-incubated with 1 mg/ml of BSA and 500 μg/ml salmon sperm DNA, for 2 hours at room temperature, while ensuring that the cells were maintained in suspension. Following this treatment, to reduce the non-specific background binding resulting from the hydrophobicity of the DBCO group, cells were pelleted and resuspended in 1 ml of SF-RPMI with 1 mg/ml fatty acid-free BSA (Sigma), which can adsorb hydrophobic molecules equilibrating between solution-phase and membrane-associated states. After 30 minutes incubation at room temperature, cells were repelleted, washed twice with 500 μl of SF-RPMI, and finally resuspended in the same SF-RPMI volume.

For the remaining steps of the surface templating and assembly of LT oligos, it was convenient to employ a magnetic bead-based strategy. Protein A magnetic beads (New England Biolabs) were initially loaded with an antibody against human MHC Class I, (W6/32) known to be expressed at high levels on HeLa cells. Since W6/32 is of the IgG2a isotype, it is compatible with Protein A binding. Washed magnetic beads were treated with an excess of W6/32 for 1 hour at 4° C., followed by 3 rounds of washing (0.5 ml per wash) with SF-RPMI using standard magnetic bead separation technology. After the preparation of the template-bearing cells, 50 μl of W6/32-beads were added to each HeLa-template preparation as above, and incubated for 30 minutes at 4° C. The cell/magnetic bead slurries were washed twice with 250 μl of SF-RPMI, then once with 250 μl of PBS, and finally resuspended in 100 μl of PBS. Capture of the HeLa cells onto these W6/32 magnetic beads was confirmed by microscopic examination.

LT1 and LT2 oligonucleotides complementary to the surface template were added in equimolar quantities to a final concentration of 0.2 pmol/μl. Sequences of these oligonucleotides: LT1: 5′-phosphate-CTTTCTTCAGGACACAGAGGTAGCGTCTCAGGT GGGAAGTTCGGATTGCCAGTAGGTG (SEQ ID NO:67); and LT2: CACTACTGTGCTC CTCTCACTGCACTCTTGCTCACTCCACTTTTGTCCAGATGTCTTTG (SEQ ID NO:68). After a 30 minute annealing step between the LT oligonucleotides and surface template at room temperature, preparations were washed twice by magnetic separation with 500 μl of PBS. 100 μl of Splint® ligase solution (New England Biolabs) was added, comprised of a buffer (50 mM Tris pH 7.4/10 mM MgCl₂/50 mM NaCl/1 mM dithiothreitol/1 mM ATP) with a final enzyme concentration of 125 units/μl). Ligation reactions were allowed to proceed for 1 hour at room temperature. Following this, cells on magnetic beads were magnetically separated, washed twice with 500 μl of PBS, and resuspended in 200 μl of PBS. Bound surface template/LT oligonucleotides were released by treatment with Proteinase K (2 units) for 30 minutes at 37° C., after which the Proteinase K was inactivated by heating at 95° C. for 10 minutes.

The resulting preparations were used for qPCR testing, using a control standard corresponding to the ligated LT1 and LT2 ligation-template oligonucleotides. The control standard (code #593) for qPCR comprised the sequence: CACTACTGTGCTCCTCTCACTG CACTCTTGCTCACTCCACTTTTGTCCAGATGTCTTTGCTTTCTTCAGGACACAGAG GTAGCGTCTCAGGTGGGAAGTTCGGATTGCCAGTAGGTG (SEQ ID NO:69). Primers for qPCR: Forward primer (Code #604): CACCTACTGGCAATCCGAACT (SEQ ID NO:70); and Reverse primer (Code #605): CACTACTGTGCTCCTCTCACT (SEQ ID NO:71). Signals obtained from triplicate samples of the HeLa cell templating tests were converted into equivalent molecule numbers by interpolation from the standard curve (see, FIG. 19). A marked increase over background (over an order of magnitude) was observed for formation of the templated LT product in cells treated with greater than 6 μM of AzNAM (see, FIG. 17).

### Example 4: Assessment of Positioning of Cell-Surface Template Via Metabolically Placed Azides Detected by Bi-Labeled FAM Complementary Probe

Specific cell surface template (positioned by means of click chemical reactions with metabolically derived surface AzNAM; as described in Example 3) can be detected with alternate methods. One such approach uses probe nucleic acid molecules that are complementary to the template of interest, where both 5′- and 3′-ends of such probes are both modified with a fluorescent group, such as FAM.

For example, HeLa cells with surface template of the same sequence as in Example 3 but with a DNA backbone were treated with a 5′/3′ bi-labeled complementary probe of sequence: TCCAGATGTCTTTGCTTTTCTTCAGGACACA (SEQ ID NO:72). The same cells were also treated with a control bi-labeled scrambled oligonucleotide of sequence CTT ACCTAATGCTGTTTATATCATCGTTCTGT (SEQ ID NO:73). For treatments, 10⁵ washed AzNAM-labeled HeLa cells in 100 μl of serum-free RPMI 1640 medium were incubated with 800 pmol of complementary or scrambled bi-labeled oligonucleotides for 30 minutes at room temperature. Cells were centrifuged (3 minutes at 2000 rpm) and washed once with 500 μl of serum-free RPMI 1640 medium and once with the same volume of PBS. Cells were finally resuspended in 200 μl of PBS and subjected to standard flow analysis.

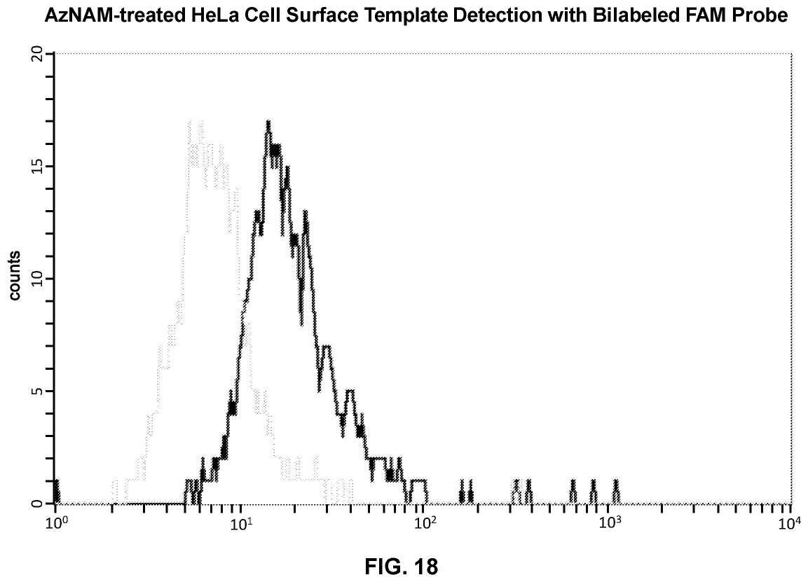

AzNAM-treated cells incubated with the complementary oligonucleotide exhibited a significantly shifted fluorescent peak relative to cells incubated with the control scrambled oligonucleotide (see, FIG. 18). Such results are consistent with hybridization of the bi-labeled complementary oligonucleotide with surface template.

### Example 5: Assaying Surface Glycan Density by Proximal Ligation Reverse Templating (Prophetic)

The principle of the reverse templating approach relies on click reactions between pairs of oligonucleotides labeled with DBCO (or other selectively-reactive moieties) with cell surface azides present as a metabolic consequence of AzNAM treatment. Subsequently, a bridging template oligonucleotide can be used to enable ligation of the surface-reacted shorter oligonucleotides, only after which is their amplification and quantitation possible (see, FIG. 4).

Cells at about 80% confluency can initially be treated for 20 hours with 100 μM of AzNAM and harvested. After washing in serum-free RPMI medium and adjusting to a standard concentration (10⁶ cells/ml), 0.5 ml cells can be treated with an equimolar mixture (500 pmol each) of two ligation template oligonucleotides, where asterisks denote a phosphorothioate backbone: 5′-phosphate-C*T*T*T*C*T*T*C*A*G*G*A*C*A*C*A*G* A*G*G*T*A*G*C*G*T*C*T*C*A*G*G*T*G*G*G*A*A*G*T*T*C*G*G*A*T*T*G*C *C*A*G*T*A*G*G*T*G*-3′ DBCO (SEQ ID NO:74); and 5′DBCO-C*A*C*T*A*C*T* G*T*G*C*T*C*C*T*C*T*C*A*C*T*G*C*A*C*T*C*T*T*G*C*T*C*A*C*T*C*C*A* C*T*T*T*T*G*T*C*C*A*G*A*T*G*T*C*T*T*T*G (SEQ ID NO:75). Reactions can be performed in the presence of 500 μg/ml salmon sperm DNA and 1 mg/ml BSA. After 2 hours reaction at room temperature, cells can be washed in serum-free RPMI medium twice and resuspended in 250 μl of the same medium. A 2′-O-methyl bridging oligonucleotide can be added to a final concentration of 1 pmol/μl, for another 30 minutes at room temperature incubation. Cells can then be centrifuged and rewashed (500 μl) twice with PBS, once more with the same volume of modified Splint® ligase buffer (50 mM Tris pH 7.5/10 mM MgCl₂/1 mM DTT/1 mM ATP), and finally resuspended in 100 μl of the same splint ligase buffer also containing 125 units/ml of Splint® ligase (New England Biolabs). After 30 minutes at room temperature, cells can be centrifuged, washed twice with PBS, and resuspended in 100 μl of PBS.

Samples (1 μl) of evenly resuspended cells can be subjected to standard PCR analysis, using primers CACCTACTGGCAATCCGAACT (SEQ ID NO:70) and CACTAC TGTGCTCCTCTCACT (SEQ ID NO:71). Amplification sensitivity can be increased by re-amplifying small samples ( 1/50) of the resulting products with internal nested primers TCC CACCTGAGACGCTACCT (SEQ ID NO:76) and GCACTCTTGCTCACTCCACTT (SEQ ID NO:77). qPCR can be readily applied using standard reagents for incorporation of SYBR-Green (Thermo) during amplification cycles. Signals from test samples can be used to interpolate the corresponding molecular levels from a standard curve run at the same time, where the standard itself is a synthetic DNA oligonucleotide corresponding in sequence to the above Splint® ligase ligation product: CACTACTGTGCTCCTCTCACTGCACTCTTGC TCACTCCACTTTTGTCCAGATGTCTTTGCTTTCTTCAGGACACAGAGGTAGCGTCT CAGGTGGGAAGTTCGGATTGCCAGTAGGTG (SEQ ID NO:69). Quantitation of cell surface-templated molecules can be related to the known numbers of input cells in the assay in order to provide an index of surface density.

### Example 6: Placement of Proteins of Interest on Cell Surfaces Via Surface MFL4-DB (Prophetic)

Cells that have been metabolically labeled with surface azides can be subsequently reacted with compounds composed in part of DBCO or other selectively-reactive moieties. After such reactions have occurred and excess compound is removed, the remainder (non-click reactive) portion of the original compound can be displayed on cell surfaces for subsequent reactions or functions of interest. Alternately, the non-click portion of bifunctional molecules can first be allowed to react or form a complex with a protein target, after which the exposed selectively-reactive portion of the same molecule is used for targeting the protein complex to an azide-modified cell surface.

The compounds MFL4-DB or MFL5-PBCN, or related analogs of either, can be used for the purposes of cell surface positioning of any protein of interest that is fused with an FKBP domain. For instance, cells displaying azide moieties on surface glycan molecules (as in Example 1) can be treated with 1 mM of MFL4-DB (initially solubilized in DMSO as a 100 mM stock solution and diluted accordingly to the final desired concentration) in serum-free RPMI medium for 2 hours at room temperature in the presence of 1 mg/ml BSA and 500 μg/ml salmon sperm DNA. This treatment is followed by centrifugation (5 minutes at 2000 rpm in an Eppendorf centrifuge), and then two washes with serum-free RPMI medium, with resuspension in 100 μl of the same medium. Full-length Gaussia luciferase bearing a C-terminal fusion with a modified FKBP domain with mutations C22V and F36V (see, PCT Publication WO 18/94195) can be added to the MFL4-DB-modified cells at a concentration of 1 pmol/μl, for a 1 hour incubation at room temperature. The cell preparations can be repelleted, washed twice with serum-free RPMI medium and once with PBS, with final resuspension in 100 μl of PBS. Cell surface binding of Gaussia luciferase can be examined directly for luminescence with 2 μl samples in a standard luminometer. Controls for this experiment can include: (1) cells without an initial AzNAM treatment for azide display, but treated with MFL4-DB; and (2) azide-bearing (AzNAM pre-treated) cells without the initial MFL4-DB treatment. Both (1) and (2) controls can likewise be treated with Gaussia-FKBP as above, where resulting luminescence constitutes background (non-specific) binding.

In an alternate process, the full-length Gaussia-FKBP fusion can be pre-incubated with excess MFL4-DB prior to exposure to target cells displaying surface azide. Gaussia-FKBP in PBS (100 pmol) can be incubated with a 10-fold molar excess of MFL4-DB for 1 hour at room temperature, followed by passage through a PBS-equilibrated P6 desalting column (Bio-Rad) to remove excess MFL4-DB. The resulting protein-MFL4-DB complex can be used to treat cells with and without initial AzNAM treatments, followed by washing steps as described above. Luminescence of the AzNAM-treated azide-displaying cells (targets) and the non-AzNAM controls can be assessed, also in the same manner as above.

For Examples 7-11, metabolic labeling was monitored by treatment of cultured cells with the sugar analog peracetylated N-azidoacetylmannosamine (AzNAM), followed after harvest with treatment with the bifunctional click/fluorescent compound DBCO-PEG3-FAM (DPEG-FAM). Surface azide groups resulting from the metabolic labeling process react with the DBCO group (dibenzocyclooctyne) of DPEG-FAM, and were monitored by flow cytometry.

In each experiment, it was observed that the relative magnitudes of this labeling signal varied between different cell lines, for which several explanations can be proposed. But variation in the levels or activities of the enzymes of the sialic acid pathway which control the processing of AzNAM intracellularly (after deacetylation by esterases) could potentially parallel levels of labeling itself. If a specific treatment selectively favored such up-regulation in transformed tumor cells (of any lineage), it could potentially be of therapeutic significance in conjunction with TAPER targeting.

Accordingly, both interferon-beta itself and also agents known to act as agonists for an important upstream regulator of interferon pathways, STING (Stimulator of Interferon Genes) were used in these experiments. STING is an early responder to foreign nucleic acids, via cGAS signaling, and functions upstream of IFN-β itself.

### Example 7

Jurkat cells (human T lymphoma; 105 cells/ml) were treated with or without 100 ng/ml human interferon-beta (IFNβ), for three days in culture. 22 hr prior to harvest, cell media were supplemented with 100 mM AzNAM or control solvent only (DMSO). After harvest and washing, cells (106 cells/ml) were treated with 2 mM DPEG-FAM, washed, and subjected to flow cytometric analysis. It was found (see FIGS. 20A-20D) that the geometric mean fluorescence of AzNAM-cells was increased by IFNβ treatment (a 1.4-fold enhancement relative to the untreated value). Although control DMSO-treated cells also showed such a shift, it was of reduced magnitude compared to the AzNAM cells. FIGS. 20A-20D show flow cytometric analysis of human Jurkat cells after treatment with 100 ng/ml human IFN-beta for three days, with addition of 100 μM AzNAM at 22 hr prior to harvest. Cells were treated with DPEG-FAM prior to flow analysis. FIG. 20A shows Jurkats/AzNAM+IFNβ; GM=150.15. FIG. 20B shows Jurkats/DMSO+IFNβ; GM=16.66. FIG. 20C shows Jurkats/AzNAM (Ctrl); GM=106.33. FIG. 20D shows Jurkats/DMSO Ctrl; GM=3.49.

### Example 8

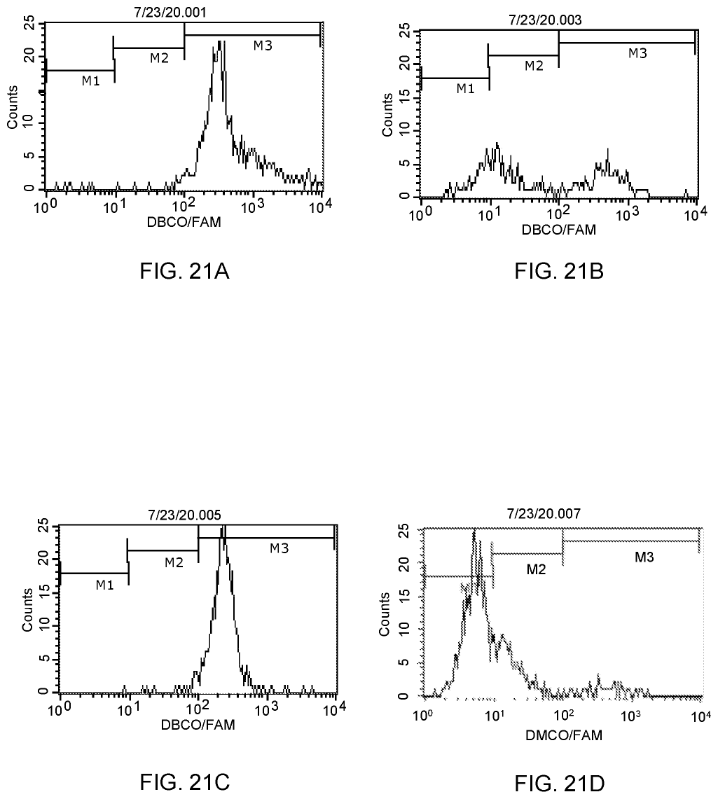

At 100 ng/ml of IFNβ, certain toxicity was seen with Jurkat cells. With a lowered IFNβ dose of 75 ng/ml and the same general conditions as for Example 7, a similar pattern was seen (see FIGS. 21A-21D; 1.9-fold enhancement relative to the untreated value). As in Example 7, a biphasic response in the DMSO control cells was seen, but in many circumstances the smaller observed peak with stronger fluorescence is attributable to non specific uptake of the DPEG-FAM fluor, through a partial permeabilization effect of the treatments. FIGS. 21A-21D show flow cytometric analysis of human Jurkat cells after treatment with 75 ng/ml human IFN-beta for 3 days, with addition of 100 μM AzNAM at 22 hr prior to harvest. Cells were treated with DPEG-FAM prior to flow analysis. FIG. 21A shows Jurkats/AzNAM+IFNβ; GM=385.16. FIG. 21B shows Jurkats/DMSO+IFNβ; GM=19.40. FIG. 21C shows Jurkats/AzNAM (Ctrl); GM=207.3. FIG. 21D shows Jurkats/DMSO Ctrl; GM=8.20.

### Example 9

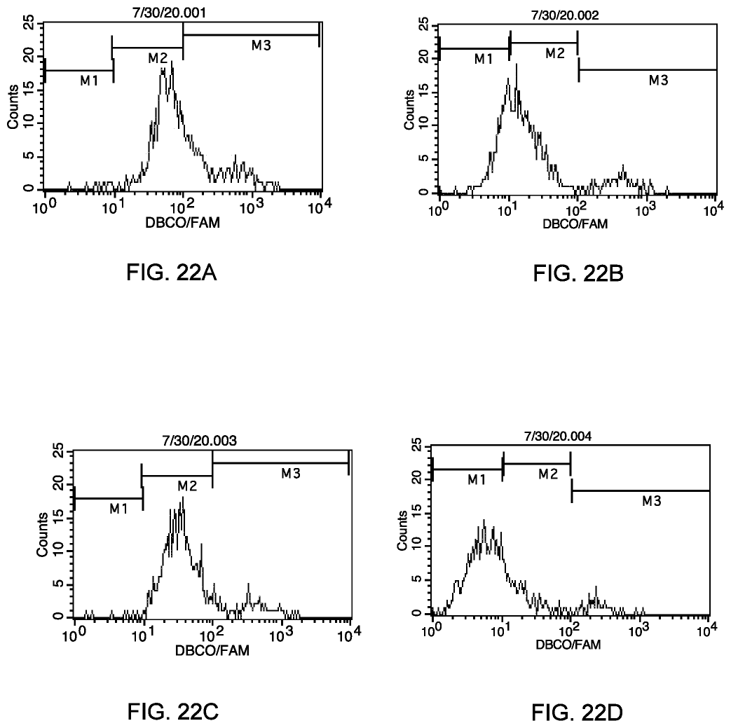

The murine ovarian cancer cell line ID8 represent an example of a cell type giving a relatively low metabolic labeling response, as assessed by AzNAM/DPEG-FAM as usual, results shown in FIGS. 22A-22D. Subconfluent ID8 cells were treated with or without 100 ng/ml murine interferon-beta (mIFNβ), and then treated with AzNAM and DPEG-FAM in a similar manner as for the Jurkat cells of Examples 7 and 8. Treatment with mIFNβ produced a greater labeling effect on AzNAM-treated cells (2.1-fold enhancement relative to the untreated value; see FIGS. 22A-22D), while control DMSO cells showed a much reduced labeling increase. FIGS. 22A-22D show flow cytometric analysis of murine ID8 cells after treatment with 100 ng/ml murine IFN-beta for 3 days, with addition of 100 μM AzNAM at 22 hr prior to harvest. Cells were treated with DPEG-FAM prior to flow analysis. FIG. 22A shows ID8/AzNAM+mIFNβ; GM=80.58. FIG. 22B shows ID8/DMSO+mIFNβ; GM=16.49. FIG. 22C shows ID8/AzNAM (Ctrl); GM=37.68. FIG. 22D shows ID8/DMSO (Ctrl); GM=7.94.

### Example 10

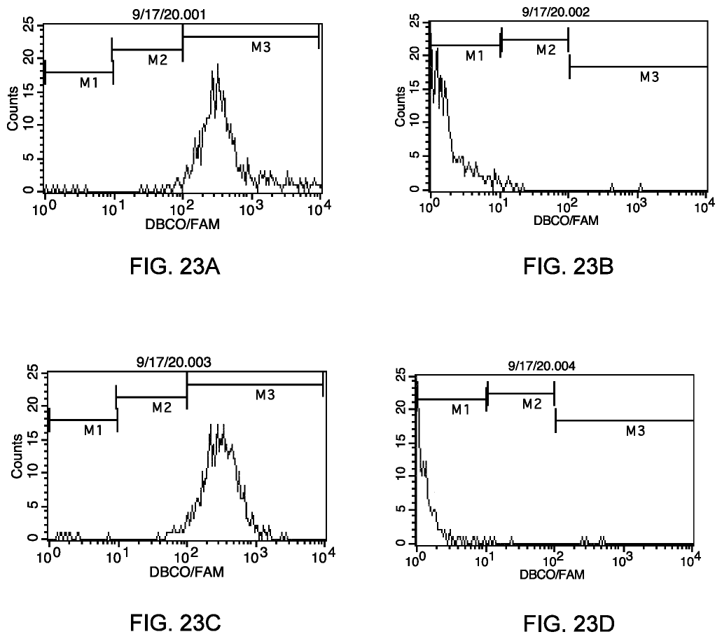

The human STING agonist-1 (STAG-1′; MedChemExpress LLC) was tested for Jurkat cells in a similar experimental arrangement as for Examples 7 and 8. A metabolic labeling enhancement with STAG1 was seen, though modest (see FIGS. 23A-23D; 1.2-fold enhancement relative to the untreated value). The impact on control DMSO-treated cells was minimal. FIGS. 23A-23D show flow cytometric analysis of human Jurkat cells after treatment with 50 μM STING agonist 1 (STAG1) for 3 days, with addition of 100 μM AzNAM at 22 hr prior to harvest. Cells were treated with DPEG-FAM prior to flow analysis. FIG. 23A shows Jurkats/AzNAM+STAG1; GM=325.69. FIG. 23B shows Jurkats/DMSO+STAG1; GM=1.47. FIG. 23C shows Jurkats/AzNAM (Ctrl); GM=282.70. FIG. 23D shows Jurkats/DMSO Ctrl; GM=1.12.

### Example 11

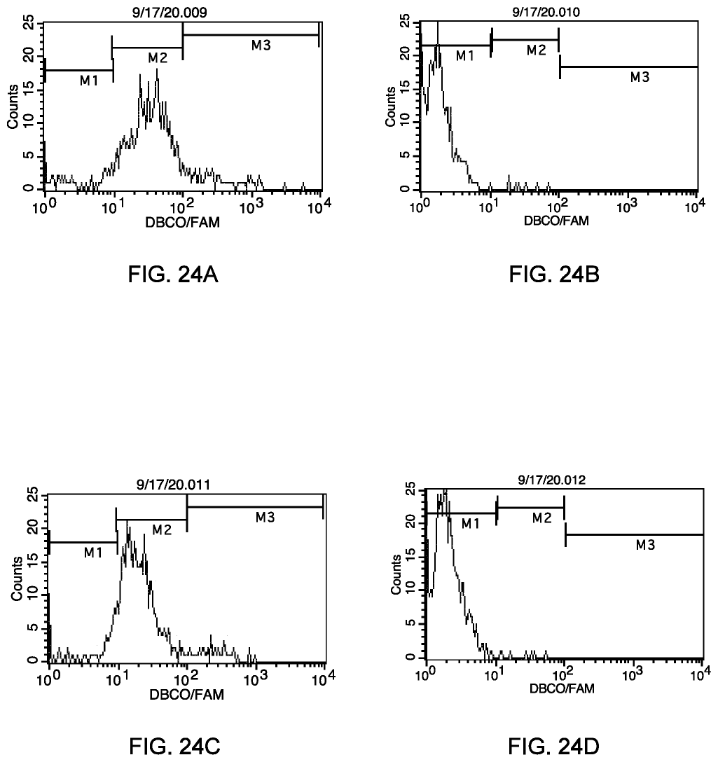

The murine-specific STING agonist DMXAA (R&D Systems) was used to test ID8 cells with a similar experimental arrangement as for Example 9. It was found (see FIGS. 24A-24D) that DMXAA elicited an increased AzNAM/DPEG-FAM response (1.8-fold enhancement relative to the untreated value), while no effect was seen with the comparably-treated DMSO controls. FIGS. 24A-24D show flow cytometric analysis of murine ID8 cells after treatment with 20 μM murine STING agonist DMXAA for 3 days, with addition of 100 μM AzNAM at 22 hr prior to harvest. Cells were treated with DPEG-FAM prior to flow analysis. FIG. 24A shows ID8/AzNAM+DMXAA; GM=33.73. FIG. 24B shows ID8/Ctrl+DMXAA; GM=1.67. FIG. 24C shows ID8/AzNAM (Ctrl); GM=19.14. FIG. 24D shows ID8/DMSO (Ctrl); GM=1.98.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method of labeling a cell with a substrate for a templated assembly reaction, the method comprising the steps:
a) contacting the cell with an azide-modified sugar; and
b) contacting the cell with the substrate for the templated assembly reaction, wherein the substrate comprises:
i) a nucleic acid template; and
ii) an azide reactive molecule linked to the nucleic acid template at the 5′- or 3′-end of the nucleic acid template, wherein the azide reactive molecule is chemically reactable with the azide of the azide-modified sugar.

2. The method according to claim 1, wherein the azide reactive molecule is dibenzocyclooctyne (DBCO), bicyclo[6.1.0]nonyne (BCN), methyltetrazine, or trans-cyclooctene (TCO).

3. The method according to claim 1 or claim 2, wherein the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA).

4. The method according to any one of claims 1 to 3, wherein the azide-modified sugar is acetylated at 1, 2, 3, or 4 positions.

5. The method according to any one of claims 1 to 4, wherein the nucleic acid template is chosen from a cancer-specific polynucleotide, a viral polynucleotide, a microbial-specific polynucleotide, a differentially expressed gene, and a disease-specific polynucleotide.

6. The method according to any one of claims 1 to 5, further comprising performing a Template Assembly by Proximity-Enhanced Reactivity (TAPER) reaction using the nucleic acid template as a target nucleic acid sequence for the TAPER reaction.

7. The method according to claim 6, wherein the TAPER reaction comprises contacting the cell with a first haplomer and a second haplomer, wherein:
the first haplomer comprises:
a first polynucleotide that is complementary to a first region of the nucleic acid template;
a first effector partial moiety, wherein the first effector partial moiety is linked to the first polynucleotide; and
a first selectively-reactive moiety, wherein the first selectively-reactive moiety is linked to the first effector partial moiety;
the second haplomer comprises:
a second polynucleotide that is complementary to a second region of the nucleic acid template;
a second effector partial moiety, wherein the second effector partial moiety is linked to the second polynucleotide; and
a second selectively-reactive moiety, wherein the second selectively-reactive moiety is linked to the second effector partial moiety;
wherein:
the first selectively-reactive moiety and the second selectively-reactive moiety chemically react with each other when in sufficient proximity;
the first region of the nucleic acid template is in sufficient proximity to the second region of the nucleic acid template to allow the first selectively-reactive moiety and the second selectively-reactive moiety to chemically react with each other; and
the first effector partial moiety and the second effector partial moiety form an active effector agent when in sufficient proximity.

8. The method according to claim 7, wherein the first selectively-reactive moiety and the second selectively-reactive moiety are a chemically reactable pair of selectively-reactive moieties chosen from an azide, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, and a quadricyclane.

9. The method according to claim 8, wherein the first selectively-reactive moiety and the second selectively-reactive moiety are an azide-alkyne pair, an azide-phosphine pair, or a tetrazine-norbornene/trans-cyclooctene pair.

10. The method according to any one of claims 7 to 9, wherein the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

11. The method according to any one of claims 7 to 10, wherein the first effector partial moiety and the second effector partial moiety are fragments of an active effector agent chosen from a peptide, protein, or a therapeutic agent.

12. The method according to claim 11, wherein the peptide or protein is a toxin, a pro-apoptotic agent, or a ligand for an antibody or antibody fragment.

13. The method according to any one of claims 1 to 5, further comprising performing a Ligand Directed TAPER (LD-TAPER) reaction using the nucleic acid template as a target nucleic acid sequence for the LD-TAPER reaction.

14. The method according to claim 13, wherein the LD-TAPER reaction comprises:
contacting the nucleic acid template with a first haplomer-ligand complex, wherein the first haplomer-ligand complex comprises a first haplomer, wherein the first haplomer comprises a first polynucleotide, and a first small molecule ligand linked to the 5′ or 3′ terminus of the first haplomer, wherein the first small molecule ligand comprises a first small molecule ligand partner binding site;
contacting the nucleic acid template with a second haplomer-ligand complex, wherein the second haplomer-ligand complex comprises a second haplomer, wherein the second haplomer comprises a second polynucleotide, and a second small molecule ligand linked to the 5′ or 3′ terminus of the second haplomer, wherein the second small molecule ligand comprises a second small molecule ligand partner binding site;
contacting the first haplomer-ligand complex with a first fusion protein, wherein the first fusion protein comprises a first fragment of a protein of interest fused to a first ligand binding domain for a small molecule ligand;
contacting the second haplomer-ligand complex with a second fusion protein, wherein the second fusion protein comprises a second fragment of the protein of interest fused to a second ligand binding domain for a small molecule ligand;
wherein the first ligand of the first haplomer-ligand complex is linked to the 5′ terminus of the first polynucleotide of the first haplomer-ligand complex;
wherein the second ligand of the second haplomer-ligand complex is linked to the 3′ terminus of the second polynucleotide of the second haplomer-ligand complex;
wherein the first polynucleotide of the first haplomer-ligand complex is substantially complementary to the nucleic acid template;
wherein the second polynucleotide of the second haplomer-ligand complex is substantially complementary to the nucleic acid template at a site in spatial proximity to the first polynucleotide of the first haplomer-ligand complex;
wherein the first small molecule ligand of the first haplomer-ligand complex and the first ligand binding domain of the first fusion protein can interact; and
wherein the second small molecule ligand of the second haplomer-ligand complex and the second ligand binding domain of the second fusion protein can interact;
thereby resulting in the folding or dimerization of the first fragment of the protein of interest of the first fusion protein with the second fragment of the protein of interest of the second fusion protein.

15. The method according to claim 14, wherein:
the first small molecule ligand is an FKBP binding compound, and the first ligand binding domain for the first small molecule ligand is an FKBP domain or a FRB domain; and
the second small molecule ligand is an FKBP binding compound, and the second ligand binding domain for the second small molecule ligand is an FKBP domain or a FRB domain.

16. The method according to claim 13, wherein the LD-TAPER reaction comprises:
contacting the nucleic acid template with a first haplomer-ligand complex, wherein the first haplomer-ligand complex comprises a first haplomer, wherein the first haplomer comprises a first polynucleotide, and a first ligand linked to the 5′ or 3′ terminus of the first haplomer, wherein the first ligand is a first interactive protein domain and comprises a first ligand partner binding site;
contacting the nucleic acid template with a second haplomer-ligand complex, wherein the second haplomer-ligand complex comprises a second haplomer, wherein the second haplomer comprises a second polynucleotide, and a second ligand linked to the 5′ or 3′ terminus of the second haplomer, wherein the second ligand is a second interactive protein domain and comprises a second ligand partner binding site;
contacting the first haplomer-ligand complex with a first fusion protein, wherein the first fusion protein comprises a first fragment of a protein of interest fused to a third interactive protein domain;
contacting the second haplomer-ligand complex with a second fusion protein, wherein the second fusion protein comprises a second fragment of the protein of interest fused to a fourth interactive protein domain;
wherein the first ligand of the first haplomer-ligand complex is linked to the 5′ terminus of the first polynucleotide of the first haplomer-ligand complex;
wherein the second ligand of the second haplomer-ligand complex is linked to the 3′ terminus of the second polynucleotide of the second haplomer-ligand complex;
wherein the first polynucleotide of the first haplomer-ligand complex is substantially complementary to the nucleic acid template;
wherein the second polynucleotide of the second haplomer-ligand complex is substantially complementary to the nucleic acid template at a site in spatial proximity to the first polynucleotide of the first haplomer-ligand complex;
wherein the first interactive protein domain of the first haplomer-ligand complex and the third interactive protein domain of the first fusion protein can interact; and
wherein the second interactive protein domain of the second haplomer-ligand complex and the fourth interactive protein domain of the second fusion protein can interact; and
thereby resulting in the folding or dimerization of the first fragment of the protein of interest of the first fusion protein with the second fragment of the protein of interest of the second fusion protein.

17. The method according to claim 16, wherein:
the first interactive protein domain and the third interactive protein domain are jun/fos, mad/max, myc/max, or NZ/CZ interacting zipper motifs; and
the second interactive protein domain and the fourth interactive protein domain are jun/fos, mad/max, myc/max, or NZ/CZ interacting zipper motifs.

18. The method according to claim 13, wherein the LD-TAPER reaction comprises:
contacting the nucleic acid template with a complex formed by the interaction of a first haplomer-ligand complex with a first fusion protein, wherein:
the first haplomer-ligand complex comprises a first haplomer, wherein the first haplomer comprises a first polynucleotide, and a first small molecule ligand linked to the 5′ or 3′ terminus of the first polynucleotide, wherein the first small molecule ligand comprises a first ligand partner binding site;
the first fusion protein comprises a first fragment of a protein of interest fused to a first ligand binding domain for the first small molecule ligand; and
the first small molecule ligand of the first haplomer-ligand complex interacts with the first ligand binding domain for the first small molecule ligand of the first fusion protein; and
contacting the nucleic acid template with a complex formed by the interaction of a second haplomer-ligand complex with a second fusion protein, wherein:
the second haplomer-ligand complex comprises a second haplomer, wherein the second haplomer comprises a second polynucleotide, and a second small molecule ligand linked to the 5′ or 3′ terminus of the second polynucleotide, wherein the second small molecule ligand comprises a second ligand partner binding site;
the second fusion protein comprises a second fragment of the protein of interest fused to a second ligand binding domain for the second small molecule ligand;
the second small molecule ligand of the second haplomer-ligand complex interacts with the second ligand binding domain for the second small molecule ligand of the second fusion protein;
thereby resulting in the folding or dimerization of the fragment of the protein of interest of the first fusion protein with the fragment of the protein of interest of the second fusion protein.

19. The method according to claim 18, wherein:
the first small molecule ligand is an FKBP binding compound, and the first ligand binding domain for the first small molecule ligand is an FKBP domain or a FRB domain; and
the second small molecule ligand is an FKBP binding compound, and the second ligand binding domain for the second small molecule ligand is an FKBP domain or a FRB domain.

20. The method according to claim 13, wherein the LD-TAPER reaction comprises:
contacting the nucleic acid template with a complex formed by the interaction of a first haplomer-ligand complex with a first fusion protein, wherein:
the first haplomer-ligand complex comprises a first haplomer, wherein the first haplomer comprises a first polynucleotide, and a first ligand linked to the 5′ or 3′ terminus of the first polynucleotide, wherein the first ligand is a first interactive protein domain and comprises a first ligand partner binding site;
the first fusion protein comprises a first fragment of a protein of interest fused to a third interactive protein domain;
the first interactive protein domain of the first haplomer-ligand complex and the third interactive protein domain of the first fusion protein can interact; and
contacting the nucleic acid template with a complex formed by the interaction of a second haplomer-ligand complex with a second fusion protein, wherein:
the second haplomer-ligand complex comprises a second haplomer, wherein the second haplomer comprises a second polynucleotide, and a second ligand linked to the 5′ or 3′ terminus of the second polynucleotide, wherein the second ligand is a second interactive protein domain and comprises a second ligand partner binding site;
the second fusion protein comprises a second fragment of a protein of interest fused to a fourth interactive protein domain;
the second interactive protein domain of the second haplomer-ligand complex and the fourth interactive protein domain of the second fusion protein can interact;
thereby resulting in the folding or dimerization of the first fragment of the protein of interest of the first fusion protein with the second fragment of the protein of interest of the second fusion protein.

21. The method according to claim 20, wherein:
the first interactive protein domain and the third interactive protein domain are jun/fos, mad/max, myc/max, or NZ/CZ interacting zipper motifs; and
the second interactive protein domain and the fourth interactive protein domain are jun/fos, mad/max, myc/max, or NZ/CZ interacting zipper motifs.

22. The method according to claim 13, wherein the LD-TAPER reaction comprises:
contacting the nucleic acid template with a bottle haplomer-ligand complex, wherein the bottle haplomer-ligand complex comprises:
a) a bottle haplomer, wherein the bottle haplomer comprises a first polynucleotide, wherein the first polynucleotide comprises:
i) a first stem portion comprising from about 10 to about 20 nucleotide bases;
ii) an anti-target loop portion comprising from about 16 to about 40 nucleotide bases and having a first end to which the first stem portion is linked, wherein the anti-target loop portion is substantially complementary to the nucleic acid template; and
iii) a second stem portion comprising from about 10 to about 20 nucleotide bases linked to a second end of the anti-target loop portion, wherein the first stem portion is substantially complementary to the second stem portion; and
b) a first small molecule ligand linked to the terminal end of either the first stem portion or the second stem portion, wherein the first small molecule ligand comprises a first small molecule ligand partner binding site;
wherein the Tₘ of the anti-target loop portion:nucleic acid template is greater than the Tₘ of the first stem portion: second stem portion;
contacting the nucleic acid template with a second haplomer-ligand complex, wherein the second haplomer-ligand complex comprises a second haplomer, wherein the second haplomer comprises a second polynucleotide, and a second small molecule ligand linked to the 5′ or 3′ terminus of the second polynucleotide, wherein the second small molecule ligand comprises a second small molecule ligand partner binding site; wherein the second haplomer-ligand complex comprises a nucleotide portion that is substantially complementary to the stem portion of the bottle haplomer-ligand complex that is linked to the first small molecule ligand of the bottle haplomer-ligand complex;
contacting the bottle haplomer-ligand complex with a first fusion protein, wherein the first fusion protein comprises a first fragment of a protein of interest fused to a first ligand binding domain for the first small molecule ligand, wherein the first small molecule ligand of the bottle haplomer-ligand complex and the first small molecule ligand binding domain of the first fusion protein can interact; and
contacting the second haplomer-ligand complex with a second fusion protein, wherein the second fusion protein comprises a second fragment of the protein of interest fused to a second small molecule ligand binding domain for the second small molecule ligand, wherein the second small molecule ligand of the second haplomer-ligand complex and the second small molecule ligand binding domain of the second fusion protein can interact;
thereby resulting in the folding or dimerization of the first fragment of the protein of interest of the first fusion protein with the second fragment of the protein of interest of the second fusion protein.

23. The method according to claim 22, wherein:
the first small molecule ligand is an FKBP binding compound, and the first ligand binding domain for the first small molecule ligand is an FKBP domain or a FRB domain; and
the second small molecule ligand is an FKBP binding compound, and the second ligand binding domain for the second small molecule ligand is an FKBP domain or a FRB domain.

24. The method according to claim 13, wherein the LD-TAPER reaction comprises:
contacting the nucleic acid template with a bottle haplomer-ligand complex, wherein the bottle haplomer-ligand complex comprises:
a) a bottle haplomer, wherein the bottle haplomer comprises a first polynucleotide, wherein the first polynucleotide comprises:
i) a first stem portion comprising from about 10 to about 20 nucleotide bases;
ii) an anti-target loop portion comprising from about 16 to about 40 nucleotide bases and having a first end to which the first stem portion is linked, wherein the anti-target loop portion is substantially complementary to the nucleic acid template; and
iii) a second stem portion comprising from about 10 to about 20 nucleotide bases linked to a second end of the anti-target loop portion, wherein the first stem portion is substantially complementary to the second stem portion; and
b) a first ligand linked to the terminal end of either the first stem portion or the second stem portion, wherein the first ligand is a first interactive protein domain and comprises a first ligand partner binding site;
wherein the Tₘ of the anti-target loop portion:nucleic acid template is greater than the Tₘ of the first stem portion: second stem portion;
contacting the nucleic acid template with a second haplomer-ligand complex, wherein the second haplomer-ligand complex comprises a second haplomer, wherein the second haplomer comprises a second polynucleotide, and a second ligand linked to the 5′ or 3′ terminus of the second polynucleotide, wherein the second ligand is a second interactive protein domain and comprises a second ligand partner binding site; wherein the second haplomer-ligand complex comprises a nucleotide portion that is substantially complementary to the stem portion of the bottle haplomer-ligand complex that is linked to the first ligand of the bottle haplomer-ligand complex;
contacting the bottle haplomer-ligand complex with a first fusion protein, wherein the first fusion protein comprises a first fragment of a protein of interest fused to a third interactive protein domain; wherein the first interactive protein domain of the bottle haplomer-ligand complex and the third interactive protein domain of the first fusion protein can interact; and
contacting the second haplomer-ligand complex with a second fusion protein, wherein the second fusion protein comprises a second fragment of the protein of interest fused to a fourth interactive protein domain; wherein the second interactive protein domain of the second haplomer-ligand complex and the fourth interactive protein domain of the second fusion protein can interact;
thereby resulting in the folding or dimerization of the first fragment of the protein of interest of the first fusion protein with the second fragment of the protein of interest of the second fusion protein.

25. The method according to claim 24, wherein:
the first interactive protein domain and the third interactive protein domain are jun/fos, mad/max, myc/max, or NZ/CZ interacting zipper motifs; and
the second interactive protein domain and the fourth interactive protein domain are jun/fos, mad/max, myc/max, or NZ/CZ interacting zipper motifs.

26. The method according to any one of claims 14 to 25, wherein the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

27. The method according to any one of claims 14 to 26, wherein the first fragment of the protein of interest and the second fragment of the protein of interest are fragments of an active effector agent chosen from a peptide, protein, or a therapeutic agent.

28. The method according to claim 27, wherein the peptide or protein is a toxin, a pro-apoptotic agent, or a ligand for an antibody or antibody fragment.

29. The method according to any one of claims 1 to 5, further comprising performing a Split Protein TAPER (SP-TAPER) reaction using the nucleic acid template as a target nucleic acid sequence for the TAPER reaction.

30. The method according to claim 29, wherein the SP-TAPER reaction comprises:
contacting the cell with a first haplomer comprising a first polynucleotide linked to the C-terminus of an N-terminal protein fragment; and
contacting the cell with a second haplomer comprising a second polynucleotide linked to the N-terminus of a C-terminal protein fragment;
wherein:
the polynucleotide of one of the first or second haplomers is linked at its 5′ terminus to the protein fragment, and the other of the first and second haplomers is linked at its 3′ terminus to the protein fragment;
the N-terminal protein fragment and the C-terminal protein fragment are derived from a single active effector agent; and
wherein:
the first polynucleotide of the first haplomer is substantially complementary to the nucleic acid template, and the second polynucleotide of the second haplomer is substantially complementary to the nucleic acid template at a site in spatial proximity to the first polynucleotide of the first haplomer; or
the first polynucleotide of the first haplomer is substantially complementary to a portion of the nucleic acid template 5′ adjacent to a stem-loop structure, and the second polynucleotide of the second haplomer is substantially complementary to a portion of the nucleic acid template 3′ adjacent to the stem-loop structure; or
the first polynucleotide of the first haplomer is substantially complementary to a 5′ portion of a loop of a stem-loop structure of the nucleic acid template, and the second polynucleotide of the second haplomer is substantially complementary to a 3′ portion of the loop of the stem-loop structure of the nucleic acid template;
thereby resulting in the assembly of the protein from the N-terminal protein fragment and the C-terminal protein fragment.

31. The method according to claim 30, wherein:
the N-terminal fragment comprises the amino acid sequence of APIVTCRKLDGRE KPFKVDVATAQAQARKAGLTTGKSGDPHRYFAGDHIRWGVNNCDKADAILWEYPI YWVGKNAEWAKDVKTSQQKG (SEQ ID NO:34), and the C-terminal fragment comprises the amino acid sequence of GPTPIRVVYANSRGAVQYCGVMTHSKVDKNNQ GKEFFEKCD (SEQ ID NO:35);
the N-terminal fragment comprises the amino acid sequence of APIVTCRPKLDG (SEQ ID NO:36), and the C-terminal fragment comprises the amino acid sequence of REKPF KVDVATAQAQARKAGLTTGKSGDPHRYFAGDHIRWGVNNCDKADAILWEYPIYW VGKNAEWAKDVKTSQQKGGPTPIRVVYANSRGAVQYCGVMTHSKVDKNNQGKEF FEKCD (SEQ ID NO:37);
the N-terminal fragment comprises the amino acid sequence of APIVTCRPKLDGR EKPFKVDVATAQAQARKAGLTTGK (SEQ ID NO:38), and the C-terminal fragment comprises the amino acid sequence of SGDPHRYFAGDHIRWGVNNCDKADAILWEYPI YWVGKNAEWAKDVKTSQQKGGPTPIRVVYANSRGAVQYCGVMTHSKVDKNNQG KEFFEKCD (SEQ ID NO:39);
the N-terminal fragment comprises the amino acid sequence of APIVTCRPKLDGR EKPFKVDVATAQAQARKAGLTTGKSGDPHRYFAGDHIRWGVNNCDKAD (SEQ ID NO:40), and the C-terminal fragment comprises the amino acid sequence of AILWEYPIYW VGKNAEWAKDVKTSQQKGGPTPIRVVYANSRGAVQYCGVMTHSKVDKNNQGKEF FEKCD (SEQ ID NO:41);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVG (SEQ ID NO:42), and the C-terminal fragment comprises the amino acid sequence of KNAEWAKDVKTSQQKGGPTPIRVVYANSRGAVQYCGVMTHSKVDKNNQGKEFF EKCD (SEQ ID NO:43);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVGKNAEWAKD (SEQ ID NO:44), and the C-terminal fragment comprises the amino acid sequence of VKTSQQKGGPTPIRVVYANSRGAVQYCGVMTHSKVDKNNQGKEFF EKCD (SEQ ID NO:45);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVGKNAEWAKDVKTSQ (SEQ ID NO:46), and the C-terminal fragment comprises the amino acid sequence of QKGGPTPIRVVYANSRGAVQYCGVMTHSKVDKNNQGKEFFE KCD (SEQ ID NO:47);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVGKNAEWAKDVKTSQQKGGPTPIRVVYANSRG (SEQ ID NO:48), and the C-terminal fragment comprises the amino acid sequence of AVQYCGVMTHSKVDKNNQGK EFFEKCD (SEQ ID NO:49);
the N-terminal fragment comprises the amino acid sequence of APIVTCRPKLDG REKPFKVDVATAQAQARKAGLTTGKSGDPHRYFAGDHIRWGVNNCDKADAILWEY PIYWVGKNAEWAKDVKTSQQKGGPTPIRVVYANSRGAVQYCGVMTHSKVDKN (SEQ ID NO:50), and the C-terminal fragment comprises the amino acid sequence of NQGK EFFEKCD (SEQ ID NO:51); or
the N-terminal fragment comprises the amino acid sequence of APIVTCRPKLDGR EKPFKVDVATAQAQARKAGLT; (SEQ ID NO:52), and the C-terminal fragment comprises the amino acid sequence of TGKSGDPHRYFAGDHIRWGVNNCDKADAILWE YPIYWVGKNAEWAKDVKTSQQKGGPTPIRVVYANSRGAVQYCGVMTHSKVDKNN QGKEFFEKCD (SEQ ID NO:53).

32. The method according to claim 29, wherein the SP-TAPER reaction comprises: comprising:
a) contacting the nucleic acid template with a bottle: haplomer, wherein the bottle haplomer comprises a first polynucleotide comprising:
i) a first 3′ stem portion comprising from about 10 to about 20 nucleotide bases;
ii) an anti-target loop portion comprising from about 16 to about 40 nucleotide bases linked to the first 3′ stem portion, wherein the anti-target loop portion is substantially complementary to the nucleic acid template; and
iii) a second 5′ stem portion comprising from about 10 to about 20 nucleotide bases linked to the anti-target loop portion, wherein the first 3′ stem portion is substantially complementary to the second 5′ stem portion;
wherein the 5′ terminus of the first polynucleotide comprises an —SH moiety and is linked to the C-terminus of an N-terminal protein fragment, wherein the C-terminus comprises a cysteine or a selenocysteine; and
b) contacting the bottle haplomer with a second haplomer comprising a second polynucleotide linked to the N-terminus of a C-terminal protein fragment, wherein the second polynucleotide of the second haplomer is substantially complementary to the second 5′ stem portion of the first polynucleotide of the bottle haplomer;
wherein:
the N-terminal protein fragment and the C-terminal protein fragment are derived from a single active effector agent;
the Tₘ of the anti-target loop portion:nucleic acid template is greater than the Tₘ of the first stem portion:second stem portion; and
the Tₘ of the duplex formed by the second haplomer and the second stem portion of the bottle haplomer subtracted from the Tₘ of the first stem portion:second stem portion is from about 0° C. to about 20° C.;
thereby resulting in the assembly of the protein from the N-terminal protein fragment and the C-terminal protein fragment.

33. The method according to claim 32, wherein:
the Tₘ of the first stem portion:second stem portion subtracted from the Tₘ of the anti-target loop portion:nucleic acid template is from about 10° C. to about 40° C.;
the Tₘ of the first stem portion:second stem portion is from about 40° C. to about 50° C.;
the Tₘ of the anti-target loop portion:nucleic acid template is from about 60° C. to about 80° C.; and/or
the Tₘ of the first stem portion:second stem portion subtracted from theTₘ of the anti-target loop portion:nucleic acid template is from about 10° C. to about 20° C.

34. The method according to claim 32 or claim 33, wherein:
the first stem portion comprises from about 12 to about 18 nucleotide bases;
the anti-target loop portion comprises from about 18 to about 35 nucleotide bases; and/or
the second stem portion comprises from about 12 to about 18 nucleotide bases.

35. The method according to any one of claims 30 to 34, wherein the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

36. The method according to any one of claims 30 to 35, wherein the N-terminal protein fragment and the C-terminal protein fragment are fragments of an active effector agent chosen from a peptide, protein, or a therapeutic agent.

37. The method according to claim 36, wherein the peptide or protein is a toxin, a pro-apoptotic agent, or a ligand for an antibody or antibody fragment.

38. The method according to any one of claims 1 to 5, further comprising performing a Locked TAPER reaction using the nucleic acid template as a target nucleic acid sequence for the TAPER reaction.

39. The method according to claim 38, wherein the Locked TAPER reaction comprises:
contacting the nucleic acid template with a first haplomer, wherein the first haplomer comprises:
a) a first polynucleotide comprising:
i) a first stem portion comprising from about 10 to about 20 nucleotide bases;
ii) an anti-target loop portion comprising from about 16 to about 40 nucleotide bases and having a first end to which the first stem portion is linked, wherein the anti-target loop portion is substantially complementary to the nucleic acid template; and
iii) a second stem portion comprising from about 10 to about 20 nucleotide bases linked to a second end of the anti-target loop portion, wherein the first stem portion is substantially complementary to the second stem portion; and
b) a first effector partial moiety linked to either the first stem portion or the second stem portion;
wherein the Tₘ of the anti-target loop portion:nucleic acid template is greater than the Tₘ of the first stem portion: second stem portion; and
contacting the first haplomer with a second haplomer, wherein the second haplomer comprises:
a) a second polynucleotide comprising a nucleotide portion that is substantially complementary to the stem portion of the first polynucleotide that is linked to the first effector partial moiety; and
b) a second effector partial moiety linked to the second polynucleotide, wherein the second effector partial moiety can chemically interact with the first effector partial moiety of the first haplomer;
wherein the Tₘ of the second polynucleotide:first or second stem portion linked to the first effector partial moiety is less than or equal to the Tₘ of the first stem portion:second stem portion;
wherein the first effector partial moiety and the second effector partial moiety form an active effector agent when in sufficient proximity.

40. The method according to claim 39, wherein
the Tₘ of the first stem portion:second stem portion subtracted from the Tₘ of the anti-target loop portion:nucleic acid template is from about 10° C. to about 40° C.; and/or
the Tₘ of the first stem portion:second stem portion is from about 40° C. to about 50° C.; and/or
the Tₘ of the anti-target loop portion:nucleic acid template is from about 60° C. to about 80° C.; and/or
the Tₘ of the first stem portion:second stem portion subtracted from the Tₘ of the anti-target loop portion:nucleic acid template is from about 10° C. to about 20° C.

41. The method according to claim 39 or claim 40, wherein:
the first stem portion comprises from about 12 to about 18 nucleotide bases; and/or
the anti-target loop portion comprises from about 18 to about 35 nucleotide bases; and/or
the second stem portion comprises from about 12 to about 18 nucleotide bases.

42. The method according to any one of claims 39 to 41, wherein the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

43. The method according to any one of claims 39 to 42, wherein the first effector partial moiety and the second effector partial moiety are fragments of an active effector agent chosen from a peptide, protein, or a therapeutic agent.

44. The method according to claim 43, wherein the peptide or protein is a toxin, a pro-apoptotic agent, or a ligand for an antibody or antibody fragment.

45. The method according to any one of claims 39 to 44, wherein the first effector partial moiety and the second effector partial moiety each farther comprise a selectively-reactive moiety.

46. The method according to claim 45, wherein the first selectively-reactive moiety and the second selectively-reactive moiety are a chemically reactable pair of selectively-reactive moieties chosen from an azide, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, and a quadricyclane.

47. The method according to claim 46, wherein the first selectively-reactive moiety and the second selectively-reactive moiety are an azide-alkyne pair, an azide-phosphine pair, or a tetrazine-norbornene/trans-cyclooctene pair.

48. The method according to any one of claims 39 to 47, wherein the anti-target loop portion further comprises an internal hinge region, wherein the hinge region comprises one or more nucleotides that are not complementary to the nucleic acid template.

49. The method according to claim 48, wherein the hinge region comprises from about 1 nucleotide to about 6 nucleotides.

50. The method according to any one of claims 39 to 49, wherein:
the first polynucleotide comprises the nucleotide sequence 5′-ACTCGAGACGT CTCCTTGTCTTTGCTTTTCTTCAGGACACAGTGGCGAGACGTCTCGAGT-3′ (SEQ ID NO:7), and the second polynucleotide comprises the nucleotide sequence 5′-AGCTCTC GAGT-3′ (SEQ ID NO:9); or
the first polynucleotide comprises the nucleotide sequence 5′-ACTCGAGACGTC TCCTTCCTGCCCCTCCTCCTGCTCCGAGACGTCTCGAGT-3′ (SEQ ID NO: 8), and the second polynucleotide comprises the nucleotide sequence 5′-GACGTCTCGAGT-3′ (SEQ ID NO:10).

51. A method of labeling a cell surface with a quantifiable reverse template, the method comprising the steps:
a) contacting the cell with an azide-modified sugar;
b) contacting the cell with two ligation-template oligonucleotides, wherein:
i) the first ligation-template oligonucleotide (LT1) comprises a 5′-azide reactive molecule that is chemically reactable with an azide group; and
ii) the second ligation-template oligonucleotide (LT2) comprises a 5′-phosphate, and a 3′-azide reactive molecule that is chemically reactable with an azide group;
c) contacting the cell with a nuclease resistant oligonucleotide, wherein the nuclease resistant oligonucleotide is non-overlap complementary to both LT1 and LT2; wherein upon close proximity, the 3′—OH of LT1 and the 5′-phosphate of LT2 are ligatable; and
d) contacting the cell with a ligase, thereby generating a reverse template formed from LT1 and LT2 that can be amplified and quantified.

52. The method according to claim 51, wherein the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA).

53. The method according to claim 51 or claim 52, wherein the azide-modified sugar is acetylated at 1, 2, 3, or 4 positions.

54. The method according to any one of claims 51 to 53, wherein one or both of the LT1 and/or LT2 comprise a phosphorothioate backbone, a phosphoramidate backbone, a morpholino backbone, a bridged nucleic acid backbone, or a locked nucleic acid (LNA) backbone.

55. The method according to any one of claims 51 to 54, wherein the cell is contacted with an equimolar mixture of the two ligation-template oligonucleotides.

56. The method according to any one of claims 51 to 55, wherein the nuclease resistant oligonucleotide comprises a plurality of 2′ modifications chosen from —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are, independently, from 0 to about 10.

57. The method according to claim 56, wherein the nuclease resistant oligonucleotide comprises a plurality of 2′-O-methyl groups.

58. The method according to any one of claims 51 to 57, wherein the azide reactive molecules are, independently, dibenzocyclooctyne (DBCO), bicyclo[6.1.0]nonyne (BCN), methyltetrazine, or trans-cyclooctene (TCO).

59. The method according to any one of claims 51 to 58, wherein the ligase is Splint ligase.

60. The method according to any one of claims 51 to 59, further comprising amplifying and quantifying the reverse template.

61. A method of labeling a cell with a substrate for a templated assembly reaction, the method comprising the steps:
a) contacting the cell with an azide-modified sugar; and
b) contacting the cell with a nucleic acid template, wherein the nucleic acid template comprises:
i) a first hybridization region and a second hybridization region separated by a loop region, wherein the first hybridization region is complementary to the second hybridization region; and
ii) a azide reactive molecule at the 3′-end of the nucleic acid template and at the 5′-end of the nucleic acid template, wherein both azide reactive molecules are chemically reactable with an azide group.

62. The method according to claim 61, wherein the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA).

63. The method according to claim 61 or claim 62, wherein the azide-modified sugar is acetylated at 1, 2, 3, or 4 positions.

64. The method according to any one of claims 61 to 63, wherein the azide reactive molecule is dibenzocyclooctyne (DBCO), bicyclo[6.1.0]nonyne (BCN), methyltetrazine, or trans-cyclooctene (TCO).

65. The method according to any one of claims 61 to 64, wherein the nucleic acid template is chosen from a cancer-specific polynucleotide, a viral polynucleotide, a microbial-specific polynucleotide, a differentially expressed gene, and a disease-specific polynucleotide.

66. The method according to any one of claims 61 to 65, further comprising performing a Template Assembly by Proximity-Enhanced Reactivity (TAPER) reaction using the nucleic acid template as a target nucleic acid sequence for the TAPER reaction.

67. The method according to claim 66, wherein the TAPER reaction comprises contacting the cell with a first haplomer and a second haplomer, wherein:
the first haplomer comprises:
a first polynucleotide that is complementary to a first region of the nucleic acid template;
a first effector partial moiety, wherein the first effector partial moiety is linked to the first polynucleotide; and
a first selectively-reactive moiety, wherein the first selectively-reactive moiety is linked to the first effector partial moiety;
the second haplomer comprises:
a second polynucleotide that is complementary to a second region of the nucleic acid template;
a second effector partial moiety, wherein the second effector partial moiety is linked to the second polynucleotide; and
a second selectively-reactive moiety, wherein the second selectively-reactive moiety is linked to the second effector partial moiety;
wherein:
the first selectively-reactive moiety and the second selectively-reactive moiety chemically react with each other when in sufficient proximity;
the first region of the nucleic acid template is in sufficient proximity to the second region of the nucleic acid template to allow the first selectively-reactive moiety and the second selectively-reactive moiety to chemically react with each other; and
the first effector partial moiety and the second effector partial moiety form an active effector agent when in sufficient proximity.

68. The method according to claim 67, wherein the first selectively-reactive moiety and the second selectively-reactive moiety are a chemically reactable pair of selectively-reactive moieties chosen from an azide, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, and a quadricyclane.

69. The method according to claim 68, wherein the first selectively-reactive moiety and the second selectively-reactive moiety are an azide-alkyne pair, an azide-phosphine pair, or a tetrazine-norbornene/trans-cyclooctene pair.

70. The method according to any one of claims 67 to 69, wherein the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

71. The method according to any one of claims 67 to 70, wherein the first effector partial moiety and the second effector partial moiety are fragments of an active effector agent chosen from a peptide, protein, or a therapeutic agent.

72. The method according to claim 71, wherein the peptide or protein is a toxin, a pro-apoptotic agent, or a ligand for an antibody or antibody fragment.

73. The method according to any one of claims 61 to 65, further comprising performing a Ligand Directed TAPER (LD-TAPER) reaction using the nucleic acid template as a target nucleic acid sequence for the LD-TAPER reaction.

74. The method according to claim 73, wherein the LD-TAPER reaction comprises:
contacting the nucleic acid template with a first haplomer-ligand complex, wherein the first haplomer-ligand complex comprises a first haplomer, wherein the first haplomer comprises a first polynucleotide, and a first small molecule ligand linked to the 5′ or 3′ terminus of the first haplomer, wherein the first small molecule ligand comprises a first small molecule ligand partner binding site;
contacting the nucleic acid template with a second haplomer-ligand complex, wherein the second haplomer-ligand complex comprises a second haplomer, wherein the second haplomer comprises a second polynucleotide, and a second small molecule ligand linked to the 5′ or 3′ terminus of the second haplomer, wherein the second small molecule ligand comprises a second small molecule ligand partner binding site;
contacting the first haplomer-ligand complex with a first fusion protein, wherein the first fusion protein comprises a first fragment of a protein of interest fused to a first ligand binding domain for a small molecule ligand;
contacting the second haplomer-ligand complex with a second fusion protein, wherein the second fusion protein comprises a second fragment of the protein of interest fused to a second ligand binding domain for a small molecule ligand;
wherein the first ligand of the first haplomer-ligand complex is linked to the 5′ terminus of the first polynucleotide of the first haplomer-ligand complex;
wherein the second ligand of the second haplomer-ligand complex is linked to the 3′ terminus of the second polynucleotide of the second haplomer-ligand complex;
wherein the first polynucleotide of the first haplomer-ligand complex is substantially complementary to the nucleic acid template;
wherein the second polynucleotide of the second haplomer-ligand complex is substantially complementary to the nucleic acid template at a site in spatial proximity to the first polynucleotide of the first haplomer-ligand complex;
wherein the first small molecule ligand of the first haplomer-ligand complex and the first ligand binding domain of the first fusion protein can interact; and
wherein the second small molecule ligand of the second haplomer-ligand complex and the second ligand binding domain of the second fusion protein can interact;
thereby resulting in the folding or dimerization of the first fragment of the protein of interest of the first fusion protein with the second fragment of the protein of interest of the second fusion protein.

75. The method according to claim 74, wherein:
the first small molecule ligand is an FKBP binding compound, and the first ligand binding domain for the first small molecule ligand is an FKBP domain or a FRB domain; and
the second small molecule ligand is an FKBP binding compound, and the second ligand binding domain for the second small molecule ligand is an FKBP domain or a FRB domain.

76. The method according to claim 73, wherein the LD-TAPER reaction comprises:
contacting the nucleic acid template with a first haplomer-ligand complex, wherein the first haplomer-ligand complex comprises a first haplomer, wherein the first haplomer comprises a first polynucleotide, and a first ligand linked to the 5′ or 3′ terminus of the first haplomer, wherein the first ligand is a first interactive protein domain and comprises a first ligand partner binding site;
contacting the nucleic acid template with a second haplomer-ligand complex, wherein the second haplomer-ligand complex comprises a second haplomer, wherein the second haplomer comprises a second polynucleotide, and a second ligand linked to the 5′ or 3′ terminus of the second haplomer, wherein the second ligand is a second interactive protein domain and comprises a second ligand partner binding site;
contacting the first haplomer-ligand complex with a first fusion protein, wherein the first fusion protein comprises a first fragment of a protein of interest fused to a third interactive protein domain;
contacting the second haplomer-ligand complex with a second fusion protein, wherein the second fusion protein comprises a second fragment of the protein of interest fused to a fourth interactive protein domain;
wherein the first ligand of the first haplomer-ligand complex is linked to the 5′ terminus of the first polynucleotide of the first haplomer-ligand complex;
wherein the second ligand of the second haplomer-ligand complex is linked to the 3′ terminus of the second polynucleotide of the second haplomer-ligand complex;
wherein the first polynucleotide of the first haplomer-ligand complex is substantially complementary to the nucleic acid template;
wherein the second polynucleotide of the second haplomer-ligand complex is substantially complementary to the nucleic acid template at a site in spatial proximity to the first polynucleotide of the first haplomer-ligand complex;
wherein the first interactive protein domain of the first haplomer-ligand complex and the third interactive protein domain of the first fusion protein can interact; and
wherein the second interactive protein domain of the second haplomer-ligand complex and the fourth interactive protein domain of the second fusion protein can interact; and
thereby resulting in the folding or dimerization of the first fragment of the protein of interest of the first fusion protein with the second fragment of the protein of interest of the second fusion protein.

77. The method according to claim 76, wherein:
the first interactive protein domain and the third interactive protein domain are jun/fos, mad/max, myc/max, or NZ/CZ interacting zipper motifs; and
the second interactive protein domain and the fourth interactive protein domain are jun/fos, mad/max, myc/max, or NZ/CZ interacting zipper motifs.

78. The method according to claim 73, wherein the LD-TAPER reaction comprises:
contacting the nucleic acid template with a complex formed by the interaction of a first haplomer-ligand complex with a first fusion protein, wherein:
the first haplomer-ligand complex comprises a first haplomer, wherein the first haplomer comprises a first polynucleotide, and a first small molecule ligand linked to the 5′ or 3′ terminus of the first polynucleotide, wherein the first small molecule ligand comprises a first ligand partner binding site;
the first fusion protein comprises a first fragment of a protein of interest fused to a first ligand binding domain for the first small molecule ligand; and
the first small molecule ligand of the first haplomer-ligand complex interacts with the first ligand binding domain for the first small molecule ligand of the first fusion protein; and
contacting the nucleic acid template with a complex formed by the interaction of a second haplomer-ligand complex with a second fusion protein, wherein:
the second haplomer-ligand complex comprises a second haplomer, wherein the second haplomer comprises a second polynucleotide, and a second small molecule ligand linked to the 5′ or 3′ terminus of the second polynucleotide, wherein the second small molecule ligand comprises a second ligand partner binding site;
the second fusion protein comprises a second fragment of the protein of interest fused to a second ligand binding domain for the second small molecule ligand;
the second small molecule ligand of the second haplomer-ligand complex interacts with the second ligand binding domain for the second small molecule ligand of the second fusion protein;
thereby resulting in the folding or dimerization of the fragment of the protein of interest of the first fusion protein with the fragment of the protein of interest of the second fusion protein.

79. The method according to claim 78, wherein:
the first small molecule ligand is an FKBP binding compound, and the first ligand binding domain for the first small molecule ligand is an FKBP domain or a FRB domain; and
the second small molecule ligand is an FKBP binding compound, and the second ligand binding domain for the second small molecule ligand is an FKBP domain or a FRB domain.

80. The method according to claim 73, wherein the LD-TAPER reaction comprises:
contacting the nucleic acid template with a complex formed by the interaction of a first haplomer-ligand complex with a first fusion protein, wherein:
the first haplomer-ligand complex comprises a first haplomer, wherein the first haplomer comprises a first polynucleotide, and a first ligand linked to the 5′ or 3′ terminus of the first polynucleotide, wherein the first ligand is a first interactive protein domain and comprises a first ligand partner binding site;
the first fusion protein comprises a first fragment of a protein of interest fused to a third interactive protein domain;
the first interactive protein domain of the first haplomer-ligand complex and the third interactive protein domain of the first fusion protein can interact; and
contacting the nucleic acid template with a complex formed by the interaction of a second haplomer-ligand complex with a second fusion protein, wherein:
the second haplomer-ligand complex comprises a second haplomer, wherein the second haplomer comprises a second polynucleotide, and a second ligand linked to the 5′ or 3′ terminus of the second polynucleotide, wherein the second ligand is a second interactive protein domain and comprises a second ligand partner binding site;
the second fusion protein comprises a second fragment of a protein of interest fused to a fourth interactive protein domain;
the second interactive protein domain of the second haplomer-ligand complex and the fourth interactive protein domain of the second fusion protein can interact;
thereby resulting in the folding or dimerization of the first fragment of the protein of interest of the first fusion protein with the second fragment of the protein of interest of the second fusion protein.

81. The method according to claim 80, wherein:
the first interactive protein domain and the third interactive protein domain are jun/fos, mad/max, myc/max, or NZ/CZ interacting zipper motifs; and
the second interactive protein domain and the fourth interactive protein domain are jun/fos, mad/max, myc/max, or NZ/CZ interacting zipper motifs.

82. The method according to claim 73, wherein the LD-TAPER reaction comprises:
contacting the nucleic acid template with a bottle haplomer-ligand complex, wherein the bottle haplomer-ligand complex comprises:
a) a bottle haplomer, wherein the bottle haplomer comprises a first polynucleotide, wherein the first polynucleotide comprises:
i) a first stem portion comprising from about 10 to about 20 nucleotide bases;
ii) an anti-target loop portion comprising from about 16 to about 40 nucleotide bases and having a first end to which the first stem portion is linked, wherein the anti-target loop portion is substantially complementary to the nucleic acid template; and
iii) a second stem portion comprising from about 10 to about 20 nucleotide bases linked to a second end of the anti-target loop portion, wherein the first stem portion is substantially complementary to the second stem portion; and
b) a first small molecule ligand linked to the terminal end of either the first stem portion or the second stem portion, wherein the first small molecule ligand comprises a first small molecule ligand partner binding site;
wherein the Tₘ of the anti-target loop portion:nucleic acid template is greater than the Tₘ of the first stem portion: second stem portion;
contacting the nucleic acid template with a second haplomer-ligand complex, wherein the second haplomer-ligand complex comprises a second haplomer, wherein the second haplomer comprises a second polynucleotide, and a second small molecule ligand linked to the 5′ or 3′ terminus of the second polynucleotide, wherein the second small molecule ligand comprises a second small molecule ligand partner binding site; wherein the second haplomer-ligand complex comprises a nucleotide portion that is substantially complementary to the stem portion of the bottle haplomer-ligand complex that is linked to the first small molecule ligand of the bottle haplomer-ligand complex;
contacting the bottle haplomer-ligand complex with a first fusion protein, wherein the first fusion protein comprises a first fragment of a protein of interest fused to a first ligand binding domain for the first small molecule ligand, wherein the first small molecule ligand of the bottle haplomer-ligand complex and the first small molecule ligand binding domain of the first fusion protein can interact; and
contacting the second haplomer-ligand complex with a second fusion protein, wherein the second fusion protein comprises a second fragment of the protein of interest fused to a second small molecule ligand binding domain for the second small molecule ligand, wherein the second small molecule ligand of the second haplomer-ligand complex and the second small molecule ligand binding domain of the second fusion protein can interact;
thereby resulting in the folding or dimerization of the first fragment of the protein of interest of the first fusion protein with the second fragment of the protein of interest of the second fusion protein.

83. The method according to claim 82, wherein:
the first small molecule ligand is an FKBP binding compound, and the first ligand binding domain for the first small molecule ligand is an FKBP domain or a FRB domain; and
the second small molecule ligand is an FKBP binding compound, and the second ligand binding domain for the second small molecule ligand is an FKBP domain or a FRB domain.

84. The method according to claim 73, wherein the LD-TAPER reaction comprises:
contacting the nucleic acid template with a bottle haplomer-ligand complex, wherein the bottle haplomer-ligand complex comprises:
a) a bottle haplomer, wherein the bottle haplomer comprises a first polynucleotide, wherein the first polynucleotide comprises:
i) a first stem portion comprising from about 10 to about 20 nucleotide bases;
ii) an anti-target loop portion comprising from about 16 to about 40 nucleotide bases and having a first end to which the first stem portion is linked, wherein the anti-target loop portion is substantially complementary to the nucleic acid template; and
iii) a second stem portion comprising from about 10 to about 20 nucleotide bases linked to a second end of the anti-target loop portion, wherein the first stem portion is substantially complementary to the second stem portion; and
b) a first ligand linked to the terminal end of either the first stem portion or the second stem portion, wherein the first ligand is a first interactive protein domain and comprises a first ligand partner binding site;
wherein the Tₘ of the anti-target loop portion:nucleic acid template is greater than the Tₘ of the first stem portion: second stem portion;
contacting the nucleic acid template with a second haplomer-ligand complex, wherein the second haplomer-ligand complex comprises a second haplomer, wherein the second haplomer comprises a second polynucleotide, and a second ligand linked to the 5′ or 3′ terminus of the second polynucleotide, wherein the second ligand is a second interactive protein domain and comprises a second ligand partner binding site; wherein the second haplomer-ligand complex comprises a nucleotide portion that is substantially complementary to the stem portion of the bottle haplomer-ligand complex that is linked to the first ligand of the bottle haplomer-ligand complex;
contacting the bottle haplomer-ligand complex with a first fusion protein, wherein the first fusion protein comprises a first fragment of a protein of interest fused to a third interactive protein domain; wherein the first interactive protein domain of the bottle haplomer-ligand complex and the third interactive protein domain of the first fusion protein can interact; and
contacting the second haplomer-ligand complex with a second fusion protein, wherein the second fusion protein comprises a second fragment of the protein of interest fused to a fourth interactive protein domain; wherein the second interactive protein domain of the second haplomer-ligand complex and the fourth interactive protein domain of the second fusion protein can interact;
thereby resulting in the folding or dimerization of the first fragment of the protein of interest of the first fusion protein with the second fragment of the protein of interest of the second fusion protein.

85. The method according to claim 84, wherein:
the first interactive protein domain and the third interactive protein domain are jun/fos, mad/max, myc/max, or NZ/CZ interacting zipper motifs; and
the second interactive protein domain and the fourth interactive protein domain are jun/fos, mad/max, myc/max, or NZ/CZ interacting zipper motifs.

86. The method according to any one of claims 74 to 85, wherein the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

87. The method according to any one of claims 74 to 86, wherein the first fragment of the protein of interest and the second fragment of the protein of interest are fragments of an active effector agent chosen from a peptide, protein, or a therapeutic agent.

88. The method according to claim 87, wherein the peptide or protein is a toxin, a pro-apoptotic agent, or a ligand for an antibody or antibody fragment.

89. The method according to any one of claims 61 to 65, further comprising performing a Split Protein TAPER (SP-TAPER) reaction using the nucleic acid template as a target nucleic acid sequence for the TAPER reaction.

90. The method according to claim 89, wherein the SP-TAPER reaction comprises:
contacting the cell with a first haplomer comprising a first polynucleotide linked to the C-terminus of an N-terminal protein fragment; and
contacting the cell with a second haplomer comprising a second polynucleotide linked to the N-terminus of a C-terminal protein fragment;
wherein:
the polynucleotide of one of the first or second haplomers is linked at its 5′ terminus to the protein fragment, and the other of the first and second haplomers is linked at its 3′ terminus to the protein fragment;
the N-terminal protein fragment and the C-terminal protein fragment are derived from a single active effector agent; and
wherein:
the first polynucleotide of the first haplomer is substantially complemental), to the nucleic acid template, and the second polynucleotide of the second haplomer is substantially complementary to the nucleic acid template at a site in spatial proximity to the first polynucleotide of the first haplomer; or
the first polynucleotide of the first haplomer is substantially complementary to a portion of the nucleic acid template 5′ adjacent to a stem-loop structure, and the second polynucleotide of the second haplomer is substantially complementary to a portion of the nucleic acid template 3′ adjacent to the stem-loop structure; or
the first polynucleotide of the first haplomer is substantially complementary to a 5′ portion of a loop of a stem-loop structure of the nucleic acid template, and the second polynucleotide of the second haplomer is substantially complementary to a 3′ portion of the loop of the stem-loop structure of the nucleic acid template;
thereby resulting in the assembly of the protein from the N-terminal protein fragment and the C-terminal protein fragment.

91. The method according to claim 90, wherein:
the N-terminal fragment comprises the amino acid sequence of APIVTCRKLDGRE KPFKVDVATAQAQARKAGLTTGKSGDPHRYFAGDHIRWGVNNCDKADAILWEYPI YWVGKNAEWAKDVKTSQQKG (SEQ ID NO:34), and the C-terminal fragment comprises the amino acid sequence of GPTPIRVVYANSRGAVQYCGVMTHSKVDKNNQ GKEFFEKCD (SEQ ID NO:35);
the N-terminal fragment comprises the amino acid sequence of APIVTCRPKLDG (SEQ ID NO:36), and the C-terminal fragment comprises the amino acid sequence of REKP FKVDVATAQAQARKAGLTTGKSGDPHRYFAGDHIRWGVNNCDKADAILWEYPIYW VGKNAEWAKDVKTSQQKGGPTPIRVVYANSRGAVQYCGVMTHSKVDKNNQGKEF FEKCD (SEQ ID NO:37);
the N-terminal fragment comprises the amino acid sequence of APIVTCRPKLDGR EKPFKVDVATAQAQARKAGLTTGK (SEQ ID NO:38), and the C-terminal fragment comprises the amino acid sequence of SGDPHRYFAGDHIRWGVNNCDKADAILWEYPIY WVGKNAEWAKDVKTSQQKGGPTPIRVVYANSRGAVQYCGVMTHSKVDKNNQGK EFFEKCD (SEQ ID NO:39);
the N-terminal fragment comprises the amino acid sequence of APIVTCRPKLDG REKPFKVDVATAQAQARKAGLTTGKSGDPHRYFAGDHIRWGVNNCDKAD (SEQ ID NO:40), and the C-terminal fragment comprises the amino acid sequence of AILWEYPIYW VGKNAEWAKDVKTSQQKGGPTPIRVVYANSRGAVQYCGVMTHSKVDKNNQGKEF FEKCD (SEQ ID NO:41);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVG (SEQ ID NO:42), and the C-terminal fragment comprises the amino acid sequence of KNAEWAKDVKTSQQKGGPTPIRVVYANSRGAVQYCGVMTHSKVDKNNQGKEFF EKCD (SEQ ID NO:43);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVGKNAEWAKD (SEQ ID NO:44), and the C-terminal fragment comprises the amino acid sequence of VKTSQQKGGPTPIRVVYANSRGAVQYCGVMTHSKVDKNNQGKEFF EKCD (SEQ ID NO:45);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVGKNAEWAKDVKTSQ (SEQ ID NO:46), and the C-terminal fragment comprises the amino acid sequence of QKGGPTPIRVVYANSRGAVQYCGVMTHSKVDKNNQGKEFFE KCD (SEQ ID NO:47);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVGKNAEWAKDVKTSQQKGGPTPIRVVYANSRG (SEQ ID NO:48), and the C-terminal fragment comprises the amino acid sequence of AVQYCGVMTHSKVDKNNQGK EFFEKCD (SEQ ID NO:49);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVGKNAEWAKDVKTSQQKGGPTPIRVVYANSRGAVQYCGVMTHSKVDKN (SEQ ID NO:50), and the C-terminal fragment comprises the amino acid sequence of NQGK EFFEKCD (SEQ ID NO:51); or
the N-terminal fragment comprises the amino acid sequence of APIVTCRPKLDGR EKPFKVDVATAQAQARKAGLT; (SEQ ID NO:52), and the C-terminal fragment comprises the amino acid sequence of TGKSGDPHRYFAGDHIRWGVNNCDKADAILW EYPIYWVGKNAEWAKDVKTSQQKGGPTPIRVVYANSRGAVQYCGVMTHSKVDKN NQGKEFFEKCD (SEQ ID NO:53).

92. The method according to claim 89, wherein the SP-TAPER reaction comprises: comprising:
a) contacting the nucleic acid template with a bottle haplomer, wherein the bottle haplomer comprises a first polynucleotide comprising:
i) a first 3′ stem portion comprising from about 10 to about 20 nucleotide bases;
ii) an anti-target loop portion comprising from about 16 to about 40 nucleotide bases linked to the first 3′ stem portion, wherein the anti-target loop portion is substantially complementary to the nucleic acid template; and
iii) a second 5′ stem portion comprising from about 10 to about 20 nucleotide bases linked to the anti-target loop portion, wherein the first 3′ stem portion is substantially complementary to the second 5′ stem portion;
wherein the 5′ terminus of the first polynucleotide comprises an SH moiety and is linked to the C-terminus of an N-terminal protein fragment, wherein the C-terminus comprises a cysteine or a selenocysteine; and
b) contacting the bottle haplomer with a second haplomer comprising a second polynucleotide linked to the N-terminus of a C-terminal protein fragment, wherein the second polynucleotide of the second haplomer is substantially complementary to the second 5′ stem portion of the first polynucleotide of the bottle haplomer;
wherein:
the N-terminal protein fragment and the C-terminal protein fragment are derived from a single active effector agent;
the Tₘ of the anti-target loop portion:nucleic acid template is greater than the Tₘ of the first stem portion:second stem portion; and
the Tₘ of the duplex formed by the second haplomer and the second stem portion of the bottle haplomer subtracted from the Tₘ of the first stem portion:second stem portion is from about 0° C. to about 20° C.;
thereby resulting in the assembly of the protein from the N-terminal protein fragment and the C-terminal protein fragment.

93. The method according to claim 92, wherein:
the Tₘ of the first stem portion:second stem portion subtracted from the Tₘ of the anti-target loop portion:nucleic acid template is from about 10° C. to about 40° C.;
the Tₘ of the first stem portion:second stem portion is from about 40° C. to about 50° C.;
the Tₘ of the anti-target loop portion:nucleic acid template is from about 60° C. to about 80° C.; and/or
the Tₘ of the first stem portion:second stem portion subtracted from the Tₘ of the anti-target loop portion:nucleic acid template is from about 10° C. to about 20° C.

94. The method according to claim 92 or claim 93, wherein:
the first stem portion comprises from about 12 to about 18 nucleotide bases;
the anti-target loop portion comprises from about 18 to about 35 nucleotide bases; and/or
the second stem portion comprises from about 12 to about 18 nucleotide bases.

95. The method according to any one of claims 90 to 94, wherein the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

96. The method according to any one of claims 90 to 95, wherein the N-terminal protein fragment and the C-terminal protein fragment are fragments of an active effector agent chosen from a peptide, protein, or a therapeutic agent.

97. The method according to claim 96, wherein the peptide or protein is a toxin, a pro-apoptotic agent, or a ligand for an antibody or antibody fragment.

98. The method according to any one of claims 61 to 65, further comprising performing a Locked TAPER reaction using the nucleic acid template as a target nucleic acid sequence for the TAPER reaction.

99. The method according to claim 98, wherein the Locked TAPER reaction comprises:
contacting the nucleic acid template with a first haplomer, wherein the first haplomer comprises:
a) a first polynucleotide comprising:
i) a first stem portion comprising from about 10 to about 20 nucleotide bases;
ii) an anti-target loop portion comprising from about 16 to about 40 nucleotide bases and having a first end to which the first stem portion is linked, wherein the anti-target loop portion is substantially complementary to the nucleic acid template; and
iii) a second stem portion comprising from about 10 to about 20 nucleotide bases linked to a second end of the anti-target loop portion, wherein the first stem portion is substantially complementary to the second stem portion; and
b) a first effector partial moiety linked to either the first stem portion or the second stem portion;
wherein the Tₘ of the anti-target loop portion:nucleic acid template is greater than the Tₘ of the first stem portion: second stem portion; and
contacting the first haplomer with a second haplomer, wherein the second haplomer comprises:
a) a second polynucleotide comprising a nucleotide portion that is substantially complementary to the stem portion of the first polynucleotide that is linked to the first effector partial moiety; and
b) a second effector partial moiety linked to the second polynucleotide, wherein the second effector partial moiety can chemically interact with the first effector partial moiety of the first haplomer;
wherein the Tₘ of the second polynucleotide:first or second stem portion linked to the first effector partial moiety is less than or equal to the Tₘ of the first stem portion:second stem portion;
wherein the first effector partial moiety and the second effector partial moiety form an active effector agent when in sufficient proximity.

100. The method according to claim 99, wherein
the Tₘ of the first stem portion:second stem portion subtracted from the Tₘ of the anti-target loop portion:nucleic acid template is from about 10° C. to about 40° C.; and/or
the Tₘ of the first stem portion:second stem portion is from about 40° C. to about 50° C.; and/or
the Tₘ of the anti-target loop portion:nucleic acid template is from about 60° C. to about 80° C.; and/or
the Tₘ of the first stem portion:second stem portion subtracted from the Tₘ of the anti-target loop portion:nucleic acid template is from about 10° C. to about 20° C.

101. The method according to claim 99 or claim 100, wherein:
the first stem portion comprises from about 12 to about 18 nucleotide bases; and/or
the anti-target loop portion comprises from about 18 to about 35 nucleotide bases; and/or
the second stem portion comprises from about 12 to about 18 nucleotide bases.

102. The method according to any one of claims 99 to 101, wherein the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

103. The method according to any one of claims 99 to 102, wherein the first effector partial moiety and the second effector partial moiety are fragments of an active effector agent chosen from a peptide, protein, or a therapeutic agent.

104. The method according to claim 103, wherein the peptide or protein is a toxin, a pro-apoptotic agent, or a ligand for an antibody or antibody fragment.

105. The method according to any one of claims 99 to 104, wherein the first effector partial moiety and the second effector partial moiety each further comprise a selectively-reactive moiety.

106. The method according to claim 105, wherein the first selectively-reactive moiety and the second selectively-reactive moiety are a chemically reactable pair of selectively-reactive moieties chosen from an azide, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, and a quadricyclane.

107. The method according to claim 106, wherein the first selectively-reactive moiety and the second selectively-reactive moiety are an azide-alkyne pair, an azide-phosphine pair, or a tetrazine-norbornene/trans-cyclooctene pair.

108. The method according to any one of claims 99 to 107, wherein the anti-target loop portion further comprises an internal hinge region, wherein the hinge region comprises one or more nucleotides that are not complementary to the nucleic acid template.

109. The method according to claim 108, wherein the hinge region comprises from about 1 nucleotide to about 6 nucleotides.

110. The method according to any one of claims 99 to 109, wherein:
the first polynucleotide comprises the nucleotide sequence 5′-ACTCGAGACGTCT CCTTGTCTTTGCTTTTCTTCAGGACACAGTGGCGAGACGTCTCGAGT-3′ (SEQ ID NO:7), and the second polynucleotide comprises the nucleotide sequence 5′-AGCTCTCGA GT-3′ (SEQ ID NO:9); or
the first polynucleotide comprises the nucleotide sequence 5′-ACTCGAGACGTCT CCTTCCTGCCCCTCCTCCTGCTCCGAGACGTCTCGAGT-3′ (SEQ ID NO:8), and the second polynucleotide comprises the nucleotide sequence 5′-GACGTCTCGAGT-3′ (SEQ ID NO:10).

111. A method of metabolically labeling the surface of a specific target cell, the method comprising the steps:
a) contacting the cell with a nucleic acid molecule, wherein the nucleic acid molecule comprises:
i) a short terminal RNA segment comprising a terminal azide-modified sugar; and
ii) a longer modified RNA segment linked to the short terminal RNA segment, wherein the terminal end of the longer modified RNA segment is complementary to the short terminal RNA segment;
wherein the longer modified RNA segment is complementary to a specific transcript target within the specific targeted cell; and
wherein the longer modified RNA segment is modified to be nuclease resistant.

112. The method according to claim 111, wherein the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA).

113. The method according to claim 111 or 112, wherein the azide-modified sugar is acetylated at 1, 2, 3, or 4 positions.

114. The method according to any one of claims 111 to 113, wherein the longer modified RNA segment comprises a phosphorothioate backbone, a phosphoramidate backbone, a morpholino backbone, a bridged nucleic acid backbone, a locked nucleic acid (LNA) backbone or a 2′ modification.

115. The method according to claim 114, wherein the 2′ modification is chosen from —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are, independently, from 0 to about 10.

116. The method according to claim 115, wherein the 2′ modification is a 2′-O-methyl group.

117. The method according to any one of claims 111 to 116, further comprising contacting the cell with an oligonucleotide, wherein the oligonucleotide is complementary to the short terminal RNA segment, and wherein the oligonucleotide is modified to be nuclease resistant.

118. The method according to claim 117, wherein the oligonucleotide comprises a phosphorothioate backbone, a phosphoramidate backbone, a morpholino backbone, a bridged nucleic acid backbone, a locked nucleic acid (LNA) backbone or a 2′ modification.

119. The method according to claim 118, wherein the 2′ modification is chosen from —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are, independently, from 0 to about 10.

120. The method according to claim 119, wherein the 2′ modification is a 2′-O-methyl group.

121. A method of metabolically labeling the surface of a cell, the method comprising the steps:
a) contacting the cell with a nucleic acid molecule, wherein the nucleic acid molecule comprises:
i) a short terminal RNA segment comprising a terminal azide-modified sugar;
ii) a longer modified RNA segment, wherein a first terminal end of the longer modified RNA segment is complementary to the short terminal RNA segment; and
iii) a short linker nucleic acid having a first terminal end linked to the terminal end of the short terminal RNA segment that does not comprise the azide-modified sugar wherein the short linker nucleic acid is complementary to a second terminal end of the longer modified RNA segment that is not complementary to the short terminal RNA segment;
wherein the longer modified RNA segment is complementary to a specific transcript target; and
wherein the longer modified RNA segment is modified to be nuclease resistant.

122. The method according to claim 121, wherein the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA).

123. The method according to claim 121 or claim 122, wherein the azide-modified sugar is acetylated at 1, 2, 3, or 4 positions.

124. The method according to any one of claims 121 to 123, wherein the longer modified RNA segment comprises a phosphorothioate backbone, a phosphoramidate backbone, a morpholino backbone, a bridged nucleic acid backbone, a locked nucleic acid (LNA) backbone or a 2′ modification.

125. The method according to claim 124, wherein the 2′ modification is chosen from —O[(CH₂)ₙO]ₙCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are, independently, from 0 to about 10.

126. The method according to claim 125, wherein the 2′ modification is a 2′-O-methyl group.

127. The method according to any one of claims 111 to 126, further comprising contacting the cell with a substrate for a templated assembly reaction, wherein the substrate comprises:
i) a nucleic acid template; and
ii) an azide reactive molecule linked to the nucleic acid template at the 5′- or 3′-end of the nucleic acid template, wherein the azide reactive molecule is chemically reactable with the azide of the azide-modified sugar on the surface of the cell.

128. The method according to claim 127, wherein the nucleic acid template is chosen from a cancer-specific polynucleotide, a viral polynucleotide, a microbial-specific polynucleotide, a differentially expressed gene, and a disease-specific polynucleotide.

129. The method according to claim 127 or claim 128, further comprising performing a Template Assembly by Proximity-Enhanced Reactivity (TAPER) reaction using the nucleic acid template as a target nucleic acid sequence for the TAPER reaction.

130. The method according to claim 129, wherein the TAPER reaction comprises contacting the cell with a first haplomer and a second haplomer, wherein:
the first haplomer comprises:
a first polynucleotide that is complementary to a first region of the nucleic acid template;
a first effector partial moiety, wherein the first effector partial moiety is linked to the first polynucleotide; and
a first selectively-reactive moiety, wherein the first selectively-reactive moiety is linked to the first effector partial moiety;
the second haplomer comprises:
a second polynucleotide that is complementary to a second region of the nucleic acid template;
a second effector partial moiety, wherein the second effector partial moiety is linked to the second polynucleotide; and
a second selectively-reactive moiety, wherein the second selectively-reactive moiety is linked to the second effector partial moiety;
wherein:
the first selectively-reactive moiety and the second selectively-reactive moiety chemically react with each other when in sufficient proximity;
the first region of the nucleic acid template is in sufficient proximity to the second region of the nucleic acid template to allow the first selectively-reactive moiety and the second selectively-reactive moiety to chemically react with each other; and
the first effector partial moiety and the second effector partial moiety form an active effector agent when in sufficient proximity.

131. The method according to claim 130, wherein the first selectively-reactive moiety and the second selectively-reactive moiety are a chemically reactable pair of selectively-reactive moieties chosen from an azide, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, and a quadricyclane.

132. The method according to claim 131, wherein the first selectively-reactive moiety and the second selectively-reactive moiety are an azide-alkyne pair, an azide-phosphine pair, or a tetrazine-norbornene/trans-cyclooctene pair.

133. The method according to any one of claims 130 to 132, wherein the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

134. The method according to any one of claims 130 to 133, wherein the first effector partial moiety and the second effector partial moiety are fragments of an active effector agent chosen from a peptide, protein, or a therapeutic agent.

135. The method according to claim 134, wherein the peptide or protein is a toxin, a pro-apoptotic agent, or a ligand for an antibody or antibody fragment.

136. The method according to claim 127 or claim 128, further comprising performing a Ligand Directed TAPER (LD-TAPER) reaction using the nucleic acid template as a target nucleic acid sequence for the LD-TAPER reaction.

137. The method according to claim 136, wherein the LD-TAPER reaction comprises:
contacting the nucleic acid template with a first haplomer-ligand complex, wherein the first haplomer-ligand complex comprises a first haplomer, wherein the first haplomer comprises a first polynucleotide, and a first small molecule ligand linked to the 5′ or 3′ terminus of the first haplomer, wherein the first small molecule ligand comprises a first small molecule ligand partner binding site;
contacting the nucleic acid template with a second haplomer-ligand complex, wherein the second haplomer-ligand complex comprises a second haplomer, wherein the second haplomer comprises a second polynucleotide, and a second small molecule ligand linked to the 5′ or 3′ terminus of the second haplomer, wherein the second small molecule ligand comprises a second small molecule ligand partner binding site;
contacting the first haplomer-ligand complex with a first fusion protein, wherein the first fusion protein comprises a first fragment of a protein of interest fused to a first ligand binding domain for a small molecule ligand;
contacting the second haplomer-ligand complex with a second fusion protein, wherein the second fusion protein comprises a second fragment of the protein of interest fused to a second ligand binding domain for a small molecule ligand;
wherein the first ligand of the first haplomer-ligand complex is linked to the 5′ terminus of the first polynucleotide of the first haplomer-ligand complex;
wherein the second ligand of the second haplomer-ligand complex is linked to the 3′ terminus of the second polynucleotide of the second haplomer-ligand complex;
wherein the first polynucleotide of the first haplomer-ligand complex is substantially complementary to the nucleic acid template;
wherein the second polynucleotide of the second haplomer-ligand complex is substantially complementary to the nucleic acid template at a site in spatial proximity to the first polynucleotide of the first haplomer-ligand complex;
wherein the first small molecule ligand of the first haplomer-ligand complex and the first ligand binding domain of the first fusion protein can interact; and
wherein the second small molecule ligand of the second haplomer-ligand complex and the second ligand binding domain of the second fusion protein can interact;
thereby resulting in the folding or dimerization of the first fragment of the protein of interest of the first fusion protein with the second fragment of the protein of interest of the second fusion protein.

138. The method according to claim 137, wherein:
the first small molecule ligand is an FKBP binding compound, and the first ligand binding domain for the first small molecule ligand is an FKBP domain or a FRB domain; and
the second small molecule ligand is an FKBP binding compound, and the second ligand binding domain for the second small molecule ligand is an FKBP domain or a FRB domain.

139. The method according to claim 136, wherein the LD-TAPER reaction comprises:
contacting the nucleic acid template with a first haplomer-ligand complex, wherein the first haplomer-ligand complex comprises a first haplomer, wherein the first haplomer comprises a first polynucleotide, and a first ligand linked to the 5′ or 3′ terminus of the first haplomer, wherein the first ligand is a first interactive protein domain and comprises a first ligand partner binding site;
contacting the nucleic acid template with a second haplomer-ligand complex, wherein the second haplomer-ligand complex comprises a second haplomer, wherein the second haplomer comprises a second polynucleotide, and a second ligand linked to the 5′ or 3′ terminus of the second haplomer, wherein the second ligand is a second interactive protein domain and comprises a second ligand partner binding site;
contacting the first haplomer-ligand complex with a first fusion protein, wherein the first fusion protein comprises a first fragment of a protein of interest fused to a third interactive protein domain;
contacting the second haplomer-ligand complex with a second fusion protein, wherein the second fusion protein comprises a second fragment of the protein of interest fused to a fourth interactive protein domain;
wherein the first ligand of the first haplomer-ligand complex is linked to the 5′ terminus of the first polynucleotide of the first haplomer-ligand complex;
wherein the second ligand of the second haplomer-ligand complex is linked to the 3′ terminus of the second polynucleotide of the second haplomer-ligand complex;
wherein the first polynucleotide of the first haplomer-ligand complex is substantially complementary to the nucleic acid template;
wherein the second polynucleotide of the second haplomer-ligand complex is substantially complementary to the nucleic acid template at a site in spatial proximity to the first polynucleotide of the first haplomer-ligand complex;
wherein the first interactive protein domain of the first haplomer-ligand complex and the third interactive protein domain of the first fusion protein can interact; and
wherein the second interactive protein domain of the second haplomer-ligand complex and the fourth interactive protein domain of the second fusion protein can interact; and
thereby resulting in the folding or dimerization of the first fragment of the protein of interest of the first fusion protein with the second fragment of the protein of interest of the second fusion protein.

140. The method according to claim 139, wherein:
the first interactive protein domain and the third interactive protein domain are jun/fos, mad/max, myc/max, or NZ/CZ interacting zipper motifs; and
the second interactive protein domain and the fourth interactive protein domain are jun/fos, mad/max, myc/max, or NZ/CZ interacting zipper motifs.

141. The method according to claim 136, wherein the LD-TAPER reaction comprises:
contacting the nucleic acid template with a complex formed by the interaction of a first haplomer-ligand complex with a first fusion protein, wherein:
the first haplomer-ligand complex comprises a first haplomer, wherein the first haplomer comprises a first polynucleotide, and a first small molecule ligand linked to the 5′ or 3′ terminus of the first polynucleotide, wherein the first small molecule ligand comprises a first ligand partner binding site;
the first fusion protein comprises a first fragment of a protein of interest fused to a first ligand binding domain for the first small molecule ligand; and
the first small molecule ligand of the first haplomer-ligand complex interacts with the first ligand binding domain for the first small molecule ligand of the first fusion protein; and
contacting the nucleic acid template with a complex formed by the interaction of a second haplomer-ligand complex with a second fusion protein, wherein:
the second haplomer-ligand complex comprises a second haplomer, wherein the second haplomer comprises a second polynucleotide, and a second small molecule ligand linked to the 5′ or 3′ terminus of the second polynucleotide, wherein the second small molecule ligand comprises a second ligand partner binding site;
the second fusion protein comprises a second fragment of the protein of interest fused to a second ligand binding domain for the second small molecule ligand;
the second small molecule ligand of the second haplomer-ligand complex interacts with the second ligand binding domain for the second small molecule ligand of the second fusion protein;
thereby resulting in the folding or dimerization of the fragment of the protein of interest of the first fusion protein with the fragment of the protein of interest of the second fusion protein.

142. The method according to claim 141, wherein:
the first small molecule ligand is an FKBP binding compound, and the first ligand binding domain for the first small molecule ligand is an FKBP domain or a FRB domain; and
the second small molecule ligand is an FKBP binding compound, and the second ligand binding domain for the second small molecule ligand is an FKBP domain or a FRB domain.

143. The method according to claim 136, wherein the LD-TAPER reaction comprises:
contacting the nucleic acid template with a complex formed by the interaction of a first haplomer-ligand complex with a first fusion protein, wherein:
the first haplomer-ligand complex comprises a first haplomer, wherein the first haplomer comprises a first polynucleotide, and a first ligand linked to the 5′ or 3′ terminus of the first polynucleotide, wherein the first ligand is a first interactive protein domain and comprises a first ligand partner binding site;
the first fusion protein comprises a first fragment of a protein of interest fused to a third interactive protein domain;
the first interactive protein domain of the first haplomer-ligand complex and the third interactive protein domain of the first fusion protein can interact; and
contacting the nucleic acid template with a complex formed by the interaction of a second haplomer-ligand complex with a second fusion protein, wherein:
the second haplomer-ligand complex comprises a second haplomer, wherein the second haplomer comprises a second polynucleotide, and a second ligand linked to the 5′ or 3′ terminus of the second polynucleotide, wherein the second ligand is a second interactive protein domain and comprises a second ligand partner binding site;
the second fusion protein comprises a second fragment of a protein of interest fused to a fourth interactive protein domain;
the second interactive protein domain of the second haplomer-ligand complex and the fourth interactive protein domain of the second fusion protein can interact;
thereby resulting in the folding or dimerization of the first fragment of the protein of interest of the first fusion protein with the second fragment of the protein of interest of the second fusion protein.

144. The method according to claim 143, wherein:
the first interactive protein domain and the third interactive protein domain are jun/fos, mad/max, myc/max, or NZ/CZ interacting zipper motifs; and
the second interactive protein domain and the fourth interactive protein domain are jun/fos, mad/max, myc/max, or NZ/CZ interacting zipper motifs.

145. The method according to claim 136, wherein the LD-TAPER reaction comprises:
contacting the nucleic acid template with a bottle haplomer-ligand complex, wherein the bottle haplomer-ligand complex comprises:
a) a bottle haplomer, wherein the bottle haplomer comprises a first polynucleotide, wherein the first polynucleotide comprises:
i) a first stem portion comprising from about 10 to about 20 nucleotide bases;
ii) an anti-target loop portion comprising from about 16 to about 40 nucleotide bases and having a first end to which the first stem portion is linked, wherein the anti-target loop portion is substantially complementary to the nucleic acid template; and
iii) a second stem portion comprising from about 10 to about 20 nucleotide bases linked to a second end of the anti-target loop portion, wherein the first stem portion is substantially complementary to the second stem portion; and
b) a first small molecule ligand linked to the terminal end of either the first stem portion or the second stem portion, wherein the first small molecule ligand comprises a first small molecule ligand partner binding site;
wherein the Tₘ of the anti-target loop portion:nucleic acid template is greater than the Tₘ of the first stem portion: second stem portion;
contacting the nucleic acid template with a second haplomer-ligand complex, wherein the second haplomer-ligand complex comprises a second haplomer, wherein the second haplomer comprises a second polynucleotide, and a second small molecule ligand linked to the 5′ or 3′ terminus of the second polynucleotide, wherein the second small molecule ligand comprises a second small molecule ligand partner binding site; wherein the second haplomer-ligand complex comprises a nucleotide portion that is substantially complementary to the stem portion of the bottle haplomer-ligand complex that is linked to the first small molecule ligand of the bottle haplomer-ligand complex;
contacting the bottle haplomer-ligand complex with a first fusion protein, wherein the first fusion protein comprises a first fragment of a protein of interest fused to a first ligand binding domain for the first small molecule ligand, wherein the first small molecule ligand of the bottle haplomer-ligand complex and the first small molecule ligand binding domain of the first fusion protein can interact; and
contacting the second haplomer-ligand complex with a second fusion protein, wherein the second fusion protein comprises a second fragment of the protein of interest fused to a second small molecule ligand binding domain for the second small molecule ligand, wherein the second small molecule ligand of the second haplomer-ligand complex and the second small molecule ligand binding domain of the second fusion protein can interact;
thereby resulting in the folding or dimerization of the first fragment of the protein of interest of the first fusion protein with the second fragment of the protein of interest of the second fusion protein.

146. The method according to claim 145, wherein:
the first small molecule ligand is an FKBP binding compound, and the first ligand binding domain for the first small molecule ligand is an FKBP domain or a FRB domain; and
the second small molecule ligand is an FKBP binding compound, and the second ligand binding domain for the second small molecule ligand is an FKBP domain or a FRB domain.

147. The method according to claim 136, wherein the LD-TAPER reaction comprises:
contacting the nucleic acid template with a bottle haplomer-ligand complex, wherein the bottle haplomer-ligand complex comprises:
a) a bottle haplomer, wherein the bottle haplomer comprises a first polynucleotide, wherein the first polynucleotide comprises:
i) a first stem portion comprising from about 10 to about 20 nucleotide bases;
ii) an anti-target loop portion comprising from about 16 to about 40 nucleotide bases and having a first end to which the first stem portion is linked, wherein the anti-target loop portion is substantially complementary to the nucleic acid template; and
iii) a second stem portion comprising from about 10 to about 20 nucleotide bases linked to a second end of the anti-target loop portion, wherein the first stem portion is substantially complementary to the second stem portion; and
b) a first ligand linked to the terminal end of either the first stem portion or the second stem portion, wherein the first ligand is a first interactive protein domain and comprises a first ligand partner binding site;
wherein the Tₘ of the anti-target loop portion:nucleic acid template is greater than the Tₘ of the first stem portion: second stem portion;
contacting the nucleic acid template with a second haplomer-ligand complex, wherein the second haplomer-ligand complex comprises a second haplomer, wherein the second haplomer comprises a second polynucleotide, and a second ligand linked to the 5′ or 3′ terminus of the second polynucleotide, wherein the second ligand is a second interactive protein domain and comprises a second ligand partner binding site; wherein the second haplomer-ligand complex comprises a nucleotide portion that is substantially complementary to the stem portion of the bottle haplomer-ligand complex that is linked to the first ligand of the bottle haplomer-ligand complex;
contacting the bottle haplomer-ligand complex with a first fusion protein, wherein the first fusion protein comprises a first fragment of a protein of interest fused to a third interactive protein domain; wherein the first interactive protein domain of the bottle haplomer-ligand complex and the third interactive protein domain of the first fusion protein can interact; and
contacting the second haplomer-ligand complex with a second fusion protein, wherein the second fusion protein comprises a second fragment of the protein of interest fused to a fourth interactive protein domain; wherein the second interactive protein domain of the second haplomer-ligand complex and the fourth interactive protein domain of the second fusion protein can interact;
thereby resulting in the folding or dimerization of the first fragment of the protein of interest of the first fusion protein with the second fragment of the protein of interest of the second fusion protein.

148. The method according to claim 147, wherein:
the first interactive protein domain and the third interactive protein domain are jun/fos, mad/max, myc/max, or NZ/CZ interacting zipper motifs; and
the second interactive protein domain and the fourth interactive protein domain are jun/fos, mad/max, myc/max, or NZ/CZ interacting zipper motifs.

149. The method according to any one of claims 137 to 148, wherein the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

150. The method according to any one of claims 137 to 149, wherein the first fragment of the protein of interest and the second fragment of the protein of interest are fragments of an active effector agent chosen from a peptide, protein, or a therapeutic agent.

151. The method according to claim 150, wherein the peptide or protein is a toxin, a pro-apoptotic agent, or a ligand for an antibody or antibody fragment.

152. The method according to claim 127 or claim 128, further comprising performing a Split Protein TAPER (SP-TAPER) reaction using the nucleic acid template as a target nucleic acid sequence for the TAPER reaction.

153. The method according to claim 152, wherein the SP-TAPER reaction comprises:
contacting the cell with a first haplomer comprising a first polynucleotide linked to the C-terminus of an N-terminal protein fragment; and
contacting the cell with a second haplomer comprising a second polynucleotide linked to the N-terminus of a C-terminal protein fragment;
wherein:
the polynucleotide of one of the first or second haplomers is linked at its 5′ terminus to the protein fragment, and the other of the first and second haplomers is linked at its 3′ terminus to the protein fragment;
the N-terminal protein fragment and the C-terminal protein fragment are derived from a single active effector agent; and
wherein:
the first polynucleotide of the first haplomer is substantially complementary to the nucleic acid template, and the second polynucleotide of the second haplomer is substantially complementary to the nucleic acid template at a site in spatial proximity to the first polynucleotide of the first haplomer; or
the first polynucleotide of the first haplomer is substantially complementary to a portion of the nucleic acid template 5′ adjacent to a stem-loop structure, and the second polynucleotide of the second haplomer is substantially complementary to a portion of the nucleic acid template 3′ adjacent to the stem-loop structure; or
the first polynucleotide of the first haplomer is substantially complementary to a 5′ portion of a loop of a stem-loop structure of the nucleic acid template, and the second polynucleotide of the second haplomer is substantially complementary to a 3′ portion of the loop of the stem-loop structure of the nucleic acid template;
thereby resulting in the assembly of the protein from the N-terminal protein fragment and the C-terminal protein fragment.

154. The method according to claim 153, wherein:
the N-terminal fragment comprises the amino acid sequence of APIVTCRKLDGRE KPFKVDVATAQAQARKAGLTTGKSGDPHRYFAGDHIRWGVNNCDKADAILWEYPI YWVGKNAEWAKDVKTSQQKG (SEQ ID NO:34), and the C-terminal fragment comprises the amino acid sequence of GPTPIRVVYANSRGAVQYCGVMTHSKVDKNNQ GKEFFEKCD (SEQ ID NO:35);
the N-terminal fragment comprises the amino acid sequence of APIVTCRPKLDG (SEQ ID NO:36), and the C-terminal fragment comprises the amino acid sequence of REKP FKVDVATAQAQARKAGLTTGKSGDPHRYFAGDHIRWGVNNCDKADAILWEYPIYW VGKNAEWAKDVKTSQQKGGPTPIRVVYANSRGAVQYCGVMTHSKVDKNNQGKEF FEKCD (SEQ ID NO:37);
the N-terminal fragment comprises the amino acid sequence of APIVTCRPKLDGR EKPFKVDVATAQAQARKAGLTTGK (SEQ ID NO:38), and the C-terminal fragment comprises the amino acid sequence of SGDPHRYFAGDHIRWGVNNCDKADAILWEYPIY WVGKNAEWAKDVKTSQQKGGPTPIRVVYANSRGAVQYCGVMTHSKVDKNNQGK EFFEKCD (SEQ ID NO:39);
the N-terminal fragment comprises the amino acid sequence of APIVTCRPKLDGR EKPFKVDVATAQAQARKAGLTTGKSGDPHRYFAGDHIRWGVNNCDKAD (SEQ ID NO:40), and the C-terminal fragment comprises the amino acid sequence of AILWEYPIYW VGKNAEWAKDVKTSQQKGGPTPIRVVYANSRGAVQYCGVMTHSKVDKNNQGKEF FEKCD (SEQ ID NO:41);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVG (SEQ ID NO:42), and the C-terminal fragment comprises the amino acid sequence of KNAEWAKDVKTSQQKGGPTPIRVVYANSRGAVQYCGVMTHSKVDKNNQGKEFF EKCD (SEQ ID NO:43);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVGKNAEWAKD (SEQ ID NO:44), and the C-terminal fragment comprises the amino acid sequence of VKTSQQKGGPTPIRVVYANSRGAVQYCGVMTHSKVDKNNQGKEFF EKCD (SEQ ID NO:45);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVGKNAEWAKDVKTSQ (SEQ ID NO:46), and the C-terminal fragment comprises the amino acid sequence of QKGGPTPIRVVYANSRGAVQYCGVMTHSKVDKNNQGKEFFE KCD (SEQ ID NO:47);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVGKNAEWAKDVKTSQQKGGPTPIRVVYANSRG (SEQ ID NO:48), and the C-terminal fragment comprises the amino acid sequence of AVQYCGVMTHSKVDKNNQGK EFFEKCD (SEQ ID NO:49);
the N-terminal fragment comprises the amino acid sequence of APIVTCRPKLDGR EKPFKVDVATAQAQARKAGLTTGKSGDPHRYFAGDHIRWGVNNCDKADAILWEYP IYWVGKNAEWAKDVKTSQQKGGPTPIRVVYANSRGAVQYCGVMTHSKVDKN (SEQ ID NO:50), and the C-terminal fragment comprises the amino acid sequence of NQGK EFFEKCD (SEQ ID NO:51); or
the N-terminal fragment comprises the amino acid sequence of APIVTCRPKLDGR EKPFKVDVATAQAQARKAGLT; (SEQ ID NO:52), and the C-terminal fragment comprises the amino acid sequence of TGKSGDPHRYFAGDHIRWGVNNCDKADAILW EYPIYWVGKNAEWAKDVKTSQQKGGPTPIRVVYANSRGAVQYCGVMTHSKVDKN NQGKEFFEKCD (SEQ ID NO:53).

155. The method according to claim 152, wherein the SP-TAPER reaction comprises: comprising:
a) contacting the nucleic acid template with a bottle: haplomer, wherein the bottle haplomer comprises a first polynucleotide comprising:
i) a first 3′ stem portion comprising from about 10 to about 20 nucleotide bases;
ii) an anti-target loop portion comprising from about 16 to about 40 nucleotide bases linked to the first 3′ stem portion, wherein the anti-target loop portion is substantially complementary to the nucleic acid template; and
iii) a second 5′ stem portion comprising from about 10 to about 20 nucleotide bases linked to the anti-target loop portion, wherein the first 3′ stem portion is substantially complementary to the second 5′ stem portion;
wherein the 5′ terminus of the first polynucleotide comprises an —SH moiety and is linked to the C-terminus of an N-terminal protein fragment, wherein the C-terminus comprises a cysteine or a selenocysteine; and
b) contacting the bottle haplomer with a second haplomer comprising a second polynucleotide linked to the N-terminus of a C-terminal protein fragment, wherein the second polynucleotide of the second haplomer is substantially complementary to the second 5′ stem portion of the first polynucleotide of the bottle haplomer;
wherein:
the N-terminal protein fragment and the C-terminal protein fragment are derived from a single active effector agent;
the Tₘ of the anti-target loop portion:nucleic acid template is greater than the Tₘ of the first stem portion:second stem portion; and
the Tₘ of the duplex formed by the second haplomer and the second stem portion of the bottle haplomer subtracted from the Tₘ of the first stem portion:second stem portion is from about 0° C. to about 20° C.;
thereby resulting in the assembly of the protein from the N-terminal protein fragment and the C-terminal protein fragment.

156. The method according to claim 155, wherein:
the Tₘ of the first stem portion:second stem portion subtracted from the Tₘ of the anti-target loop portion:nucleic acid template is from about 10° C. to about 40° C.;
the Tₘ of the first stem portion:second stem portion is from about 40° C. to about 50° C.;
the Tₘ of the anti-target loop portion:nucleic acid template is from about 60° C. to about 80° C.; and/or
the Tₘ of the first stem portion:second stem portion subtracted from the Tₘ of the anti-target loop portion:nucleic acid template is from about 10° C. to about 20° C.

157. The method according to claim 155 or claim 156, wherein:
the first stem portion comprises from about 12 to about 18 nucleotide bases;
the anti-target loop portion comprises from about 18 to about 35 nucleotide bases; and/or
the second stem portion comprises from about 12 to about 18 nucleotide bases.

158. The method according to any one of claims 153 to 157, wherein the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

159. The method according to any one of claims 153 to 158, wherein the N-terminal protein fragment and the C-terminal protein fragment are fragments of an active effector agent chosen from a peptide, protein, or a therapeutic agent.

160. The method according to claim 159, wherein the peptide or protein is a toxin, a pro-apoptotic agent, or a ligand for an antibody or antibody fragment.

161. The method according to claim 127 or claim 128, further comprising performing a Locked TAPER reaction using the nucleic acid template as a target nucleic acid sequence for the TAPER reaction.

162. The method according to claim 161, wherein the Locked TAPER reaction comprises:
contacting the nucleic acid template with a first haplomer, wherein the first haplomer comprises:
a) a first polynucleotide comprising:
i) a first stem portion comprising from about 10 to about 20 nucleotide bases;
ii) an anti-target loop portion comprising from about 16 to about 40 nucleotide bases and having a first end to which the first stem portion is linked, wherein the anti-target loop portion is substantially complementary to the nucleic acid template; and
iii) a second stem portion comprising from about 10 to about 20 nucleotide bases linked to a second end of the anti-target loop portion, wherein the first stem portion is substantially complementary to the second stem portion; and
b) a first effector partial moiety linked to either the first stem portion or the second stem portion;
wherein the Tₘ of the anti-target loop portion:nucleic acid template is greater than the Tₘ of the first stem portion: second stem portion; and
contacting the first haplomer with a second haplomer, wherein the second haplomer comprises:
a) a second polynucleotide comprising a nucleotide portion that is substantially complementary to the stem portion of the first polynucleotide that is linked to the first effector partial moiety; and
b) a second effector partial moiety linked to the second polynucleotide, wherein the second effector partial moiety can chemically interact with the first effector partial moiety of the first haplomer;
wherein the Tₘ of the second polynucleotide:first or second stem portion linked to the first effector partial moiety is less than or equal to the Tₘ of the first stem portion:second stem portion;
wherein the first effector partial moiety and the second effector partial moiety form an active effector agent when in sufficient proximity.

163. The method according to claim 162, wherein
the Tₘ of the first stem portion:second stem portion subtracted from the Tₘ of the anti-target loop portion:nucleic acid template is from about 10° C. to about 40° C.; and/or
the Tₘ of the first stem portion:second stem portion is from about 40° C. to about 50° C.; and/or
the Tₘ of the anti-target loop portion:nucleic acid template is from about 60° C. to about 80° C.; and/or
the Tₘ of the first stem portion:second stem portion subtracted from the Tₘ of the anti-target loop portion:nucleic acid template is from about 10° C. to about 20° C.

164. The method according to claim 162 or claim 163, wherein:
the first stem portion comprises from about 12 to about 18 nucleotide bases; and/or
the anti-target loop portion comprises from about 18 to about 35 nucleotide bases; and/or
the second stem portion comprises from about 12 to about 18 nucleotide bases.

165. The method according to any one of claims 162 to 164, wherein the first polynucleotide and the second polynucleotide comprise DNA nucleotides, RNA nucleotides, phosphorothioate-modified nucleotides, 2′-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, and other nucleic acid analogues capable of base-pair formation, or any combination thereof.

166. The method according to any one of claims 162 to 165, wherein the first effector partial moiety and the second effector partial moiety are fragments of an active effector agent chosen from a peptide, protein, or a therapeutic agent.

167. The method according to claim 166, wherein the peptide or protein is a toxin, a pro-apoptotic agent, or a ligand for an antibody or antibody fragment.

168. The method according to any one of claims 162 to 167, wherein the first effector partial moiety and the second effector partial moiety each further comprise a selectively-reactive moiety.

169. The method according to claim 168, wherein the first selectively-reactive moiety and the second selectively-reactive moiety are a chemically reactable pair of selectively-reactive moieties chosen from an azide, a cyclooctyne, a nitrone, a norbornene, an oxanorbornadiene, a phosphine, a dialkyl phosphine, a trialkyl phosphine, a phosphinothiol, a phosphinophenol, a cyclooctene, a nitrile oxide, a thioester, a tetrazine, an isonitrile, a tetrazole, and a quadricyclane.

170. The method according to claim 169, wherein the first selectively-reactive moiety and the second selectively-reactive moiety are an azide-alkyne pair, an azide-phosphine pair, or a tetrazine-norbornene/trans-cyclooctene pair.

171. The method according to any one of claims 162 to 170, wherein the anti-target loop portion further comprises an internal hinge region, wherein the hinge region comprises one or more nucleotides that are not complementary to the nucleic acid template.

172. The method according to claim 171, wherein the hinge region comprises from about 1 nucleotide to about 6 nucleotides.

173. The method according to any one of claims 162 to 172, wherein:
the first polynucleotide comprises the nucleotide sequence 5′-ACTCGAGACGTCT CCTTGTCTTTGCTTTTCTTCAGGACACAGTGGCGAGACGTCTCGAGT-3′ (SEQ ID NO:7), and the second polynucleotide comprises the nucleotide sequence 5′-AGCTCTCGA GT-3′ (SEQ ID NO:9); or
the first polynucleotide comprises the nucleotide sequence 5′-ACTCGAGACGTCT CCTTCCTGCCCCTCCTCCTGCTCCGAGACGTCTCGAGT-3′ (SEQ ID NO:8), and the second polynucleotide comprises the nucleotide sequence 5′-GACGTCTCGAGT-3′ (SEQ ID NO:10).


SEQUENCE LISTING (84 sequences)

SEQ ID NO: 1 - protein, 14 aa, Artificial Sequence
  Description of Artificial Sequence Synthetic peptide
KLAKLAKKLAKLAK

SEQ ID NO: 2 - protein, 47 aa, Artificial Sequence
  Description of Artificial Sequence Synthetic polypeptide
CSGGASLERIARLEEKVKTLKAQNSELASTANMLREQVAQLKQKGAP

SEQ ID NO: 3 - protein, 49 aa, Artificial Sequence
  Description of Artificial Sequence Synthetic polypeptide
SGASLERIARLEEKVKTLKAQNSELASTANMLREQVAQLKQKGAPSGGC

SEQ ID NO: 4 - protein, 41 aa, Artificial Sequence
  Description of Artificial Sequence Synthetic polypeptide
ASRELTDTLQAETDQLEDEKSALQTEIANLLKEKEKLEGAP

SEQ ID NO: 5 - protein, 47 aa, Artificial Sequence
  Description of Artificial Sequence Synthetic polypeptide
CSGGASLERIARLEEKVKSFKAQNSENASTANMLREQVAQLKQKGAP

SEQ ID NO: 6 - protein, 49 aa, Artificial Sequence
  Description of Artificial Sequence Synthetic polypeptide
SGASLERIARLEEKVKSFKAQNSENASTANMLREQVAQLKQKGAPSGGC

SEQ ID NO: 7 - DNA, 60 bp, Artificial Sequence
  Description of Artificial Sequence Synthetic oligonucleotide
actcgagacgtctccttgtctttgcttttcttcaggacacagtggcgagacgtctcgagt

SEQ ID NO: 8 - DNA, 51 bp, Artificial Sequence
  Description of Artificial Sequence Synthetic oligonucleotide
actcgagacgtctccttcctgcccctcctcctgctccgagacgtctcgagt

SEQ ID NO: 9 - DNA, 11 bp, Artificial Sequence
  Description of Artificial Sequence Synthetic oligonucleotide
agctctcgagt

SEQ ID NO: 10 - DNA, 12 bp, Artificial Sequence
  Description of Artificial Sequence Synthetic oligonucleotide
gacgtctcgagt

SEQ ID NO: 11 - protein, 29 aa, Artificial Sequence
  Description of Artificial Sequence Synthetic peptide
ALKKELQANKKELAQLKWELQALKKELAQ

SEQ ID NO: 12 - protein, 30 aa, Artificial Sequence
  Description of Artificial Sequence Synthetic polypeptide
EQLEKKLQALEKKLAQLEWKNQALEKKLAQ

SEQ ID NO: 13 - protein, 46 aa, Artificial Sequence
  Description of Artificial Sequence Synthetic polypeptide
CSGASLERIARLEEKVKSFKAQNSENASTANMLREQVAQLKQKGAP

SEQ ID NO: 14 - protein, 43 aa, Artificial Sequence
  Description of Artificial Sequence Synthetic polypeptide
SGASRELTDTLQAETDQLEDEKSALQTEIANLLKEKEKLEGAP

SEQ ID NO: 15 - protein, 97 aa, Artificial Sequence
  Description of Artificial Sequence Synthetic polypeptide
GVQVETISPGDGRTFPKRGQTCVVHYTGMLEDGKKVDSSRDRNKPFKFMLGKQEVIRGWEEGVAQMCPDYAYGATGHPGIIPPHATLVFDVELLKLE

SEQ ID NO: 16 - protein, 108 aa, Artificial Sequence
  Description of Artificial Sequence Synthetic polypeptide
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 17 - protein, 10 aa, Artificial Sequence
  Description of Artificial Sequence Synthetic peptide
AGSSAAGSGS

SEQ ID NO: 18 - protein, 18 aa, Artificial Sequence
  Description of Artificial Sequence Synthetic peptide
GGSGGGSGGGSGGGSGGG

SEQ ID NO: 19 - protein, 22 aa, Artificial Sequence
  Description of Artificial Sequence Synthetic peptide
GGSGGGSGGGSGGGSGGGSGGG

SEQ ID NO: 20 - protein, 26 aa, Artificial Sequence
  Description of Artificial Sequence Synthetic peptide
GGSGGGSGGGSGGGSGGGSGGGSGGG

SEQ ID NO: 21 - protein, 15 aa, Artificial Sequence
  Description of Artificial Sequence Synthetic peptide
SGGGGSGGGGSGGGG

SEQ ID NO: 22 - protein, 20 aa, Artificial Sequence
  Description of Artificial Sequence Synthetic peptide
SGGGGSGGGGSGGGGSGGGG

SEQ ID NO: 23 - protein, 25 aa, Artificial Sequence
  Description of Artificial Sequence Synthetic peptide
SGGGGSGGGGSGGGGSGGGGSGGGG

SEQ ID NO: 24 - protein, 5 aa, Artificial Sequence
  Description of Artificial Sequence Synthetic peptide
SGGGS

SEQ ID NO: 25 - protein, 4 aa, Artificial Sequence
  Description of Artificial Sequence Synthetic peptide
SGSG

SEQ ID NO: 26 - protein, 6 aa, Artificial Sequence
  Description of Artificial Sequence Synthetic peptide
SGGGGS

SEQ ID NO: 27 - protein, 5 aa, Artificial Sequence
  Description of Artificial Sequence Synthetic peptide
SGSGG

SEQ ID NO: 28 - protein, 156 aa, Unknown
  Description of Unknown superfolder GFP sequence
MRKGELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQ

SEQ ID NO: 29 - protein, 81 aa, Unknown
  Description of Unknown superfolder GFP sequence
KNGIKANFKIRHNVEDGSVQLADHYQQNTPIGDGPVLLPDNHYLSTQSVLSKDPNEKRDHMVLLEFVTAAGITHGMDELYK

SEQ ID NO: 30 - protein, 229 aa, Renilla reniformis
MASKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGG

SEQ ID NO: 31 - protein, 82 aa, Renilla reniformis
KPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 32 - protein, 93 aa, Gaussia princeps
MKPTENNEDFNIVAVASNFATTDLDADRGKLPGKKLPLEVLKEMEANARKAGCTRGCLICLSHIKCTPKMKKFIPGRCHTYEGDKESAQGGIG

SEQ ID NO: 33 - protein, 76 aa, Gaussia princeps
EAIVDIPEIPGFKDLEPMEQFIAQVDLCVDCTTGCLKGLANVQCSDLLKKWLPQRCATFASKIQGQVDKIKGAGGD

SEQ ID NO: 34 - protein, 88 aa, Artificial Sequence
  Description of Artificial Sequence Synthetic polypeptide
APIVTCRKLDGREKPFKVDVATAQAQARKAGLTTGKSGDPHRYFAGDHIRWGVNNCDKADAILWEYPIYWVGKNAEWAKDVKTSQQKG

SEQ ID NO: 35 - protein, 41 aa, Artificial Sequence
  Description of Artificial Sequence Synthetic polypeptide
GPTPIRVVYANSRGAVQYCGVMTHSKVDKNNQGKEFFEKCD

SEQ ID NO: 36 - protein, 12 aa, Artificial Sequence
  Description of Artificial Sequence Synthetic peptide
APIVTCRPKLDG

SEQ ID NO: 37 - protein, 118 aa, Artificial Sequence
  Description of Artificial Sequence Synthetic polypeptide
REKPFKVDVATAQAQARKAGLTTGKSGDPHRYFAGDHIRWGVNNCDKADAILWEYPIYWVGKNAEWAKDVKTSQQKGGPTPIRVVYANSRGAVQYCGVMTHSKVDKNNQGKEFFEKCD

SEQ ID NO: 38 - protein, 37 aa, Artificial Sequence
  Description of Artificial Sequence Synthetic polypeptide
APIVTCRPKLDGREKPFKVDVATAQAQARKAGLTTGK

SEQ ID NO: 39 - protein, 93 aa, Artificial Sequence
  Description of Artificial Sequence Synthetic polypeptide
SGDPHRYFAGDHIRWGVNNCDKADAILWEYPIYWVGKNAEWAKDVKTSQQKGGPTPIRVVYANSRGAVQYCGVMTHSKVDKNNQGKEFFEKCD

SEQ ID NO: 40 - protein, 61 aa, Artificial Sequence
  Description of Artificial Sequence Synthetic polypeptide
APIVTCRPKLDGREKPFKVDVATAQAQARKAGLTTGKSGDPHRYFAGDHIRWGVNNCDKAD

SEQ ID NO: 41 - protein, 69 aa, Artificial Sequence
  Description of Artificial Sequence Synthetic polypeptide
AILWEYPIYWVGKNAEWAKDVKTSQQKGGPTPIRVVYANSRGAVQYCGVMTHSKVDKNNQGKEFFEKCD

SEQ ID NO: 42 - protein, 73 aa, Artificial Sequence
  Description of Artificial Sequence Synthetic polypeptide
APIVTCRPKLDGREKPFKVDVATAQAQARKAGLTTGKSGDPHRYFAGDHIRWGVNNCDKADAILWEYPIYWVG

SEQ ID NO: 43 - protein, 57 aa, Artificial Sequence
  Description of Artificial Sequence Synthetic polypeptide
KNAEWAKDVKTSQQKGGPTPIRVVYANSRGAVQYCGVMTHSKVDKNNQGKEFFEKCD

SEQ ID NO: 44 - protein, 81 aa, Artificial Sequence
  Description of Artificial Sequence Synthetic polypeptide
APIVTCRPKLDGREKPFKVDVATAQAQARKAGLTTGKSGDPHRYFAGDHIRWGVNNCDKADAILWEYPIYWVGKNAEWAKD

SEQ ID NO: 45 - protein, 49 aa, Artificial Sequence
  Description of Artificial Sequence Synthetic polypeptide
VKTSQQKGGPTPIRVVYANSRGAVQYCGVMTHSKVDKNNQGKEFFEKCD

SEQ ID NO: 46 - protein, 86 aa, Artificial Sequence
  Description of Artificial Sequence Synthetic polypeptide
APIVTCRPKLDGREKPFKVDVATAQAQARKAGLTTGKSGDPHRYFAGDHIRWGVNNCDKADAILWEYPIYWVGKNAEWAKDVKTSQ

SEQ ID NO: 47 - protein, 44 aa, Artificial Sequence
  Description of Artificial Sequence Synthetic polypeptide
QKGGPTPIRVVYANSRGAVQYCGVMTHSKVDKNNQGKEFFEKCD

SEQ ID NO: 48 - protein, 103 aa, Artificial Sequence
  Description of Artificial Sequence Synthetic polypeptide
APIVTCRPKLDGREKPFKVDVATAQAQARKAGLTTGKSGDPHRYFAGDHIRWGVNNCDKADAILWEYPIYWVGKNAEWAKDVKTSQQKGGPTPIRVVYANSRG

SEQ ID NO: 49 - protein, 27 aa, Artificial Sequence
  Description of Artificial Sequence Synthetic peptide
AVQYCGVMTHSKVDKNNQGKEFFEKCD

SEQ ID NO: 50 - protein, 119 aa, Artificial Sequence
  Description of Artificial Sequence Synthetic polypeptide
APIVTCRPKLDGREKPFKVDVATAQAQARKAGLTTGKSGDPHRYFAGDHIRWGVNNCDKADAILWEYPIYWVGKNAEWAKDVKTSQQKGGPTPIRVVYANSRGAVQYCGVMTHSKVDKN

SEQ ID NO: 51 - protein, 11 aa, Artificial Sequence
  Description of Artificial Sequence Synthetic peptide
NQGKEFFEKCD

SEQ ID NO: 52 - protein, 34 aa, Artificial Sequence
  Description of Artificial Sequence Synthetic polypeptide
APIVTCRPKLDGREKPFKVDVATAQAQARKAGLT

SEQ ID NO: 53 - protein, 96 aa, Artificial Sequence
  Description of Artificial Sequence Synthetic polypeptide
TGKSGDPHRYFAGDHIRWGVNNCDKADAILWEYPIYWVGKNAEWAKDVKTSQQKGGPTPIRVVYANSRGAVQYCGVMTHSKVDKNNQGKEFFEKCD

SEQ ID NO: 54 - DNA, 34 bp, Artificial Sequence
  Description of Artificial Sequence Synthetic oligonucleotide
aagccactgtgtcctgaagaaaagcaaagacatc

SEQ ID NO: 55 - protein, 22 aa, Artificial Sequence
  Description of Artificial Sequence Synthetic peptide
  MOD_RES (7)..(7): D-Phenylalanine
SYSMEHFRWGKPVGGGSSGGGC

SEQ ID NO: 56 - protein, 22 aa, Artificial Sequence
  Description of Artificial Sequence Synthetic peptide
  MOD_RES (4)..(4): Norleucine
  MOD_RES (7)..(7): D-Phenylalanine
SYSXEHFRWGKPVGGGSSGGGC

SEQ ID NO: 57 - protein, 23 aa, Artificial Sequence
  Description of Artificial Sequence Synthetic peptide
  MOD_RES (17)..(17): D-Phenylalanine
CSGGGSSGGGSYSMEHFRWGKPV

SEQ ID NO: 58 - protein, 23 aa, Artificial Sequence
  Description of Artificial Sequence Synthetic peptide
  MOD_RES (14)..(14): Norleucine
  MOD_RES (17)..(17): D-Phenylalanine
CSGGGSSGGGSYSXEHFRWGKPV

SEQ ID NO: 59 - protein, 90 aa, Artificial Sequence
  Description of Artificial Sequence Synthetic polypeptide
APIVTCRPKLDGREKPFKVDVATAQAQARKAGLTTGKSGDPHRYFAGDHIRWGVNNCDKADAILWEYPIYWVGKNAEWAKDVKTSQQKGC

SEQ ID NO: 60 - protein, 42 aa, Artificial Sequence
  Description of Artificial Sequence Synthetic polypeptide
CGPTPIRVVYANSRGAVQYCGVMTHSKVDKNNQGKEFFEKCD

SEQ ID NO: 61 - protein, 4 aa, Unknown
  Description of Unknown Factor Xa protease cleavage site sequence
IEGR

SEQ ID NO: 62 - protein, 9 aa, Artificial Sequence
  Description of Artificial Sequence Synthetic peptide
GSGSGHHHH

SEQ ID NO: 63 - protein, 34 aa, Saccharomyces cerevisiae
MQIFVKTLTGKTITLEVESSDTIDNVKSKIQDKE

SEQ ID NO: 64 - protein, 42 aa, Saccharomyces cerevisiae
GIPPDQQRLIFAGKQLEDGRTLSDYNIQKESTLHLVLRLRGG

SEQ ID NO: 65 - protein, 157 aa, Unknown
  Description of Unknown superfolder GFP sequence
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQ

SEQ ID NO: 66 - RNA, 40 nt, Artificial Sequence
  Description of Artificial Sequence Synthetic oligonucleotide
uuuuuccuguguccugaagaaagcaaagacaucuggacaa

SEQ ID NO: 67 - DNA, 58 bp, Artificial Sequence
  Description of Artificial Sequence Synthetic oligonucleotide
ctttcttcaggacacagaggtagcgtctcaggtgggaagttcggattgccagtaggtg

SEQ ID NO: 68 - DNA, 59 bp, Artificial Sequence
  Description of Artificial Sequence Synthetic oligonucleotide
cactactgtgctcctctcactgcactcttgctcactccacttttgtccagatgtctttg

SEQ ID NO: 69 - DNA, 117 bp, Artificial Sequence
  Description of Artificial Sequence Synthetic polynucleotide
cactactgtgctcctctcactgcactcttgctcactccacttttgtccagatgtctttgctttcttcaggacacagaggtagcgtctcaggtgggaagttcggattgccagtaggtg

SEQ ID NO: 70 - DNA, 21 bp, Artificial Sequence
  Description of Artificial Sequence Synthetic primer
cacctactggcaatccgaact

SEQ ID NO: 71 - DNA, 21 bp, Artificial Sequence
  Description of Artificial Sequence Synthetic primer
cactactgtgctcctctcact

SEQ ID NO: 72 - DNA, 31 bp, Artificial Sequence
  Description of Artificial Sequence Synthetic probe
tccagatgtctttgcttttcttcaggacaca

SEQ ID NO: 73 - DNA, 32 bp, Artificial Sequence
  Description of Artificial Sequence Synthetic oligonucleotide
cttacctaatgctgtttatatcatcgttctgt

SEQ ID NO: 74 - DNA, 58 bp, Artificial Sequence; identical to SEQ ID NO: 67
  Description of Artificial Sequence Synthetic oligonucleotide

SEQ ID NO: 75 - DNA, 59 bp, Artificial Sequence; identical to SEQ ID NO: 68
  Description of Artificial Sequence Synthetic oligonucleotide

SEQ ID NO: 76 - DNA, 20 bp, Artificial Sequence
  Description of Artificial Sequence Synthetic primer
tcccacctgagacgctacct

SEQ ID NO: 77 - DNA, 21 bp, Artificial Sequence
  Description of Artificial Sequence Synthetic primer
gcactcttgctcactccactt

SEQ ID NO: 78 - protein, 48 aa, Artificial Sequence
  Description of Artificial Sequence Synthetic polypeptide
GASLERIARLEEKVKTLKAQNSELASTANMLREQVAQLKQKGAPSGGC

SEQ ID NO: 79 - protein, 40 aa, Artificial Sequence
  Description of Artificial Sequence Synthetic polypeptide
ASRETDTLQAETDQLEDEKSALQTEIANLLKEKEKLEGAP

SEQ ID NO: 80 - protein, 157 aa, Unknown
  Description of Unknown superfolder GFP sequence
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQ

SEQ ID NO: 81 - protein, 83 aa, Renilla reniformis
KPDVVQIVRNYNAYLRASDDLPKMFIESDPGFFSNAIVEGAKKFPNTEFVKVKGLHFSQEDAPDEMGKYIKSFVERVLKNEQZ

SEQ ID NO: 82 - protein, 6 aa, Artificial Sequence
  Description of Artificial Sequence Synthetic peptide
  MOD_RES (6)..(6): D-Methionine
WKYMVM

SEQ ID NO: 83 - protein, 6 aa, Artificial Sequence
  Description of Artificial Sequence Synthetic 6xHis tag
HHHHHH

SEQ ID NO: 84 - protein, 4 aa, Artificial Sequence
  Description of Artificial Sequence Synthetic 4xHis tag
HHHH